(19) United States
(12) Patent Application Publication
(10) Pub. No.: US 2025/0222125 A1
(43) Pub. Date: Jul. 10, 2025

(54) Anti-FGFR2b Antibodies, Conjugates and Methods of Use

(71) Applicant: BeiGene, Ltd., Grand Cayman (KY)

(72) Inventors: Charng-Sheng TSAI, Beijing (CN); Mei-Hsuan TSAI, Shanghai (CN); Xiaodong WEI, Shanghai (CN); Mengran QIAN, Shanghai (CN); Qun WANG, Shanghai (CN); Ruyue JI, Shanghai (CN); Jie PAN, Shanghai (CN); Yibin XU, Shanghai (CN); Maomao HE, Shanghai (CN); Zewei WANG, Shanghai (CN); Wei LUO, Shanghai (CN); Bing LI, Shanghai (CN); Liming WU, Shanghai (CN)

(73) Assignee: BeiGene, Ltd., Grand Cayman (KY)

(21) Appl. No.: 19/008,844

(22) Filed: Jan. 3, 2025

(30) Foreign Application Priority Data
Jan. 5, 2024 (WO) PCT/CN24/70825

(51) Int. Cl. A61K 47/68; A61P 35/00; C07K 16/28

(52) U.S. Cl. CPC A61K 47/68037; A61K 47/6849; A61K 47/6889; A61P 35/00; C07K 16/2803; C07K 16/2863; C07K 2317/41; C07K 2317/52; C07K 2317/565

(57) ABSTRACT

Anti-FGFR2b antibodies and antigen-binding fragments thereof are provided herein, as are immunoconjugates comprising the same, and compositions comprising the antibodies and immunoconjugates. Also provided are methods of making and using the antibodies and immunoconjugates, such as to treat an FGFR2b-related disease or disorder. The antibodies of the disclosure bind to human FGFR2b and can block the interaction between the KGF ligand and FGFR2b.

### CROSS-REFERENCE TO RELATED APPLICATION

This application claims the benefit of priority of PCT Application No. PCT/CN2024/070825, filed Jan. 5, 2024, entitled “Anti-FGFR2b Antibodies, Conjugates and Methods of Use,” which is hereby incorporated by reference in its entirety.

### REFERENCE TO ELECTRONIC SEQUENCE LISTING

The application contains a Sequence Listing which has been submitted electronically in .XML format and is hereby incorporated by reference in its entirety. Said .XML copy, created on Dec. 17, 2024, is named “01368-0001-00US.xml” and is 116,698 bytes in size. The sequence listing contained in this .XML file is part of the specification and is hereby incorporated by reference herein in its entirety.

### FIELD

The present disclosure relates to anti-FGFR2b antibodies, antigen-binding fragments thereof, immunoconjugates comprising the antibodies or antigen-binding fragments thereof, compositions comprising the antibodies, antigen-binding fragments thereof, or immunoconjugates, and methods for treating disorders responsive to FGFR2b antagonism.

### BACKGROUND

Fibroblast growth factor 7 (FGF-7), also known as keratinocyte growth factor (KGF), is an epithelial cell mitogen that acts exclusively through a subset of FGF transmembrane tyrosine kinase receptor isoforms (the FGFR2b isoforms). The KGF receptor, FGFR2b, is expressed primarily on the surface of epithelial cells. KGF binds FGFR2b with high selectivity and specificity.

FGFR2b signaling drives downstream pathways, including the mitogen-activated protein kinase (MAPK) and AKT pathways, which are crucial for cell proliferation, differentiation, survival and migration. Overexpression of FGFR2b RNA has been detected in a wide range of tumor tissues, including colon, prostate, and stomach. J. Nat. Canc. Inst., Vol. 98, Issues 1-12, Part 1 (2006). Thus, FGFR2b is a clinically validated target with high prevalence in multiple tumor types. Preclinical and clinical studies have demonstrated that FGFR-amplified tumors are sensitive to FGFR inhibition and therefore susceptible to therapeutic targeting. Treatment of FGFR2 positive tumors with anti-FGFR2 therapies such as Bemarituzumab (aka HGS1036, HGS 1036, FP-1039, FPA144, GSK3052230) leads to improvements in early survival and advanced disease. Gemo et al., Cancer Res (2014) 74 (19_Supplement):5446. However, Bemarituzumab only benefits a subset of patients with relatively higher FGFR2b over-expression. And use of Bemarituzumab has resulted in an observed treatment discontinuation rate of 26% caused by corneal toxicity. Wainberg, Zev A., et al. NCT03694522 (2021): 160-160.

Various FGFR2b antibodies have been conjugated to cytotoxic payloads, including thorium-227 (Aprutumab, aka BAY1179470, BAY2304058) and auristatin (Aprutumab ixadotin, aka BAY1187982, BAY1179470 ADC). Aprutumab-TTC (anti-FGFR2 antibody, a chelator moiety covalently conjugated to the antibody, and the alpha particle-emitting radionuclide thorium-227) reportedly inhibited tumor growth in several xenograft models. Wickstroem et al. (2019) Int J Radiat Oncol Biol Phys 105(2):410-422; Wittemer-Rump et al. (2014) “Pharmacokinetic and Pharmacodynamic (PK/PD) Modeling of Preclinical Data of a Novel Anti-Fibroblast Growth Factor Receptor 2 (FGFR2) Antibody (BAY1179470) to Guide Dosing in Phase 1,” AACR Abstract ID 672.

Yet, in clinical testing, Aprutumab ixodotin was poorly tolerated, with a minimum tolerated dose found to be below the therapeutic threshold estimated preclinically; therefore, the trial was terminated early. Sommer et al. (2016) Cancer Res 76(21):6331-6339; Sommer et al. (2014) “FGFR2-ADC Potently and Selectively Inhibits Growth of Gastric and Breast Cancer Xenograft Models,” AACR Abstract ID 4491.

There remains a significant unmet medical need for improved anti-cancer drugs that are effective in FGFR2b-expressing cancers. The present disclosure provides anti-FGFR2b antibodies and immunoconjugates that show various desirable characteristics for the treatment of a disease such as cancer in a subject, e.g., a human.

### SUMMARY OF THE DISCLOSURE

The present disclosure is directed to anti-FGFR2b antibodies and antigen-binding fragments thereof that specifically bind FGFR2b, and methods of their production and use. Humanized, mouse, or chimeric anti-FGFR2b antibodies are provided alone and as immunoconjugates. The antibodies bind to human glycosylated and aglycosylated FGFR2b with optimized binding kinetics, disrupt the human KGF:FGFR2b interaction, and find use in various therapeutic and preventive methods. The present disclosure includes isolated antibodies, derivatives, fragments, and conjugates thereof, pharmaceutical formulations comprising one or more of the anti-FGFR2b antibodies and immunoconjugates, and cell lines that produce the antibodies. Also provided are amino acid and nucleotide sequences of the antibodies.

In embodiments, an antibody or antigen-binding fragment thereof specifically binds to human FGFR2b and comprises a heavy chain complementarity determining region (HCDR) 1, HCDR2, and HCDR3, and a light chain complementarity determining region (LCDR) 1, LCDR2, and LCDR3, in which
HCDR1 comprises the sequence IYWX₁N (SEQ ID NO: 77),
HCDR2 comprises the sequence IYPENX₂DTNYX₃GKFKG (SEQ ID NO: 78),
HCDR3 comprises the sequence GGFDY (SEQ ID NO: 5),
LCDR1 comprises the sequence RASENIYSNLA (SEQ ID NO: 6),
LCDR2 comprises the sequence TATNLAX₄(SEQ ID NO: 79), and
LCDR3 comprises the sequence QHFYGILYT (SEQ ID NO: 8);
wherein X₁ is M or L; X₂ is G or A; X₃ is S or N; and X₄ is D or E.

In embodiments, an antibody or antigen-binding fragment thereof specifically binds to human FGFR2b and comprises HCDR1, HCDR2, and HCDR3, and LCDR1, LCDR2, and LCDR3, in which
HCDR1 comprises the sequence DTYIH (SEQ ID NO: 15),
HCDR2 comprises the sequence RIDPAX₁GNTMFASEFQG (SEQ ID NO: 80),
HCDR3 comprises the sequence SKIHYDYDEGFAY (SEQ ID NO: 17),
LCDR1 comprises the sequence RASESVDDYGYSFLH (SEQ ID NO: 18),
LCDR2 comprises the sequence RASNLES (SEQ ID NO: 19), and
LCDR3 comprises the sequence QQSNQNPRT (SEQ ID NO: 20);
wherein X₁ is E or N.

In embodiments, an antibody or antigen-binding fragment thereof that specifically binds to human FGFR2b comprises:
(i) a heavy chain variable region that comprises:
(a) a HCDR1 having the sequence IYWLN (SEQ ID NO: 3),
(b) a HCDR2 having the sequence QIYPENADTNYSGKFKG (SEQ ID NO: 4), and
(c) a HCDR3 having the sequence GGFDY (SEQ ID NO: 5); and
(ii) a light chain variable region that comprises:
(d) a LCDR1 having the sequence RASENIYSNLA (SEQ ID NO: 6),
(e) a LCDR2 having the sequence TATNLAE (SEQ ID NO: 7), and
(f) a LCDR3 having the sequence QHFYGILYT (SEQ ID NO: 8).

In embodiments, an antibody or antigen-binding fragment thereof that specifically binds to human FGFR2b comprises:
(i) a heavy chain variable region that comprises:
(a) a HCDR1 having the sequence DTYIH (SEQ ID NO: 15),
(b) a HCDR2 having the sequence RIDPAEGNTMFASEFQG (SEQ ID NO: 16), and
(c) a HCDR3 having the sequence SKIHYDYDEGFAY (SEQ ID NO: 17), and
(ii) a light chain variable region that comprises:
(d) a LCDR1 having the sequence RASESVDDYGYSFLH (SEQ ID NO: 18),
(e) a LCDR2 having the sequence RASNLES (SEQ ID NO: 19), and
(f) a LCDR3 having the sequence QQSNQNPRT (SEQ ID NO: 20).

In embodiments, an antibody or antigen-binding fragment thereof that specifically binds to human FGFR2b comprises:
(i) a heavy chain variable region (VH) comprising an amino acid sequence having at least 95% identity to SEQ ID NO: 9, and a light chain variable region (VL) comprising an amino acid sequence having at least 95% identity to SEQ ID NO: 10; or
(ii) a heavy chain variable region (VH) comprising an amino acid sequence having at least 95% identity to SEQ ID NO: 21, and a light chain variable region (VL) comprising an amino acid sequence having at least 95% identity to SEQ ID NO: 22.

In embodiments, one, two, three, four, five, six, seven, eight, nine, or ten amino acids within SEQ ID NOs: 9 and/or 10, and/or within SEQ ID NOs: 21 and/or 22 have been inserted, deleted, or substituted in an antibody or antigen-binding fragment thereof that specifically binds to human FGFR2b.

In embodiments, an antibody or antigen-binding fragment thereof that specifically binds to human FGFR2b is a monoclonal antibody, a chimeric antibody, a humanized antibody, a human engineered antibody, a single chain antibody (scFv), a Fab fragment, a Fab′ fragment, or a F(ab′)₂ fragment.

In embodiments, an antibody or antigen-binding fragment thereof that specifically binds to human FGFR2b has reduced glycosylation or no glycosylation or is hypofucosylated.

In embodiments, an antibody or antigen-binding fragment thereof that specifically binds to human FGFR2b comprises increased bisecting GlcNac structures.

In embodiments, an antibody or antigen-binding fragment thereof that specifically binds to human FGFR2b has an Fc domain of an IgG1.

In embodiments, a pharmaceutical composition comprises an antibody or antigen-binding fragment thereof that specifically binds to human FGFR2b, and a pharmaceutically acceptable carrier.

In embodiments, an isolated nucleic acid encodes an antibody or antigen-binding fragment thereof that specifically binds to human FGFR2b.

In embodiments, a vector comprises a nucleic acid that encodes an antibody or antigen-binding fragment thereof that specifically binds to human FGFR2b.

In embodiments, a host cell comprises a nucleic acid that encodes an antibody or antigen-binding fragment thereof that specifically binds to human FGFR2b or the vector comprising such a nucleic acid. In embodiments, a process for producing an antibody or antigen-binding fragment thereof that specifically binds to human FGFR2b comprises cultivating such a host cell in culture media and recovering the antibody or antigen-binding fragment thereof from the culture media. In embodiments, a purified composition comprises an anti-human FGFR2b antibody or antigen-binding fragment thereof produced by such a process.

In embodiments, an immunoconjugate, or a pharmaceutically acceptable salt, solvate, or hydrate thereof, comprises an antibody or antigen-binding fragment thereof that specifically binds to human FGFR2b, and a cytotoxic agent.

In embodiments, such an immunoconjugate comprises the formula:

Ab-(C-L-(D)ₘ)ₙ
or a pharmaceutically acceptable salt, solvate, or hydrate thereof; wherein
Ab is an antibody or antigen-binding fragment thereof that specifically binds to human FGFR2b;
C is a conjugator;
L is a linker;
D is the cytotoxic agent;
m is an integer from 1 to 8; and
n is from 1 to 10.

In embodiments, m is 1.

In embodiments, n is 3, 4, 5, 6, 7, 8, 9, or 10.

In embodiments, C has formula (C-I), (C-Ia), (C-Ib), (C-II), (C-III), (C-IIIa), (C-IIIb), or (C-IV):

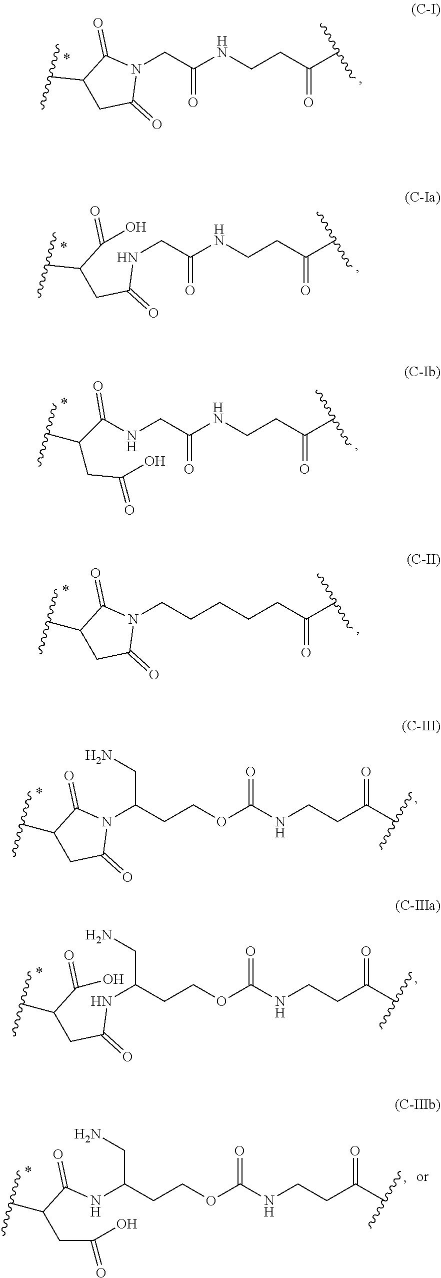

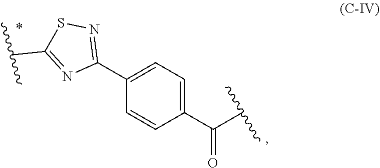 [SMILES: *C(=O)c1ccc(-c2nsc(*)n2)cc1]

wherein * marks the bond where the conjugator connects to Ab.

In embodiments, L has formula (L-I), (L-II), or (L-IIII):

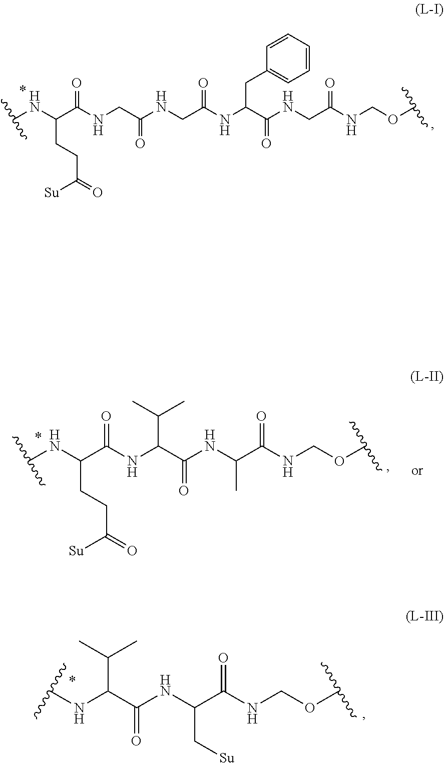

wherein Su is a hydrophilic residue; and
* marks the bond where the linker connects to the conjugator.

In embodiments, Su is

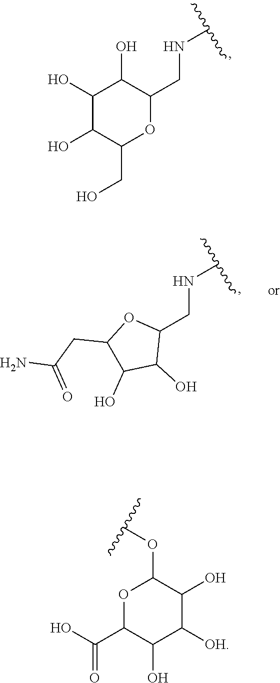 [SMILES: *NCC1OC(CO)C(O)C(O)C1O ; *NCC1OC(CC(N)=O)C(O)C1O ; *OC1OC(C(=O)O)C(O)C(O)C1O]

In embodiments, Su is

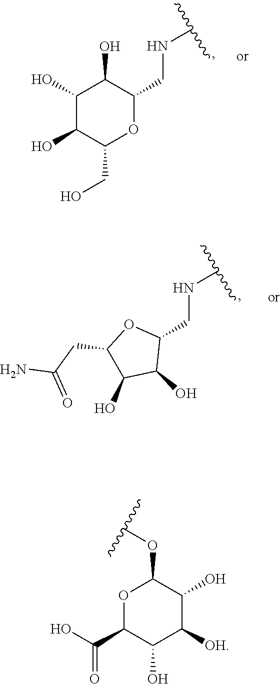 [SMILES: *NC[C@@H]1O[C@H](CO)[C@@H](O)[C@H](O)[C@H]1O ; *NC[C@H]1O[C@@H](CC(N)=O)[C@H](O)[C@@H]1O ; *O[C@@H]1O[C@H](C(=O)O)[C@@H](O)[C@H](O)[C@H]1O]

In embodiments, the cytotoxic agent (D) is selected from the group consisting of:

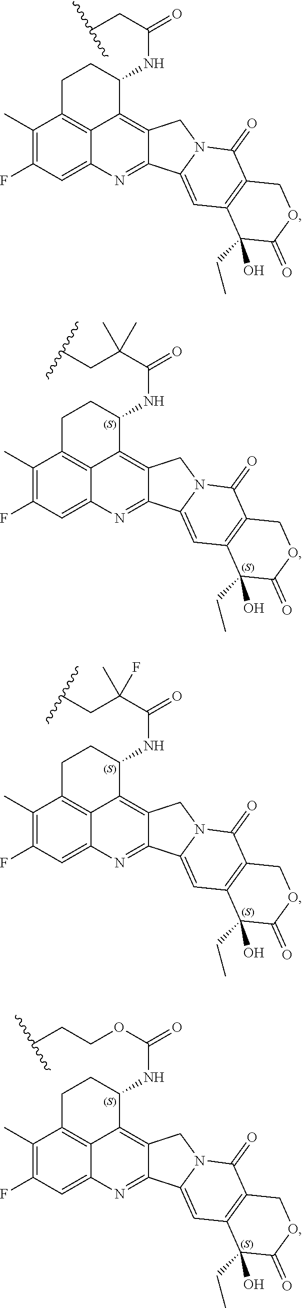

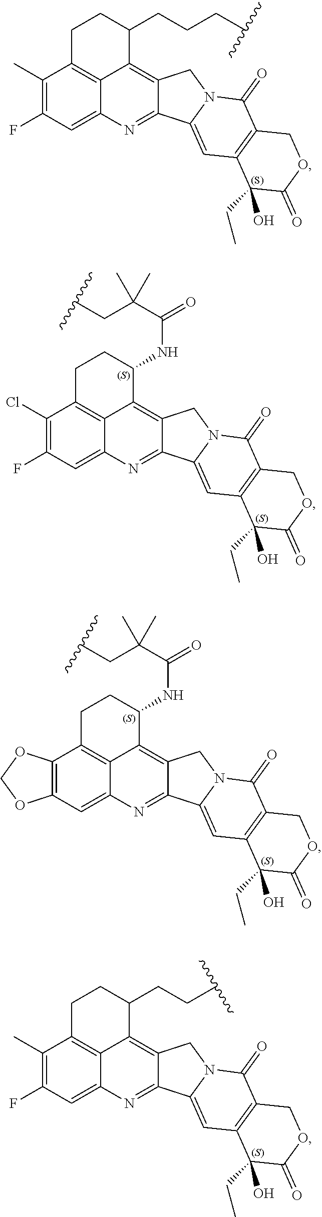

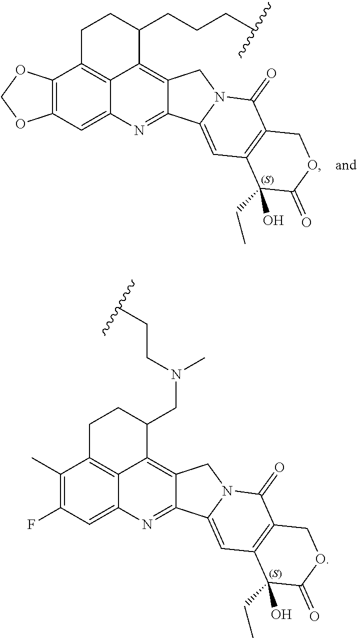

In embodiments, the cytotoxic agent (D) is selected from the group consisting of:

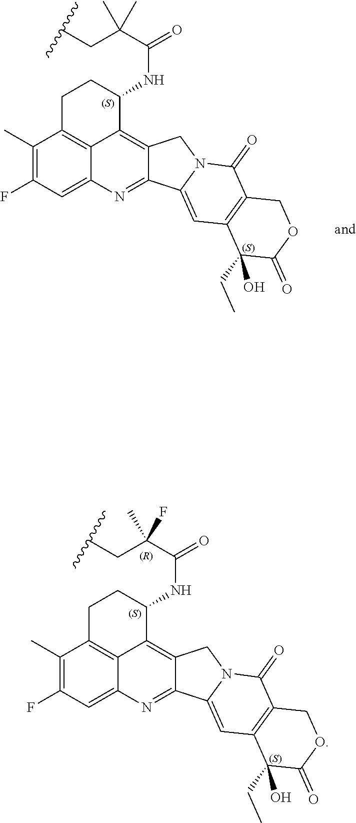

In embodiments, the immunoconjugate is represented by one of the following formulas:

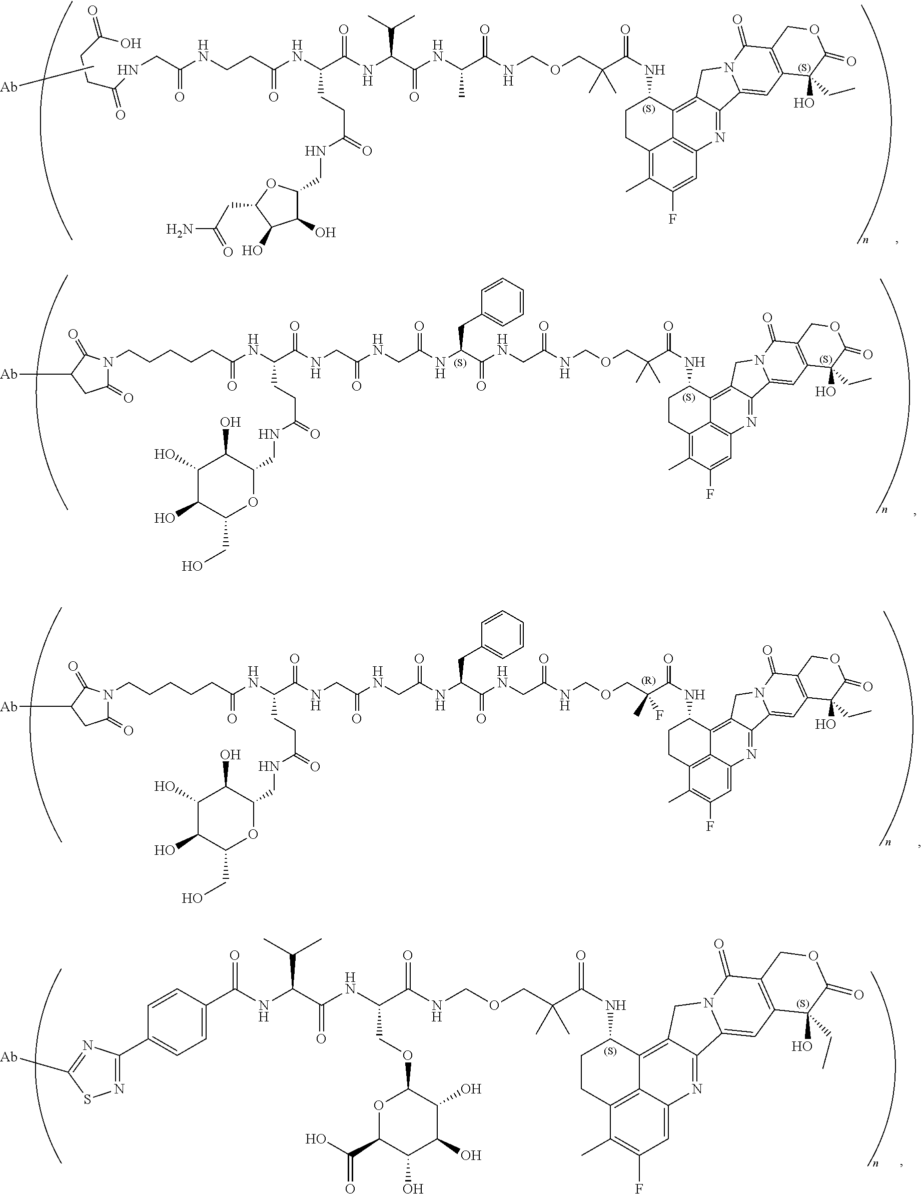

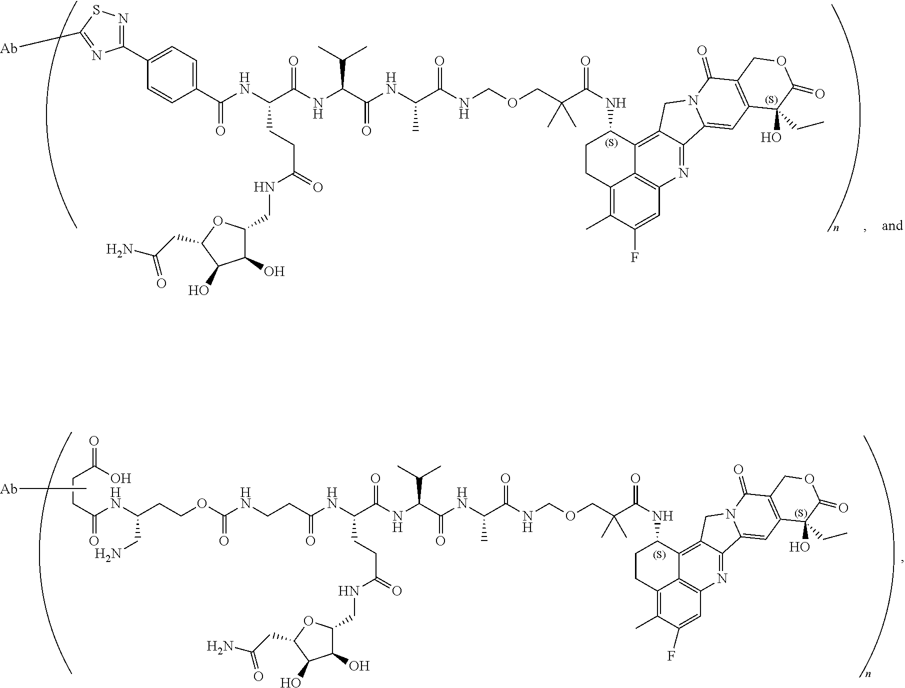

or a pharmaceutically acceptable salt, solvate, and/or stereoisomer thereof.

In embodiments, n is 3, 4, 5, 6, 7, 8, 9, or 10.

In embodiments, n is about 8.

In embodiments, the immunoconjugate is represented by one of the following formulas:

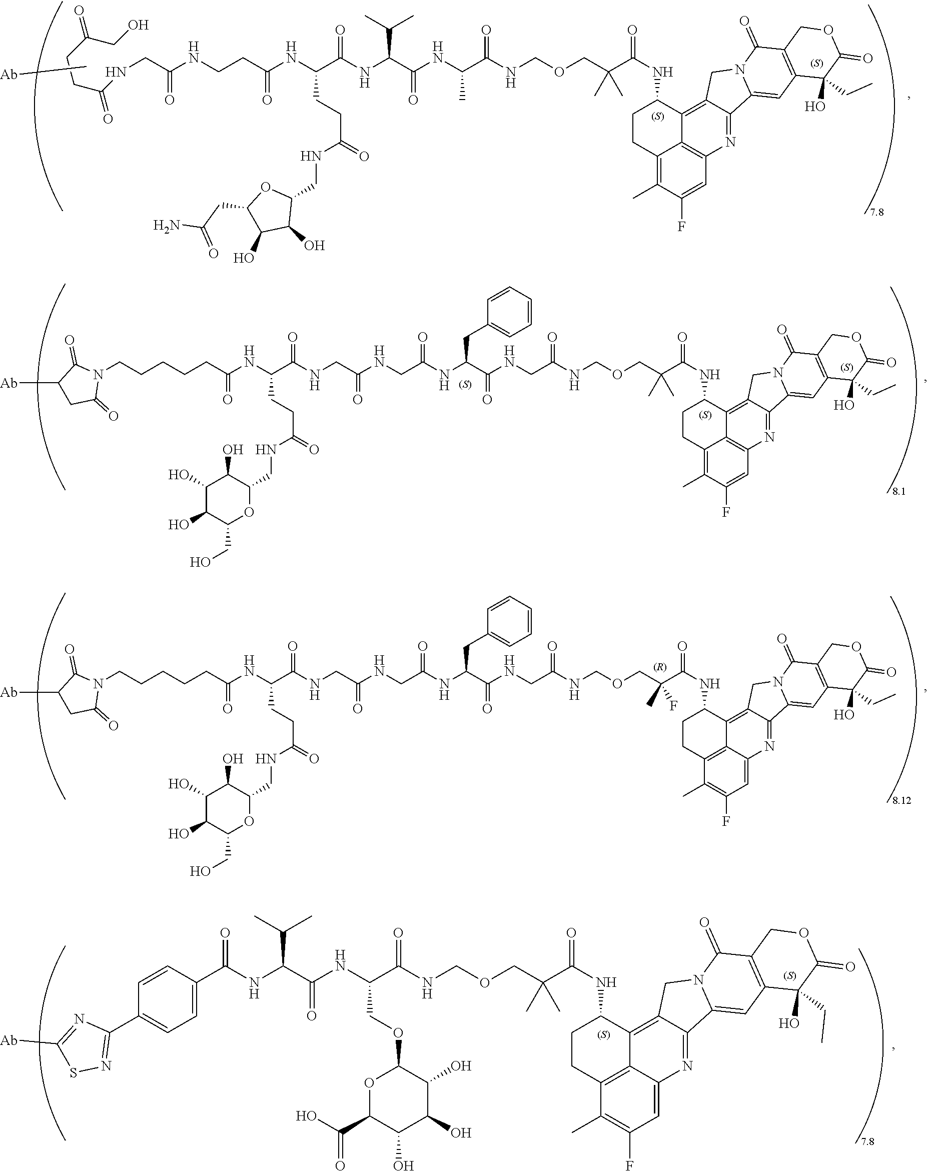

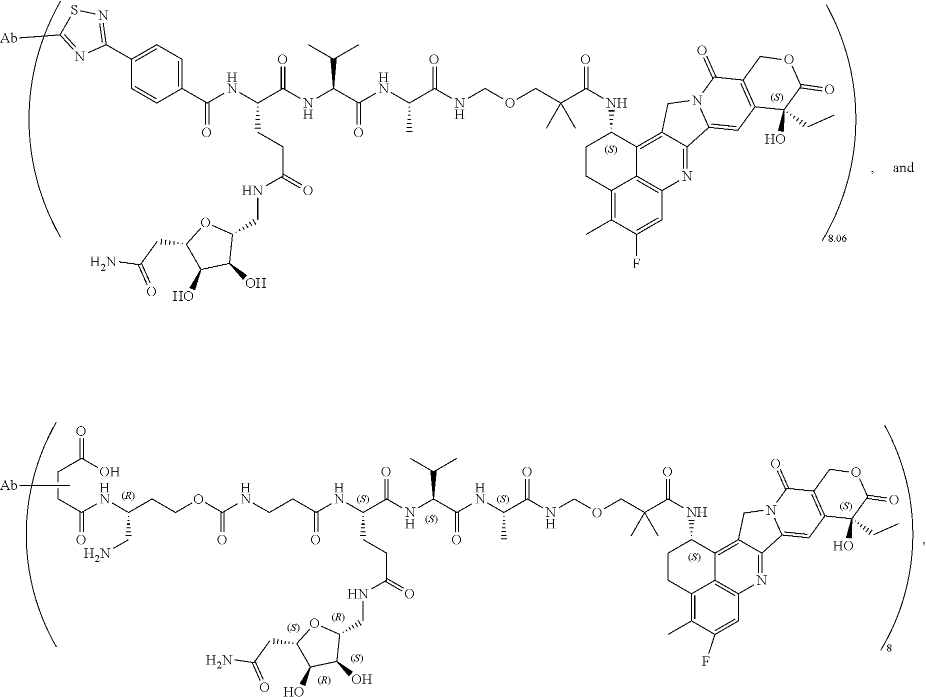

or a pharmaceutically acceptable salt, solvate, and/or stereoisomer thereof.

In embodiments, the immunoconjugate is represented by the following formula:

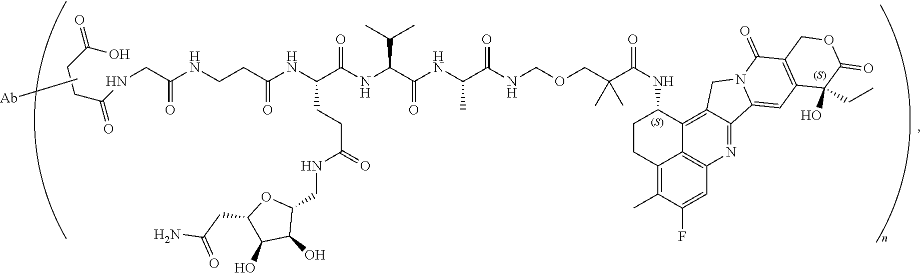

or a pharmaceutically acceptable salt, solvate, and/or stereoisomer thereof, wherein n is about 8 and the Ab comprises:
(i) a heavy chain variable region (VH) comprising the amino acid of SEQ ID NO: 9, and a light chain variable region (VL) comprising the amino acid sequence of SEQ ID NO: 10; or
(ii) a heavy chain variable region (VH) comprising the amino acid sequence of SEQ ID NO: 21, and a light chain variable region (VL) comprising the amino acid sequence of SEQ ID NO: 22.

In embodiments, the immunoconjugate is represented by the following formula:

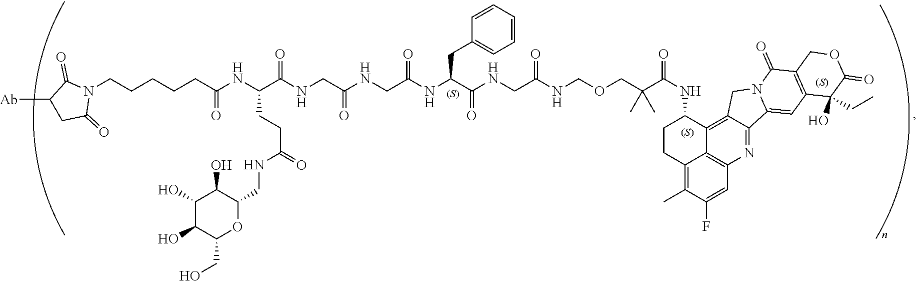

or a pharmaceutically acceptable salt, solvate, and/or stereoisomer thereof, wherein n is about 8 and the Ab comprises:
(i) a heavy chain variable region (VH) comprising the amino acid of SEQ ID NO: 9, and a light chain variable region (VL) comprising the amino acid sequence of SEQ ID NO: 10; or
(ii) a heavy chain variable region (VH) comprising the amino acid sequence of SEQ ID NO: 21, and a light chain variable region (VL) comprising the amino acid sequence of SEQ ID NO: 22.

In embodiments, the immunoconjugate is represented by the following formula:

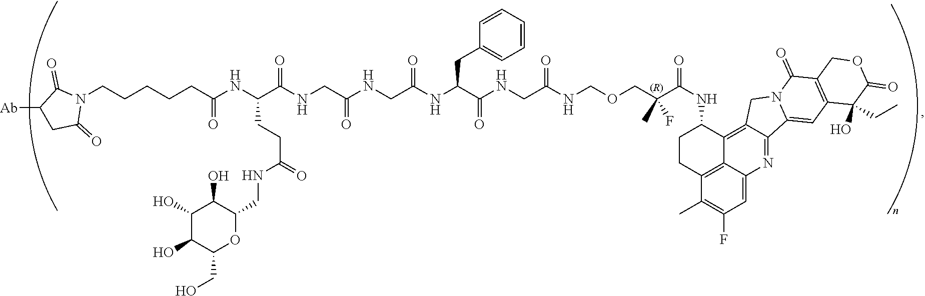

or a pharmaceutically acceptable salt, solvate, and/or stereoisomer thereof, wherein n is about 8 and the Ab comprises:
(i) a heavy chain variable region (VH) comprising the amino acid of SEQ ID NO: 9, and a light chain variable region (VL) comprising the amino acid sequence of SEQ ID NO: 10; or
(ii) a heavy chain variable region (VH) comprising the amino acid sequence of SEQ ID NO: 21, and a light chain variable region (VL) comprising the amino acid sequence of SEQ ID NO: 22.

In embodiments, the immunoconjugate is represented by the following formula:

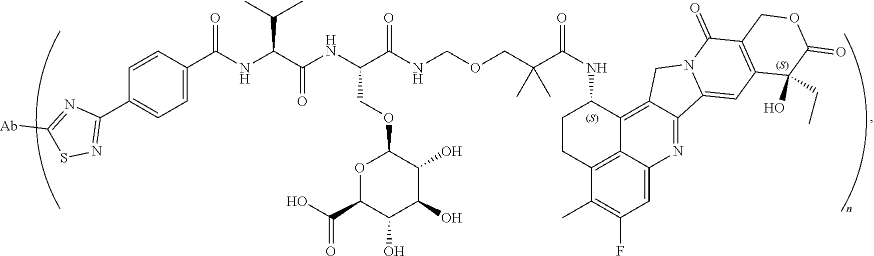

or a pharmaceutically acceptable salt, solvate, and/or stereoisomer thereof, wherein n is about 8 and the Ab comprises:
(i) a heavy chain variable region (VH) comprising the amino acid of SEQ ID NO: 9, and a light chain variable region (VL) comprising the amino acid sequence of SEQ ID NO: 10; or
(ii) a heavy chain variable region (VH) comprising the amino acid sequence of SEQ ID NO: 21, and a light chain variable region (VL) comprising the amino acid sequence of SEQ ID NO: 22.

In embodiments, the immunoconjugate is represented by the following formula:

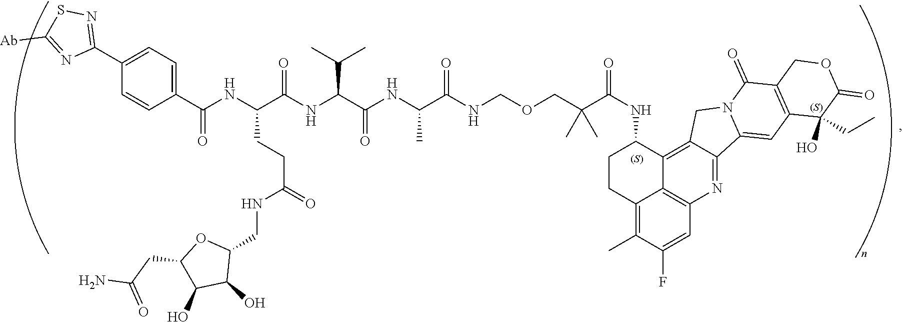

or a pharmaceutically acceptable salt, solvate, and/or stereoisomer thereof, wherein n is about 8 and the Ab comprises:
(i) a heavy chain variable region (VH) comprising the amino acid of SEQ ID NO: 9, and a light chain variable region (VL) comprising the amino acid sequence of SEQ ID NO: 10; or
(ii) a heavy chain variable region (VH) comprising the amino acid sequence of SEQ ID NO: 21, and a light chain variable region (VL) comprising the amino acid sequence of SEQ ID NO: 22.

In embodiments, the immunoconjugate is represented by the following formula:

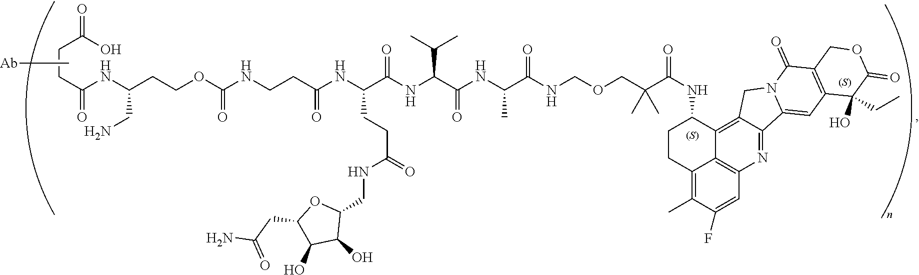

or a pharmaceutically acceptable salt, solvate, and/or stereoisomer thereof, wherein n is about 8 and the Ab comprises:
(i) a heavy chain variable region (VH) comprising the amino acid of SEQ ID NO: 9, and a light chain variable region (VL) comprising the amino acid sequence of SEQ ID NO: 10; or
(ii) a heavy chain variable region (VH) comprising the amino acid sequence of SEQ ID NO: 21, and a light chain variable region (VL) comprising the amino acid sequence of SEQ ID NO: 22.

In embodiments, a pharmaceutical composition comprises the immunoconjugate and a pharmaceutically acceptable carrier.

In embodiments, a kit comprises the antibody or antigen-binding fragment thereof that specifically binds to FGFR2b, the pharmaceutical composition, or the immunoconjugate, and instructions for using the same.

In embodiments of a kit, the antibody or antigen-binding fragment thereof forms a complex with FGFR2b that is detected by an assay comprising an enzyme linked immunosorbent assay (ELISA), radioimmune assay (RIA), and/or Western blot.

In embodiments, a method of treating small cell lung cancer or gastric cancer comprises administering to a patient in need thereof an effective amount of the pharmaceutical composition, purified composition, or immunoconjugate.

In embodiments, a method of treating a tubulin inhibitor-resistant tumor comprises administering an effective amount of the pharmaceutical composition, purified composition, or immunoconjugate to a subject having an accumulation or overexpression of FGFR2b in a biological sample.

In embodiments, the pharmaceutical composition, purified composition, or immunoconjugate is used in the manufacture of a medicament for treating small cell lung cancer or gastric cancer in a patient.

In embodiments, the pharmaceutical composition, purified composition, or immunoconjugate is used for treating small cell lung cancer or gastric cancer in a patient.

In embodiments, the pharmaceutical composition, purified composition, or immunoconjugate is administered in combination with at least one other therapeutic agent.

In embodiments, the other therapeutic agent is a chemotherapeutic agent.

In embodiments, the other therapeutic agent comprises at least one immune checkpoint inhibitor.

In embodiments, the immune checkpoint inhibitor is an anti-PD1 or anti-PD-L1 antibody.

In embodiments, the anti-PD1 antibody is Tislelizumab.

In embodiments, a method of producing the immunoconjugate comprises:
(i) culturing a host cell which has been transformed by an isolated nucleic acid comprising a sequence encoding an anti-FGFR2b antibody or antigen-binding fragment thereof, wherein the antibody comprises a heavy chain comprising an amino acid sequence of SEQ ID NO: 9 and a light chain comprising an amino acid of SEQ ID NO: 10, or a heavy chain comprising an amino acid sequence of SEQ ID NO: 21 and a light chain comprising an amino acid of SEQ ID NO: 22;
(ii) expressing said antibody or antigen-binding fragment thereof;
(iii) recovering the expressed antibody or antigen-binding fragment thereof; and
(iv) conjugating at least one cytotoxic agent (D) to the antibody or antigen-binding fragment thereof using the linker (L) and conjugator (C) such that an immunoconjugate is formed.

This Summary is neither intended as, nor should it be construed as, being representative of the full extent and scope of the present disclosure. Moreover, references made herein to “the present disclosure,” or aspects thereof, should be understood to mean certain embodiments of the present disclosure and should not be construed as limiting all embodiments to a particular description. The present disclosure is set forth in various levels of detail in this Summary as well as in the Detailed Description and accompanying drawings, and no limitation as to the scope of the present disclosure is intended by either the inclusion or non-inclusion of elements, components, etc. in this Summary. Features from any of the disclosed embodiments may be used in combination with one another, without limitation. In addition, other features and advantages of the present disclosure will become apparent to those of ordinary skill in the art through consideration of the following Detailed Description and the accompanying drawings.

### BRIEF DESCRIPTION OF THE DRAWINGS

FIG. 1 is modified from Tiong, K. H. et al. (2013) December; 18:1447-68 and shows the basic structure of fibroblast growth factor receptor (FGFR), which consists of three extracellular immunoglobulin-like (Ig-like) domains (D1, D2, and D3), a single transmembrane helix, an intracellular split tyrosine kinase domain (TK1 and TK2) and an acidic box. Ig-like domain II (D2) and Ig-like domain III (D3) form the ligand-binding pocket and have distinct domains. FGFR splice variants are shown, which form from alternative splicing of D3. The IgIIIb and IgIIIc splice variants are commonly observed in the FGFR2 gene. Epithelial tissues predominantly express the IgIIIb isoform and mesenchymal tissues express IgIIIc. The hatched box represents a truncated carboxyl terminal.

FIG. 2 shows the amino acid sequence of human FGFR2 (SEQ ID NO:1), provided at NCBI Accession number P21802. In bold is the beta IIIb (D2+D3) domain, which is approximately Pro253-Glu378, and which was used for immunization. The amino acid sequence of human keratinocyte growth factor (SEQ ID NO:2), accession number AAB21431.1, is also provided.

FIGS. 3A-3D show the comparison of native protein binding activities of different humanized anti-FGFR2b antibodies using FGFR2b-overexpressed Expi293 cells. FIG. 3A shows comparable human FGFR2b binding among BGAm1823, BGAh9239, BGAm1371, and BGAh9179; FIG. 3B shows comparable cyno FGFR2b binding among BGAm1823, BGAh9239, BGAm1371, and BGAh9179; FIG. 3C shows comparable mouse FGFR2b binding among BGAm1823, BGAh9239, BGAm1371, and BGAh9179; and FIG. 3D shows comparable human FGFR2c binding among BGAm1823, BGAh9239, BGAm1371, and BGAh9179.

FIGS. 4A-4C show the comparison of recombinant protein binding activities of different humanized anti-FGFR2b antibodies by ELISA. FIG. 4A shows the ELISA results of BGAm1823, BGAh9239, BGAm1371, and BGAh9179 against human FGFR2b protein; FIG. 4B shows the ELISA results of BGAm1823, BGAh9239, BGAm1371, and BGAh9179 against cyno FGFR2b protein; and FIG. 4C shows the ELISA results of BGAm1823, BGAh9239, BGAm1371, and BGAh9179 against human FGFR2c protein.

FIGS. 5A-5D show SPR binding kinetics of selected chimeric FGFR2b binders.

FIGS. 6 and 7 show the results of Ba/F3 proliferation assays. The net growth on Ba/F3 cells overexpressing FGFR2 is reported after treatment with anti-FGFR2b antibodies of the present disclosure.

FIG. 8 is a primary structure alignment of the amino acid sequences of the top anti-FGFR2b antibody candidates. Normal sequence diversity is shown.

FIG. 9 shows the anti-tumor efficacy of different concentrations of BGA3457 and BGA9823, relative to comparator BGA8723, in a cell-line derived xenograft (CDX) model using SNU-16 (FGFR2b high expression) cells.

FIG. 10 shows the anti-tumor efficacy of different concentrations of BGA3457 and BGA9823, relative to comparator BGA8723, in a cell-line derived xenograft (CDX) model using HSC-39 (gastric signet ring cell adenocarcinoma; FGFR2b medium expression) cells.

FIGS. 11A-11B show the antitumor activity of BGA3457 in co-inoculated HSC-39 (gastric signet ring cell adenocarcinoma; human FGFR2b medium to low overexpressed cell line)+SNU-5 (stomach cancer) xenograft models at a ratio of 2:1 or 5:1, respectively, relative to comparator BGA1421.

FIGS. 12A-12B show the antitumor activity of BGA9823 in co-inoculated HSC-39 (gastric signet ring cell adenocarcinoma, human FGFR2b medium to low overexpressed cell line)+SNU-5 (stomach cancer) xenograft models at a ratio of 2:1 or 5:1, respectively, relative to comparator BGA1421.

FIGS. 13A-13D show the cellular killing by immunoconjugates BGA9823, BGA3457, and comparator BGA8723 in multiple cell lines with different expression levels. The following cell lines were evaluated: SNU16 (gastric cancer, FGFR2b high overexpression, FIG. 13A); SNU601-h2b (gastric cancer, FGFR2b medium overexpression, FIG. 13B); H1048-h2b (small cell lung cancer, FGFR2b medium to low overexpression, FIG. 13C); and HSC-39 (gastric signet ring cell adenocarcinoma, FGFR2b medium to low overexpression, FIG. 13D).

FIG. 14A-14D shows that BGA3457 demonstrated nanomolar-range killing in cell lines with diverse FGFR2b expression.

FIGS. 15A-15B show that BGA3457 demonstrated robust anti-tumor efficacy in FGFR2b^{high} SNU-16 (15A) and FGFR2b^{medium} HSC-39 (15B) CDX models, relative to comparator BGA8723. No reduction in body weight was observed in either experiment.

FIG. 16 shows that BGA3457 demonstrated a statistically significant bystander killing effect in vitro.

FIGS. 17A-17B show that BGA3457 and BGA9823 demonstrated good plasma stability in human and murine models with a low free payload release (<1%) and stable DAR value at 8.

FIGS. 18A-18B show that BGA3457 has an acceptable PK profile in tumor-bearing (FIG. 18A) and hFcRn (FIG. 18B) mice. BGA3457 showed nonlinear PK from 1-10 mg/kg in mice, suggesting target-mediated clearance (TMDD).

FIG. 19 shows that BGA3457 demonstrated overall good PK profiles in cyno models at 1, 3, and 10 mg/kg (mpk).

FIGS. 20A and 20B are Western blots showing the effects of anti-FGFR2b antibodies on FGF7-induced or FGF10-induced FGFR phosphorylation. The anti-FGFR2b antibodies weakly inhibited FGF7-induced signaling and had little to no impact on FGF10-induced signaling.

FIG. 21 shows the anti-tumor efficacy of BGA3457 in a patient-derived xenograft (PDX) model relative to vehicle and comparator BGA8723.

FIG. 22 shows the effects of treatment with BGA 3457 or comparator BGA1421 on murine corneal thickness.

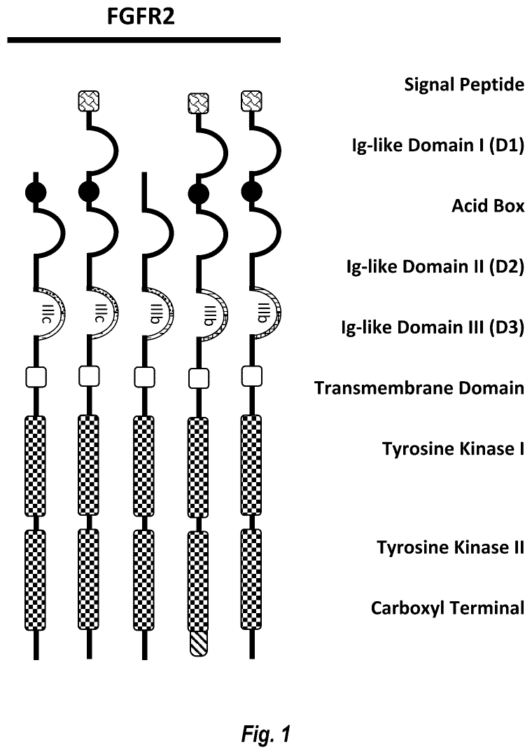

### ABBREVIATIONS

As used throughout the specification and appended claims, the following abbreviations apply:
API active pharmaceutical ingredient
CDR complementarity determining region in the immunoglobulin
CHO Chinese hamster ovary
DS drug substance
EC50 concentration resulting in 50% efficacy or binding
ELISA enzyme-linked immunosorbent assay
FFPE formalin-fixed, paraffin-embedded
FGF fibroblast growth factor
FR framework region
HC heavy chain
IC50 concentration resulting in 50% inhibition
IgG immunoglobulin G
IV intravenous
IHC immunohistochemistry or immunohistochemical
KGF keratinocyte growth factor
mAb monoclonal antibody
NCBI National Center for Biotechnology Information
PD-1 programmed death 1
PD-L1 programmed death 1 ligand 1
VH immunoglobulin heavy chain variable region
VK immunoglobulin kappa light chain variable region
VL immunoglobulin light chain variable region
v/v volume per volume
w/v weight per volume

### Definitions

The section headings used herein are for organizational purposes only and are not to be construed as limiting the subject matter described.

Unless specifically defined below or elsewhere in this document, all technical and scientific terms used herein have the meaning commonly understood by one of ordinary skill in the art.

“FGFR2b” refers to a cell receptor. The amino acid sequence of human FGFR2b (SEQ ID NO: 1) can also be found at accession number P21802 (FGFR2b_HUMAN) or UniProtKB/Swiss-Prot: P21802.1. The bolded region in the following sequence is the beta IIIb (D2+D3) domain, which is approximately Pro253-Glu378, which was used for immunization to create the antibodies disclosed herein.

| (SEQ ID NO: 1) | |
|---|---|
| 1 | MVSWGRFICL VVVTMATLSL ARPSFSLVED TTLEPEEPPT KYQISQPEVY VAAPGESLEV |
| 61 | RCLLKDAAVI SWTKDGVHLG PNNRTVLIGE YLQIKGATPR DSGLYACTAS RTVDSETWYF |
| 121 | MVNVTDAISS GDDEDDTDGA EDFVSENSNN KRAPYWINTE KMEKRLHAVP AANTVKFRCP |
| 181 | AGGNPMPTMR WLKNGKEFKQ EHRIGGYKVR NQHWSLIMES VVPSDKGNYT CVVENEYGSI |
| 241 | NHTYHLDVVE RSPHRPILQA GLPANASTVV GGDVEFVCKV YSDAQPHIQW IKHVEKNGSK |
| 301 | YGPDGLPYLK VLKAAGVNTT DKEIEVLYIR NVTFEDAGEY TCLAGNSIGI SFHSAWLTVL |
| 361 | PAPGREKEIT ASPDYLEIAI YCIGVFLIAC MVVTVILCRM KNTTKKPDFS SQPAVHKLTK |
| 421 | RIPLRRQVTV SAESSSSMNS NTPLVRITTR LSSTADTPML AGVSEYELPE DPKWEFPRDK |
| 481 | LTLGKPLGEG CFGQVVMAEA VGIDKDKPKE AVTVAVKMLK DDATEKDLSD LVSEMEMMKM |
| 541 | IGKHKNIINL LGACTQDGPL YVIVEYASKG NLREYLRARR PPGMEYSYDI NRVPEEQMTF |
| 601 | KDLVSCTYQL ARGMEYLASQ KCIHRDLAAR NVLVTENNVM KIADFGLARD INNIDYYKKT |
| 661 | TNGRLPVKWM APEALFDRVY THQSDVWSFG VLMWEIFTLG GSPYPGIPVE ELFKLLKEGH |
| 721 | RMDKPANCTN ELYMMMRDCW HAVPSQRPTF KQLVEDLDRI LTLTTNEEYL DLSQPLEQYS |
| 781 | PSYPDTRSSC SSGDDSVFSP DPMPYEPCLP QYPHINGSVK T |

A ligand for FGFR2b is known as keratinocyte growth factor (“KGF”) and the amino acid sequence, which follows, can also be found at accession number AAB21431.1 (SEQ ID NO: 2).

| (SEQ ID NO: 2) | |
|---|---|
| 1 | MHKWILTWIL PTLLYRSCFH IICLVGTISL ACNDMTPEQM ATNVNCSSPE RHTRSYDYME |
| 61 | GGDIRVRRLF CRTQWYLRID KRGKVKGTQE MKNNYNIMEI RTVAVGIVAI KGVESEFYLA |
| 121 | MNKEGKLYAK KECNEDCNFK ELILENHYNT YASAKWTHNG GEMFVALNQK GIPVRGKKTK |
| 181 | KEQKTAHELP MAIT |

The terms “anti-FGFR2b antibody” and “an antibody that binds to FGFR2b” refer to an antibody that is capable of binding FGFR2b with sufficient affinity such that the antibody is useful as a diagnostic and/or therapeutic agent in targeting FGFR2b.

“FGFR2b expression positive” refers to an elevated level of FGFR2b expression (protein and/or mRNA) by malignant cells within a tumor compared to an appropriate control. The level of FGFR2b mRNA expression may be compared to the mRNA expression levels of one or more reference genes that are frequently used in quantitative RT-PCR. In some embodiments, a level of FGFR2b expression (protein and/or mRNA) by malignant cells and/or by infiltrating immune cells within a tumor is determined to be “overexpressed” or “elevated” based on comparison with the level of FGFR2b expression (protein and/or mRNA) by an appropriate control. For example, a control FGFR2b protein or mRNA expression level may be the level quantified in nonmalignant cells of the same type or in a section from a matched normal tissue. In some preferred embodiments, FGFR2b expression in a tumor sample is determined to be elevated if FGFR2b protein (and/or FGFR2b mRNA) in the sample is at least 10%, 20%, or 30% greater than in the control. “KGF expression positive” is similarly defined.

Units, prefixes, and symbols used herein are provided using their Systeme International de Unites (SI) accepted form.

As used herein, including in the appended claims, the singular forms of words such as “a,” “an,” and “the” include their corresponding plural forms unless the context clearly indicates otherwise.

The term “or” is used to mean, and is used interchangeably with, the term “and/or” unless the context clearly indicates otherwise.

The term “and/or” used herein is to be taken as specific disclosure of each of the specified features or components with or without the other(s). Thus, the term “and/or” as used in a phrase such as “A and/or B” is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.” and “i.e.” as used herein are used merely by way of example, without limitation intended, and should not be construed as referring to only those items explicitly enumerated in the specification.

Unless specifically stated or evident from context, as used herein, the terms “about” and “approximately” refer to a value or composition that is within an acceptable error range for the particular value or composition as determined by one of ordinary skill in the art, which will depend in part on how the value or composition is measured or determined, i.e., the limitations of the measurement system. For example, “about” can mean within one or more than one standard deviation per the practice in the art. “About” can mean a range of up to 10% (i.e., ±10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” should be assumed to be within an acceptable error range for that particular value or composition.

The terms “at least,” “more than,” “or more,” and the like, e.g., “at least one,” are understood to include, but not be limited to, at least 1, 2, 3, 4, etc. more than the stated value. Also included is any greater number or fraction in between. Conversely, the term “no more than” includes each value less than the stated value. For example, “no more than 10 nucleotides” includes 10, 9, 8, 7, 6, 5, 4, 3, 2, 1, and 0 nucleotides. Also included is any lesser number or fraction in between. The terms “plurality,” “at least two,” “two or more,” “at least second,” and the like are understood to include, but not be limited to, at least 2, 3, 4, 5, etc. or more than the stated value. Also included is any greater number or fraction in between.

Ranges can be expressed herein as from one particular value to “about” another particular value. When such a range is expressed, the one particular value and/or to the other particular value may be included. Similarly, when values are expressed as approximations, by use of the antecedent “about,” it will be understood that the particular value forms another case. It will be further understood that the endpoints of each of the ranges may be significant both in relation to the other endpoint and independently of the other endpoint. As described herein, any concentration range, percentage range, ratio range, or integer range is to be understood to be inclusive of the value of any integer within the recited range and, when appropriate, fractions thereof (such as one-tenth and one-hundredth of an integer), unless otherwise indicated.

The term “reference” describes a comparator, which is used as a standard or control relative to which a comparison is performed. For example, in some embodiments, an agent, animal, individual, population, sample, sequence, or value of interest is compared with a reference or control that is an agent, animal, individual, population, sample, sequence, or value. In some embodiments, a reference or control is tested, measured, and/or determined substantially simultaneously with the testing, measuring, or determination of interest. In some embodiments, a reference or control is a historical reference or control, optionally embodied in a tangible medium. Generally, a reference or control is determined or characterized under comparable conditions or circumstances to those under assessment.

The terms “improve,” “increase,” “inhibit,” and “reduce” indicate values that are relative to a baseline or other reference measurement. In some embodiments, an appropriate reference measurement may comprise a measurement in certain system (e.g., in a single individual) under otherwise comparable conditions absent presence of (e.g., prior to and/or after) an agent or treatment, or in presence of an appropriate comparable reference agent. In some embodiments, an appropriate reference measurement may comprise a measurement in a comparable system known or expected to respond in a comparable way, in the presence of the relevant agent or treatment.

The term “isolated” refers to a substance that (1) has been separated from at least some components with which it was associated at an earlier time or with which the substance would otherwise be associated, and/or (2) is present in a composition that comprises a limited or defined amount or concentration of one or more known or unknown contaminants. An isolated substance, in some embodiments, may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% (e.g., 85-90%, 85-95%, 85-100%, 90-95%, 90-100%, or 95-100%) of other non-substance components with which the substance was associated at an earlier time, e.g., other components or contaminants with which the substance was previously or otherwise would be associated. In certain instances, a substance is isolated if it is present in a composition that comprises a limited or reduced amount or concentration of molecules of a same or similar type. For example, in certain instances, a nucleic acid, DNA, or RNA substance is isolated if it is present in a composition that comprises a limited or reduced amount or concentration of non-substance nucleic acid, DNA, or RNA molecules. As another example, a polypeptide substance is isolated if it is present in a composition that comprises a limited or reduced amount or concentration of non-substance polypeptide molecules. In certain embodiments, an amount may be, e.g., an amount measured relative to the amount of a desired substance present in a composition. In certain embodiments, a limited amount may be an amount that is no more than 100% of the amount of substance in a composition, e.g., no more than 1%, 5%, 10%, 20%, 30%, 40%, 50%, 60%, 70%, 80%, 90%, or 95% of the amount of substance in a composition (e.g., 85-90%, 85-95%, 85-100%, 90-95%, 90-100%, or 95-100%). In certain instances, a composition is pure or substantially pure with respect to a selected substance. In some embodiments, an isolated substance is about 80%, about 85%, about 90%, about 91%, about 92%, about 93%, about 94%, about 95%, about 96%, about 97%, about 98%, about 99%, or more than about 99% pure (e.g., 85-90%, 85-95%, 85-100%, 90-95%, 90-100%, or 95-100%).

The term “agent” may refer to a molecule or entity of any class comprising, or a plurality of molecules or entities, any of which may be, for example, a polypeptide, nucleic acid, saccharide, lipid, small molecule, metal, conjugate (for example, an antibody drug conjugate), cell, or organism (for example, a fraction or extract thereof) or component thereof. In some embodiments, an agent may be utilized in isolated or pure form. In some embodiments, an agent may be utilized in a crude or impure form. In some embodiments, an agent may be provided as a population, collection, or library, for example that may be screened to identify or characterize members present therein.

“Contacting” is used in accordance with its plain ordinary meaning and refers to the process of allowing at least two distinct species (e.g., chemical compounds including biomolecules or cells) to become sufficiently proximal to react, interact, or physically touch. The two species may be, for example, an anti-FGFR2b antibody as described herein and a FGFR2b antigen. In certain embodiments, contacting includes, for example, allowing an anti-FGFR2b antibody as described herein to interact with a FGFR2b antigen.

The terms “administration” and “administering,” as used herein, when applied to an animal, human, subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell.

The term “subject” or “patient” herein includes any organism, preferably an animal, more preferably a mammal (e.g., rat, mouse, dog, cat, rabbit, primate) and most preferably a human (e.g., a patient having, or at risk of having, a disorder described herein).

“Treating” any disease or disorder refers in one aspect to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically (e.g., stabilization of a discernible symptom), physiologically (e.g., stabilization of a physical parameter), or both. The disease or disorder may be associated with FGFR2b binding to its ligand KGF (“FGFR2b-related disease”).

The term “effective amount” or “therapeutically effective amount” in connection with a compound means an amount capable of alleviating, in whole or in part, symptoms, or slowing or halting further progression or worsening of those symptoms. As will be apparent to those skilled in the art, it is to be expected that the effective amount of a compound disclosed herein may vary depending on the severity of the indication being treated.

An “anti-tumor effect” or “anti-tumor efficacy,” as used herein, refers to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor.

The term “sample” generally refers to an aliquot of material obtained or derived from a source of interest. In some embodiments, a source of interest is a biological or environmental source. In some embodiments, a source of interest may comprise a cell or an organism, such as a cell population, tissue, or animal (e.g., a human). In some embodiments, a source of interest comprises biological tissue or fluid. In some embodiments, a biological tissue or fluid may comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humor, vomit, and/or combinations or component(s) thereof. In some embodiments, a biological fluid may comprise an intracellular fluid, an extracellular fluid, an intravascular fluid (blood plasma), an interstitial fluid, a lymphatic fluid, and/or a transcellular fluid. In some embodiments, a biological tissue or sample may be obtained, for example, by aspirate, biopsy (e.g., fine needle or tissue biopsy), swab (e.g., oral, nasal, skin, or vaginal swab), scraping, surgery, washing, or lavage (e.g., bronchioalveolar, ductal, nasal, ocular, oral, uterine, vaginal, or other washing or lavage). In some embodiments, a biological sample comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. Such a “processed sample” may comprise, for example nucleic acids or proteins extracted from a sample or obtained by subjecting a primary sample to one or more techniques such as amplification or reverse transcription of nucleic acid, isolation, and/or purification of certain components, etc.

An “antigen” refers to any molecule that provokes an immune response or is capable of being bound by an antibody or an antigen-binding molecule. The immune response may involve antibody production, the activation of specific immunologically competent cells, or both. A person of skill in the art would readily understand that any macromolecule, including virtually all proteins or peptides, can serve as an antigen. An antigen can be endogenously expressed, i.e., expressed by genomic DNA, or can be recombinantly expressed. An antigen can be specific to a certain tissue, such as a cancer cell, or it can be broadly expressed. In addition, fragments of larger molecules can act as antigens. In one embodiment, antigens are tumor antigens. In one particular embodiment, the antigen is all or a fragment of FGFR2b.

The term “antibody” as used herein refers to a polypeptide of the immunoglobulin family that can bind a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH or HCVR) and a heavy chain constant region. The heavy chain constant region is comprised of three domains, CH1, CH2, and CH3. Each light chain is comprised of a light chain variable region (abbreviated herein as VL, Vκ, or LCVR) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs arranged from amino-terminus to carboxyl-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

The positions of the CDRs and framework regions can be determined using various well-known definitions in the art, e.g., Kabat, Chothia, AbM and IMGT as described in more detail below.

The term “antibody” includes, but is not limited to, monoclonal antibodies, human antibodies, humanized antibodies, human engineered antibodies, chimeric antibodies, and anti-idiotypic (anti-Id) antibodies. The antibodies can be of any isotype/class (e.g., IgG, IgE, IgM, IgD, IgA, and IgY), or subclass (e.g., IgG1, IgG2, IgG3, IgG4, IgA1, and IgA2).

The term “monoclonal antibody” or “mAb” or “Mab” herein means a population of substantially homogeneous antibodies, i.e., the antibody molecules in the population are identical in amino acid sequence except for possible naturally occurring mutations that can be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies and is not to be construed as requiring production of the antibody by any particular method. Monoclonal antibodies can be obtained by methods known to those skilled in the art. See, for example, Kohler et al., Nature 1975 256:495-497; U.S. Pat. No. 4,376,110; Ausubel et al., CURRENT PROTOCOLS IN MOLECULAR BIOLOGY 1992; Harlow et al., ANTIBODIES: a LABORATORY MANUAL, Cold Spring Harbor Laboratory 1988; and Colligan et al., CURRENT PROTOCOLS IN IMMUNOLOGY 1993. The antibodies disclosed herein can be of any immunoglobulin class including IgG, IgM, IgD, IgE, IgA, and any subclass thereof such as IgG1, IgG2, IgG3, IgG4. A hybridoma producing a monoclonal antibody can be cultivated in vitro or in vivo. High titers of monoclonal antibodies can be obtained in in vivo production where cells from the individual hybridomas are injected intraperitoneally into mice, such as pristine-primed Balb/c mice to produce ascites fluid containing high concentrations of the desired antibodies. Monoclonal antibodies of isotype IgM or IgG can be purified from such ascites fluids, or from culture supernatants, using column chromatography methods well known to those of skill in the art.

Unless otherwise indicated, an “antigen-binding fragment” means antigen-binding fragments of antibodies, i.e., antibody fragments that retain the ability to bind specifically to the antigen bound by the full-length antibody, e.g., fragments that retain one or more CDR regions. Examples of antigen-binding fragments include, but are not limited to, Fab, Fab′, F(ab′)2, and Fv fragments; diabodies; linear antibodies; single-chain antibody molecules, e.g., single chain Fv (ScFv); VHH (e.g., from species such as Camelidae or cartilaginous fish); nanobodies and antibodies formed from antibody fragments; and bicyclic peptides (Hurov, K. et al., 2021. Journal for Immuno Therapy of Cancer, 9(11)).

An antigen-binding fragment may be produced by any means. For example, in some embodiments, an antigen-binding fragment may be enzymatically or chemically produced by fragmentation of an intact antibody. In some embodiments, an antigen-binding fragment may be recombinantly produced (i.e., by expression of an engineered nucleic acid sequence. In some embodiments, an antigen-binding fragment may be wholly or partially synthetically produced. In some embodiments, an antigen-binding fragment may have a length of at least about 50, 60, 70, 80, 90, 100, 110, 120, 130, 140, 150, 160, 170, 180, or 190 amino acids or more (e.g., 50-100, 50-150, 50-200, or 100-200 amino acids). In some embodiments an antigen-binding fragment may have a length of at least about 200 amino acids.

A “target” is any molecule bound by a binding motif, antigen-binding system, or binding agent, e.g., an antibody. In some embodiments, a target is an antigen or epitope of the present disclosure. A “target cell” may express an antigen or epitope.

The term “affinity” as used herein refers to the strength of interaction between antibody and antigen. The variable regions of an antibody interact through non-covalent forces with an antigen at numerous sites. In general, the more interactions, the stronger the affinity.

An “affinity matured” antibody refers to an antibody with one or more alterations in one or more hypervariable regions (e.g., one or more CDRs) compared to a parent antibody that does not possess such alterations, such alterations resulting in an improvement in the affinity of the antibody for antigen. This process was used herein to humanize the parental mouse clones.

The term “binding” generally refers to a non-covalent association between or among two or more entities. “Direct” binding involves physical contact between entities or moieties. “Indirect” binding involves physical interaction by way of physical contact with one or more intermediate entities. Binding between two or more entities may be assessed in any of a variety of contexts, e.g., where interacting entities or moieties are studied in isolation or in the context of more complex systems (e.g., while covalently or otherwise associated with a carrier entity and/or in a biological system such as a cell).

“Binding affinity” generally refers to the strength of the sum total of non-covalent interactions between a single binding site of a molecule (e.g., an antibody) and its binding partner (e.g., an antigen). Binding affinity can be measured and/or expressed in a number of ways known in the art, including, but not limited to, equilibrium dissociation constant (K_{D}) and equilibrium association constant (K_{A}). Generally, the affinity of a molecule X for its partner Y can be represented by the equilibrium dissociation constant (K_{D}).

The term “equilibrium dissociation constant (K_{D})” refers to the dissociation rate constant (k_{off}(kd), time⁻¹) divided by the association rate constant (kₒₙ (ka), time⁻¹, M⁻¹). The kₒₙ and k_{off} can be determined by techniques known to one of ordinary skill in the art, such as BIACORE® or KinExA. Thus, the dissociation constant (K_{D}) is the concentration of a given antibody, such as an anti-FGFR2b antibody, or an antigen-binding fragment thereof, at which half of the available binding sites of FGFR2b target epitopes are occupied in the system at equilibrium. The smaller the K_{D} in molar concentration, the greater the affinity an antibody exhibits against its target antigen. Equilibrium dissociation constants can be measured using any known method in the art.

As used herein, an antibody or antigen-binding antibody fragment “specifically binds” or “selectively binds” to an antigen (e.g., a protein), meaning the antibody exhibits preferential binding to that target as compared to other proteins, but this specificity does not require absolute binding specificity. A “specific” or “selective” binding reaction is determinative of the presence of the antigen in a heterogeneous population of proteins and other biologics, for example, in a blood, serum, plasma, or tissue sample. Thus, under certain designated immunoassay conditions, the antibodies or antigen-binding fragments thereof specifically bind to a particular antigen at least two times greater when compared to the background level and do not specifically bind in a significant amount to other antigens present in the sample. In one aspect, under designated immunoassay conditions, the antibody or antigen-binding fragment thereof specifically binds to a particular antigen at least ten times greater when compared to the background level of binding and does not specifically bind in a significant amount to other antigens present in the sample.

In a specific embodiment, molecules that specifically bind to an antigen bind to the antigen with a K_{A} that is at least 2 logs, 2.5 logs, 3 logs, 4 logs, or greater than the K_{A} when the molecules bind to another antigen.

In further embodiments, a molecule selectively binds a target if binding between the molecule and the target is greater than 2-fold, greater than 5-fold, greater than 10-fold, 20-fold, 30-fold, 40-fold, 50-fold, 60-fold, 70-fold, 80-fold, 90-fold, or greater than 100-fold as compared with binding of the molecule and a non-target. In some embodiments, a molecule selectively binds a target if the binding affinity is less than about 10⁻⁵ M, less than about 10⁻⁶ M, less than about 10⁻⁷ M, less than about 10⁻⁷ M, or less than about 10⁻⁹ M.

In another embodiment, molecules that specifically bind to a target bind with a dissociation constant (K_{D}) of about 1×10⁻⁷ M. In some embodiments, an antigen-binding molecule specifically binds an antigen with “high affinity” when the K_{D} is about 1×10⁻⁹ M to about 5×10⁻⁹ M. In some embodiments, an antigen-binding molecule specifically binds an antigen with “very high affinity” when the K_{D} is 1×10⁻¹⁰ M to about 5×10⁻¹⁰ M. In one embodiment, an antigen-binding molecule has a K_{D} of 10⁻⁹ M. In one embodiment, the off-rate is less than about 1×10⁻⁵.

The term “human antibody” herein means an antibody that comprises only human immunoglobulin protein sequences. A human antibody can contain murine carbohydrate chains if produced in a mouse, in a mouse cell, or in a hybridoma derived from a mouse cell. Similarly, “mouse antibody” or “rat antibody” mean an antibody that comprises only mouse or rat immunoglobulin protein sequences, respectively.

The term “humanized” or “humanized antibody” means forms of antibodies that contain sequences from non-human (e.g., murine) antibodies as well as human antibodies. Such antibodies contain minimal sequence derived from non-human immunoglobulin. In general, a humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu,” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions can be included to increase affinity, increase stability of the humanized antibody, remove a post-translational modification, or for other reasons.

As used herein, “binding agent” or “binder” refers to any molecule, e.g., antibody, capable of binding with specificity to a given binding partner, e.g., antigen.

The term “identity” refers to the overall relatedness between polymeric molecules, e.g., between nucleic acid molecules (e.g., DNA molecules and/or RNA molecules) and/or between polypeptide molecules. The term “identical” or “percent identity” is a numeric score determined for a pair of aligned amino acid or nucleic acid sequences. Percent identity measures the number of identical residues (“identity”) between two sequences in relation to the length of the alignment across a “comparison window.” The number shows the % of amino acid residues or nucleotides that are the same between two sequences and indicates the degree of primary structure similarity.

Examples of algorithms that are suitable for determining percent sequence identity and sequence similarity are the BLAST algorithms, which are described in Altschul et al., Nuc. Acids Res. 25:3389-3402, 1977; and Altschul et al., J. Mol. Biol. 215:403-410, 1990. Software for performing BLAST analyses is publicly available through the National Center for Biotechnology Information.

The percent identity between two amino acid sequences can also be determined using the algorithm of E. Meyers and W. Miller, Comput. Appl. Biosci. 4:11-17, (1988), which has been incorporated into the ALIGN program (version 2.0), using a PAM120 weight residue table, a gap length penalty of 12, and a gap penalty of 4. In addition, the percent identity between two amino acid sequences can be determined using the Needleman and Wunsch, J. Mol. Biol. 48:444-453, (1970), algorithm which has been incorporated into the GAP program in the GCG software package using either a BLOSUM62 matrix or a PAM250 matrix, and a gap weight of 16, 14, 12, 10, 8, 6, or 4 and a length weight of 1, 2, 3, 4, 5, or 6.

As used herein, the term “amino acid” refers to an organic compound that contains amine (—NH₂) and carboxyl (—COOH) functional groups, along with a side chain (R group), which is specific to each amino acid. The term “amino acid” includes those that are natural or unnatural, proteinogenic or non-proteinogenic, synthetic, D or L optical isomers, and amino acid analogs and peptidomimetics. Naturally occurring amino acids are those encoded by the genetic code, as well as those amino acids that are later modified, e.g., hydroxyproline, γ-carboxyglutamate, and O-phosphoserine. By “proteinogenic” it is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein, or is not directly produced by cellular machinery (e.g., is the product of post-translational modification). Non-limiting examples of non-proteinogenic amino acids include gamma-aminobutyric acid (GABA), taurine (2-aminoethanesulfonic acid), theanine (L-γ-glutamylethylamide), hydroxyproline, beta-alanine, ornithine, and citrulline. Amino acid analogs refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Amino acid mimetics refer to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics that are not found in nature.

Families of amino acid residues having side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In certain embodiments, one or more amino acid residues within a CDR(s) or within a framework region(s) of an antibody or antigen-binding molecule thereof can be replaced with an amino acid residue with a similar side chain.

Exemplary amino acid categorizations are summarized in Tables 1 and 2 below.

TABLE 1
Amino Acid Categorizations

| | 3- | 1- | | | Hydropathy |
|---|---|---|---|---|---|
| Amino Acid | Letter | Letter | Property | Property | Index |
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

TABLE 2
Amino Acid Categorizations

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As to amino acid sequences, one of skill will recognize that individual substitutions, deletions or additions to a nucleic acid, peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. Such conservatively modified variants are in addition to and do not exclude polymorphic variants, interspecies homologs, and alleles.

The most commonly occurring exchanges are isoleucine/valine, tyrosine/phenylalanine, aspartic acid/glutamic acid, lysine/arginine, methionine/leucine, aspartic acid/asparagine, glutamic acid/glutamine, leucine/isoleucine, methionine/isoleucine, threonine/serine, tryptophan/phenylalanine, tyrosine/histidine, tyrosine/tryptophan, glutamine/arginine, histidine/asparagine, histidine/glutamine, lysine/asparagine, lysine/glutamine, lysine/glutamic acid, phenylalanine/leucine, phenylalanine/methionine, serine/alanine, serine/asparagine, valine/leucine, and valine/methionine. The following eight groups each contain exemplary amino acids that may be considered conservative substitutions for one another (see, e.g., Creighton, Proteins (1984)).
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)

TABLE 3
Individual Exemplary Conservative Amino Acid Substitutions

| Original residue | Conservative substitution |
|---|---|
| Ala (A) | Gly; Ser |
| Arg (R) | Lys, His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr(T) | Ser |
| Trp (W) | Tyr; Phe |
| Tyr (Y) | Trp; Phe |
| Val (V) | Ile; Leu |

A “conservative amino acid substitution” refers to substitutions of amino acids in a protein with other amino acids having similar characteristics (e.g., charge, side-chain size, hydrophobicity/hydrophilicity, backbone conformation and rigidity, etc.) such that the changes can frequently be made without altering the biological activity or other desired property of the protein, such as antigen affinity and/or specificity. Those of skill in the art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth above in Table 3.

As used herein “peptide,” in its various grammatical forms, is defined in its broadest sense to refer to a compound of two or more subunit amino acids, amino acid analogs, or other peptidomimetics. The subunits may be linked by peptide bonds or by other bonds, for example, ester, ether, and the like. If the peptide chain is short, e.g., two, three, or more amino acids, it is commonly called an oligopeptide. If the peptide chain is longer, the peptide is typically called a polypeptide or a protein. Full-length proteins, analogs, mutants, and fragments thereof are encompassed by the definition. The terms also include post-expression modifications of the polypeptide, for example, glycosylation, acetylation, phosphorylation and the like. Furthermore, as ionizable amino and carboxyl groups are present in the molecule, a particular peptide may be obtained as an acidic or basic salt, or in neutral form. A peptide may be obtained directly from the source organism, or may be recombinantly or synthetically produced.

The amino acid sequence of an antibody can be numbered using any known numbering scheme, including those described by Kabat et al., 1979, Dep't Health, Educ., Welfare, Pub. Health Serv., Nat'l Inst. Health (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme).

Example numbering of CDR amino acid residues in the heavy chain variable domain and the light chain variable domain for several numbering schemes are shown below in Table 4.

When an antibody is said to comprise CDRs by a certain definition of CDRs (e.g., Kabat) that definition specifies the minimum number of CDR residues present in the antibody (i.e., the Kabat CDRs). It does not exclude the possibility that other residues falling within another conventional CDR definition but outside the specified definition are also present. For example, an antibody comprising CDRs defined by Kabat may include, among other possibilities, an antibody in which the CDRs contain Kabat CDR residues and no other CDR residues, and an antibody in which HCDRl is a composite Chothia-Kabat HCDR1 and other CDRs contain Kabat CDR residues and no additional CDR residues based on other definitions.

Unless otherwise specified, the numbering scheme used herein is the Kabat numbering scheme. However, selection of a numbering scheme is not intended to imply differences in sequences where they do not exist, and one of skill in the art can readily confirm a sequence position by examining the amino acid sequence of one or more antibodies. Unless stated otherwise, the “EU numbering scheme” is generally used when referring to a residue in an antibody heavy chain constant region (e.g., as reported in Kabat et al., supra).

TABLE 4
Example CDR Numbering

| Loop | Kabat | AbM | Chothia | Contact |
|---|---|---|---|---|
| L1 | L24--L34 | L24--L34 | L24--L34 | L30--L36 |
| L2 | L50--L56 | L50--L56 | L50--L56 | L46--L55 |
| L3 | L89--L97 | L89--L97 | L89--L97 | L89--L96 |
| H1 | H31--H35B | H26--H35B | H26--H32 . . . 34 | H30--H35B |
| | (Kabat | | | |
| | Numbering) | | | |
| H1 | H31--H35 | H26--H35 | H26--H32 | H30--H35 |
| | (Chothia | | | |
| | Numbering) | | | |
| H2 | H50--H65 | H50--H58 | H52--H56 | H47--H58 |
| H3 | H95--H102 | H95--H102 | H95--H102 | H93--H101 |

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

“Effector cell” refers to a cell of the immune system that expresses one or more Fc receptors and mediates one or more effector functions. In some embodiments, effector cells may comprise, without limitation, one or more of monocytes, macrophages, neutrophils, dendritic cells, eosinophils, mast cells, platelets, large granular lymphocytes, Langerhans' cells, natural killer (NK) cells, T-lymphocytes, and B-lymphocytes. Effector cells may be of any organism including, without limitation, humans, mice, rats, rabbits, and monkeys.

“Effector function” refers to a biological result of an interaction of an antibody Fc region with an Fc receptor or ligand. Effector functions include, without limitation, antibody-dependent cell-mediated cytotoxicity (ADCC), antibody-dependent cell-mediated phagocytosis (ADCP), and complement-mediated cytotoxicity (CMC). An effector function may be antigen-binding dependent, antigen-binding independent, or both. ADCC refers to lysis of antibody-bound target cells by immune effector cells. Without wishing to be bound by any theory, ADCC is generally understood to involve Fc receptor (FcR)-bearing effector cells recognizing and subsequently killing antibody-coated target cells (e.g., cells that express on their surface antigens to which an antibody is bound). Effector cells that mediate ADCC may comprise immune cells, comprising yet not limited to, one or more of natural killer (NK) cells, macrophages, neutrophils, and eosinophils.

As used herein, the term “cell-killing activity” refers to the activity that decreases or reduces the cell viability of the tested cell line.

As used herein, the term “bystander killing” refers to the situation in which the drug from an ADC is released either from the target cell following internalization and degradation of the ADC, or release of the drug within the extracellular space. In both cases, the drug is then taken up by and kills surrounding or bystander cells, which themselves may or may not express the ADC target antigen.

As used herein, the term “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal, or epidermal administration (e.g., by injection or infusion).

The term “buffer” encompasses those agents which maintain the solution pH of the compositions of the present disclosure in an acceptable range.

The term “vector” refers to a recipient nucleic acid molecule modified to comprise or incorporate a provided nucleic acid sequence. One type of vector is a “plasmid,” which refers to a circular double stranded DNA molecule into which additional DNA may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) may be integrated into the genome of a host cell upon introduction into the host cell, and thereby are replicated along with the host genome. Moreover, certain vectors comprise sequences that direct expression of inserted genes to which they are operatively linked. Such vectors may be referred to herein as “expression vectors.” Standard techniques may be used for engineering of vectors, e.g., as found in Sambrook et al., Molecular Cloning: A Laboratory Manual (2d ed., Cold Spring Harbor Laboratory Press, Cold Spring Harbor, N.Y. (1989)).

As used herein, a “host cell” refers to any cell of any organism that is used for the purpose of producing a recombinant protein encoded by an expression vector or propagating the expression vector introduced into the host cell. A “mammalian recombinant host cell” refers to a mammalian host cell that comprises a heterologous expression vector, which may or may not be integrated into the host cell chromosome. A “bacterial recombinant host cell” refers to a bacterial host cell that comprises a heterologous expression vector, which may or may not be integrated into the host cell chromosome.

The term “toxin” or “payload” or “cytotoxic agent” is used herein to reference a molecule that inhibits or reduces the expression of molecules in cells, inhibits or reduces the function of cells, induces apoptosis of cells, and/or causes death of cells. The term includes radioactive isotopes, chemotherapeutic agents, and toxins such as small molecule toxins or enzymatically active toxins of bacterial, fungal, plant, or animal origin, including fragments and/or variants thereof. Examples of cytotoxic agents include, but are not limited to, auristatins (e.g., auristatin E, auristatin F, MMAE, and MMAF), auromycins, maytansinoids, pyrrolobenzodiazepine (PBD), ricin, ricin A-chain, combrestatin, duocarmycins, dolastatins, doxorubicin, daunorubicin, taxols, cisplatin, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, and calicheamicin, as well as radioisotopes such as At211, I131, I125, Y90, Re186, Re188, Sm153, Bi212 or 213, P32, and Lu177.

As used herein, the term “residue” refers to the chemical moiety within a compound that remains after a chemical reaction. For example, the term “amino acid residue” or “N-alkyl amino acid residue” refers to the product of an amide coupling or peptide coupling of an amino acid or a N-alkyl amino acid to a suitable coupling partner; wherein, for example, a water molecule is expelled after the amide or peptide coupling of the amino acid or the N-alkylamino acid, resulting in the product having the amino acid residue or N-alkyl amino acid residue incorporated therein.

As used herein, “sugar” or “sugar group” or “sugar residue” refers to a carbohydrate moiety which may comprise 3-carbon (triose) units, 4-carbon (tetrose) units, 5-carbon (pentose) units, 6-carbon (hexose) units, 7-carbon (heptose) units, or combinations thereof, and may be a monosaccharide, a disaccharide, a trisaccharide, a tetrasaccharide, a pentasaccharide, an oligosaccharide, or any other polysaccharide. In some instances, a “sugar” or “sugar group” or “sugar residue” comprises furanoses (e.g., ribofuranose, fructofuranose) or pyranoses (e.g., glucopyranose, galactopyranose), or a combination thereof. In some instances, a “sugar” or “sugar group” or “sugar residue” comprises aldoses or ketoses, or a combination thereof. Non-limiting examples of monosaccharides include ribose, deoxyribose, xylose, arabinose, glucose, mannose, galactose, and fructose. Non-limiting examples of disaccharides include sucrose, maltose, lactose, lactulose, and trehalose. Other “sugars” or “sugar groups” or “sugar residues” include polysaccharides and/or oligosaccharides, including, and not limited to, amylose, amylopectin, glycogen, inulin, and cellulose. In some instances, a “sugar” or “sugar group” or “sugar residue” is an amino-sugar. In some instances, a “sugar” or “sugar group” or “sugar residue” is a glucamine residue (1-amino-1-deoxy-D-glucitol) linked to the rest of a molecule via its amino group to form an amide linkage with the rest of the molecule (i.e., a glucamide).

An “alkyl” group is a saturated straight chain or branched non-cyclic hydrocarbon having from 1 to 10 carbon atoms, typically from 1 to 8 carbons or, in some embodiments, from 1 to 6, 1 to 4, or 2 to 6 carbon atoms. Representative alkyl groups include -methyl, -ethyl, -n-propyl, -n-butyl, -n-pentyl and n-hexyl; while saturated branched alkyls include -isopropyl, -sec-butyl, -isobutyl, -tert-butyl, -isopentyl, 2-methylpentyl, 3methylpentyl, 4-methylpentyl, 2,3-dimethylbutyl and the like. An alkyl group can be substituted or unsubstituted. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

An “alkenyl” group is a straight chain or branched non-cyclic hydrocarbon having from 2 to 10 carbon atoms, typically from 2 to 8 carbon atoms, and including at least one carbon-carbon double bond. Representative straight chain and branched (C₂-C₈)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, 2-hexenyl, -3-hexenyl, -1-heptenyl, -2-heptenyl, -3-heptenyl, -1-octenyl, -2-octenyl, -3-octenyl and the like. The double bond of an alkenyl group can be unconjugated or conjugated to another unsaturated group. An alkenyl group can be unsubstituted or substituted.

As used herein, “alkynyl” refers to a monovalent hydrocarbon radical moiety containing at least two carbon atoms and one or more carbon-carbon triple bonds. Alkynyl is optionally substituted and can be linear, branched, or cyclic. Alkynyl includes, but is not limited to, those radicals having 2-20 carbon atoms, i.e., C₂₋₂₀ alkynyl; 2-12 carbon atoms, i.e., C₂₋₁₂ alkynyl; 2-8 carbon atoms, i.e., C₂₋₈ alkynyl; 2-6 carbon atoms, i.e., C₂₋₆ alkynyl; and 2-4 carbon atoms, i.e., C₂₋₄ alkynyl. Examples of alkynyl moieties include, but are not limited to ethynyl, propynyl, and butynyl.

A “cycloalkyl” group is a saturated or a partially saturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as adamantyl and the like. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanone and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

As used herein, “aryloxy” refers to a monovalent moiety that is a radical of an aromatic compound wherein the ring atoms are carbon atoms and wherein the ring is substituted with an oxygen radical, i.e., the aromatic compound includes a single bond to an oxygen atom and wherein the radical is localized on the oxygen atom, e.g., C₆H₅—O—, for phenoxy. Aryloxy substituents bond to the compound which they substitute through this oxygen atom. Aryloxy is optionally substituted. Aryloxy includes, but is not limited to, those radicals having 6 to 20 ring carbon atoms, i.e., C₆₋₂₀ aryloxy; 6 to 15 ring carbon atoms, i.e., C₆₋₁₅ aryloxy; and 6 to 10 ring carbon atoms, i.e., C₆₋₁₀ aryloxy. Examples of aryloxy moieties include, but are not limited to phenoxy, naphthoxy, and anthroxy.

A “heteroaryl” group is an aryl ring system having one to four heteroatoms as ring atoms in a heteroaromatic ring system, wherein the remainder of the atoms are carbon atoms. In some embodiments, heteroaryl groups contain 5 to 6 ring atoms, and in others from 6 to 9 or even 6 to 10 atoms in the ring portions of the groups. Suitable heteroatoms include oxygen, sulfur, and nitrogen. In certain embodiments, the heteroaryl ring system is monocyclic or bicyclic. Non-limiting examples include, but are not limited to, groups such as pyrrolyl, pyrazolyl, imidazolyl, triazolyl, tetrazolyl, oxazolyl, isoxazolyl, thiazolyl, pyrrolyl, pyridyl, pyridazinyl, pyrimidinyl, pyrazinyl, thiophenyl, benzothiophenyl, furanyl, benzofuranyl (for example, isobenzofuran-1,3-diimine), indolyl, azaindolyl (for example, pyrrolopyridyl or 1H-pyrrolo[2,3-b]pyridyl), indazolyl, benzimidazolyl (for example, 1H-benzo[d]imidazolyl), imidazopyridyl (for example, azabenzimidazolyl, 3Himidazo[4,5-b]pyridyl, or 1H-imidazo[4,5-b]pyridyl), pyrazolopyridyl, triazolopyridyl, benzotriazolyl, benzoxazolyl, benzothiazolyl, benzothiadiazolyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups.

A “heterocyclyl” is a non-aromatic cycloalkyl in which one to four of the ring carbon atoms are independently replaced with a heteroatom independently selected from the group consisting of O, S, and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. Heterocyclyl groups encompass unsaturated, partially saturated, and saturated ring systems, such as, for example, imidazolyl, imidazolinyl and imidazolidinyl groups. The term “heterocyclyl” includes fused ring species, including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, pyrrolidyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl (for example, tetrahydro-2H-pyranyl), tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[l,3]dioxolyl, pyrazolopyridyl, imidazopyridyl (azabenzimidazolyl; for example, 1H-imidazo[4,5-b]pyridyl, or 1H-imidazo[4,5-b]pyridin-2(3H)-onyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

An “alkoxy” or “alkoxyl” group is —O(alkyl), wherein alkyl is defined above.

An “alkoxyalkyl” group is -(alkyl)O(alkyl), wherein each alkyl is independently as defined above.

An “amine” group is a radical of the formula: —NH₂.

A “hydroxyl amine” group is a radical of the formula: N(R^{#})OH or NHOH, wherein R^{#} is a substituted or unsubstituted alkyl, cycloalkyl, cycloalkylalkyl, aryl, aralkyl, heterocyclyl, or heterocyclylalkyl group as defined herein.

An “alkoxyamine” group is a radical of the formula: —N(R^{#})O-alkyl or —NHO-alkyl, wherein R^{#} is as defined above.

An “aralkoxyamine” group is a radical of the formula: N(R^{#})O-aryl or NHOaryl, wherein R^{#} is as defined above.

An “aminocarbonyl” group is a radical of the formula: —C(═O)N(R^{#})₂, —C(═O)NH(R^{#}), or C(═O)NH₂, wherein each R^{#} is as defined above.

An “acylamino” group is a radical of the formula: NHC(═O)(R^{#}) or N(alkyl)C(═O)(R^{#}), wherein each alkyl and R^{#} are independently as defined above.

An “O(alkyl)aminocarbonyl” group is a radical of the formula: —O(alkyl)C(═O)N(R^{#})₂, —O(alkyl)C(═O)NH(R^{#}), or —O(alkyl)C(═O)NH₂, wherein each R^{#} is independently as defined above.

An “N-oxide” group is a radical of the formula: —N⁺—O⁻.

A “carboxy” group is a radical of the formula: C(═O)OH.

A “ketone” group is a radical of the formula: C(═O)(R^{#}), wherein R^{#} is as defined above.

An “aldehyde” group is a radical of the formula: —CH(═O).

An “ester” group is a radical of the formula: C(═O)O(R^{#}) or OC(═O)(R^{#}), wherein R^{#} is as defined above.

A “urea” group is a radical of the formula: —N(alkyl)C(═O)N(R^{#})₂, —N(alkyl)C(═O)NH(R^{#}), —N(alkyl)C(═O)NH₂, —NHC(═O)N(R^{#})₂, —NHC(═O)NH(R^{#}), or NHC(═O)NH₂^{#}, wherein each alkyl and R^{#} are independently as defined above.

An “imine” group is a radical of the formula: —N═C(R^{#})₂ or —C(R^{#})═N(R^{#}), wherein each R^{#} is independently as defined above.

An “imide” group is a radical of the formula: —C(═O)N(R#)C(═O)(R^{#}) or N((C═O)(R^{#}))₂, wherein each R^{#} is independently as defined above.

A “urethane” group is a radical of the formula: —OC(═O)N(R^{#})₂, —OC(═O)NH(R^{#}), —N(R^{#})C(═O)O(R^{#}), or —NHC(═O)O(R^{#}), wherein each R^{#} is independently as defined above.

An “amidine” group is a radical of the formula: —C(═N(R^{#}))N(R^{#})₂, —C(═N(R^{#}))NH(R^{#}), —C(═N(R^{#}))NH₂, —C(═NH)N(R^{#})₂, —C(═NH)NH(R^{#}), —C(═NH)NH₂, —N═C(R^{#})N(R^{#})₂, —N═C(R^{#})NH(R^{#}), —N═C(R^{#})NH₂, —N(R^{#})C(R^{#})═N(R^{#}), —NHC(R^{#})═N(R^{#}), —N(R^{#})C(R^{#})═NH, or —NHC(R^{#})═NH, wherein each R^{#} is independently as defined above.

A “guanidine” group is a radical of the formula: —N(R^{#})C(═N(R^{#}))N(R^{#})₂, —NHC(═N(R^{#}))N(R^{#})₂, —N(R^{#})C(═NH)N(R^{#})₂, —N(R^{#})C(═N(R^{#}))NH(R^{#}), —N(R^{#})C(═N(R^{#}))NH₂, —NHC(═NH)N(R^{#})₂, —NHC(═N(R^{#}))NH(R^{#}), —NHC(═N(R^{#}))NH₂, —NHC(═NH)NH(R^{#}), —NHC(═NH)NH₂, —N═C(N(R^{#})₂)₂, —N═C(NH(R^{#}))₂, or —N═C(NH₂)₂, wherein each R^{#} is independently as defined above.

An “enamine” group is a radical of the formula: —N(R^{#})C(R^{#})═C(R^{#})₂, —NHC(R^{#})═C(R^{#})₂, —C(N(R^{#})₂)═C(R^{#})₂, —C(NH(R^{#}))═C(R^{#})₂, —C(NH₂)═C(R^{#})₂, —C(R^{#})═C(R^{#})(N(R^{#})₂), C(R^{#})═C(R^{#})(NH(R^{#})) or —C(R^{#})═C(R^{#})(NH₂), wherein each R^{#} is independently as defined above.

An “oxime” group is a radical of the formula: —C(═NO(R^{#}))(R^{#}), —C(═NOH)(R^{#}), —CH(═NO(R^{#})), or —CH(═NOH), wherein each R^{#} is independently as defined above.

A “hydrazide” group is a radical of the formula: —C(═O)N(R^{#})N(R^{#})₂, —C(═O)NHN(R^{#})₂, —C(═O)N(R^{#})NH(R^{#}), —C(═O)N(R^{#})NH₂, —C(═O)NHNH(R^{#})₂, or —C(═O)NHNH₂, wherein each R^{#} is independently as defined above.

A “hydrazine” group is a radical of the formula: —N(R^{#})N(R^{#})₂, —NHN(R^{#})₂, —N(R^{#})NH(R^{#}), —N(R^{#})NH₂, —NHNH(R^{#})₂, or —NHNH₂, wherein each R^{#} is independently as defined above.

A “hydrazone” group is a radical of the formula: —C(═N—N(R^{#})₂)(R^{#})₂, —C(═NNH(R^{#}))(R^{#})₂, —C(═N—NH₂)(R^{#})₂, —N(R^{#})(N═C(R^{#})₂), or —NH(N═C(R^{#})₂), wherein each R^{#} is independently as defined above.

An “azide” group is a radical of the formula: —N₃.

An “isocyanate” group is a radical of the formula: N═C═O.

An “isothiocyanate” group is a radical of the formula: N═C═S.

A “cyanate” group is a radical of the formula: OCN.

A “thiocyanate” group is a radical of the formula: SCN.

A “thioether” group is a radical of the formula; —S(R^{#}), wherein R^{#} is as defined above.

A “thiocarbonyl” group is a radical of the formula: —C(═S)(R^{#}), wherein R^{#} is as defined above.

A “sulfinyl” group is a radical of the formula: —S(═O)(R^{#}), wherein R^{#} is as defined above.

A “sulfone” group is a radical of the formula: —S(═O)₂(R^{#}), wherein R^{#} is as defined above.

A “sulfonamide” group is a radical of the formula: —S(═O)₂N(R^{#})₂, —S(═O)₂NH(R^{#}), or —S(═O)₂NH₂, wherein each R^{#} is independently as defined above.

A “phosphonate” group is a radical of the formula: —P(═O)(O(R^{#}))₂, —P(═O)(OH)₂, —OP(═O)(O(R^{#}))(R^{#}), or —OP(═O)(OH)(R^{#}), wherein each R^{#} is independently as defined above.

A “phosphine” group is a radical of the formula: —P(R^{#})₂, wherein each R^{#} is independently as defined above.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl) aryloxy; aralkyloxy; heterocyclyloxy; and heterocyclyl alkoxy.

As used herein, the term “pharmaceutically acceptable salt(s)” refers to a salt prepared from a pharmaceutically acceptable non-toxic acid or base including an inorganic acid and base and an organic acid and base.

As used herein and unless otherwise indicated, the term “solvate” means a compound, or a salt thereof, that further includes a stoichiometric or non-stoichiometric amount of a solvent bound by non-covalent intermolecular forces. In one embodiment, the solvate is a hydrate.

As used herein and unless otherwise indicated, the term “hydrate” means a compound, or a salt thereof, that further includes a stoichiometric or non-stoichiometric amount of water bound by non-covalent intermolecular forces.

All pharmaceutically acceptable salts, solvates, and/or hydrates of compounds depicted herein are within the scope of the present disclosure.

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof. The use of stereomerically pure forms of such compounds, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley Interscience, New York, 1981); Wilen, S. H., et al., Tetrahedron 33:2725 (1977); Eliel, E. L., Stereochemistry of Carbon Compounds (McGrawHill, NY, 1962); and Wilen, S. H., Tables of Resolving Agents and Optical Resolutions p. 268 (E. L. Eliel, Ed., Univ. of Notre Dame Press, Notre Dame, IN, 1972).

It should also be noted the compounds can include E and Z isomers, or a mixture thereof, and cis and trans isomers or a mixture thereof. In certain embodiments, the compounds are isolated as either the cis or trans isomer. In other embodiments, the compounds are a mixture of the cis and trans isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in an aqueous solution, pyrazoles may exhibit the following isomeric forms, which are referred to as tautomers of each other:

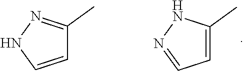

As readily understood by one skilled in the art, a wide variety of functional groups and other structures may exhibit tautomerism and all tautomers of the compounds are within the scope of the present disclosure.

It should also be noted the compounds can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds.

Certain groups, moieties, substituents, and atoms are depicted with a wiggly line that intersects a bond or bonds to indicate the atom through which the groups, moieties, substituents, or atoms are bonded. For example, a phenyl group that is substituted with a propyl group depicted as:

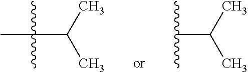

has the following structure:

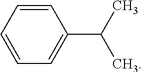

As used herein, illustrations showing substituents bonded to a cyclic group (e.g., aromatic, heteroaromatic, fused ring, and saturated or unsaturated cycloalkyl or heterocycloalkyl) through a bond between ring atoms are meant to indicate, unless specified otherwise, that the cyclic group may be substituted with that substituent at any ring position in the cyclic group or on any ring in the fused ring group, according to techniques set forth herein or which are known in the field to which the instant disclosure pertains.

Illustrations showing substituents bonded to a non-cyclic group through a bond between two atoms are meant to indicate, unless specified otherwise, that the substituent may be bonded to either atom of the bond through which the substituent bond passes, according to techniques set forth herein or which are known in the field to which the instant disclosure pertains. Thus, for example,

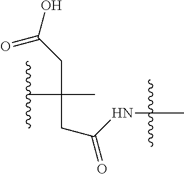 [SMILES: *NC(=O)CC(*)(C)CC(=O)O]

encompasses

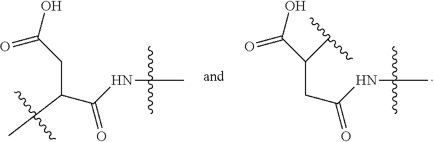 [SMILES: *NC(=O)C(*)CC(=O)O ; *NC(=O)CC(*)C(=O)O]

It should be noted that if there is a discrepancy between a depicted structure and a name for that structure, the depicted structure is to be accorded more weight.

In the claims which follow and in the preceding description, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the disclosure.

### DETAILED DESCRIPTION

The present disclosure provides for antibodies, antigen-binding fragments thereof, and antibody drug conjugates that specifically bind human FGFR2b. Furthermore, the present disclosure provides antibodies, antigen-binding fragments thereof, and antibody drug conjugates that have desirable pharmacokinetic characteristics and other desirable attributes, and thus can be used for reducing the likelihood of or treating cancer. The present disclosure further provides pharmaceutical compositions comprising the antibodies antigen-binding fragments thereof, and antibody drug conjugates and methods of making and using such pharmaceutical compositions for the prevention and treatment of FGFR2b-associated disorders.

### Anti-FGFR2b Antibodies

The present disclosure provides for antibodies and antigen-binding fragments thereof that specifically bind to FGFR2b. The antibodies and antigen-binding fragments may be, for example, monoclonal antibodies, chimeric antibodies, humanized antibodies, human engineered antibodies, single chain antibodies (scFv), Fab fragments, Fab′ fragments, or F(ab′)₂ fragments. Antibodies and antigen-binding fragments of the present disclosure include, but are not limited to, each of the antibodies or antigen-binding fragments thereof described and generated as described below.

In embodiments, an antibody or antigen-binding fragment thereof provided herein binds to a target human antigen, e.g., human FGFR2b, with higher affinity than to another species of the target antigen, e.g., a non-human FGFR2b. In embodiments, an antibody, or an antigen-binding fragment thereof, that binds to human FGFR2b with a 5%, 10%, 15%, 20%, 25%, 30%, 35%, 40%, 45%, 50%, 55%, 60%, 65%, 70% or higher affinity than to another species of FGFR2b as measured by, e.g., a radioimmunoassay, surface plasmon resonance, or kinetic exclusion assay. In embodiments, an antibody, or an antigen-binding fragment thereof, described herein, which binds to a target human antigen, will bind to another species of the target antigen with less than 10%, 15%, or 20% of the binding of the antibody or an antigen-binding fragment thereof, to the human antigen as measured by, e.g., a radioimmunoassay, surface plasmon resonance, or kinetic exclusion assay.

In embodiments, the antigen-binding molecule binds human FGFR2b with a K_{D} of between about 1×10⁻⁷ M and about 1×10⁻¹³ M. In another embodiment, the antigen-binding molecule binds human FGFR2b with a K_{D} of about 1×10⁻¹⁰ M to about 5×10⁻¹⁰ M. In some embodiments, the antigen-binding molecule binds human FGFR2b with a K_{D} of between about 1×10⁻⁷ M and about 1×10⁻¹³ M. In yet another embodiment, the antigen-binding molecule binds human FGFR2b with a K_{D} of about 1×10⁻¹⁰ M to about 5×10⁻¹⁰ M.

In one embodiment, the extent of binding of an anti-FGFR2b antibody to an unrelated, non-FGFR2b protein is less than about 10% of the binding of the antibody to FGFR2b as measured, e.g., by a radioimmunoassay (RIA). In certain embodiments, an antibody that binds to FGFR2b has a dissociation constant (K_{D}) of ≤1 M, ≤100 nM, ≤10 nM, ≤5 nM, ≤4 nM, ≤3 nM, ≤2 nM, ≤1 nM, ≤0.1 nM, ≤0.01 nM, or ≤0.001 nM (e.g., 10⁻⁸ M or less, from 10⁻⁸ M to 10⁻¹³ M, from 10⁻⁹ M to 10⁻¹³ M).

In embodiments, the disclosed antibodies and antigen-binding fragments thereof that specifically bind to FGFR2b comprise a heavy chain variable region comprising one, two, or three of the following: a heavy chain complementarity determining region (HCDR) 1 comprising the amino acid sequence IYWX₁N, in which X₁ is M or L; an HCDR2 comprising the amino acid sequence IYPENX₂DTNYX₃GKFKG, in which X₂ is G or A and X₃ is S or N; and an HCDR3 comprising the amino acid sequence GGFDY. In embodiments, the disclosed antibodies and antigen-binding fragments thereof that specifically bind to FGFR2b comprise a heavy chain variable region comprising one, two, or three of the following: an HCDR 1 comprising the amino acid sequence IYWLN (SEQ ID NO: 3); an HCDR2 comprising the amino acid sequence QIYPENADTNYSGKFKG (SEQ ID NO: 4); and an HCDR3 comprising the amino acid sequence GGFDY (SEQ ID NO: 5).

In embodiments, the disclosed antibodies and antigen-binding fragments thereof that specifically bind to FGFR2b comprise a light chain variable region comprising one, two, or three of the following: a light chain complementarity determining region (LCDR) 1 comprising the amino acid sequence RASENIYSNLA; an LCDR2 comprising the amino acid sequence TATNLAX₄, in which X₄ is D or E; and an LCDR3 comprising the amino acid sequence QHFYGILYT. In embodiments, the disclosed antibodies and antigen-binding fragments thereof that specifically bind to FGFR2b comprise a light chain variable region comprising one, two, or three of the following: an LCDR1 comprising the amino acid sequence RASENIYSNLA (SEQ ID NO: 6); an LCDR2 comprising the amino acid sequence TATNLAE (SEQ ID NO: 7); and an LCDR3 comprising the amino acid sequence QHFYGILYT (SEQ ID NO: 8).

In embodiments, the disclosed antibodies and antigen-binding fragments thereof that specifically bind to FGFR2b comprise (a) a heavy chain variable region comprising HCDR 1 comprising the amino acid sequence IYWLN (SEQ ID NO: 3), HCDR2 comprising the amino acid sequence QIYPENADTNYSGKFKG (SEQ ID NO: 4), and an HCDR3 comprising the amino acid sequence GGFDY (SEQ ID NO: 5); and (b) a light chain variable region comprising LCDR1 comprising the amino acid sequence RASENIYSNLA (SEQ ID NO: 6); LCDR2 comprising the amino acid sequence TATNLAE (SEQ ID NO: 7); and LCDR3 comprising the amino acid sequence QHFYGILYT (SEQ ID NO: 8).

In embodiments, the disclosed antibodies and antigen-binding fragments thereof that specifically bind to FGFR2b comprise a heavy chain variable region comprising one, two, or three of the following: an HCDR 1 comprising the amino acid sequence DTYIH; an HCDR2 comprising the amino acid sequence RIDPAX₁GNTMFASEFQG, in which X₁ is E or N; and an HCDR3 comprising the amino acid sequence SKIHYDYDEGFAY. In embodiments, the disclosed antibodies and antigen-binding fragments thereof that specifically bind to FGFR2b comprise a heavy chain variable region comprising one, two, or three of the following: an HCDR1 having the amino acid sequence DTYIH (SEQ ID NO: 15); an HCDR2 having the amino acid sequence RIDPAEGNTMFASEFQG (SEQ ID NO: 16); and an HCDR3 having the amino acid sequence SKIHYDYDEGFAY (SEQ ID NO: 17).

In embodiments, the disclosed antibodies and antigen-binding fragments thereof that specifically bind to FGFR2b comprise a light chain variable region comprising one, two, or three of the following: an LCDR 1 comprising the amino acid sequence RASESVDDYGYSFLH (SEQ ID NO: 18); an LCDR2 comprising the amino acid sequence RASNLES (SEQ ID NO: 19); and an LCDR3 comprising the amino acid sequence QQSNQNPRT (SEQ ID NO: 20).

In some embodiments, the present disclosure provides antibodies and antigen-binding fragments that specifically bind to FGFR2b, wherein said antibodies and antibody fragments (e.g., antigen-binding fragments) comprise a VH domain comprising an amino acid sequence as set forth in SEQ ID NO: 9 or SEQ ID NO: 21 (Table 8), or an amino acid sequence having at least 85%, 90%, 95%, 96%, 97%, 98%, or 99% identity to a sequence as set forth in SEQ ID NO: 9 or SEQ ID NO: 21. In some embodiments, the present disclosure provides antibodies and antigen-binding fragments that specifically bind to FGFR2b, wherein said antibodies and antibody fragments (e.g., antigen-binding fragments) comprise a VH domain comprising an amino acid sequence as set forth in SEQ ID NO: 9 or SEQ ID NO: 21 (Table 8), or an amino acid sequence having at least 85%, 90%, 95%, 96%, 97%, 98%, or 99% identity to a sequence as set forth in SEQ ID NO: 9 or SEQ ID NO: 21, wherein the antibodies or antigen-binding fragments thereof comprise the 6 complementarity determining regions (CDRs) within SEQ ID NO: 9 or SEQ ID NO: 21 as shown in Table 8. The present disclosure also provides antibodies or antigen-binding fragments that specifically bind FGFR2b, wherein said antibodies or antigen-binding fragments comprise a heavy chain CDR (HCDR) comprising an amino acid sequence of any one of the HCDRs listed in Table 8. In one aspect, the present disclosure provides antibodies or antigen-binding fragments that specifically bind to FGFR2b, wherein said antibodies comprise (or alternatively, consist of) one, two, three, or more HCDRs comprising an amino acid sequence of any of the HCDRs listed in Table 8.

The present disclosure provides for antibodies and antigen-binding fragments that specifically bind to FGFR2b, wherein said antibodies or antigen-binding fragments comprise a VH domain comprising an amino acid sequence of SEQ ID NO: 9 or SEQ ID NO: 21 (Table 8), or an amino acid sequence having at least 85%, 90%, 95%, 96%, 97%, 98%, or 99% identity to a sequence as set forth in SEQ ID NO: 9 or SEQ ID NO: 21, and a VL domain comprising an amino acid sequence of SEQ ID NO: 10 or SEQ ID NO: 22 or an amino acid sequence having at least 85%, 90%, 95%, 96%, 97%, 98%, or 99% identity to a sequence as set forth in SEQ ID NO: 10 or SEQ ID NO: 22 (Table 8). The present disclosure also provides antibodies and antigen-binding fragments that specifically bind to FGFR2b, wherein said antibodies or antigen-binding fragments comprise the light chain CDRs (LCDRs) and HCDRs of a single antibody listed in Table 8. In particular, the disclosure provides for antibodies or antigen-binding fragments that specifically bind to FGFR2b, said antibodies or antigen-binding fragments comprising (or alternatively, consisting of) one, two, three or more LCDRs and one, two, three or more HCDRs of any one antibody listed in Table 8.

Because specific binding was achieved with heavy and light chain variable regions having as little as 78% sequence identity to each other, there is structural support for claims directed to a genus of antibodies having a heavy chain variable region (HCVR) comprising an amino acid sequence having at least 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 9 or SEQ ID NO: 21, and a light chain variable region (LCVR) comprising an amino sequence having at least 82%, 83%, 84%, 85%, 86%, 87%, 88%, 89%, 90%, 91%, 92%, 93%, 94%, 95%, 96%, 97%, 98%, or 99% sequence identity to SEQ ID NO: 10 or SEQ ID NO: 22.

In certain embodiments, a HCVR sequence having at least 82%, 83%, 84%, 85%, 86%, 87%, 88%, 89%, 90%, 91%, 92%, 93%, 94%, 95%, 96%, 97%, 98%, or 99% amino acid sequence identity to the amino acid sequence of SEQ ID NO: 9 or SEQ ID NO: 21 contains substitutions (e.g., conservative substitutions), insertions, or deletions relative to the reference sequence, but an anti-FGFR2b antibody or antigen-binding fragment thereof comprising that sequence retains the ability to specifically bind to FGFR2b. In certain embodiments, a total of 1 to 10 amino acids have been substituted, inserted, and/or deleted in SEQ ID NO: 9 or SEQ ID NO: 21. In certain embodiments, a total of 1 to 5 amino acids have been substituted, inserted, and/or deleted in SEQ ID NO: 9 or SEQ ID NO: 21. In certain embodiments, the substitutions, insertions, or deletions occur in regions outside the CDRs (i.e., in the FRs).

In certain embodiments, a LCVR sequence having at least 83%, 84%, 85%, 86%, 87%, 88%, 89%, 90%, 91%, 92%, 93%, 94%, 95%, 96%, 97%, 98%, or 99% amino acid sequence identity to the amino acid sequence of SEQ ID NO: 10 or SEQ ID NO: 22 contains substitutions (e.g., conservative substitutions), insertions, or deletions relative to the reference sequence, but an anti-FGFR2b antibody or antigen-binding fragment thereof comprising that sequence retains the ability to specifically bind to FGFR2b. In certain embodiments, a total of 1 to 10 amino acids have been substituted, inserted, and/or deleted in SEQ ID NO: 10 or SEQ ID NO: 22. In certain embodiments, a total of 1 to 5 amino acids have been substituted, inserted, and/or deleted in SEQ ID NO: 10 or SEQ ID NO: 22. In certain embodiments, the substitutions, insertions, or deletions occur in regions outside the CDRs (i.e., in the FRs).

The structural consensus sequences disclosed herein are intended to guide those skilled in the art as to how to predict other species that fall within the claimed structural/functional genus of specifically binding to FGFR2b. The disclosure provides common structural features of a genus of antibodies, or antigen-binding fragments thereof, that correlate with a recited function. The common structural features disclosed as species are representative of the full variety of the claimed genus.

### Alteration of the Framework of Fc Region

In aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al.

In another aspect, one or more amino acid residues can be replaced with one or more different amino acid residues such that the antibody has altered C1q binding and/or reduced or abolished complement dependent cytotoxicity (CDC). This approach is described in, e.g., U.S. Pat. No. 6,194,551 by Idusogie et al.

In another aspect, one or more amino acid residues are changed to thereby alter the ability of the antibody to fix complement. This approach is described in, e.g., the publication WO 94/29351 by Bodmer et al. In a specific aspect, one or more amino acids of an antibody or antigen-binding fragment thereof of the present disclosure are replaced by one or more allotypic amino acid residues for the IgG1 subclass and the kappa isotype. Allotypic amino acid residues also include, but are not limited to, the constant region of the heavy chain of the IgG1, IgG2, and IgG3 subclasses as well as the constant region of the light chain of the kappa isotype as described by Jefferis et al., MAbs. 1:332-338 (2009).

In another aspect, the Fc region is modified to increase the ability of the antibody to mediate antibody dependent cellular cytotoxicity (ADCC) and/or to increase the affinity of the antibody for an Fcγ receptor by modifying one or more amino acids. This approach is described in, e.g., the publication WO00/42072 by Presta. Moreover, the binding sites on human IgG1 for FcγRI, FcγRII, FcγRIII, and FcRn have been mapped and variants with improved binding have been described (see Shields et al., J. Biol. Chem. 276:6591-6604, 2001).

In another aspect, the glycosylation of the antibody is modified. For example, an aglycosylated antibody can be made (i.e., the antibody lacks or has reduced glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for an antigen. Such an approach is described in, e.g., U.S. Pat. Nos. 5,714,350 and 6,350,861 by Co et al.

Additionally, or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with an altered glycosylation pathway. Cells with altered glycosylation pathways have been described in the art and can be used as host cells in which to express recombinant antibodies to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. Publication WO 03/035835 by Presta describes a variant CHO cell line, Lecl3 cells, with reduced ability to attach fucose to Asn (297)-linked carbohydrates, also resulting in hypofucosylation of antibodies expressed in that host cell (see also Shields et al., (2002) J. Biol. Chem. 277:26733-26740). WO99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures, which results in increased ADCC activity of the antibodies (see also Umana et al., Nat. Biotech. 17:176-180, 1999).

In another aspect, if a reduction of ADCC is desired, human antibody subclass IgG4 was shown in many previous reports to have only modest ADCC and almost no CDC effector function (Moore G. L., et al., 2010 MAbs, 2:181-189). However, natural IgG4 was found less stable in stress conditions such as in acidic buffer or under increasing temperature (Angal, S. 1993 Mol Immunol, 30:105-108; Dall'Acqua, W. et al., 1998 Biochemistry, 37:9266-9273; Aalberse et al., 2002 Immunol, 105:9-19). Reduced ADCC can be achieved by operably linking the antibody to an IgG4 Fc engineered with combinations of alterations that reduce FcγR binding or C1q binding activities, thereby reducing or eliminating ADCC and CDC effector functions. Considering the physicochemical properties of an antibody as a biological drug, one of the less desirable, intrinsic properties of IgG4 is dynamic separation of its two heavy chains in solution to form half antibody, which lead to bi-specific antibodies generated in vivo via a process called “Fab arm exchange” (Van der Neut Kolfschoten M., et al., 2007 Science, 317:1554-157). The mutation of serine to proline at position 228 (EU numbering system) appeared inhibitory to the IgG4 heavy chain separation (Angal, S. 1993 Mol Immunol, 30:105-108; Aalberse et al., 2002 Immunol, 105:9-19). Some of the amino acid residues in the hinge and γFc region were reported to have impact on antibody interaction with Fcγ receptors (Chappel S. M., et al., 1991 Proc. Natl. Acad. Sci. USA, 88:9036-9040; Mukherjee, J. et al., 1995 FASEB J, 9:115-119; Armour, K. L. et al., 1999 Eur J Immunol, 29:2613-2624; Clynes, R. A. et al, 2000 Nature Medicine, 6:443-446; Arnold J. N., 2007 Annu Rev Immunol, 25:21-50). Furthermore, some rarely occurring IgG4 isoforms in human population can also elicit different physicochemical properties (Brusco, A. et al., 1998 Eur J Immunogenet, 25:349-55; Aalberse et al., 2002 Immunol, 105:9-19). To generate antibodies with low ADCC and CDC but with good stability, it is possible to modify the hinge and Fc region of human IgG4 and introduce a number of alterations. These modified IgG4 Fc molecules can be found in SEq ID NOs:83-88, U.S. Pat. No. 8,735,553 to Li et al.

### FGFR2b Antibody Production

Anti-FGFR2b antibodies and antigen-binding fragments thereof, including those for the disclosed ADCs, can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The present disclosure further provides polynucleotides encoding the antibodies described herein, e.g., polynucleotides encoding heavy or light chain variable regions or segments comprising the complementarity determining regions as described herein. In some aspects, the polynucleotide encoding a heavy chain variable region has at least 85%, 89%, 90%, 91%, 92%, 93%, 94%, 95%, 96%, 97%, 98%, 99%, or 100% nucleic acid sequence identity with a polynucleotide represented by SEQ ID NO: 13 or SEQ ID NO: 25. In some aspects, the polynucleotide encoding a light chain variable region has at least 85%, 89%, 90%, 91%, 92%, 93%, 94%, 95%, 96%, 97%, 98%, 99%, or 100% nucleic acid sequence identity with SEQ ID NO: 14 or SEQ ID NO: 26. In some embodiments, an antibody as described herein is encoded by a polynucleotide encoding a heavy chain variable region having at least 85%, 89%, 90%, 91%, 92%, 93%, 94%, 95%, 96%, 97%, 98%, 99%, or 100% nucleic acid sequence identity with a polynucleotide represented by SEQ ID NO: 13 or SEQ ID NO: 25, and a polynucleotide encoding a light chain variable region having at least 85%, 89%, 90%, 91%, 92%, 93%, 94%, 95%, 96%, 97%, 98%, 99%, or 100% nucleic acid sequence identity with SEQ ID NO: 14 or SEQ ID NO: 26. SEQ ID NOs: 13 and 14 may be paired as heavy and light chains, respectively, and SEQ ID NOs: 25 and 26 may be paired as heavy and light chains, respectively.

The polynucleotides of the present disclosure can encode the variable region sequence of an anti-FGFR2b antibody. They can also encode both a variable region and a constant region of the antibody. Some of the polynucleotide sequences encode a polypeptide that comprises variable regions of both the heavy chain and the light chain of the exemplified anti-FGFR2b antibodies.

Also provided in the present disclosure are expression vectors and host cells for producing the anti-FGFR2b antibodies. In embodiments, the expression vectors comprise the polynucleotides encoding the antibodies described herein. In embodiments, the host cells comprise the polynucleotides encoding the antibodies described herein. In embodiments, the host cells comprise the vectors comprising the polynucleotides encoding the antibodies described herein.

The choice of expression vector depends on the intended host cells in which the vector is to be expressed. Typically, the expression vectors contain a promoter and other regulatory sequences (e.g., enhancers) that are operably linked to the polynucleotides encoding an anti-FGFR2b antibody chain or antigen-binding fragment. In some aspects, an inducible promoter is employed to prevent expression of inserted sequences except under the control of inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements can also be included for efficient expression of an anti-FGFR2b antibody or antigen-binding fragment thereof. These elements may include an ATG initiation codon and adjacent ribosome binding site or other sequences. In addition, the efficiency of expression can be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer can be used to increase expression in mammalian host cells.

The host cells for harboring and expressing the anti-FGFR2b antibody vectors can be either prokaryotic or eukaryotic. E. coli is one prokaryotic host useful for cloning and expressing the polynucleotides of the present disclosure. Other microbial hosts suitable for use include bacilli, such as Bacillus subtilis, and other Enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters may be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like for initiating and completing transcription and translation. Other microbes, such as yeast, can also be employed to express anti-FGFR2b antibodies. Insect cells in combination with baculovirus vectors can also be used.

In other aspects, mammalian host cells are used to express and produce the anti-FGFR2b antibodies of the present disclosure. Examples include a hybridoma cell line expressing endogenous immunoglobulin genes or a mammalian cell line harboring an exogenous expression vector. These include any normal mortal or normal or abnormal immortal animal or human cells. For example, several suitable host cell lines capable of secreting intact immunoglobulins have been developed, including the CHO cell lines, various COS cell lines, HEK 293 cells, myeloma cell lines, transformed B-cells, and hybridomas. The use of mammalian tissue cell culture to express polypeptides is discussed generally in, e.g., Winnacker, From Genes to Clones, VCH Publishers, NY, N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters can be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

Any of the foregoing host cells can be modified with any of the foregoing expression vectors by transient or stable transfection. Suitable transfection methods include using a cationic lipid, polyethylenimine, Lipofectamine™, or ExpiFectamine™, or electroporation, or other means. The skilled practitioner is aware of numerous suitable means for transfecting to achieve expression of recombinant antibodies.

Upon culturing the transfected or transformed host cells, the anti-FGFR2b antibodies, or antigen-binding fragments thereof, of the present disclosure may be directly secreted into the cell culture medium (such as by employing appropriate secretory-directing signal peptides) and may be recovered therefrom.

In embodiments, the recovered anti-FGFR2b antibodies, or antigen-binding fragments thereof, may be used in a composition. In embodiments, the anti-FGFR2b antibodies, or antigen-binding fragments thereof are first conjugated or linked to at least one therapeutic agent via linkers to form conjugate, which are described in more detail below.

### Conjugated Anti-FGFR2b Antibodies and Antigen-Binding Fragments Thereof

The present disclosure provides, inter alia, anti-FGFR2b antibodies and antigen-binding fragments thereof conjugated via a linker to one or more therapeutic agents to produce conjugates, which are also referred to here as an “immunoconjugates” or “antibody drug conjugates.” In some embodiments, a therapeutic agent comprises or is a cytotoxic agent, a drug, and/or a radioisotope. Therapeutic agents are also referred to herein as “growth inhibitory agents,” “payloads,” or “toxins,” and may be abbreviated as “D.”

In embodiments, an immunoconjugate has the following Formula A:

Ab-(C-L-(D)ₘ)ₙ (A),
or a pharmaceutically acceptable salt, solvate, or hydrate thereof, wherein:
Ab is an antibody or antigen-binding fragment thereof disclosed herein;
C is a conjugator;
L is a linker;
D is a cytotoxic agent;
m is an integer from 1 to 8; and
n is from 1 to 10.

In particular embodiments, m is 1.

In embodiments, an immunoconjugate has the following Formula A-1:

Ab-(C-L-D)ₙ (A-1),
or a pharmaceutically acceptable salt, solvate, or hydrate thereof, wherein:
Ab is an antibody or antigen-binding fragment thereof disclosed herein;
C is a conjugator;
L is a linker;
D is a cytotoxic agent; and
n is from 1 to 10.

In embodiments, n is from 1 to 10 or from 3 to 10. In embodiments, n is about 3, 4, 5, 6, 7, 8, 9, or 10. In some embodiments n is from 7 to 9. In embodiments, n is about 8.

In embodiments, C has formula (C-I), (C-Ia), (C-Ib), (C-II), (C-III), (C-IIIa), (C-IIIb), or (C-IV):

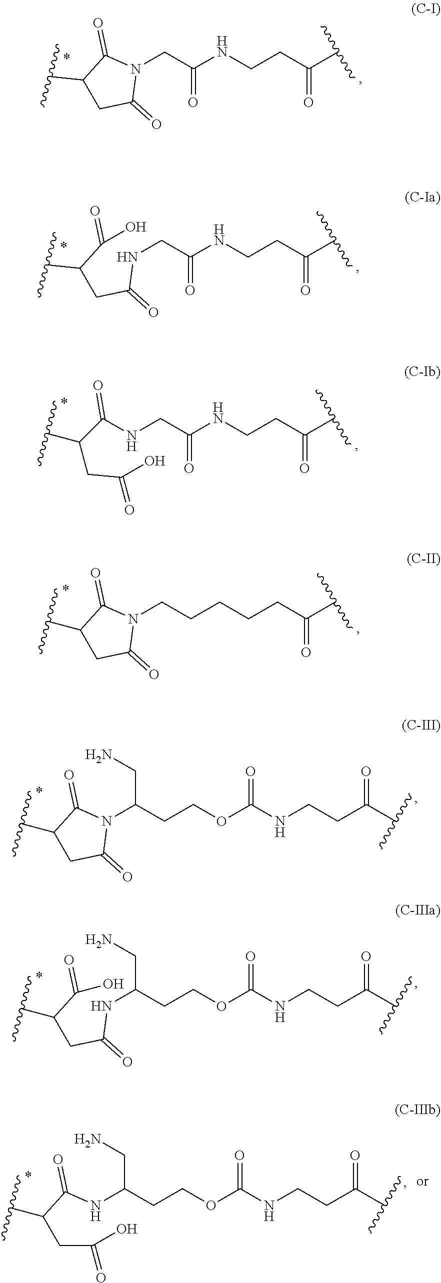

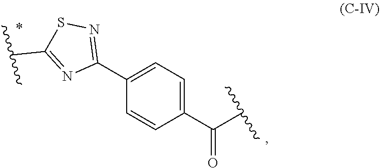 [SMILES: *C(=O)c1ccc(-c2nsc(*)n2)cc1]

wherein * marks the bond where the conjugator connects to Ab.

In embodiments, L has formula (L-I), (L-II), or (L-IIII):

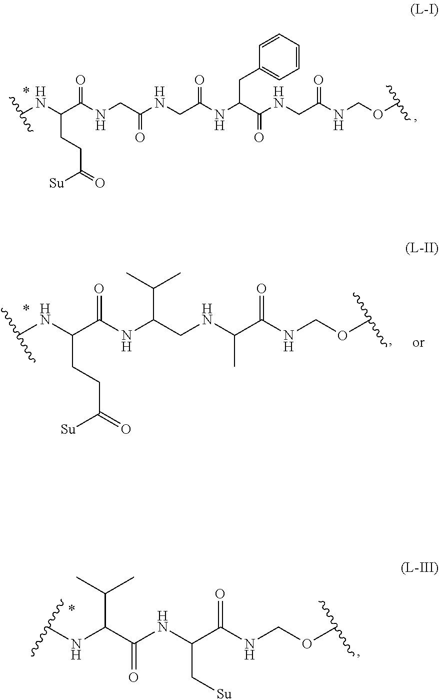

wherein Su is a hydrophilic residue; and * marks the bond where the linker connects to the conjugator.

In embodiments, Su is

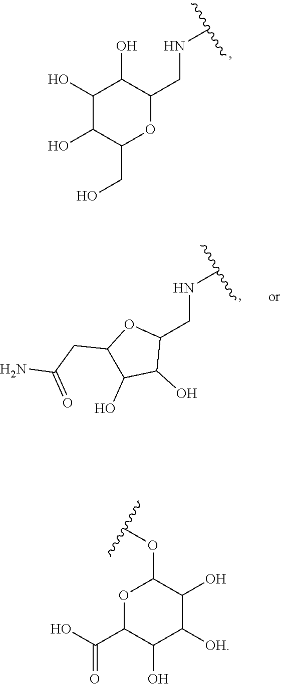 [SMILES: *NCC1OC(CO)C(O)C(O)C1O ; *NCC1OC(CC(N)=O)C(O)C1O ; *OC1OC(C(=O)O)C(O)C(O)C1O]

In embodiments, Su is

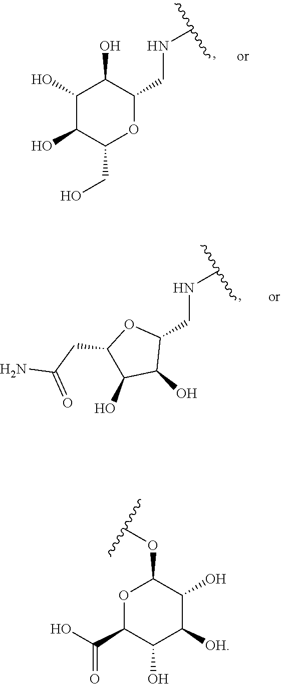 [SMILES: *NC[C@@H]1O[C@H](CO)[C@@H](O)[C@H](O)[C@H]1O ; *NC[C@H]1O[C@@H](CC(N)=O)[C@H](O)[C@@H]1O ; *O[C@@H]1O[C@H](C(=O)O)[C@@H](O)[C@H](O)[C@H]1O]

The cytotoxic agent, D, can include any agent that is detrimental to (e.g., can kill) cells. Examples of cytotoxic agents include, but are not limited to, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs of any of the foregoing.

Other therapeutic or cytotoxic agents to which the anti-FGFR2b antibodies or antigen binding fragments thereof disclosed herein can be conjugated include, but not limited to, antimetabolites (e.g., methotrexate, 6-mercaptopurine, 6-thioguanine, cytarabine, and/or 5-fluorouracil decarbazine), topoisomerase I inhibitors such as exatecan derivatives, e.g., maleimide maleimidocaproyl, alkylating agents (e.g., mechlorethamine, thiotepa chlorambucil, melphalan, carmustine (BSNU), lomustine (CCNU), cyclophosphamide, busulfan, dibromomannitol, streptozotocin, mitomycin C, and/or cis-dichlorodiamine platinum (II) (DDP) cisplatin), anthracyclines (e.g., daunorubicin (formerly daunomycin) and/or doxorubicin), antibiotics (e.g., dactinomycin, bleomycin, mithramycin, and/or anthramycin (AMC)), and anti-mitotic agents (e.g., vincristine and/or vinblastine).

Anti-FGFR2b antibodies or antigen-binding fragments thereof described herein can also be conjugated to one or more radioisotopes (e.g., radioactive iodine) to generate cytotoxic radiopharmaceuticals for treating diseases, disorders, or conditions described herein, such as cancers described herein.

The anti-FGFR2b antibodies or antigen-binding fragments thereof provided in the current disclosure may be conjugated to any one of the compounds described in WO2023/125530, which is incorporated herein by reference in its entirety, as well as with those exemplified herein, e.g., Tables 5A and 5B, and with exatecan derivatives in the prior art.

In embodiments, D is a topoisomerase inhibitor. In embodiments, D is a residue of an exatecan analogue. In some embodiments, D is represented by one of the formulas in Table 5A. In some embodiments, D is is represented by one of the formulas in Table 5B.

TABLE 5A
Exemplary Payloads for Conjugation to Antibodies

| Payload ID | Structure |
|---|---|
| P1 | 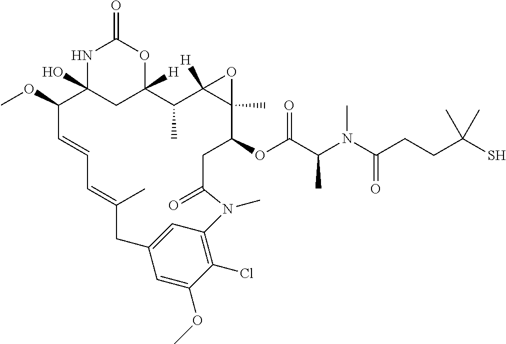 [SMILES: COc1cc2cc(c1Cl)N(C)C(=O)C[C@H](OC(=O)[C@H](C)N(C)C(=O)CCC(C)(C)S)[C@]1(C)O[C@H]1[C@H](C)[C@@H]1C[C@@](O)(NC(=O)O1)[C@H](OC)/C=C/C=C(\C)C2] |
| P2 | 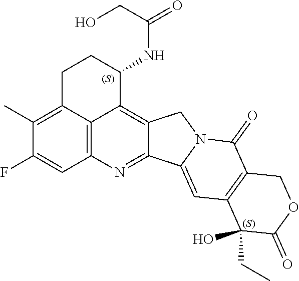 |
| P3 | 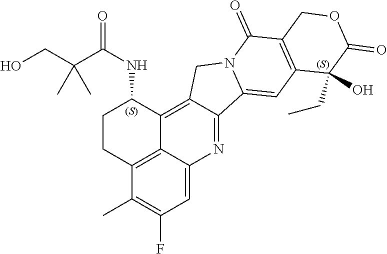 |
| P4 | 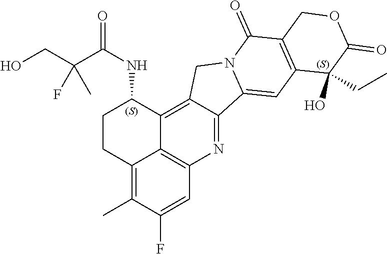 |
| P5 | 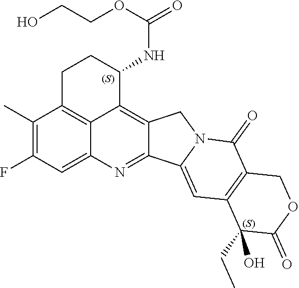 |
| P6 | 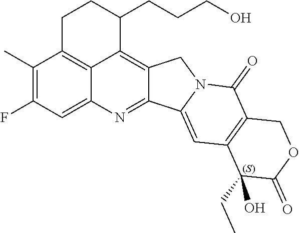 |
| P7 | 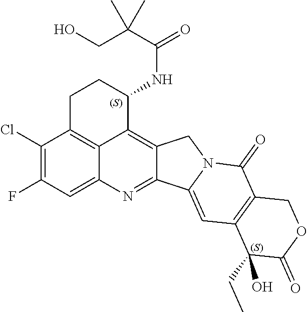 |
| P8 | 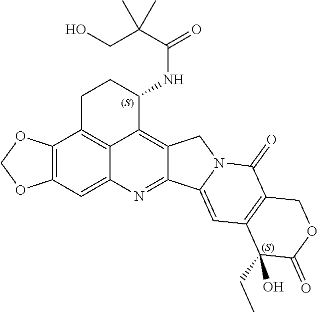 |
| P9 | 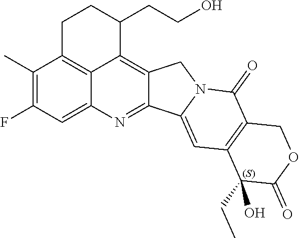 |
| P10 | 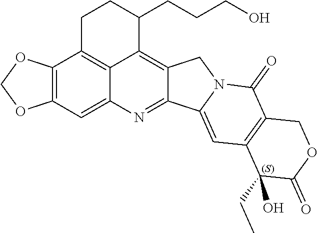 |
| P11 | 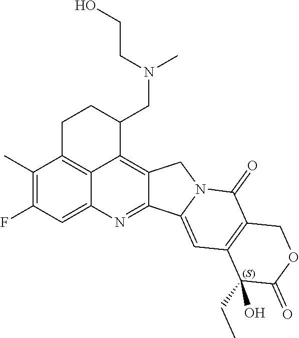 |

TABLE 5B
Exemplary Payloads for Conjugation to Antibodies

| Payload ID | Structure |
|---|---|
| P2 | 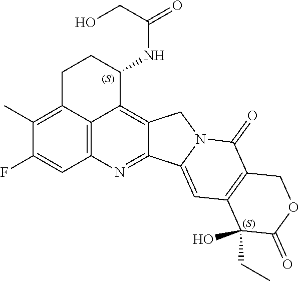 |
| P3 | 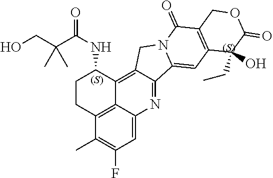 |
| P4 | 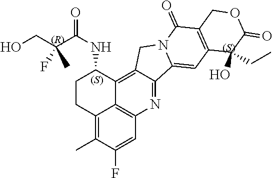 |

In some embodiments, and with reference to Tables 5A and 5B, the immunoconjugate comprises P2. In some embodiments, the immunoconjugate comprises P3. In some embodiments, the immunoconjugate comprises P4.

In embodiments, C-L-D is represented by one of the formulas in in Table 6.

TABLE 6
Exemplary Conjugators + Linker + Payloads for Conjugation to Antibodies

| Linker-Toxin | Structure |
|---|---|
| LD2-1 | 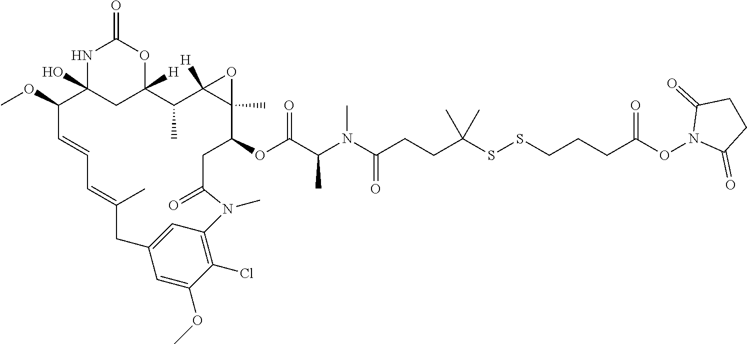 [SMILES: COc1cc2cc(c1Cl)N(C)C(=O)C[C@H](OC(=O)[C@H](C)N(C)C(=O)CCC(C)(C)SSCCCC(=O)ON1C(=O)CCC1=O)[C@]1(C)O[C@H]1[C@H](C)[C@@H]1C[C@@](O)(NC(=O)O1)[C@H](OC)/C=C/C=C(\C)C2] |
| LD2-2 | 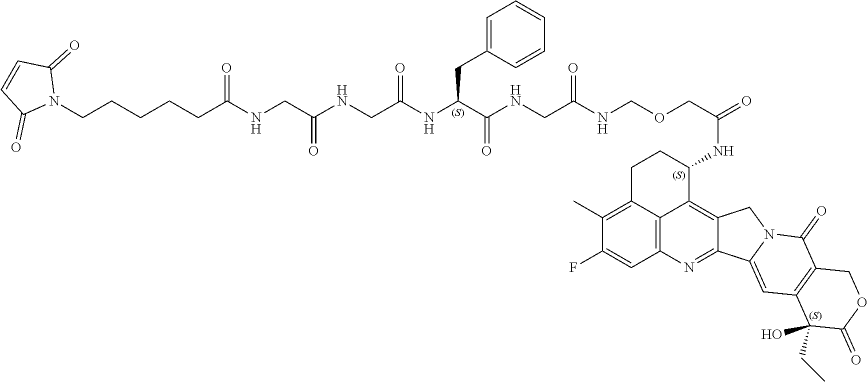 |
| LD2-3 | 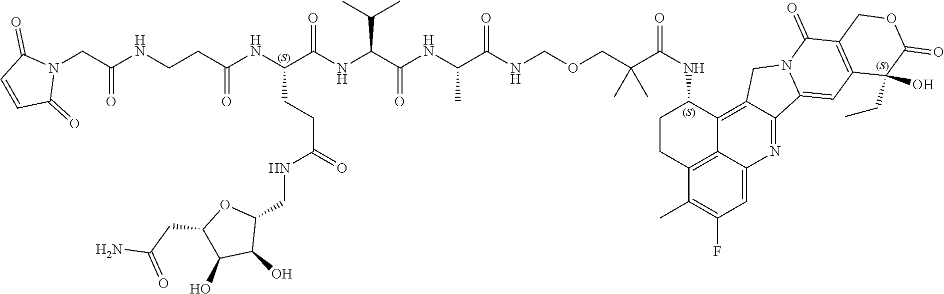 |
| LD2-4 | 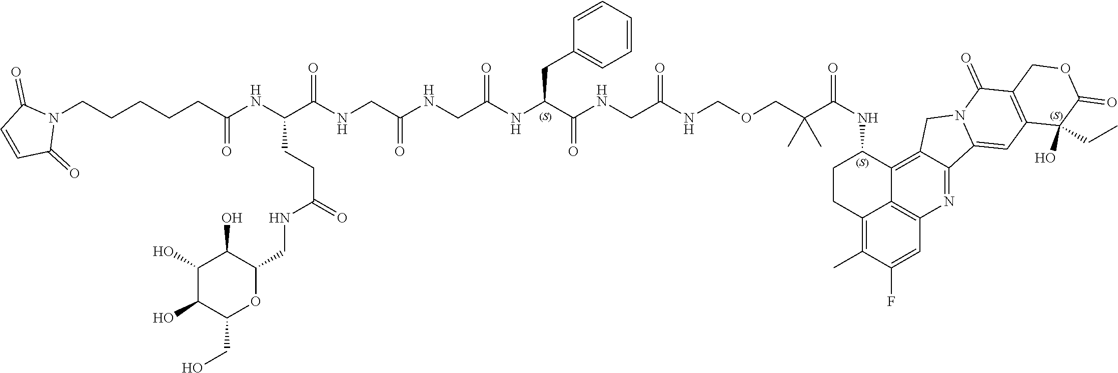 |
| LD2-5 | 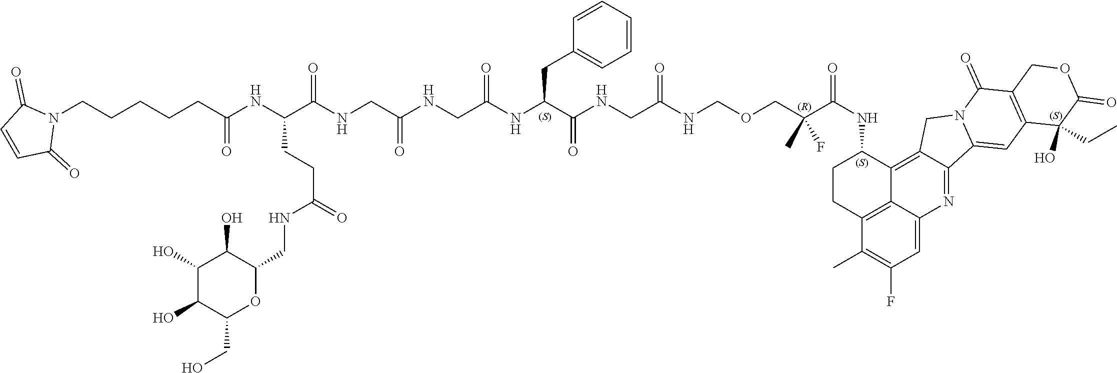 |
| LD2-6 | 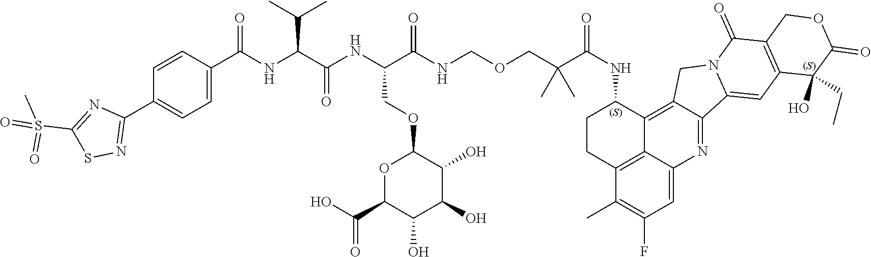 |
| LD2-7 | 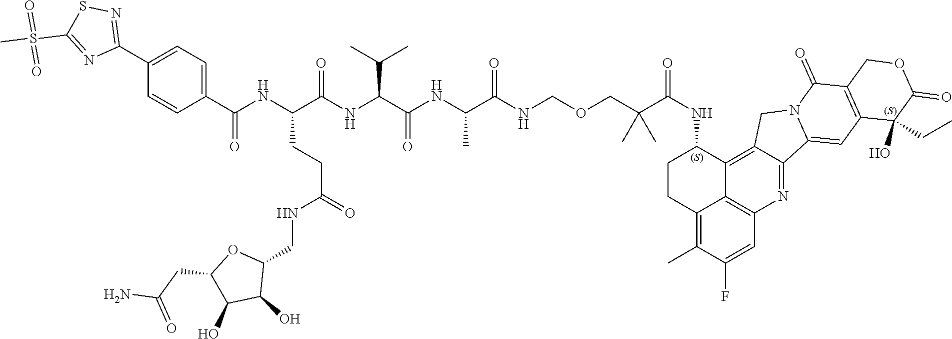 |
| LD2-8 | 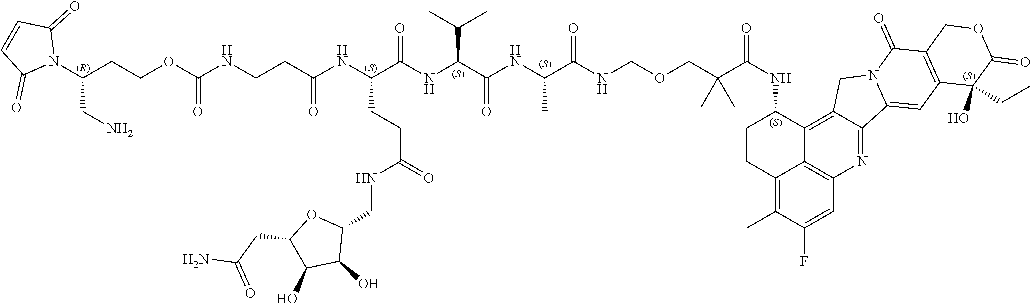 |

With reference to Table 6, LD2-1 and LD2-2 are commercially available and were purchased from MedChemExpress CO. LTD (Shanghai). Antibodies are attached via the conjugator portion, which is generally shown on the left side of the molecule except for LD2-1 (see Table 6).

As used herein, BGA3457 is an immunoconjugate that comprises anti-FGFR2b humanized antibody BGAh9179 and LD2-3.

As used herein, BGA9823 is an immunoconjugate that comprises anti-FGFR2b humanized antibody BGAh9239 and LD2-3.

As used herein, BGA8723 is an immunoconjugate that comprises anti-FGFR2b humanized antibody bemarituzumab (Amgen) and LD2-3.

To generate anti-FGFR2b immunoconjugates, the disclosed antibodies or fragments thereof may be conjugated to any of the conjugators, linkers, and/or payloads provided herein and/or as shown above. For clarity, even if a conjugator, linker, or payload is shown above in Table 6 or below in Tables 7A-7B as part of a combined construct, the individual conjugator, linker, or payload portion may be mixed and matched with any given other conjugator, linker, payload, antibody or antigen-binding fragment thereof to form an immunoconjugate.

In some embodiments, an immunoconjugate is represented by one of the formulas in Table 7A, or a pharmaceutically acceptable salt, solvate, and/or stereoisomer thereof.

TABLE 7A
Exemplary Conjugates

| Conjugate Identifier | ADC Structure |
|---|---|
| LD2-1 | 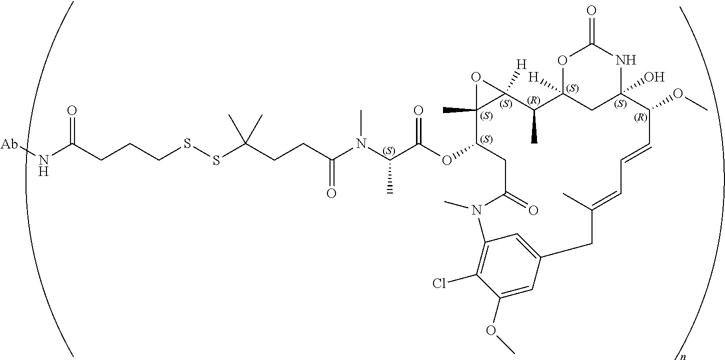 |
| LD2-2 | 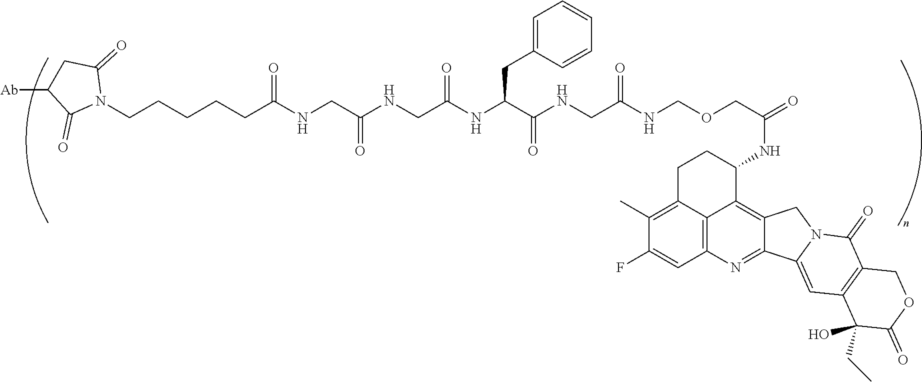 |
| LD2-3 | 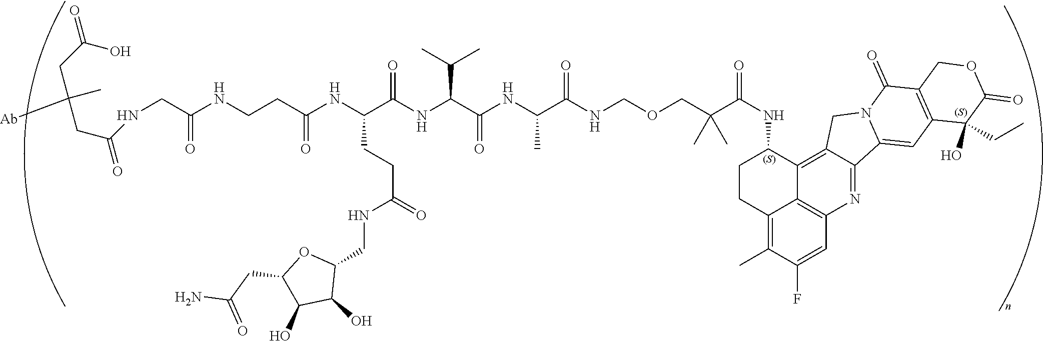 |
| LD2-4 | 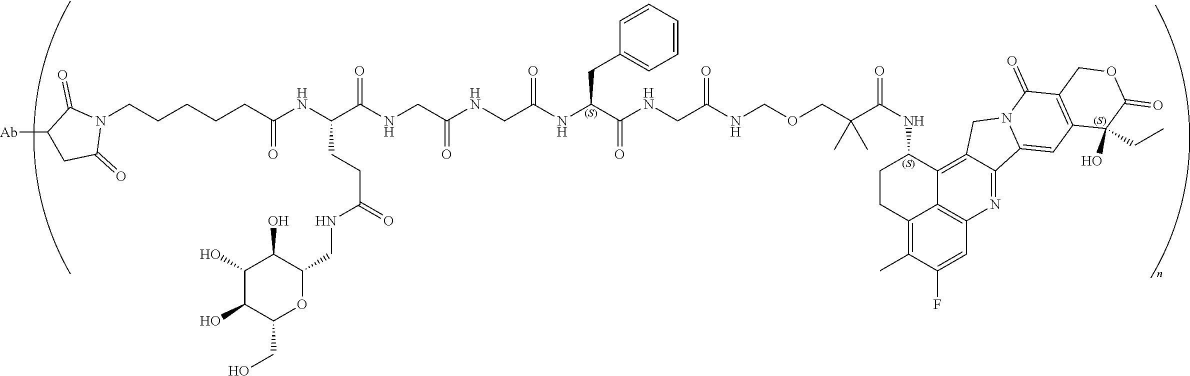 |
| LD2-5 | 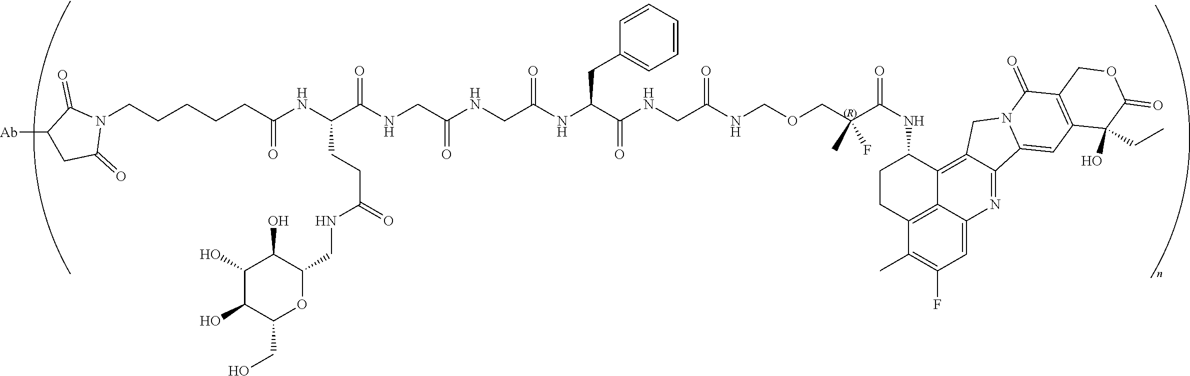 |
| LD2-6 | 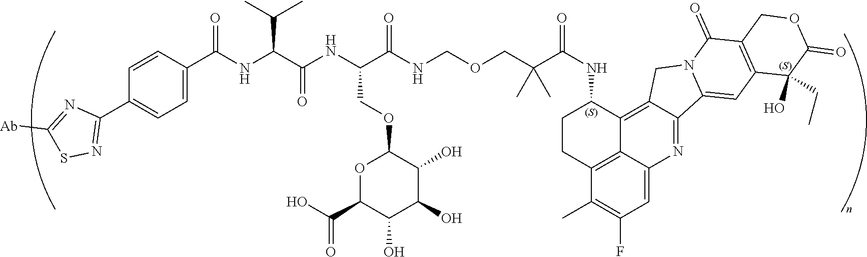 |
| LD2-7 | 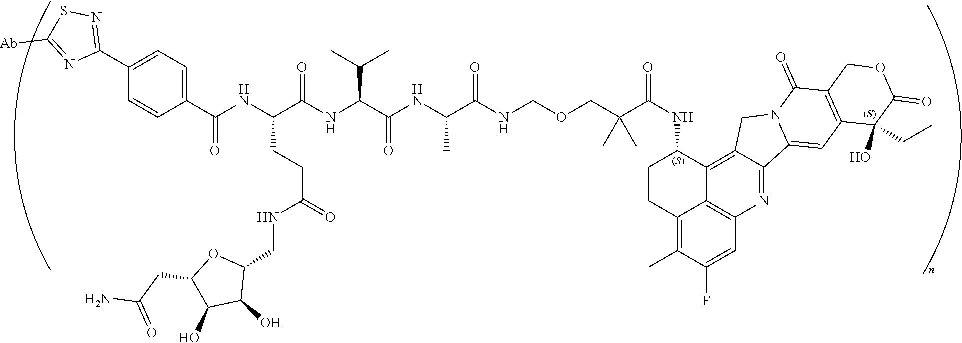 |
| LD2-8 | 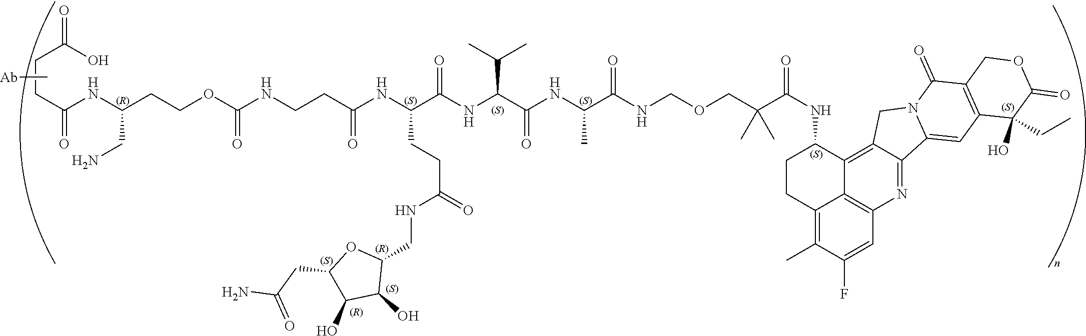 |

With reference to Table 7A, Ab is any anti-FGFR2b antibody or antigen-binding fragment thereof disclosed herein, and n is from 1 to 10 or from 3 to 10. In embodiments, n is about 3, 4, 5, 6, 7, 8, 9, or 10. In some embodiments n is from 7 to 9. In embodiments, n is about 8.

In some embodiments, an immunoconjugate is represented by one of the formulas in Table 7B, or a pharmaceutically acceptable salt, solvate, and/or stereoisomer thereof.

TABLE 7B
Exemplary Conjugates

| Conjugate Identifier | ADC Structure |
|---|---|
| LD2-1 | 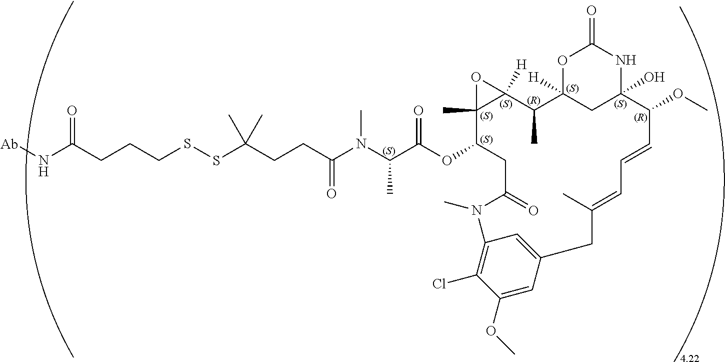 |
| LD2-2 | 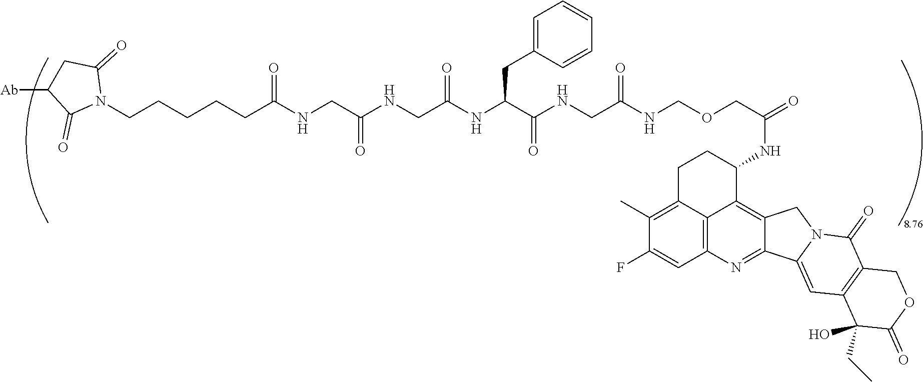 |
| LD2-3 | 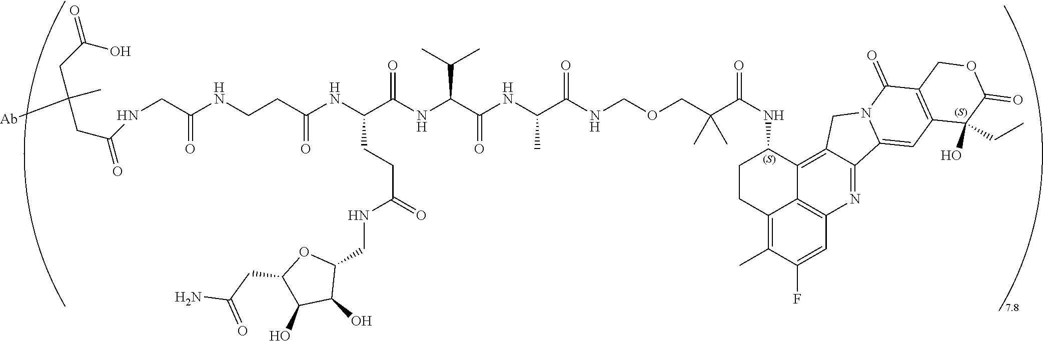 |
| LD2-4 | 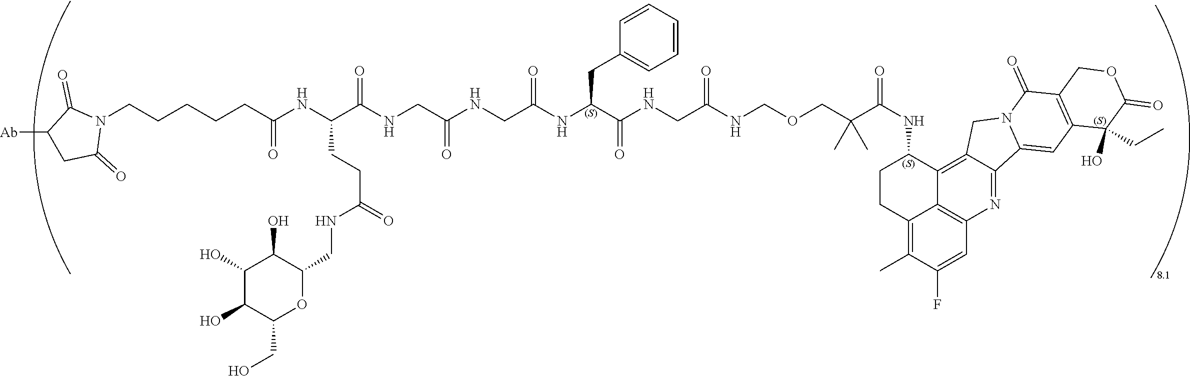 |
| LD2-5 | 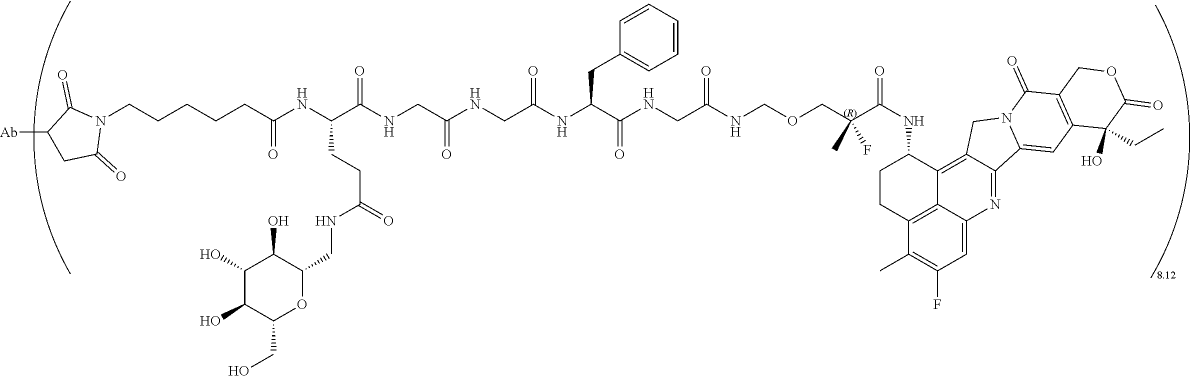 |
| LD2-6 | 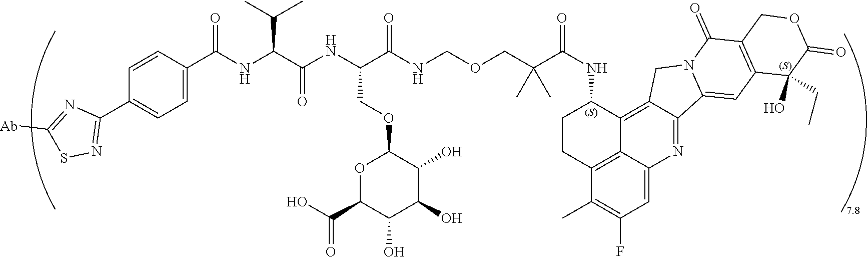 |
| LD2-7 | 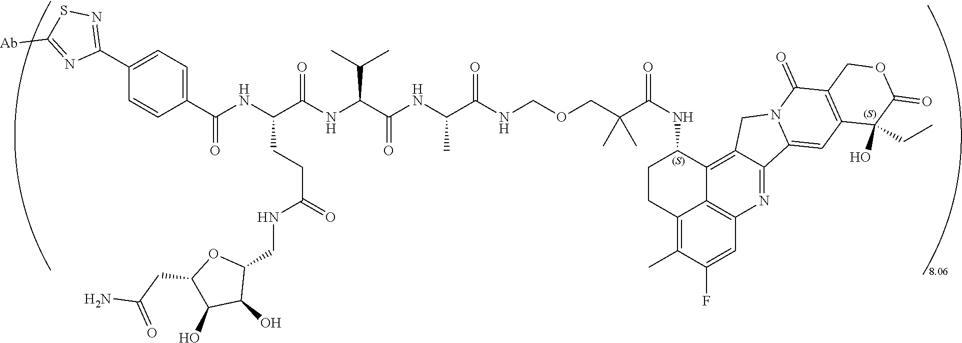 |
| LD2-8 | 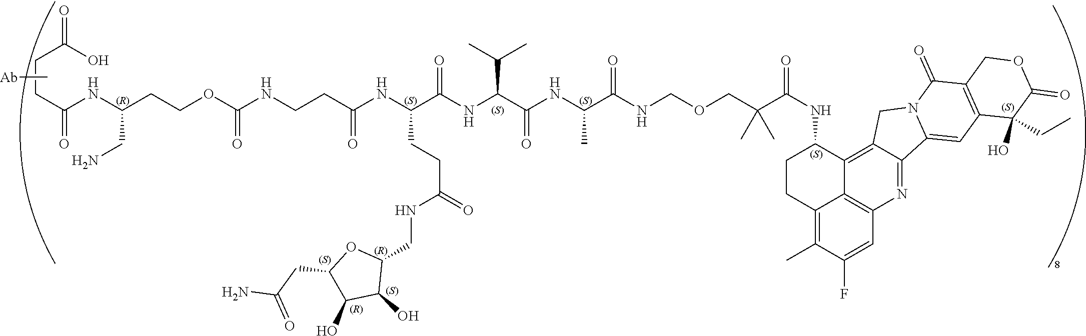 |

With reference to Table 7B, Ab is any anti-FGFR2b antibody or antigen-binding fragment thereof disclosed herein.

### Methods of Treatment

The antibodies, antigen-binding fragments, and conjugates of the present disclosure are useful in a variety of applications including, but not limited to, methods for the treatment of an FGFR2b-associated disorder or disease. In certain embodiments, the FGFR2b-associated disorder or disease is a tubulin inhibitor-resistant tumor or cancer. In some embodiments, a subject with such disorder or disease has an accumulation or overexpression of FGFR2b in a biological sample.

Accordingly, provided herein is a method of treating a cancer comprising administering to a patient in need thereof an effective amount of the antibodies, antigen-binding fragments, and conjugates provided herein, or a pharmaceutical composition provided herein. In some embodiments, the cancer is FGFR2b positive. In some embodiments, the cancer is small cell lung cancer or gastric cancer.

The antibodies, antigen-binding fragments, and conjugates disclosed herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

### Combination Therapies

In one aspect, FGFR2b antibodies and antigen-binding fragments thereof, and immunoconjugates of the present disclosure can be used in combination therapy. Combination therapy refers to those situations in which a subject is simultaneously exposed to two or more therapeutic regimens (e.g., two or more therapeutic moieties). In some embodiments, the two or more regimens may be administered simultaneously; in some embodiments, such regimens may be administered sequentially (e.g., all doses of a first regimen are administered prior to administration of any dose of a second regimen). In some embodiments, such agents are administered in overlapping dosing regimens. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity). Such administration also encompasses co-administration in multiple, or in separate containers (e.g., capsules, powders, and liquids) for each active ingredient. Powders and/or liquids can be reconstituted or diluted to a desired dose prior to administration. Thus, the phrase “in combination with” means that an anti-FGFR2b antibody, antibody-binding fragment, or antibody drug conjugate is administered to a subject at the same time as, just before, or just after administration of an additional therapeutic agent. In certain embodiments, an anti-FGFR2b antibody, antibody-binding fragment, or antibody drug conjugate is administered as a co-formulation with an additional therapeutic agent. The co-formulation may be prepared at any time, including immediately before administration to a patient.

Therapeutic agents for combination therapy with FGFR2b antibodies and antigen-binding fragments thereof, and immunoconjugates of the present disclosure include, but are not limited to, a chemotherapeutic agent (e.g., paclitaxel or a paclitaxel agent (e.g. Abraxane®), docetaxel, carboplatin, topotecan, deruxtecan, cisplatin, irinotecan, doxorubicin, lenalidomide, 5-azacytidine, ifosfamide, oxaliplatin, pemetrexed disodium, cyclophosphamide, etoposide, decitabine, fludarabine, vincristine, bendamustine, chlorambucil, busulfan, gemcitabine, melphalan, pentostatin, mitoxantrone, pemetrexed disodium), tyrosine kinase inhibitor (e.g., EGFR inhibitor (e.g., erlotinib)), multikinase inhibitor (e.g., MGCD265, RGB-286638), CD-20 targeting agent (e.g., rituximab, ofatumumab, RO5072759, LFB-R603), CD52 targeting agent (e.g., alemtuzumab), prednisolone, darbepoetin alfa, lenalidomide, Bcl-2 inhibitor (e.g., oblimersen sodium), aurora kinase inhibitor (e.g., MLN8237, TAK-901), proteasome inhibitor (e.g., bortezomib), CD-19 targeting agent (e.g., MEDI-551, MOR208), MEK inhibitor (e.g., ABT-348), JAK-2 inhibitor (e.g., INCB018424), mTOR inhibitor (e.g., temsirolimus, everolimus), BCR/ABL inhibitor (e.g., imatinib), ET-A receptor antagonist (e.g., ZD4054), TRAIL receptor 2 (TR-2) agonist (e.g., CS-1008), HGF/SF inhibitor (e.g., AMG 102), EGEN-001, or Polo-like kinase 1 inhibitor (e.g., BI 672).

Other therapeutics for use in combination therapy include, for example, immune checkpoint inhibitors. In some embodiments, the immune checkpoint inhibitor is an anti-PD1 or anti-PD-L1 antibody. Anti-PD-1 antibodies can include, without limitation, tislelizumab, pembrolizumab, and nivolumab. Tislelizumab is disclosed in U.S. Pat. No. 8,735,553. Pembrolizumab (formerly MK-3475), as disclosed by Merck in U.S. Pat. Nos. 8,354,509 and 8,900,587, is a humanized lgG4-K immunoglobulin which targets the PD1 receptor and inhibits binding of the PD1 receptor ligands PD-L1 and PD-L2. Pembrolizumab has been approved for the indications of metastatic melanoma and metastatic non-small cell lung cancer (NSCLC) and is under clinical investigation for the treatment of head and neck squamous cell carcinoma (HNSCC), and refractory Hodgkin's lymphoma (cHL). Nivolumab (as disclosed by Bristol-Meyers Squibb) is a fully human lgG4-K monoclonal antibody. Nivolumab (clone 5C4) is disclosed in U.S. Pat. No. 8,008,449 and WO 2006/121168. Nivolumab is approved for the treatment of melanoma, lung cancer, kidney cancer, and Hodgkin's lymphoma. In some embodiments, the anti-PD-1 antibody is tislelizumab.

Other immune checkpoint antibodies for use in combination therapy include anti-TIGIT antibodies. Such anti-TIGIT antibodies can include, without limitation, those disclosed in WO2019/129261, incorporated by reference herein in its entirety.

### Pharmaceutical Compositions and Formulations

Also provided are compositions, including pharmaceutical compositions, comprising an anti-FGFR2b antibody or antigen-binding fragment thereof, such as those disclosed in Table 8, polynucleotides comprising sequences encoding an anti-FGFR2b antibody or antigen-binding fragment, and immunoconjugates comprising any one of the disclosed anti-FGFR2b antibodies or antigen-binding fragments. These compositions may further comprise suitable carriers or diluents, such as pharmaceutically acceptable excipients including buffers, which are well known in the art.

### Kits

The present disclosure provides, inter alia, kits comprising anti-FGFR2b antibodies or antigen-binding fragments or conjugates thereof, such as any of the disclosed immunoconjugates, and instructions for use and/or administration. In some embodiments, a kit comprises at least one anti-FGFR2b antibody, antigen-binding fragment or conjugate thereof, and a pharmaceutically acceptable carrier, and instructions for use and/or administration.

Also provided are kits for use in various methods disclosed herein. Instructions may comprise a description of administering one or more pharmaceutical compositions described herein to a subject to achieve an intended activity in a subject. A kit may further comprise a description of selecting a human suitable for treatment based on identifying whether the human is in need of treatment. In some embodiments, instructions comprise a description of administering at least one anti-FGFR2b antibody or antigen-binding fragment or conjugate thereof to a subject who is in need of the treatment.

Instructions relating to administering one or more doses of at least one anti-FGFR2b antibody or antigen-binding fragment or conjugate thereof generally include information as to dosage, dosing schedule, and route of administration for an intended treatment. Containers in kits may be unit doses, bulk packages (e.g., multi-dose packages) or sub-unit doses. Instructions supplied in kits of the disclosure are typically written instructions on a label or package insert. A label or package insert may indicate that one or more pharmaceutical compositions described herein are used for treating, delaying the onset, and/or alleviating a disease, disorder, or condition in a subject.

In some embodiments, kits provided herein are in suitable packaging. Suitable packaging includes, but is not limited to, vials, bottles, jars, flexible packaging, and the like. Also contemplated are packages for use in combination with a specific device, such as an infusion device. A kit may have a sterile access port (for example, a container may be an intravenous solution bag or a vial having a stopper pierceable by a hypodermic injection needle). A container may also have a sterile access port.

Kits may include additional components such as buffers and interpretive information. A kit may comprise a container and a label or one or more package inserts on or associated with a container. In some embodiment, the disclosure provides articles of manufacture comprising contents of kits described above.

In embodiments, an antibody or antigen-binding fragment thereof provided in a kit forms a complex with FGFR2b that is detected by an assay. The assay may be an enzyme linked immunosorbent assay (ELISA), radioimmune assay (RIA), and/or Western blot.

### EXAMPLES

### Example 1. Generation of Mouse Anti-FGFR2b Antibodies

### Mouse Immunization

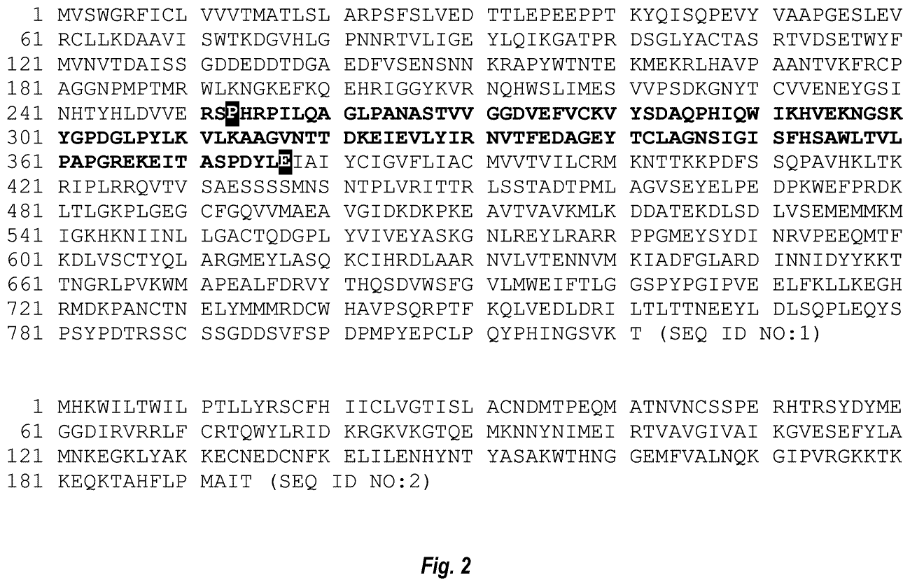

To generate antibodies against FGFR2b, BALB/c mice were immunized with 200 μl antigen mixture containing human FGFR2 beta (IIIb) Domain Protein (FGF-HM2BD, KACTUS) and adjuvants (Complete Freund's Adjuvant (F5881, Sigma) for prime immune and Sigma Adjuvant System (S6322, Sigma) for the following). FIG. 2 shows the amino acid sequence of human FGFR2 (SEQ ID NO:1), provided at NCBI Accession number P21802. In bold is the beta IIIb (D2+D3) domain, which is approximately Pro253-Glu378, and which was used for immunization. Animals were injected bi-weekly intraperitoneally and subcutaneously. Serum titers against FGFR2b protein were determined by ELISA to monitor the humoral immune response. Animals with sufficient FGFR2b specific antibody titers were given a final boost of the FGFR2b domain protein for antibody screen.

### Plasma Cell Screening by the Beacon Optofluidic System

At 3-5 days post final boost, spleens were collected and mashed into single cell suspensions. Plasma cells were isolated with mouse CD138 positive selection kit (STEMCELL) according to the manufacturer's instructions. Enriched plasma cells with a density of 6.25×10⁶/ml were imported into the channel and penned into the NanoPen chambers of OptoSelect 14K Chip™ (Berkeley Lights) according to the manufacturer's instructions. To screen huFGFR2b specific plasma cells, Human FGFR2 alpha (IIIb) Protein (FGF-HM4ABB, KACTUS) conjugated beads (520-00053, Berkeley Lights) and Alexa Fluor 488 goat anti-mouse IgG secondary antibody (Jackson ImmunoResearch) with a concentration of 5 μg/ml were imported into the channel. Following import, the freeze valve was turned on and the exposure time of FITC channel for Alexa Fluor 488 fluorophore was set to 1000 ms. Bloom-like positive signals were captured by time lapse imaging with settings of 3-minute periods and 10 cycles. Human FGFR2 alpha (IIIc) Protein (FGR-HM4CDB, KACTUS) conjugated beads assay was performed as a counter screen to roll out unwanted signals. Plasma cells that showed FGFR2b specific positive signals were individually exported into 96-well plates filled with lysis buffer.

### Example 2. Antibody VH and VL Gene Cloning, Sequencing, and Expression

First-strand cDNA was synthesized, and total cDNA was amplified with Opto Plasma B Discovery cDNA Synthesis Kit™ (Berkeley Lights) according to the manufacturer's instructions. Antibody VH and VL genes were then amplified with Opto Plasma B Discovery Sanger Prep Kit™ (Berkeley Lights) according to the manufacturer's instructions. The amplified VH and VL genes were cloned into mammalian expression vector containing human IgG1 and kappa chain constant region genes and sequenced. The amino acid sequences of the 3 HCDRs, 3 LCDRs, VH, VL, HC and LC, and the DNA sequences of the HC and LC for representative humanized antibodies BGAh9179 and BGAh9239 are listed in Table 8 as SEQ TD NOs: 3 to 26. Antibodies were expressed by Expi293™ cells and purified by affinity chromatography. Sequences of parental murine antibodies BGAm1823 and BGAm1371, and reference antibodies BGA1421, BGA2354, and BGA2311, are also shown in Table 8.

TABLE 8
Exemplary Anti-FGFR2b Antibody Sequences

| | SEQ ID | | |
|---|---|---|---|
| Antibody | NO | | SEQUENCE |
| BGAh9239 | SEQ ID | HCDR1 (Kabat) | IYWLN |
| | NO: 3 | | |
| | SEQ ID | HCDR2 (Kabat) | QIYPENADTNYSGKFKG |
| | NO: 4 | | |
| | SEQ ID | HCDR3 (Kabat) | GGFDY |
| | NO: 5 | | |
| | SEQ ID | LCDR1 (Kabat) | RASENIYSNLA |
| | NO: 6 | | |
| | SEQ ID | LCDR2 (Kabat) | TATNLAE |
| | NO: 7 | | |
| | SEQ ID | LCDR3 (Kabat) | QHFYGILYT |
| | NO: 8 | | |
| | SEQ ID | VH AA | QVQLVQSGAEVKKPGSSVKVSCKASGYTFS |
| | NO: 9 | | IYWLNWVRQAPGQGLEWMGQIYPENADTN |
| | | | YSGKFKGRVTITADKSTSTAYMELSSLRSEDT |
| | | | AVYFCARGGFDYWGQGTLVTVSS |
| | SEQ ID | VL AA | DIQMTQSPSSLSASVGDRVTITCRASENIYSN |
| | NO: 10 | | LAWYQQKPGKAPKLLIYTATNLAEGVPSRFS |
| | | | GSGSGTDFTLTISSLQPEDFATYYCQHFYGIL |
| | | | YTFGQGTRLEIK |
| | SEQ ID | HC AA | QVQLVQSGAEVKKPGSSVKVSCKASGYTFS |
| | NO: 11 | | IYWLNWVRQAPGQGLEWMGQIYPENADTN |
| | | | YSGKFKGRVTITADKSTSTAYMELSSLRSEDT |
| | | | AVYFCARGGFDYWGQGTLVTVSSASTKGPS |
| | | | VFPLAPSSKSTSGGTAALGCLVKDYFPEPVT |
| | | | VSWNSGALTSGVHTFPAVLQSSGLYSLSSVV |
| | | | TVPSSSLGTQTYICNVNHKPSNTKVDKKVEP |
| | | | KSCDKTHTCPPCPAPELLGGPSVFLFPPKPK |
| | | | DTLMISRTPEVTCVVVDVSHEDPEVKFNWY |
| | | | VDGVEVHNAKTKPREEQYNSTYRVVSVLTV |
| | | | LHQDWLNGKEYKCKVSNKALPAPIEKTISK |
| | | | AKGQPREPQVYTLPPSRDELTKNQVSLTCLV |
| | | | KGFYPSDIAVEWESNGQPENNYKTTPPVLDS |
| | | | DGSFFLYSKLTVDKSRWQQGNVFSCSVMHE |
| | | | ALHNHYTQKSLSLSPGK |
| | SEQ ID | LC AA | DIQMTQSPSSLSASVGDRVTITCRASENIYSN |
| | NO: 12 | | LAWYQQKPGKAPKLLIYTATNLAEGVPSRFS |
| | | | GSGSGTDFTLTISSLQPEDFATYYCQHFYGIL |
| | | | YTFGQGTRLEIKRTVAAPSVFIFPPSDEQLKS |
| | | | GTASVVCLLNNFYPREAKVQWKVDNALQS |
| | | | GNSQESVTEQDSKDSTYSLSSTLTLSKADYE |
| | | | KHKVYACEVTHQGLSSPVTKSFNRGEC |
| | SEQ ID | HC DNA | CAAGTGCAGCTGGTGCAGAGCGGCGCCGA |
| | NO: 13 | | GGTGAAGAAGCCCGGCAGCAGCGTGAAG |
| | | | GTGAGCTGCAAGGCTAGCGGCTACACCTT |
| | | | CAGCATCTACTGGCTGAACTGGGTGAGAC |
| | | | AAGCCCCCGGCCAAGGCCTGGAGTGGATG |
| | | | GGGCAGATCTACCCCGAGAACGCCGACAC |
| | | | CAACTACAGCGGCAAGTTCAAGGGCAGAG |
| | | | TGACCATCACCGCCGACAAGAGCACAAGC |
| | | | ACCGCCTACATGGAGCTGAGCAGCCTGAG |
| | | | AAGCGAGGACACCGCCGTGTACTTCTGCG |
| | | | CTAGAGGCGGCTTCGACTACTGGGGCCAA |
| | | | GGCACCCTGGTGACCGTGAGCAGCGCTTC |
| | | | GACCAAGGGCCCATCGGTCTTCCCCCTGG |
| | | | CACCCTCCTCCAAGAGCACCTCTGGGGGC |
| | | | ACAGCGGCCCTGGGCTGCCTGGTCAAGGA |
| | | | CTACTTCCCCGAACCGGTGACGGTGTCGT |
| | | | GGAACTCAGGCGCCCTGACCAGCGGCGTG |
| | | | CACACCTTCCCGGCTGTCCTACAGTCCTCA |
| | | | GGACTCTACTCCCTCAGCAGCGTGGTGAC |
| | | | CGTGCCCTCCAGCAGCTTGGGCACCCAGA |
| | | | CCTACATCTGCAACGTGAATCACAAGCCC |
| | | | AGCAACACCAAGGTGGACAAGAAAGTTG |
| | | | AGCCCAAATCTTGTGACAAAACTCACACA |
| | | | TGCCCACCGTGCCCAGCACCTGAACTCCT |
| | | | GGGGGGACCGTCAGTCTTCCTCTTCCCCCC |
| | | | AAAACCCAAGGACACCCTCATGATCTCCC |
| | | | GGACCCCTGAGGTCACATGCGTGGTGGTG |
| | | | GACGTGAGCCACGAAGACCCTGAGGTCAA |
| | | | GTTCAACTGGTACGTGGACGGCGTGGAGG |
| | | | TGCATAATGCCAAGACAAAGCCGCGGGAG |
| | | | GAGCAGTACAACAGCACGTACCGTGTGGT |
| | | | CAGCGTCCTCACCGTCCTGCACCAGGACT |
| | | | GGCTGAATGGCAAGGAGTACAAGTGCAAG |
| | | | GTCTCCAACAAAGCCCTCCCAGCCCCCAT |
| | | | CGAGAAAACCATCTCCAAAGCCAAAGGGC |
| | | | AGCCCCGAGAACCACAGGTGTACACCCTG |
| | | | CCCCCATCCCGGGATGAGCTGACCAAGAA |
| | | | CCAGGTCAGCCTGACCTGCCTGGTCAAAG |
| | | | GCTTCTATCCCAGCGACATCGCCGTGGAGT |
| | | | GGGAGAGCAATGGGCAGCCGGAGAACAA |
| | | | CTACAAGACCACGCCTCCCGTGCTGGACT |
| | | | CCGACGGCTCCTTCTTCCTCTACAGCAAGC |
| | | | TCACCGTGGACAAGAGCAGGTGGCAGCA |
| | | | GGGGAACGTCTTCTCATGCTCCGTGATGCA |
| | | | TGAGGCTCTGCACAACCACTACACGCAGA |
| | | | AGAGCCTCTCCCTGTCTCCGGGTAAA |
| | SEQ ID | LC DNA | GACATTCAGATGACACAGAGCCCTAGCAG |
| | NO: 14 | | CCTGTCCGCTAGCGTGGGCGACAGAGTGA |
| | | | CCATCACCTGCAGAGCTAGCGAGAACATC |
| | | | TACAGCAACCTGGCCTGGTATCAGCAGAA |
| | | | GCCCGGCAAGGCCCCCAAGCTGCTGATCT |
| | | | ACACCGCCACCAACCTGGCCGAGGGCGTG |
| | | | CCTAGCAGATTCAGCGGCAGCGGCAGCGG |
| | | | CACCGACTTCACCCTGACCATCAGCAGCC |
| | | | TGCAGCCCGAGGACTTCGCCACCTACTACT |
| | | | GTCAGCACTTCTACGGCATCCTGTACACCT |
| | | | TCGGCCAAGGCACAAGACTGGAGATCAAG |
| | | | CGTACGGTGGCTGCACCATCTGTCTTCATC |
| | | | TTCCCGCCATCTGATGAGCAGTTGAAATCT |
| | | | GGAACTGCCTCTGTTGTGTGCCTGCTGAAT |
| | | | AACTTCTATCCCAGAGAGGCCAAAGTACA |
| | | | GTGGAAGGTGGATAACGCCCTCCAATCGG |
| | | | GTAACTCCCAGGAGAGTGTCACAGAGCAG |
| | | | GACAGCAAGGACAGCACCTACAGCCTCAG |
| | | | CAGCACCCTGACGCTGAGCAAAGCAGACT |
| | | | ACGAGAAACACAAAGTCTACGCCTGCGAA |
| | | | GTCACCCATCAGGGCCTGAGCTCGCCCGT |
| | | | CACAAAGAGCTTCAACAGGGGAGAGTGT |
| BGAh9179 | SEQ D | HCDR1 (Kabat) | DTYIH |
| | NO: 15 | | |
| | SEQ ID | HCDR2 (Kabat) | RIDPAEGNTMFASEFQG |
| | NO: 16 | | |
| | SEQ ID | HCDR3 (Kabat) | SKIHYDYDEGFAY |
| | NO: 17 | | |
| | SEQ ID | LCDR1 (Kabat) | RASESVDDYGYSFLH |
| | NO: 18 | | |
| | SEQ ID | LCDR2 (Kabat) | RASNLES |
| | NO: 19 | | |
| | SEQ ID | LCDR3 (Kabat) | QQSNQNPRT |
| | NO: 20 | | |
| | SEQ ID | VH AA | QVQLVQSGAEVKKPGASVKVSCKASGYTIT |
| | NO: 21 | | DTYIHWVRQAPGQGLEWMGRIDPAEGNTM |
| | | | FASEFQGRVTMTADTSTSTVYMELSSLRSED |
| | | | TAVYYCARSKIHYDYDEGFAYWGQGTLVTV |
| | | | SS |
| | SEQ ID | VL AA | DIVLTQSPDSLAVSLGERATINCRASESVDD |
| | NO: 22 | | YGYSFLHWYQQKPGQPPKLLIYRASNLESG |
| | | | VPDRFSGSGSGTDFTLTISSLQAEDVAVYYC |
| | | | QQSNQNPRTFGGGTKVEIK |
| | SEQ ID | HC AA | QVQLVQSGAEVKKPGASVKVSCKASGYTIT |
| | NO: 23 | | DTYIHWVRQAPGQGLEWMGRIDPAEGNTM |
| | | | FASEFQGRVTMTADTSTSTVYMELSSLRSED |
| | | | TAVYYCARSKIHYDYDEGFAYWGQGTLVTV |
| | | | SSASTKGPSVFPLAPSSKSTSGGTAALGCLV |
| | | | KDYFPEPVTVSWNSGALTSGVHTFPAVLQSS |
| | | | GLYSLSSVVTVPSSSLGTQTYICNVNHKPSN |
| | | | TKVDKKVEPKSCDKTHTCPPCPAPELLGGPS |
| | | | VFLFPPKPKDTLMISRTPEVTCVVVDVSHED |
| | | | PEVKFNWYVDGVEVHNAKTKPREEQYNST |
| | | | YRVVSVLTVLHQDWLNGKEYKCKVSNKAL |
| | | | PAPIEKTISKAKGQPREPQVYTLPPSRDELTK |
| | | | NQVSLTCLVKGFYPSDIAVEWESNGQPENN |
| | | | YKTTPPVLDSDGSFFLYSKLTVDKSRWQQG |
| | | | NVFSCSVMHEALHNHYTQKSLSLSPGK |
| | SEQ ID | LC AA | DIVLTQSPDSLAVSLGERATINCRASESVDDY |
| | NO: 24 | | GYSFLHWYQQKPGQPPKLLIYRASNLESGV |
| | | | PDRFSGSGSGTDFTLTISSLQAEDVAVYYCQ |
| | | | QSNQNPRTFGGGTKVEIKRTVAAPSVFIFPPS |
| | | | DEQLKSGTASVVCLLNNFYPREAKVQWKV |
| | | | DNALQSGNSQESVTEQDSKDSTYSLSSTLTL |
| | | | SKADYEKHKVYACEVTHQGLSSPVTKSFNR |
| | | | GEC |
| | SEQ ID | HC DNA | CAAGTGCAGCTGGTGCAGAGCGGCGCCGA |
| | NO: 25 | | GGTGAAGAAGCCCGGCGCTAGCGTGAAG |
| | | | GTGAGCTGCAAGGCTAGCGGCTACACCAT |
| | | | CACCGACACCTACATCCACTGGGTGAGAC |
| | | | AAGCCCCCGGCCAAGGCCTGGAGTGGATG |
| | | | GGCAGAATCGACCCCGCCGAGGGCAACAC |
| | | | CATGTTCGCTAGCGAGTTCCAAGGCAGAG |
| | | | TGACCATGACCGCCGACACAAGCACAAGC |
| | | | ACCGTGTACATGGAGCTGAGCAGCCTGAG |
| | | | AAGCGAGGACACCGCCGTGTACTACTGCG |
| | | | CTAGAAGCAAGATCCACTACGACTACGAC |
| | | | GAGGGCTTCGCCTACTGGGGCCAAGGCAC |
| | | | CCTGGTGACCGTGAGCAGCGCTTCGACCA |
| | | | AGGGCCCATCGGTCTTCCCCCTGGCACCCT |
| | | | CCTCCAAGAGCACCTCTGGGGGCACAGCG |
| | | | GCCCTGGGCTGCCTGGTCAAGGACTACTT |
| | | | CCCCGAACCGGTGACGGTGTCGTGGAACT |
| | | | CAGGCGCCCTGACCAGCGGCGTGCACACC |
| | | | TTCCCGGCTGTCCTACAGTCCTCAGGACTC |
| | | | TACTCCCTCAGCAGCGTGGTGACCGTGCC |
| | | | CTCCAGCAGCTTGGGCACCCAGACCTACA |
| | | | TCTGCAACGTGAATCACAAGCCCAGCAAC |
| | | | ACCAAGGTGGACAAGAAAGTTGAGCCCA |
| | | | AATCTTGTGACAAAACTCACACATGCCCA |
| | | | CCGTGCCCAGCACCTGAACTCCTGGGGGG |
| | | | ACCGTCAGTCTTCCTCTTCCCCCCAAAACC |
| | | | CAAGGACACCCTCATGATCTCCCGGACCC |
| | | | CTGAGGTCACATGCGTGGTGGTGGACGTG |
| | | | AGCCACGAAGACCCTGAGGTCAAGTTCAA |
| | | | CTGGTACGTGGACGGCGTGGAGGTGCATA |
| | | | ATGCCAAGACAAAGCCGCGGGAGGAGCA |
| | | | GTACAACAGCACGTACCGTGTGGTCAGCG |
| | | | TCCTCACCGTCCTGCACCAGGACTGGCTG |
| | | | AATGGCAAGGAGTACAAGTGCAAGGTCTC |
| | | | CAACAAAGCCCTCCCAGCCCCCATCGAGA |
| | | | AAACCATCTCCAAAGCCAAAGGGCAGCCC |
| | | | CGAGAACCACAGGTGTACACCCTGCCCCC |
| | | | ATCCCGGGATGAGCTGACCAAGAACCAGG |
| | | | TCAGCCTGACCTGCCTGGTCAAAGGCTTC |
| | | | TATCCCAGCGACATCGCCGTGGAGTGGGA |
| | | | GAGCAATGGGCAGCCGGAGAACAACTACA |
| | | | AGACCACGCCTCCCGTGCTGGACTCCGAC |
| | | | GGCTCCTTCTTCCTCTACAGCAAGCTCACC |
| | | | GTGGACAAGAGCAGGTGGCAGCAGGGGA |
| | | | ACGTCTTCTCATGCTCCGTGATGCATGAGG |
| | | | CTCTGCACAACCACTACACGCAGAAGAGC |
| | | | CTCTCCCTGTCTCCGGGTAAA |
| | SEQ ID | LC DNA | GACATCGTGCTGACACAGAGCCCCGACAG |
| | NO: 26 | | CCTGGCCGTGAGCCTGGGCGAGAGAGCCA |
| | | | CCATCAACTGCAGAGCTAGCGAGAGCGTG |
| | | | GACGACTACGGCTACAGCTTCCTGCACTG |
| | | | GTATCAGCAGAAGCCCGGGCAGCCCCCCA |
| | | | AGCTGCTGATCTACAGAGCTAGCAACCTG |
| | | | GAGAGCGGCGTGCCCGACAGATTCAGCGG |
| | | | CAGCGGCAGCGGCACCGACTTCACCCTGA |
| | | | CCATCAGCAGCCTGCAAGCCGAGGACGTG |
| | | | GCCGTGTACTACTGTCAGCAGAGCAATCA |
| | | | GAACCCTAGAACCTTCGGCGGGGGCACCA |
| | | | AGGTGGAGATCAAGCGTACGGTGGCTGCA |
| | | | CCATCTGTCTTCATCTTCCCGCCATCTGATG |
| | | | AGCAGTTGAAATCTGGAACTGCCTCTGTT |
| | | | GTGTGCCTGCTGAATAACTTCTATCCCAGA |
| | | | GAGGCCAAAGTACAGTGGAAGGTGGATAA |
| | | | CGCCCTCCAATCGGGTAACTCCCAGGAGA |
| | | | GTGTCACAGAGCAGGACAGCAAGGACAG |
| | | | CACCTACAGCCTCAGCAGCACCCTGACGC |
| | | | TGAGCAAAGCAGACTACGAGAAACACAA |
| | | | AGTCTACGCCTGCGAAGTCACCCATCAGG |
| | | | GCCTGAGCTCGCCCGTCACAAAGAGCTTC |
| | | | AACAGGGGAGAGTGT |
| BGAm1823 | SEQ ID | VH DNA | GAGGTCCAGCTGCAGCAGTCTGGGGCTGA |
| | NO: 27 | | GCTGGTGAAGCCTGGGGCCTCAGTGAAGA |
| | | | TTTCCTGCAAAGCTTCTGGCTACGCATTCA |
| | | | GTATCTACTGGATGAACTGGGTGAAGCAG |
| | | | AGGCCTGGAAAGGGTCTTGAATGGATTGG |
| | | | ACAGATTTATCCTGAAAATGGTGATACTAA |
| | | | CTACAACGGAAAGTTCAAGGGCAAGGCCA |
| | | | CACTGACTGCAGACAAATCCTCCAGCACA |
| | | | GCCTACATGCACCTCAGCAGCCTGACCTCT |
| | | | GAGGACTCTGCGGTCTATTTCTGTGCAAGA |
| | | | GGGGGGTTTGACTACTGGGGCCAAGGCAC |
| | | | CACTCTCACCGTCTCGAGC |
| | SEQ ID | VH AA | EVQLQQSGAELVKPGASVKISCKASGYAFSI |
| | NO: 28 | | YWMNWVKQRPGKGLEWIGQIYPENGDTNY |
| | | | NGKFKGKATLTADKSSSTAYMHLSSLTSEDS |
| | | | AVYFCARGGFDYWGQGTTLTVSS |
| | SEQ ID | VL DNA | AATATTGTGATGACCCAGTCTCCAGCCTCC |
| | NO: 29 | | CTATCTGCATCTGTGGGAGAAACTGTCACC |
| | | | ATCACATGTCGAGCAAGTGAGAATATTTAC |
| | | | AGTAATTTAGCATGGTATCAGCAGAAACAG |
| | | | GGAAAATCTCCTCAGCTCCTGGTCTATACT |
| | | | GCAACAAATTTAGCAGATGGTGTGCCATCA |
| | | | AGGTTCAGTGGCAGTGGATCAGGCACACA |
| | | | GTTTTCTCTGAAGATCAACAGCCTGCAGCC |
| | | | TGAAGATTTTGGGAGTTATTACTGTCAACA |
| | | | TTTTTATGGTATTTTGTATACGTTCGGATCG |
| | | | GGGACCAGACTGGAAATAAAA |
| | SEQ ID | VL AA | NIVMTQSPASLSASVGETVTITCRASENIYSN |
| | NO: 30 | | LAWYQQKQGKSPQLLVYTATNLADGVPSRF |
| | | | SGSGSGTQFSLKINSLQPEDFGSYYCQHFYGI |
| | | | LYTFGSGTRLEIK |
| BGAm1371 | SEQ ID | VH DNA | CAGGTGCAACTGAAGCAGTCTGGGGCAGA |
| | NO: 31 | | GCTTGTGAAGCCAGGGGCCTCAGTCAAGT |
| | | | TGTCCTGCACAGCTTCTGGCTTCAACATTA |
| | | | AAGACACCTATATACACTGGGTGAAGCAG |
| | | | AGGCCTGAACAGGGCCTGGAGTGGATTGG |
| | | | AAGGATTGATCCTGCGAATGGTAATACTAT |
| | | | GTTTGCCTCAGAGTTCCAGGGCAAGGCCA |
| | | | CTATAACAGCATACACATCATCCAACACAG |
| | | | CCTACATGCAGCTCAGCAGCCTGACATCTG |
| | | | GGGACACTGCCGTCTATTATTGTGCTAGAT |
| | | | CCAAGATCCACTATGATTACGACGAGGGGT |
| | | | TTGCTTACTGGGGCCAAGGGACTCTGGTC |
| | | | ACCGTCTCGAGC |
| | SEQ ID | VH AA | QVQLKQSGAELVKPGASVKLSCTASGFNIK |
| | NO: 32 | | DTYIHWVKQRPEQGLEWIGRIDPANGNTMF |
| | | | ASEFQGKATITAYTSSNTAYMQLSSLTSGDTA |
| | | | VYYCARSKIHYDYDEGFAYWGQGTLVTVSS |
| | SEQ ID | VL DNA | AACATTGTGCTGACCCAATCTCCACCTTCT |
| | NO: 33 | | TTGGCTGTGTCTCTAGGGCAGAGGGCCAC |
| | | | CGTATCCTGCAGAGCCAGTGAAAGTGTTG |
| | | | ATGATTATGGCTATAGTTTTTTGCACTGGTA |
| | | | CCAGCAGAAACCAGGACAGCCACCCAAA |
| | | | CTCCTCATCTATCGTGCATCCAACCTAGAAT |
| | | | CTGGGATCCCTGCCAGGTTCAGTGGCAGT |
| | | | GGCTCTAGGACAGACTTCACCCTCACCATT |
| | | | AATCCTGTGGAGACTGATGACGTTGCAAC |
| | | | CTACTACTGTCAGCAAAGTAATCAAAATCC |
| | | | TCGGACGTTCGGTGGAGGCACAAAGTTGG |
| | | | AAATCAAA |
| | SEQ ID | VL AA | NIVLTQSPPSLAVSLGQRATVSCRASESVDD |
| | NO: 34 | | YGYSFLHWYQQKPGQPPKLLIYRASNLESGI |
| | | | PARFSGSGSRTDFTLTINPVETDDVATYYCQQ |
| | | | SNQNPRTFGGGTKLEIK |
| BGA1421 | SEQ ID | HC AA | QVQLVQSGAEVKKPGSSVKVSCKASGYIFT |
| | NO: 35 | | TYNVHWVRQAPGQGLEWIGS |
| | | | IYPDNGDTSYNQNFKGRATITADKSTSTAYM |
| | | | ELSSLRSEDTAVYYCARGD |
| | | | FAYWGQGTLVTVSSASTKGPSVFPLAPSSKS |
| | | | TSGGTAALGCLVKDYFPEP |
| | | | VTVSWNSGALTSGVHTFPAVLQSSGLYSLSS |
| | | | VVTVPSSSLGTQTYICNVN |
| | | | HKPSNTKVDKRVEPKSCDKTHTCPPCPAPEL |
| | | | LGGPSVFLFPPKPKDTLMI |
| | | | SRTPEVTCVVVDVSHEDPEVKFNWYVDGV |
| | | | EVHNAKTKPREEQYNSTYRVV |
| | | | SVLTVLHQDWLNGKEYKCKVSNKALPAPIE |
| | | | KTISKAKGQPREPQVYTLPP |
| | | | SREEMTKNQVSLTCLVKGFYPSDIAVEWESN |
| | | | GQPENNYKTTPPVLDSDGS |
| | | | FFLYSKLTVDKSRWQQGNVFSCSVMHEALH |
| | | | NHYTQKSLSLSPGK |
| | SEQ ID | LC AA | DIQMTQSPSSLSASVGDRVTITCKASQGVSN |
| | NO: 36 | | DVAWYQQKPGKAPKLLIYS |
| | | | ASYRYTGVPSRFSGSGSGTDFTFTISSLQPED |
| | | | IATYYCQQHSTTPYTFGQ |
| | | | GTKLEIKRTVAAPSVFIFPPSDEQLKSGTASV |
| | | | VCLLNNFYPREAKVQWKV |
| | | | DNALQSGNSQESVTEQDSKDSTYSLSSTLTL |
| | | | SKADYEKHKVYACEVTHQG |
| | | | LSSPVTKSFNRGEC |
| BGA2354 | SEQ ID | HC AA | EVQLLESGGGLVQPGGSLRLSCAASGFTFRT |
| | NO: 37 | | SGMHWVRQAPGKGLEWVAVISYDGSNKYY |
| | | | ADSVKGRFTISRDNSKNTLYLQMNSLRAED |
| | | | TAVYYCARESPGYDFEYWGQGTLVTVSSAS |
| | | | TKGPSVFPLAPSSKSTSGGTAALGCLVKDYF |
| | | | PEPVTVSWNSGALTSGVHTFPAVLQSSGLYS |
| | | | LSSVVTVPSSSLGTQTYICNVNHKPSNTKVD |
| | | | KKVEPKSCDKTHTCPPCPAPELLGGPSVFLF |
| | | | PPKPKDTLMISRTPEVTCVVVDVSHEDPEVK |
| | | | FNWYVDGVEVHNAKTKPREEQYNSTYRVV |
| | | | SVLTVLHQDWLNGKEYKCKVSNKALPAPIE |
| | | | KTISKAKGQPREPQVYTLPPSRDELTKNQVS |
| | | | LTCLVKGFYPSDIAVEWESNGQPENNYKTTP |
| | | | PVLDSDGSFFLYSKLTVDKSRWQQGNVFSCS |
| | | | VMHEALHNHYTQKSLSLSPGK |
| | SEQ ID | LC AA | QSVLTQPPSASGTPGQRVTISCSGSSSNIGSN |
| | NO: 38 | | TVNWYQQLPGTAPKLLIYGNSNRPSGVPDR |
| | | | FSGSKSGTSASLAISGLRSEDEADYYCAAWD |
| | | | DSLNGWVFGGGTKLTVLGQPKAAPSVTLFP |
| | | | PSSEELQANKATLVCLISDFYPGAVTVAWKA |
| | | | DSSPVKAGVETTTPSKQSNNKYAASSYLSLT |
| | | | PEQWKSHRSYSCQVTHEGSTVEKTVAPTEC |
| | | | S |
| BGA2311 | SEQ ID | HC AA | EVQLLESGGGLVQPGGSLRLSCAASGFTFSS |
| | NO: 39 | | YAMSWVRQAPGKGLEWVSAISGSGTSTYYA |
| | | | DSVKGRFTISRDNSKNTLYLQMNSLRAEDT |
| | | | AVYYCARVRYNWNHGDWFDPWGQGTLVT |
| | | | VSSASTKGPSVFPLAPSSKSTSGGTAALGCL |
| | | | VKDYFPEPVTVSWNSGALTSGVHTFPAVLQS |
| | | | SGLYSLSSVVTVPSSSLGTQTYICNVNHKPS |
| | | | NTKVDKKVEPKSCDKTHTCPPCPAPELLGG |
| | | | PSVFLFPPKPKDTLMISRTPEVTCVVVDVSH |
| | | | EDPEVKFNWYVDGVEVHNAKTKPREEQYN |
| | | | STYRVVSVLTVLHQDWLNGKEYKCKVSNK |
| | | | ALPAPIEKTISKAKGQPREPQVYTLPPSRDEL |
| | | | TKNQVSLTCLVKGFYPSDIAVEWESNGQPEN |
| | | | NYKTTPPVLDSDGSFFLYSKLTVDKSRWQQ |
| | | | GNVFSCSVMHEALHNHYTQKSLSLSPGK |
| | SEQ ID | LC AA | QSVLTQPPSASGTPGQRVTISCSGSSSNIGNN |
| | NO: 40 | | YVSWYQQLPGTAPKLLIYENYNRPAGVPDR |
| | | | FSGSKSGTSASLAISGLRSEDEADYYCSSWD |
| | | | DSLNYWVFGGGTKLTVLGQPKAAPSVTLFP |
| | | | PSSEELQANKATLVCLISDFYPGAVTVAWKA |
| | | | DSSPVKAGVETTTPSKQSNNKYAASSYLSLT |
| | | | PEQWKSHRSYSCQVTHEGSTVEKTVAPTEC |
| | | | S |

### Example 3. Humanization of the Chimeric Anti-FGFR2b Antibody

Antibodies BGAh9239 and BGAh9179 are humanized antibodies developed from parental murine antibodies BGAm1823 and BGAm1371, respectively. A multipronged approach was applied to construct antibodies that are superior to the parental murine antibodies, BGAm1371 and BGAm1823. Heavy and light chains were optimized for humanization.

For humanization of the BGAm1371 and BGAm1823 candidates, human germline IgG genes were searched for sequences that share high degrees of homology to the cDNA sequence of BGAm1371 and BGAm1823 by BLAST analysis of the human immunoglobulin gene database in IMGT (imgt.org/3Dstructure-DB/cgi/DomainGapAlign.cgi). The human IGVH and IGVL genes that are present in human antibody repertoires with high frequencies (Glanville 2009 PNAS 106:20216-20221) and are highly homologous to BGAm1371 and BGAm1823, respectively, were selected as the templates for humanization.

Humanization was carried out by CDR-grafting (Methods in Molecular Biology, Vol 248: Antibody Engineering, Methods and Protocols, Humana Press) and the humanization antibody variants (BGAh9239 and BGAh9179) were engineered into a human IgG1 format using an expression vector developed in-house. In the initial round of humanization, mutations from murine to human amino acid residues in framework regions were guided by a simulated 3D structure, and the murine framework residues of structural importance for maintaining the canonical structures of CDRs were retained in the humanized variants, the rest of the murine framework residues were back mutated to corresponding human framework residues one by one to generate a number of humanized variants, followed by purification and characterization to determine the murine framework residues that can be replaced by human framework residues, while maintaining the desired biophysical and biochemical properties. In the second round of humanization, the critical murine framework residues that were identified during the initial round of humanization were combined to generate the desired humanized versions of murine BGAm1823 and BGAm1371. Specifically, CDRs of BGAm1823 VL were grafted into the framework of human germline variable gene IGVK1-39 with no murine residue retained, resulting in the humanized VL sequence of BGAh9239 (SEQ ID NO:10). CDRs of BGAm1823 VH were grafted into the framework of human germline variable gene IGVH1-69 with 2 murine residues (Y27, F91) retained, resulting in the humanized VH sequences of BGAh9239 (SEQ ID NO:9). CDRs of BGAm1371 VL were grafted into the framework of human germline variable gene IGVK4-1, with 1 murine residue (L4) retained, resulting in the humanized VL sequence of BGAh9179 (SEQ ID NO:22). CDRs of BGAm1371 VH were grafted into the framework of human germline variable gene IGVH1-46, 2 murine residues (I29, A71) retained, resulting in the humanized VH sequences of BGAh9179 (SEQ ID NO:21).

BGAh9239 and BGAh9179 were constructed into a humanized full-length antibody format using in-house developed expression vectors that contain constant regions of a human IgG1 and kappa chain respectively, with easy adapting sub-cloning sites. Expression and purification of BGAh9239 and BGAh9179 were achieved by co-transfection of the above two constructs into Expi293™ cells and by purification via a protein A column (Cat: 17543801, GE Life Sciences). The purified antibodies were concentrated to 0.5-5 mg/mL in PBS and stored in aliquots in a −80° C. freezer.

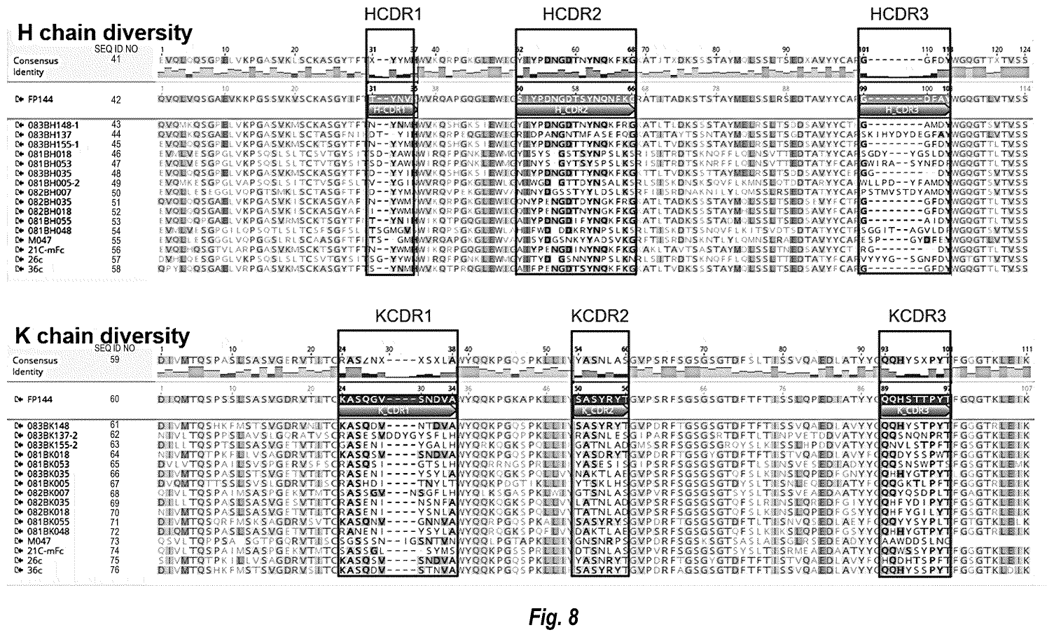

The BGAh9239 and BGAh9179 humanized antibody variants were further engineered to remove post translational modification (PTM) site(s) in the CDRs to improve molecular and biophysical properties for therapeutic use in humans. The considerations included amino acid compositions, heat stability (Tm), and surface hydrophobicity while maintaining antibody function. Normal sequence diversity was observed among top candidates (FIG. 8).

Taken together, engineered versions of humanized monoclonal antibody BGAh9239 and BGAh9179 were derived from the process described above and characterized in detail. The results showed all humanized variants were very similar in binding affinity and functional activities such as inhibiting FGFR2b-mediated downstream signaling.

### Example 4. Determination of the Binding Affinity and Specificity of Anti-FGFR2b Antibodies

The binding affinity and specificity of purified anti-FGFR2b antibodies were determined by ELISA and FACS. Briefly, 2 mg/ml of human FGFR2b (FGR-HM1BD, KACTUS) or FGFR2c (FGR-HM2CD, KACTUS) protein was coated in 96-well ELISA plates, and 50 mL of serially diluted antibodies were co-incubated for 30-60 min, washed, and incubated with goat anti-hIgG secondary antibody conjugated to HRP (Abcam, ab98624). After incubation and washing, the plates were developed with HRP substrate and absorbance was measured. Selected candidates were serially diluted and incubated with human FGFR2b, huFGFR2c, cynoFGFR2b, or mouse FGFR2b cells for 30 minutes at 4° C. After washing twice with FACS buffer, diluted Alexa Fluor 647 goat anti-human IgG secondary antibody was added and incubated with cells for 30 minutes at 4° C. in the dark. After washing twice with FACS buffer, cells were resuspended with FACS buffer and read using a Becton Dickinson LSR Fortessa™ cell analyzer. Non-specific binding of these antibodies was evaluated by FACS binding against parental 293T blank cells. Titration curves were generated using a sigmoidal dose-response of nonlinear fit from GraphPad™ Software by Dotmatics.

The sequences of the tested antibodies are shown in Table 8. BGA1421 is a comparator antibody bemarituzumab, and BGA2354 is a comparator antibody M047 (Bayer; BAY1895344). The isotype IgG control was CB-6 (also known as Etesevimab or JS016), which is a recombinant neutralizing human IgG1 anti-SARS-CoV-2 monoclonal antibody.

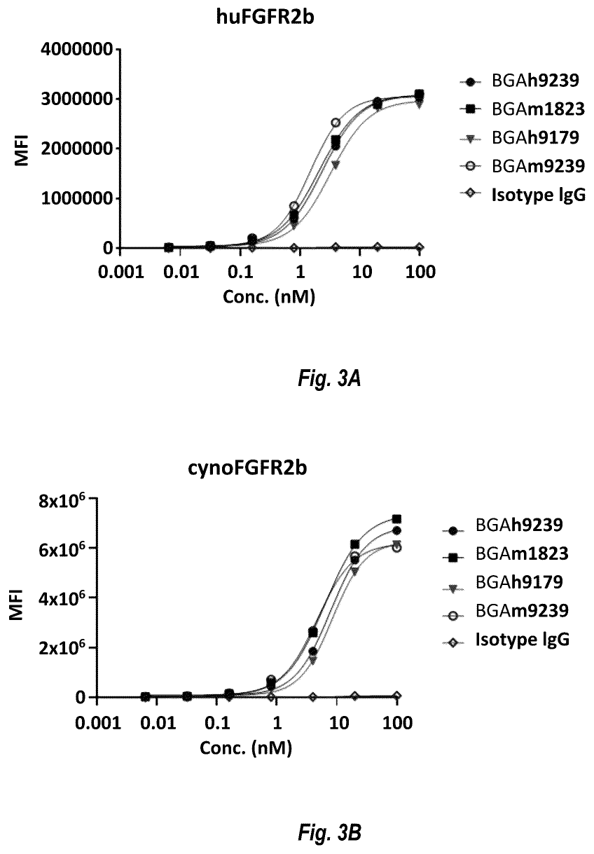

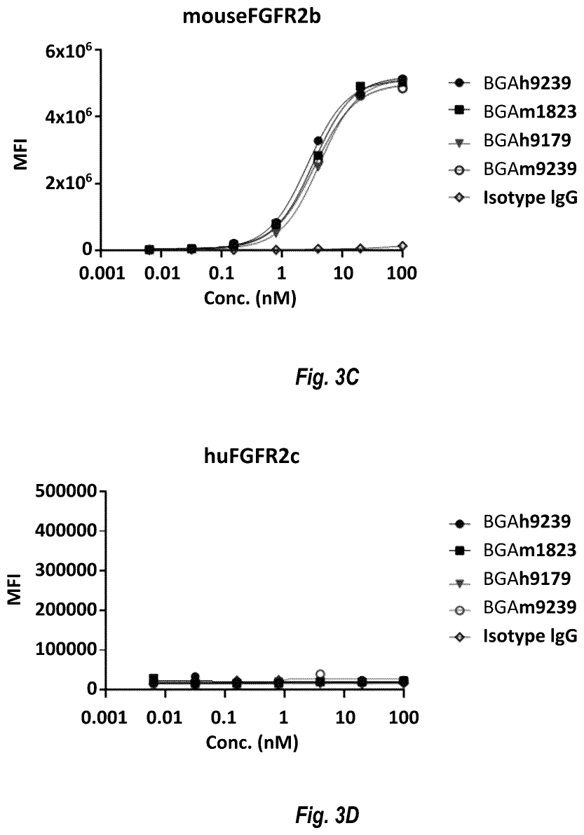

As shown in FIGS. 3A-3D and Tables 9 and 10, all selected candidates specifically bind to FGFR2b with high affinity, and cross-bind to cyno and mouse FGFR2b.

TABLE 9
Protein Binding of Chimeric FGFR2b Binders

| | Human | Human | Human |
|---|---|---|---|
| | FGFR2b | FGFR2b | FGFR2c |
| | binding EC50 | binding | binding |
| Antibody | (nM) | Emax(OD450) | Emax(OD450) |
| (BGAm1371; | 0.19 | 3.35 | 0.07 |
| parental | | | |
| mouse clone) | | | |
| BGAh9239 | 0.26 | 3.37 | 0.07 |
| BGA1421 - | 0.61 | 2.62 | 0.09 |
| Bemarituzumab - | | | |
| Comparator | | | |
| antibody alone | | | |
| (un-conjugated) | | | |
| BGA2354 | 0.04 | 3.52 | 1.66 |
| (BAY1895344 - | | | |
| Comparator | | | |
| antibody alone | | | |
| (un-conjugated) | | | |
| Isotype IgG | NA | 0.08 | 0.12 |

TABLE 10
Cellular Binding of Selected Chimeric FGFR2b Binders

| | | Cyno | | | |
|---|---|---|---|---|---|
| | Human | FGFR2b | Murine | Human | 293T |
| | FGFR2b | OE | FGFR2b | FGFR2c | blank |
| | OE cells | cells | OE cells | OE cells | cells |
| | binding | binding | binding | binding | binding |
| | EC50 | EC50 | EC50 | Emax | Emax |
| Antibody | (nM) | (nM) | (nM) | (MFI) | (MFI) |
| (BGAm1371; | 1.25 | 5.02 | 1.78 | 33 | 28 |
| parental | | | | | |
| mouse clone) | | | | | |
| BGAh9239 | 1.84 | 1.55 | 1.59 | 36 | 31 |
| BGA1421 | 0.48 | 0.49 | 0.81 | 40 | 18 |
| BGA2354 | 0.20 | 0.34 | 0.15 | 10870 | 40 |
| Isotype IgG | NA | NA | NA | 27 | 19 |

### Example 5. Binding Kinetics of Anti-FGFR2b Antibodies

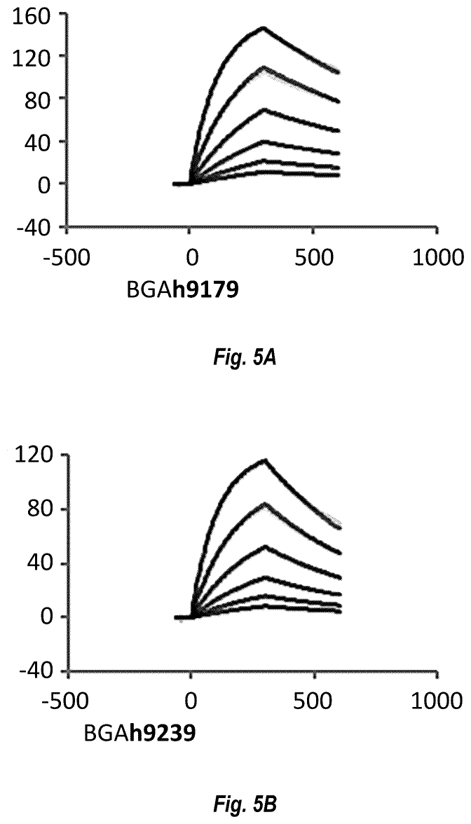

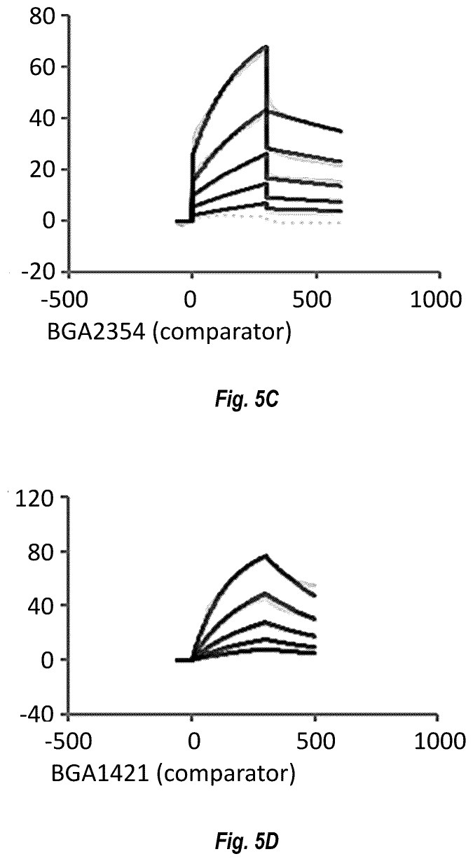

The binding affinity and kinetics of purified anti-FGFR2b antibodies were determined by surface plasmon resonance (Biacore 8K, GE Life Sciences) at room temperature. Briefly, mouse anti-human IgG Fc antibody was immobilized via amine coupling onto a activated CM5 biosensor chip (Cat. No. BR100530, GE Life Sciences). Purified monoclonal antibody candidates were flowed over the chip surface and captured by anti-human IgG antibody. Then a serial dilution of soluble human FGFR2b proteins (his tag, in-house generated) were injected over the antibodies captured surface and changes in surface plasmon resonance signals were analyzed to calculate the association rates (kₒₙ) and dissociation rates (k_{off}) by using the one-to-one Langmuir binding model (BIA Evaluation Software, GE Life Sciences). The equilibrium dissociation constant (K_{D}) was calculated as the ratio k_{off}/kₒₙ. The binding kinetics profiles of selected monoclonal anti-FGFR2b antibodies is listed below in Table 11, and detailed binding curves of selected candidates and comparators are presented in FIGS. 5A-5D.

TABLE 11
SPR Kinetics of Selected Chimeric FGFR2b Binders

| Antibody | ka (1/Ms) | kd (1/s) | KD (M) |
|---|---|---|---|
| BGAh9179 | 8.06E+04 | 1.12E−03 | 1.39E−08 |
| BGAh9239 | 7.66E+04 | 1.89E−03 | 2.47E−08 |
| BGA1421 | 1.04E+05 | 2.32E−03 | 2.22E−08 |
| BGA2354 | 4.71E+04 | 6.76E−04 | 1.43E−08 |

### Example 6. In Vitro Proliferation Inhibition Assay in BaF3_hFGFR2b OE Cells

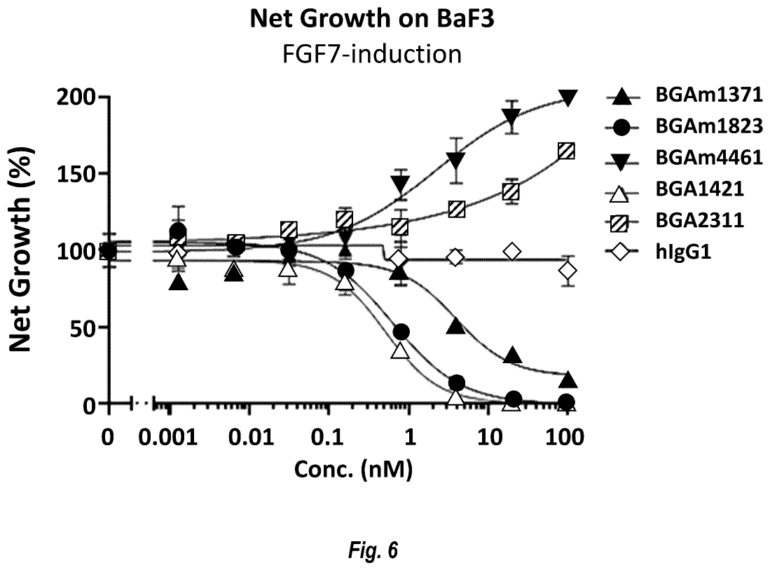

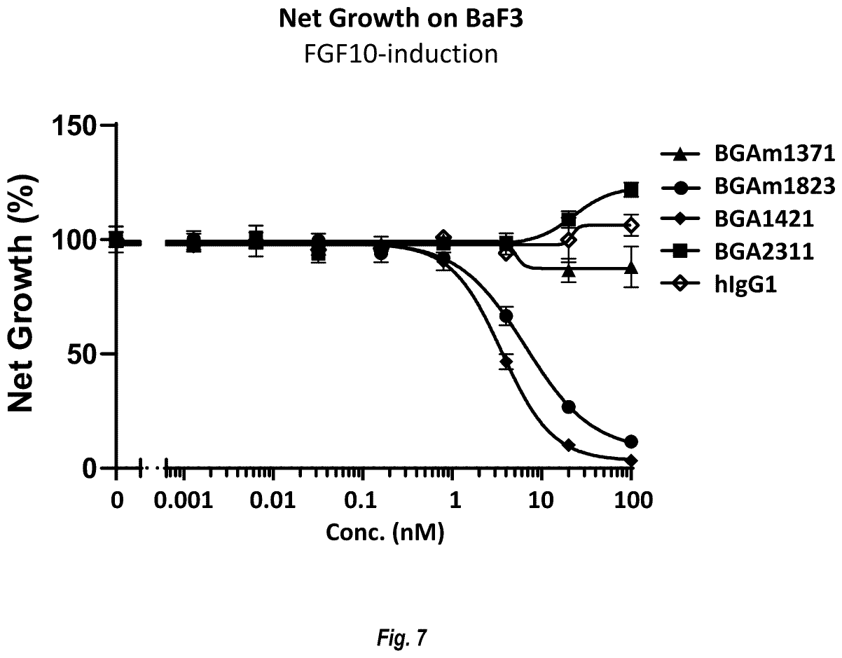

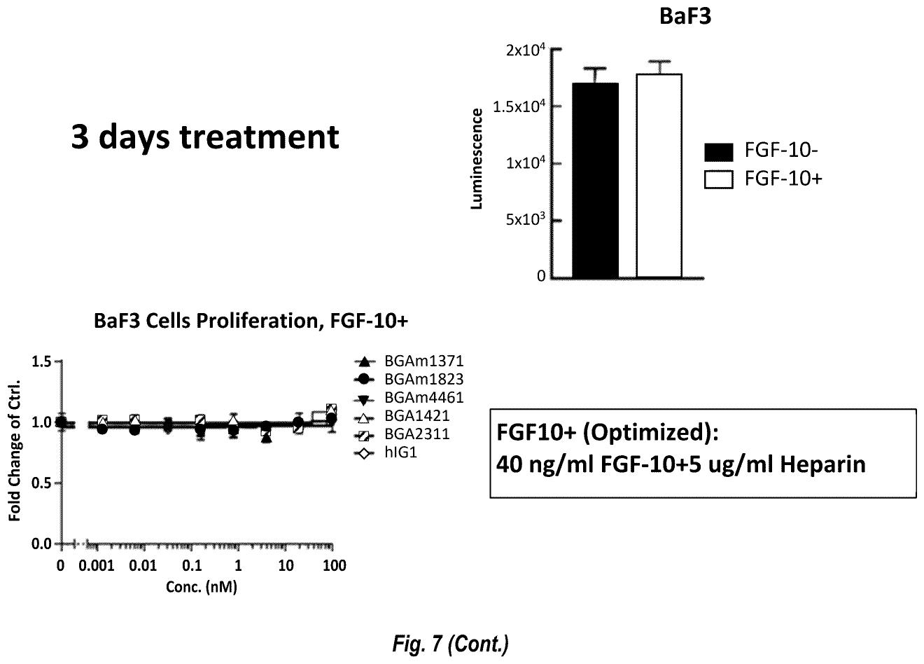

The inhibition activity of antibody on ligand-induced cell proliferation was evaluated in an FGFR2b engineered BaF3 cell line system (BaF3_hFGFR2b OE). BaF3 cells are dependent on interleukin-3 (IL-3) for growth. The BaF3 system allows for testing of kinases and kinase inhibitors. Some protein kinases can render BaF3 cells to be independent of IL-3, whereas kinase inhibitors antagonize this effect. Cells were seeded in 96-well plates at 30,000 cells/well in RPMI-1640 medium containing 10% fetal bovine serum. Anti-FGFR2b antibodies at a concentration of 10 μg/ml were added to the assay plates. Recombinant human FGF7 or FGF10 protein (40 ng/ml) in the presence of heparin (5 μg/ml) was added to the assay plates. The plates were incubated at 37° C., 5% CO₂, 95% humidity in an incubator for 72 hours. Following incubation, 50 μl Cell Titer Glo reagent was added to each well and the plates were mixed for 1 min and then incubated at RT for 10 min. Luminescence signals were detected and recorded. Comparator BGA1421 and BGAm1823 (murine parental antibody) were shown to potently inhibit both FGF7-induced and FGF10-induced BaF3_hFGFR2b cell proliferation (FIGS. 6 and 7, respectively). BGAm1371 was shown to inhibit FGF7-induced BaF3_hFGFR2b cell proliferation but is ten times less potent compared to BGA1421 and BGAm1823. BGAm4461 did not strongly inhibit FGF7-induced or FGF10-induced BaF3_hFGFR2b cell proliferation (FIG. 6 and data not shown).

The Ba/F3 system and FIGS. 6 and 7 are further described in Example 12 below.

### Example 7. In Vitro Inhibition of FGFR2 Signaling in SNU-16 Cells

Inhibition of the FGFR2 signaling pathway by the humanized antibodies was investigated. SNU-16 cells were grown in RPMI-1640 medium with 10% FBS, collected and washed twice with free medium and then seeded in 12-well plates at 1.5E6 cells/well in serum-free RPMI-1640. The plates were incubated at 37° C., 5% CO₂, and 95% humidity in an incubator overnight. Anti-FGFR2b antibodies at a concentration of 10 μg/ml were added to the assay plates. Recombinant human FGF7 or FGF10 protein (10 ng/ml) in the presence of heparin (5 μg/ml) was added to the assay plates. The plates were incubated in a 37° C., 5% CO₂, 95% humidity incubator for 2 hours. Following 2 hours incubation, cells were collected and washed twice with cold PBS. NP40 lysis buffer containing 1×PMSF and 1× Protease plus phosphatase inhibitors were added to the cell pellets. The mixture was pipetted up and down to suspend the pellet. The mixture was inverted gently and each placed for 10 minutes on ice, for a total of 30 minutes. The mixture was centrifuged at 12000× rpm for 10 minutes to pellet the cell debris. The supernatant was transferred to a new tube to quantify total protein concentration by BCA Kit. The protein samples were then split and stored at −80° C. for further Western blotting to detect the expression of pFGFR and total FGFR2.

Western blotting procedure: The completely thawed protein samples were mixed with 4×SDS loading buffer and were heated for 10 min at 37° C. Then the mixtures were centrifuged at ˜3500×rpm for 5 min. Equal amounts of proteins were loaded into an SDS-PAGE gel, whereafter proteins were transferred to PVDF membranes using iBlot. The membranes were then subjected to Western blotting analysis, focusing on phosphorylated FGFR and total FGFR2.

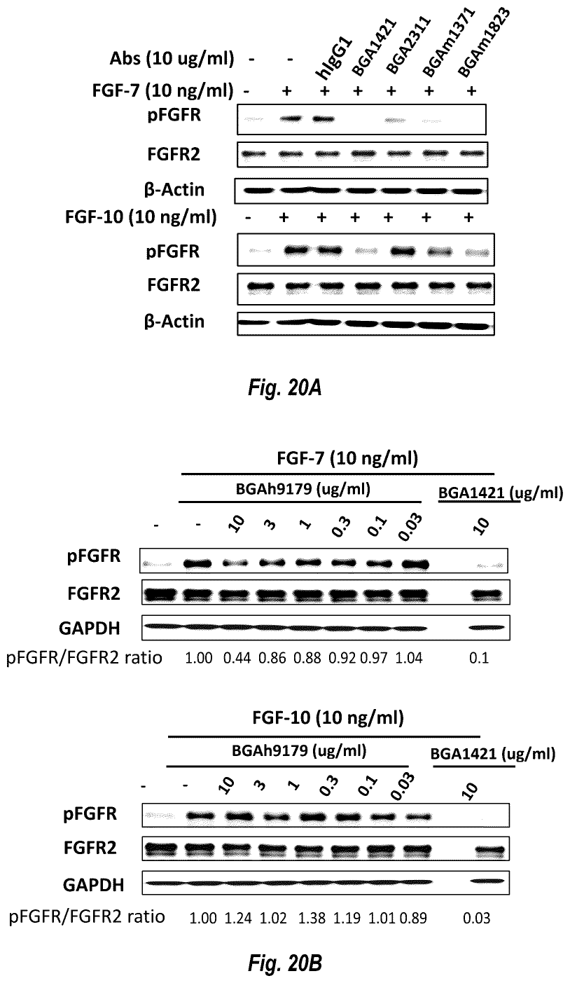

Results are shown in FIGS. 20A-20B. In FIG. 20B, n=2 for BGAh9179 and n=1 for BGA1421. Comparator BGA1421 and BGAm1823 treatment resulted in down-regulation of phosphorylated FGFR in both FGF7-induced and FGF10-induced SNU-16 cells (FIG. 20A). BGAm1371 weakly inhibited FGF7-induced FGFR phosphorylation in SNU-16 cells (FIG. 20A). BGAh9179 resulted in little or no down-regulation of FGF10-induced FGFR phosphorylation, and less down-regulation as compared to comparator BGA1421 (FIG. 20B). Comparator BGA2311 did not result in down-regulation of phosphorylated FGFR, in either FGF7-induced or FGF10-induced SNU-16 cells (FIG. 20A). Thus, the anti-FGFR2b antibodies of the present disclosure reduced the interactions of FGFR2b with its ligand, KGF.

### Example 8: BGA3457 does not Cause Corneal Dystrophy in Mice

The impact of immunoconjugate BGA3457 on corneal dystrophy was observed in Balb/c nude mice (six to eight-week-old). HSC-39 cells were inoculated in the right flank by subcutaneous injection of 5.0×10⁶ cells with matrigel (1:1 v/v ratio). Tumor volume was calculated using the formula: V=0.5×(a×b²) where a and b are the long and short diameters of the tumor, respectively. When tumor volume reached approximately 200 mm³ in size, mice were randomized into each group. Animals were intravenously administered vehicle, BGA3457 (10 mg/kg, Q2W×2), or BGA1421 (10 mg/kg, BIW×8). At the endpoint, mice were euthanized using carbon dioxide and eyes were collected and fixed in Davidson solution (95% ethanol/formaldehyde/acetic acid/distilled water: 30 mL/20 mL/10 mL/20 mL), then transferred into 75% ethanol 24 hours later. Corneal abnormalities were analyzed by H&E staining. Mouse corneal thickness was measured by Imagescope software (Leica Biosystems). The differences between the mean values of data for comparing groups were analyzed for significance using appropriate analysis by GraphPad Prism (GraphPad Software, Inc.). p<0.05 is considered to be statistically significant.

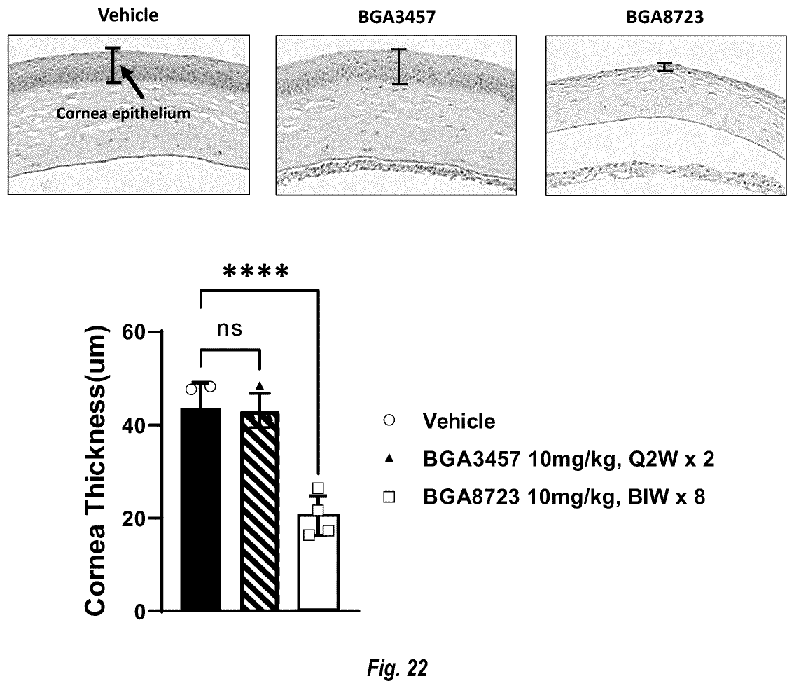

Results are shown in FIG. 22. Treatment with comparator BGA1421 (10 mg/kg, BIW×8) in mice significantly thinned the cornea compared to vehicle. However, treating mice with BGA3457 (10 mg/kg, Q2W×2) did not impact the corneal thickness. The thickness of corneas from four individual mice in each group were quantified. Thus, the anti-FGFR2b ADC (BGA3457) with lower blocking function spares corneal dystrophy in mice.

### Example 9. Affinity Determination for Humanized Anti-FGFR2b Antibodies

For affinity determination, antibodies were captured by anti-human Fc antibodies, and used in the affinity assay based on surface plasmon resonance (SPR) technology. SPR signals were analyzed to calculate the association rates (ka) and dissociation rates (kd) by using the one-to-one Langmuir binding model (BIA Evaluation Software, GE Life Sciences). The equilibrium dissociation constant (KD) was calculated as the ratio of kd/ka. Rmax represents the maximal feasible SPR signal generated by an interaction between a ligand-analyte pair and is represented in response units (RU). The results of SPR-determined binding profiles of anti-FGFR2b antibodies are summarized in Table 12 and Table 13. BGAh9239 and BGA9179 show similar binding profiles to human FGFR2b and cyno FGFR2b, and are similar to their parental antibodies BGAm1823 and BGAm1371, respectively.

TABLE 12
Binding Profiles of Anti-FGFR2b Antibodies to Human FGFR2b

| Antibody | ka (M⁻¹S⁻¹) | kd (S⁻¹) | KD(M) | Rmax |
|---|---|---|---|---|
| BGAh9239 | 8.05E+04 | 3.00E−03 | 3.73E−08 | 80.9 |
| BGAm1823 | 7.63E+04 | 1.85E−03 | 2.42E−08 | 85.7 |
| BGAh9179 | 8.16E+04 | 1.78E−03 | 2.18E−08 | 38.3 |
| BGAm1371 | 7.29E+04 | 1.19E−03 | 1.63E−08 | 96.3 |

TABLE 13
Binding Profiles of Anti-FGFR2b Antibodies to Cyno FGFR2b

| Antibody | ka (M⁻¹S⁻¹) | kd (S⁻¹) | KD(M) | Rmax |
|---|---|---|---|---|
| BGAh9239 | 7.99E+04 | 2.72E−03 | 3.40E−08 | 88.7 |
| BGAm1823 | 7.35E+04 | 1.80E−03 | 2.46E−08 | 97.5 |
| BGAh9179 | 8.37E+04 | 1.72E−03 | 2.06E−08 | 40.8 |
| BGAm1371 | 7.97E+04 | 1.25E−03 | 1.57E−08 | 102.9 |

### Example 10. Binding Activities of Humanized Anti-FGFR2b Antibodies to Native FGFR2b

To evaluate the binding activity of anti-FGFR2b antibodies to native FGFR2b on living cells, Expi293 cells were engineered to overexpress human FGFR2b, cyno FGFR2b, mouse FGFR2b, or human FGFR2c. Expi293 cells expressing antigens were seeded in 96-well plates, and were incubated with serially diluted anti-FGFR2b antibodies. Goat anti-human IgG [Alexa Fluor® 647] (Jackson ImmunoResearch, Cat. No. 109-605-003) was used as a secondary antibody to detect antibody binding to the cell surface. The change of mean fluorescent intensity (MFI) of humanized candidates BGAh9239 and BGAh9179 was measured by flow cytometry. EC50 values for dose-dependent binding to native human FGFR2b, cyno FGFR2b, mouse FGFR2b, and human FGFR2c were determined using sigmoidal dose-response curves of nonlinear fit from GraphPad Prism™. As shown in FIGS. 3A-3D and Table 14, both humanized anti-FGFR2b antibodies, BGAh9239 and BGAh9179, demonstrated high binding affinity to native human FGFR2b, cyno FGFR2b, and mouse FGFR2b on living cells, and had no detectable binding to native human FGFR2c.

TABLE 14
FACS Binding Affinities of Humanized Anti-FGFR2b Antibodies

| Antibody ID | Cell | Emax (MFI) | EC50 (nM) |
|---|---|---|---|
| BGAh9239 | Human FGFR2b | 3082608 | 2.397 |
| BGAm1823 | | 3092441 | 2.123 |
| BGAh9179 | | 2977577 | 3.285 |
| BGAm1371 | | 3052664 | 1.505 |
| BGAh9239 | Cyno FGFR2b | 6880728 | 7.926 |
| BGAm1823 | | 7359564 | 6.271 |
| BGAh9179 | | 6248725 | 8.453 |
| BGAm1371 | | 6193732 | 4.727 |
| BGAh9239 | Mouse FGFR2b | 5098781 | 2.668 |
| BGAm1823 | | 5194795 | 3.366 |
| BGAh9179 | | 5145011 | 4.247 |
| BGAm1371 | | 4984598 | 3.504 |
| BGAh9239 | Human FGFR2c | 23848 | — |
| BGAm1823 | | 22634 | — |
| BGAh9179 | | 21749 | — |
| BGAm1371 | | 23051 | — |

The “—” in the table means not tested.

### Example 11. Binding Activities of Humanized Anti-FGFR2b Antibodies to Recombinant FGFR2b

For affinity determination, antibodies were captured by anti-human Fc antibodies, and used in the affinity assay based on surface plasmon resonance (SPR) technology.

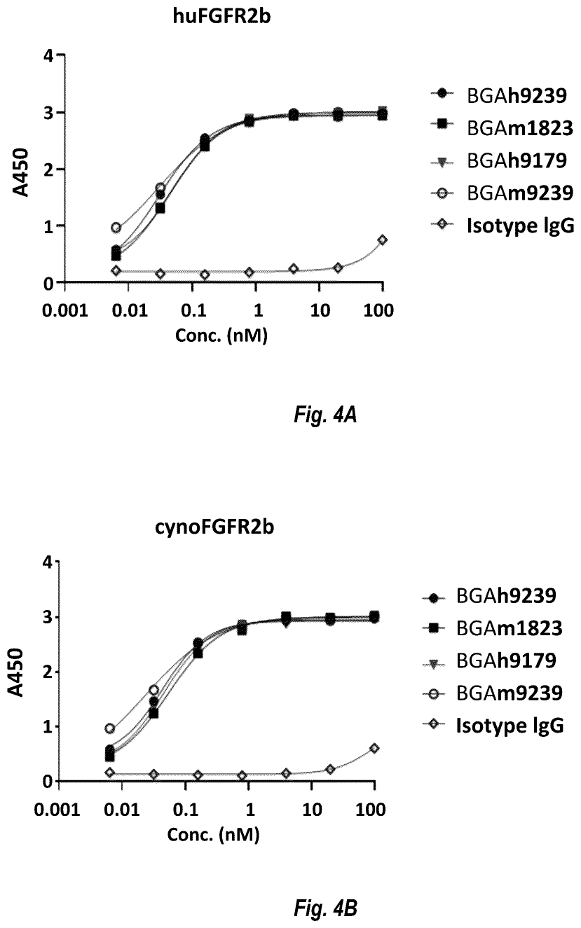

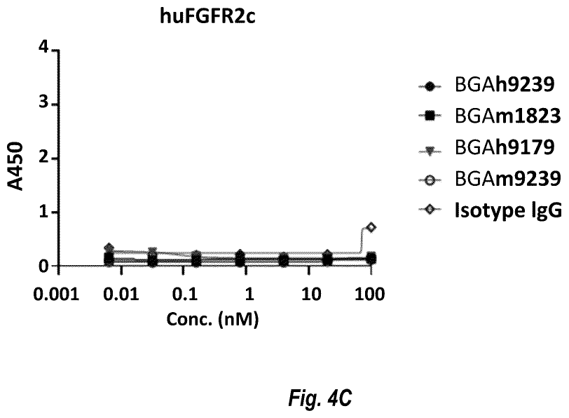

To determine whether humanized anti-FGFR2b antibodies bind to the recombinant FGFR2b proteins, ELISA plates were coated with human FGFR2b, cyno FGFR2b, or human FGFR2c overnight at 4° C. After washing and blocking, serially diluted anti-FGFR2b antibodies were added to plates and incubated for 1 hour at room temperature (an isotype human IgG, CB-6 (also known as Etesevimab or JS016), which is a recombinant neutralizing human IgG1 anti-SARS-CoV-2 monoclonal antibody, was used as a negative control). After washing, a diluted goat anti-human IgG antibody [HRP] (Abcam, Cat. No. Ab98624) was added to plates and incubated for 30 minutes at room temperature. After washing, a TMB substrate was added to plates for developing and then halted by adding a TMB substrate stop solution. The absorbance was read at 450 nm using a plate reader. Titration curves were generated using sigmoid dose-response of nonlinear fit from GraphPad, and the EC50 are shown in Table 15. As shown in Table 15 and FIGS. 4A-4C, BGAh9239, BGAm1823, BGAh9179, and BGAm1371 displayed high binding affinity against human and cyno FGFR2b proteins, but had no detectable binding to human FGFR2c.

TABLE 15
ELISA Binding Affinities of Humanized Anti-FGFR2b Antibodies

| Antibody ID | Antigen | Emax (A450) | EC50 (nM) |
|---|---|---|---|
| BGAh9239 | Human FGFR2b | 2.945 | 0.0310 |
| BGAm1823 | | 2.952 | 0.0442 |
| BGAh9179 | | 2.993 | 0.0518 |
| BGAm1371 | | 3.013 | 0.0309 |
| BGAh9239 | Cyno FGFR2b | 2.931 | 0.0442 |
| BGAm1823 | | 2.996 | 0.0551 |
| BGAh9179 | | 2.953 | 0.0469 |
| BGAm1371 | | 3.018 | 0.0245 |
| BGAh9239 | Human FGFR2c | 0.130 | — |
| BGAm1823 | | 0.116 | — |
| BGAh9179 | | 0.086 | — |
| BGAm1371 | | 0.204 | — |

The “—” in the table means not tested.

### Example 12. Blocking Studies with Anti-FGFR2b Antibodies in Ba/F3 Proliferation Assay

The Ba/F3 system is used to investigate downstream oncogenic signaling pathways and the susceptibility of driver variants to therapeutics. Driver-addicted Ba/F3 cells die when the driver-engaged signaling pathway is inhibited, and this effect can be rescued by re-introduction of FGF7 or FGF10.

FIGS. 6 and 7 show the results of Ba/F3 proliferation assays. The net growth on Ba/F3 cells overexpressing FGFR2b is reported after treatment with anti-FGFR2b antibodies of the present disclosure. Normal Ba/F3 cells die shortly after withdrawal of exogenous FGF7 or FGF10. In FIG. 6, BGAm1371 showed weaker blocking than comparator BGA1421 (a strong ligand blocker). In FIG. 7, BGAm1371 spared FGF10 blocking and did not inhibit the net growth of Ba/F3 cells overexpressing FGFR2b when induced with FGF10. Comparator BGA2311 binds at the N-terminus and is not considered to be a ligand blocker. Thus, BGAm1371 may partially block the FGFR2b signaling pathway, such as the signaling pathway mediated by FGF7.

The Ba/F3 system and FIGS. 6 and 7 are further described in Example 6 above.

### Example 13. FGFR2b Immunoconjugates BGA3457 and BGA9823 Showed Comparable Internalization

The internalization activities of FGFR2b immunoconjugates BGA3457 and BGA9823 (linker/payload structures are shown in Table 6, third row down (i.e., LD2-3), and the antibody sequences are shown in Table 8 at BGAh9179 and BGAh9239, respectively) against SNU16 (high FGFR2b expression gastric cancer cell line), SNU601-h2b (medium FGFR2b expression gastric cancer cell line), H1048-h2b (medium to low FGFR2b expression lung cancer cell line) and HSC-39 (low FGFR2b expression gastric cancer cell line) were evaluated by pHrodo™ assay. The pHrodo™ assay is a method for determining the engulfment of apoptotic cells by macrophages using a pH-sensitive fluorescently labeled succinimidyl ester pHrodo™ (Miksa, M. et al., 2009. Journal of immunological methods, 342(1-2), pp. 71-77). FACS analysis can be used to assess the levels of phagocytosis using the fluorescent label. After phagocytosis, phagosomes and lysosomes fuse. Fusion leads to decreased pH within the engulfed compartment due to the acidity of lysosomes. The low pH is detected by pH-sensitive fluorescent dyes. Once cells are engulfed, the signal becomes brighter.

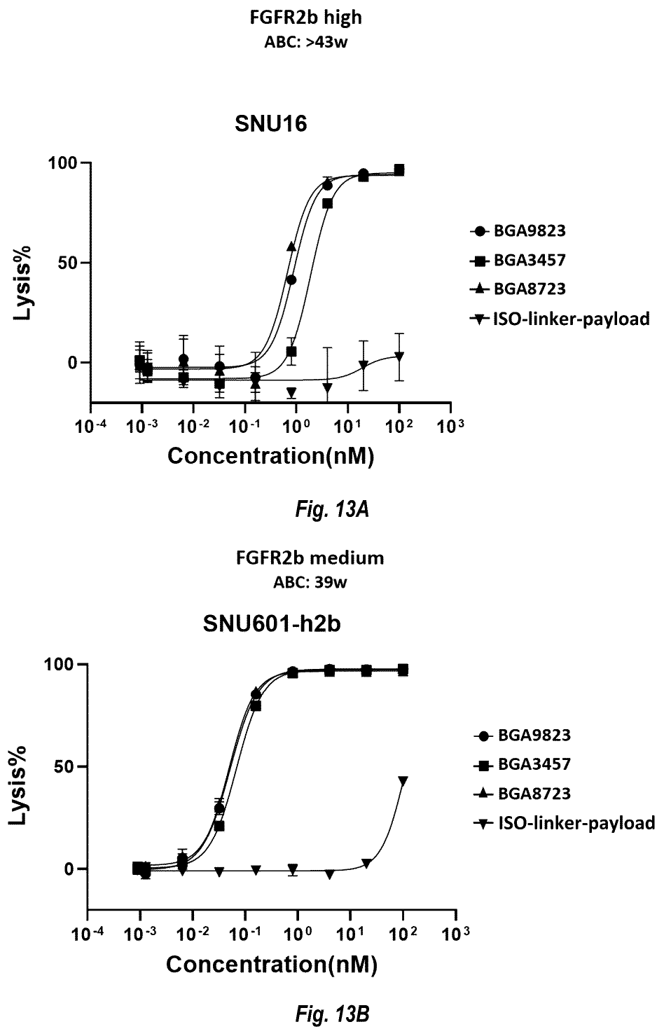

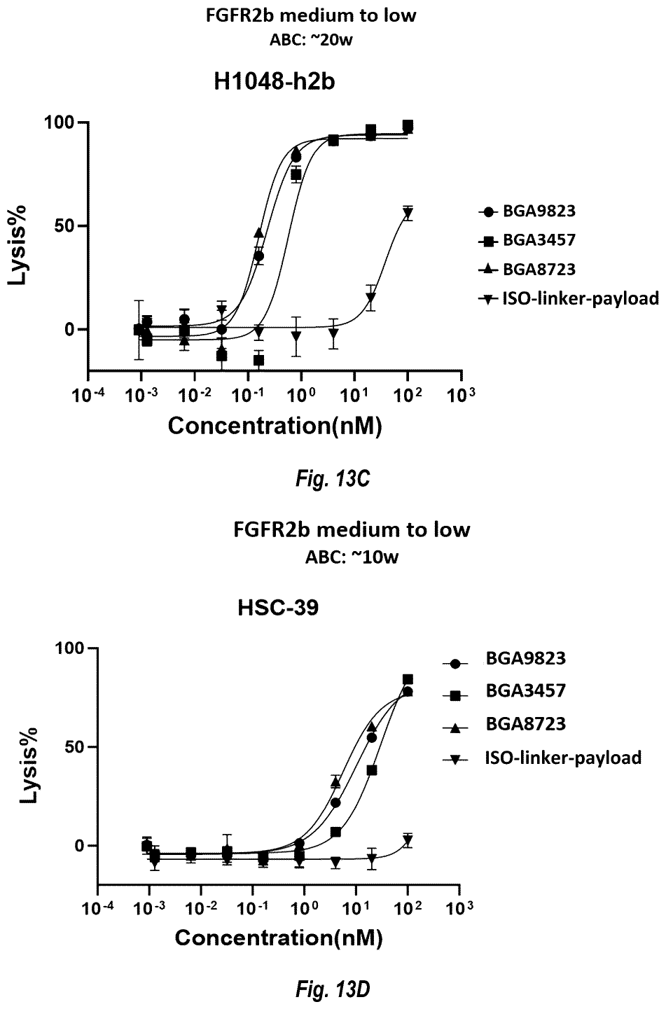

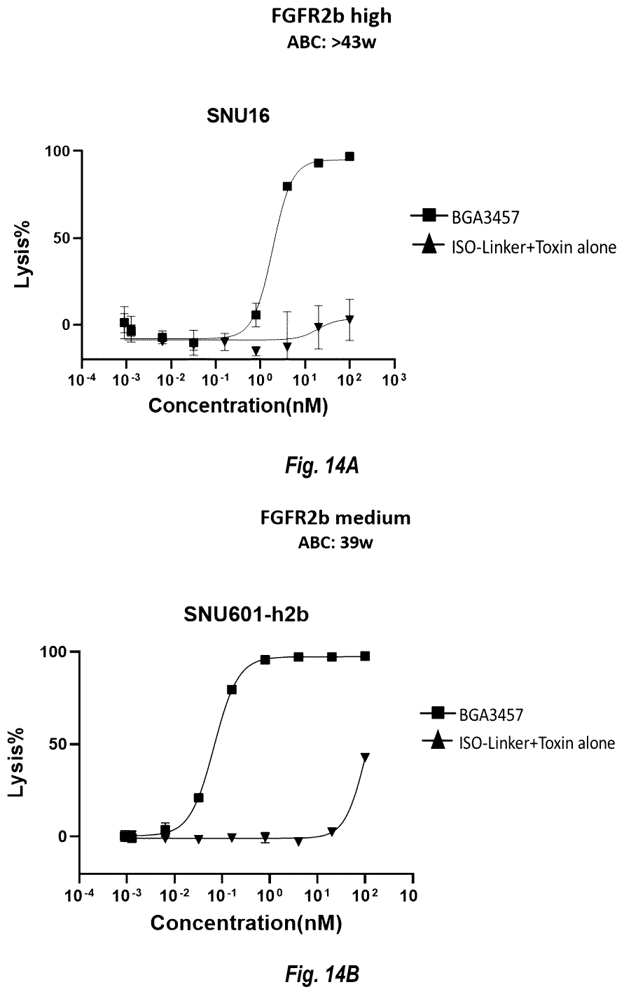

Serially diluted FGFR2b immunoconjugates and pHrodo™ labeling reagent were incubated for 5 mins at room temperature to form labeling complexes, then 1E5 cells/well were incubated with the labeling complexes at 37° C. and 5% CO₂ for 0 h, 6 h, 16 h, and 24 h. At the endpoint, cells were subjected to a FACS assay to determine the levels of pHrodo™ positive cell populations as an immunoconjugate internalized cell population. BGA3457 and BGA9823 exhibited good internalization in a high and medium FGFR2b expressing cell line, SNU16, SNU601-h2b, and H1048-h2b, (FIGS. 13A, 13B, and 13C, respectively) relative to isotype. BGA3457 and BGA9823 exhibited lower internalization in the low FGFR2b expressing cell line HSC-39 (FIG. 13D).

### Example 14. In Vitro Cell Killing Activity of FGFR2b Immunoconjugate

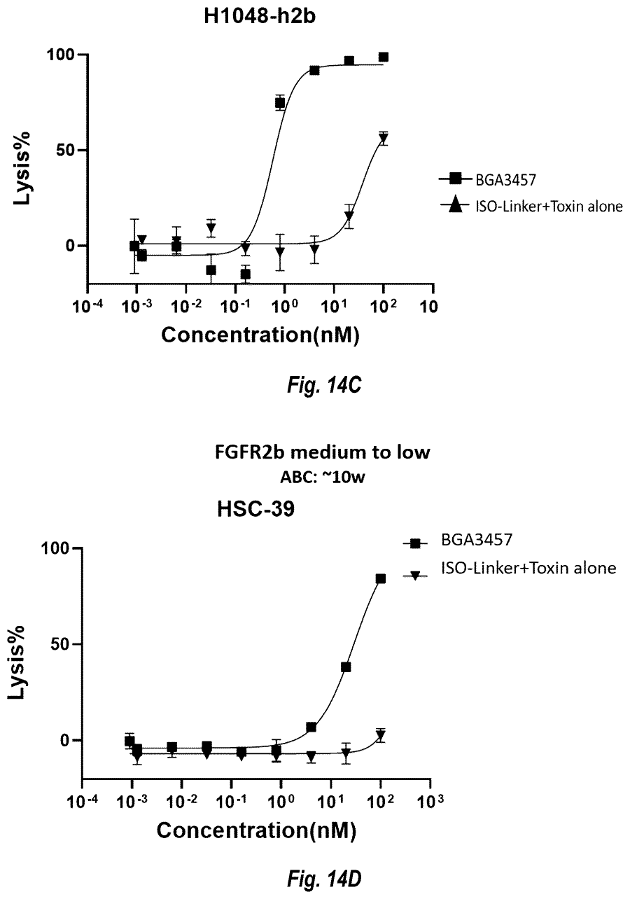

The killing activities of FGFR2b immunoconjugate BGA3457 against SNU16 (high FGFR2b expression cancer cell line), SNU601-h2b (medium FGFR2b expression cancer cell line), H1048-h2b (medium to low FGFR2b expression cancer cell line) and HSC39 (low FGFR2b expression cancer cell line) were evaluated by CellTiter-Glo assay. 2000 cells/well of SNU16, SNU601-h2b, H1048-h2b, and 1000 cells/well of HSC39 were seeded in 96-well plates and incubated at 37° C. overnight. Serial diluted FGFR2b BGA3457 was added, and the cells were then cultured for 6 days at 37° C. and 5% CO₂. Target cell killing was measured by CellTiter-Glo detection kit (Promega). Results are shown in FIG. 14. BGA3457 showed good cell killing in high to medium FGFR2b expressing cell lines at a low concentration of the FGFR2b immunoconjugate (FIGS. 14A-14C). In the low FGFR2b expressing cell line, a high concentration of FGFR2b immunoconjugate was necessary for cell killing (FIG. 14D).

### ADC In Vivo Efficacy Studies

### Example 15. Efficacy of BGA3457 and BGA9823 in Cell-Line Derived Xenograft Models

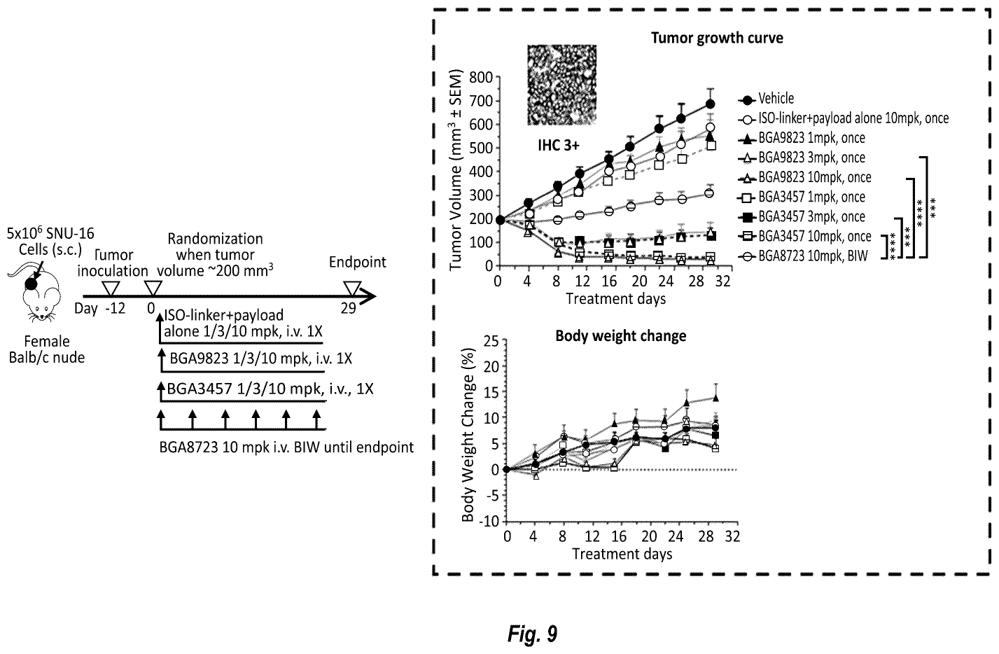

Gastric adenocarcinoma cell-line derived xenograft (CDX) models using SNU-16 (FGFR2b high expression) or HSC-39 (FGFR2b moderate expression) cells were generated in mice (FIGS. 9, 10, and 15).

SNU-16 and HSC-39 cells were cultured in RPMI-1640 medium, supplemented with 10% (v/v) fetal bovine serum, 100 U/ml penicillin, and 100 μg/mL streptomycin. On the day of implantation, cells were collected and re-suspended in cold (4° C.) serum-free RPMI-1640 medium. Cell density was adjusted to 5×10⁷ cells/mL, and the cells were placed on ice prior to inoculation.

Six- to eight-week-old female Balb/c nude mice were purchased and housed in ventilated cages, given food and water ad libitum, and allowed to acclimate for approximately 1 week prior to cell inoculation. Gastric adenocarcinoma SNU-16 and HSC-39 cells were inoculated in the right flank by subcutaneous injection of 5.0×10⁶ cells with matrigel (1:1 v/v ratio) in mice.

Tumor volume was calculated using the formula: V=0.5×(a×b²) where a and b are the long and short diameters of the tumor, respectively. When tumor volume reached approximately 200 mm³ in size, mice were randomized into study groups with 7-8 animals per group. Animals were intravenously administrated with vehicle, Iso-Control (linker+payload alone) (10 mg/kg or 3 mg/kg, single dose), BGA9823 (10 mg/kg, 3 mg/kg, or 1 mg/kg, single dose) BGA3457 (10 mg/kg, 3 mg/kg, or 1 mg/kg, single dose), or BGA8723 (10 mg/kg, BIW). Animal body weight and tumor volume were measured twice weekly. Data is presented as mean tumor volume ±standard error of the mean (SEM). Tumor growth inhibition (TGI) was calculated using the following formula:

% TGI=[1−(treated Tt−treated T0)/(vehicle Tt−vehicle T0)]×100%
treated Tt=mean tumor volume of a dosing group on Day t
treated T0=mean tumor volume of a dosing group on Day 0
vehicle Tt=mean tumor volume of vehicle group on Day t
vehicle T0=mean tumor volume of vehicle group on Day 0

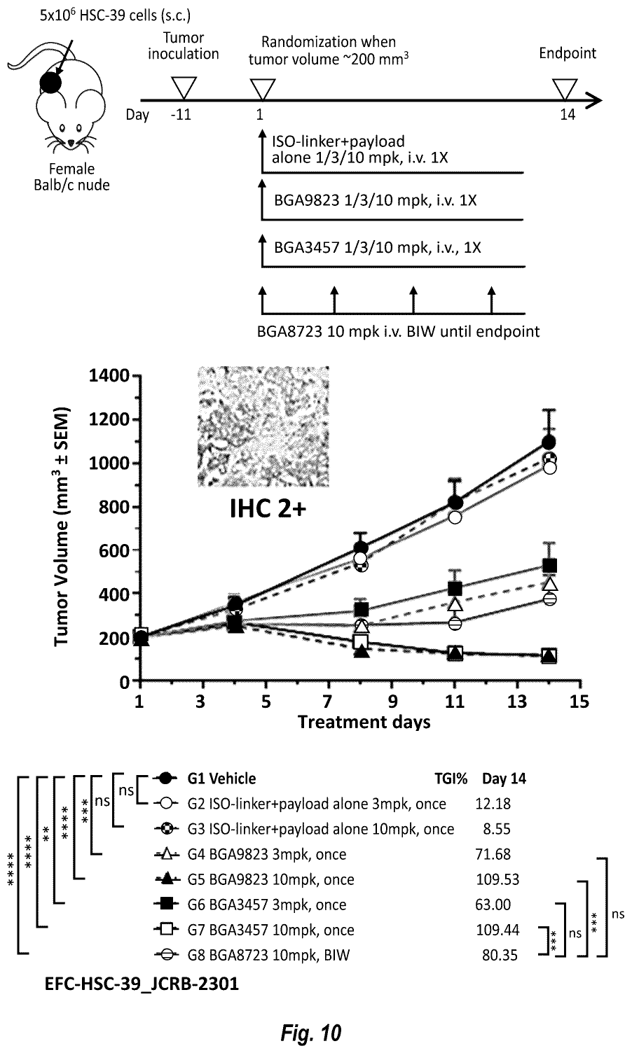

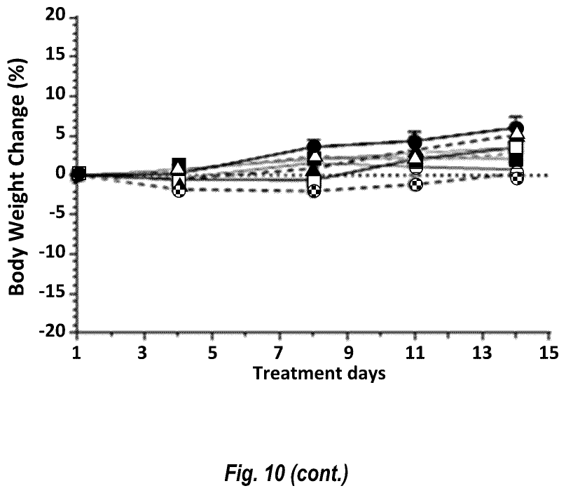

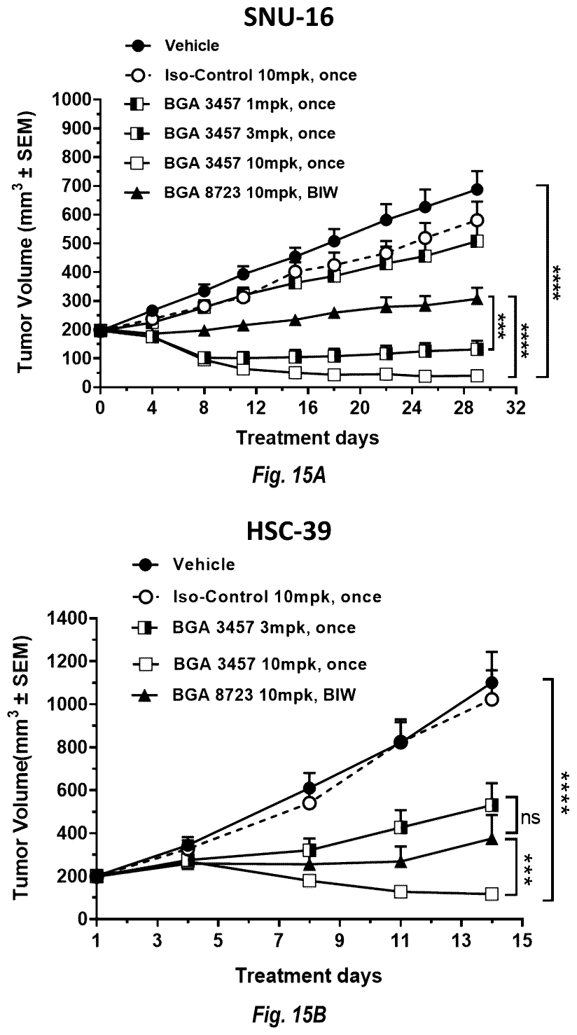

Data were analyzed using ANOVA in CDX models; ***p<0.001, ****p<0.0001. Results are presented in FIGS. 9, 10, 15A, and 15B. BGA9823 and BGA3457 demonstrated significant anti-tumor activities in SNU-16 (FIGS. 9 and 15A) and HSC-39 (FIGS. 10 and 15B) xenograft models. In the SNU-16 xenograft model, both BGA9823 and BGA3457 showed dose-dependent efficacy. At dosage levels of 3 mg/kg and 10 mg/kg, both BGA9823 and BGA3457 demonstrated superior anti-tumor efficacy relative to comparator BGA8723 (FIGS. 9 and 15A). In the HSC-39 xenograft model, both BGA9823 and BGA3457 at 3 mg/kg demonstrated comparable efficacy relative to comparator BGA8723 (FIGS. 10 and 15B). In addition, both BGA9823 and BGA3457 at 10 mg/kg significantly inhibited tumor growth and demonstrated superior anti-tumor effects relative to comparator BGA8723 (FIGS. 10 and 15B). All animals tolerated the treatment well, with no significant body weight decrease or abnormal clinical observations.

### Example 16. Efficacy of BGA3457 in Human Gastric Cancer PDX Model

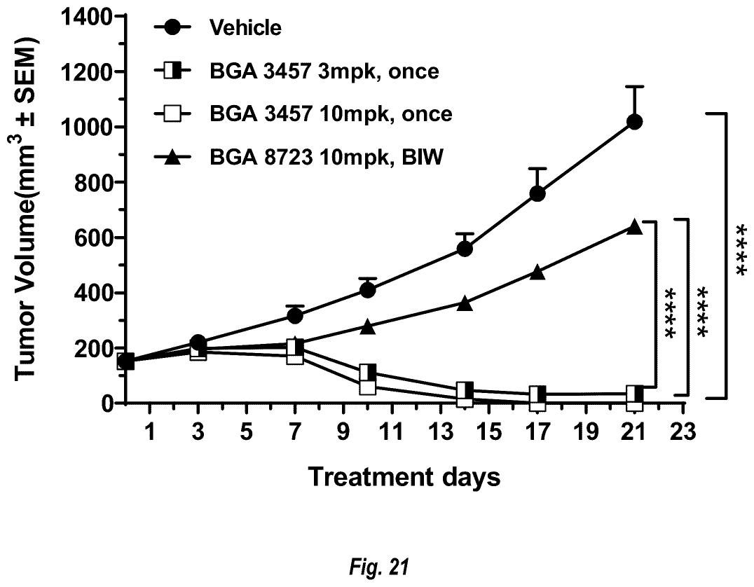

A human gastric cancer PDX model (LD1-0017-200652, FGFR2b moderate expression, Shanghai LIDE Biotech Co., Ltd) was generated. Briefly, about 50-90 mg tumor pieces were implanted subcutaneously in the right flank of female Balb/c nude mice (six- to eight-week-old). Tumor volume was calculated using the formula: V=0.5×(a×b²) where a and b are the long and short diameters of the tumor, respectively. When tumor volumes reached approximately 150 (i.e., 100-200) mm³ in size, mice were randomized into 4 groups with 3 animals in each group. Animals were intravenously administrated with vehicle, BGA3457 (3 or 10 mg/kg, single dose), or BGA8723 (10 mg/kg, BIW). Animal body weight and tumor volume were measured twice weekly. Data is presented in FIG. 21 as mean tumor volume ±standard error of the mean (SEM). Tumor growth inhibition (TGI) was calculated using the following formula:

% TGI=[1−(treated Tt−treated T0)/(vehicle Tt−vehicle T0)]×100%
treated Tt=mean tumor volume of a dosing group on Day t
treated T0=mean tumor volume of a dosing group on Day 0
vehicle Tt=mean tumor volume of vehicle group on Day t
vehicle T0=mean tumor volume of vehicle group on Day 0

Data were analyzed using to bit in PDX model; ****p<0.0001. Results are presented in FIG. 21. In a human patient-derived gastric cancer model, at dosage levels of 3 mg/kg and 10 mg/kg, BGA3457 demonstrated superior anti-tumor efficacy relative to comparator BGA8723. All animals tolerated the treatment well, with no significant body weight decrease or abnormal clinical observations.

### Example 17. In Vivo Efficacy of FGFR2b Immunoconjugates BGA3457 and BGA9823 on the Growth of Co-Inoculated HSC-39+SNU-5 Gastric Cancer Xenografts

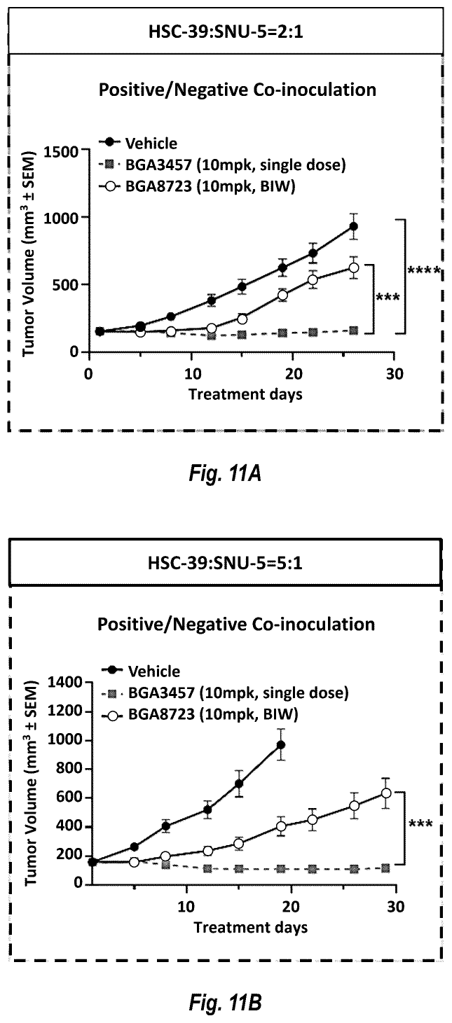

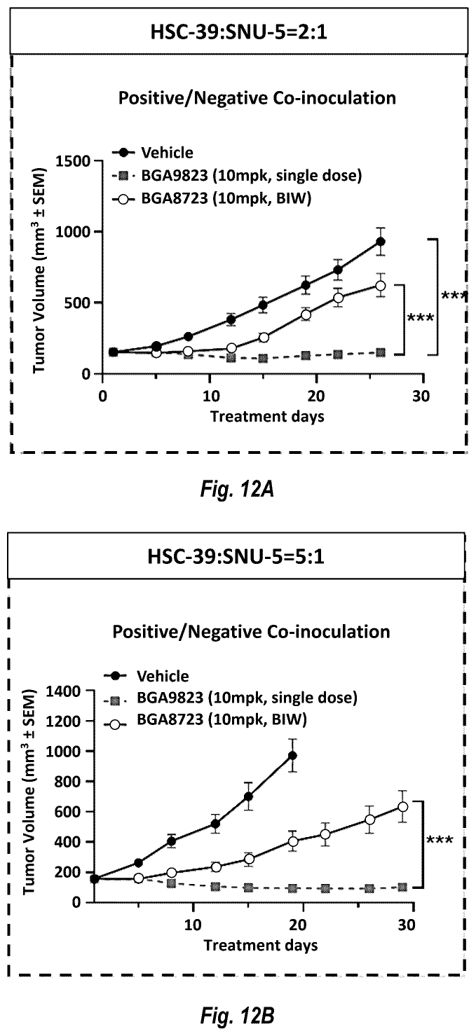

The ability of anti-FGFR2b immunoconjugates of the present disclosure, BGA9823 and BGA3457, to inhibit tumor growth was tested in a co-incubation model with HSC-39 (gastric signet ring cell adenocarcinoma, human FGFR2b overexpressed cell line) and SNU-5 (stomach cancer cell line) at a ratio of 2:1 or 5:1 (FIGS. 11A-12B).

HSC-39 and SNU-5 were cultured in Dulbecco's modified Eagle's medium (DMEM), supplemented with 10% (v/v) fetal bovine serum, 100 U/ml penicillin, and 100 μg/mL streptomycin. Cells were incubated in a humidified incubator with 5% CO₂ at 37° C. On the day of implantation, cells were collected and washed with serum-free medium. Six- to eight-week-old female BALB/c nude mice were purchased and housed in ventilated cages, given food and water ad libitum, and allowed to acclimate for approximately 1 week prior to inoculation. A total of 5×10⁶ HSC-39 and SNU-5 cancer cells mixed with matrigel at a ratio of 2:1 or 5:1 were implanted subcutaneously into the mouse flank. For tumor volume measurements, all tumors were measured with calipers and tumor volumes were calculated using the formula: V=0.5 (a×b²). When tumor volumes reached approximately 150 mm³, mice were randomized into 3 groups with 5 animals per group and received a single dose of vehicle, comparator anti-FGFR2b monoclonal antibody (BGA1421), or an FGFR2b-immunoconjugate (BGA3457, FIG. 11, or BGA9823, FIG. 12) at 10 mpk via intravenous injection on treatment day 0. Tumor volumes were measured twice weekly. Data is presented as mean tumor volume ±standard error of the mean (SEM). In the vehicle group at a 2:1 HSC-39:SNU-5 ratio, there were ˜50% FGFR2b positive cells in the tumor at the starting point with ˜40% at the end point. In the vehicle group at a 5:1 HSC-39:SNU-5 ratio, there were ˜60% FGFR2b positive cells in the tumor at the starting point with ˜20% at the end point.

As shown in FIGS. 11A, 11B, 12A, and 12B, BGA3457 and BGA9823 treatment induced complete tumor regression at ratios of 2:1 and 5:1. The comparator BGA1421 inhibited tumor growth but was less potent than the anti-FGFR2b immunoconjugates of the present disclosure, which could be attributed to limited elimination of the FGFR2b-positive population by comparator BGA1421. These results indicate that FGFR2b-immunoconjugates BGA3457 and BGA9823 showed significant killing effects against not only FGFR2b-positive tumors but also FGFR2b-negative tumors under co-inoculated conditions.

### Example 18: Bystander Killing Effects of FGFR2b Immunoconjugate BGA3457

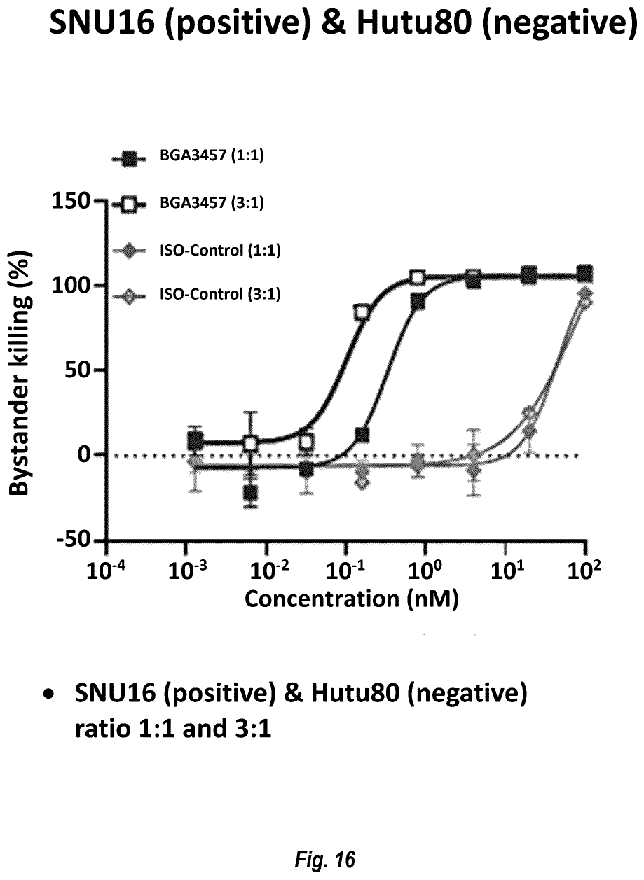

To investigate the bystander effect of BGA3457, a coculture killing assay with mixed FGFR2b-positive and -negative cells was established. Using luciferase as a readout, BGA3457 effectively killed FGFR2b-negative HuTu-80-NanoLuc cells (engineered with NanoLuc luciferase) cocultured with FGFR2b-positive SNU-16 cells (FIG. 16). The maximum killing potency reached 90.74% and the IC50 value was 34.75 ng/mL. Isotype ADC (CB-6 anti-SARS-CoV-2 monoclonal antibody conjugated to LD2-3) was used as a negative control, which showed no bystander killing effect.

### Example 19. Preparation of Toxins P3 and P4

### UPLC Analysis Methods:

Method A: Mobile phase A: 0.1% FA in water, B: MeCN; Gradient: 10% B maintain 0.2 min, 10%-95% B, 5.8 min, 95% B maintain 0.5 min; Flow rate: 0.6 mL/min; Column: ACQUITY UPLC® BEH C18 1.7 μm.

Method B: Mobile phase A: 0.1% FA in water, B: MeCN; Gradient: 10% B maintain 0.5 min, 10%-90% B, 2.5 min, 90% B maintain 0.2 min; Flow rate: 0.6 mL/min; Column: ACQUITY UPLC® BEH C18 1.7 μm.

Method C: Mobile phase A: 0.1% FA in water, B: MeCN; Gradient: 10% B maintain 0.2 min, 10%-90% B, 1.3 min, 90% B maintain 0.3 min; Flow rate: 0.6 mL/min; Column: ACQUITY UPLC® BEH C18 1.7 μm.

P1 and P2 were commercially available and purchased from MedChemExpress CO. LTD (Shanghai).

### Synthetic Procedure for P3

Provided herein are the synthetic procedures to prepare P3 and P4, which are toxins shown in Tables 5A-5B useful in the immunoconjugates of the present disclosure.

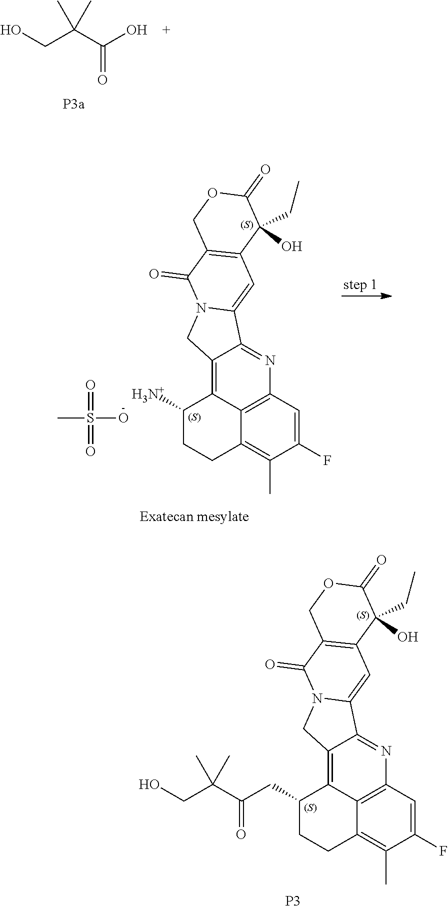

N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-3-hydroxy-2,2-dimethylpropanamide (P3).

To a mixture of P3a (5.0 mg, 0.042 mmol) and HATU (16 mg, 0.042 mmol) in DMF (1 mL) were added DIEA (21 μL, 16 mg, 0.13 mmol) and exatecan mesylate (23 mg, 0.043 mmol, purchased from MedChemExpress CO. LTD). The resulting brown mixture was stirred at r.t. for 2 h. On completion of the reaction, the mixture was purified by prep-HPLC (TFA) (Method: column: XBridge Prep C18 OBD 5 μm 19*150 mm; Mobile phase: A-water (0.1% TFA): B-acetonitrile; Flow rate: 20 mL/min). The fraction was lyophilized to give P3 (15 mg, 65.5% yield) as a white powder.

MS (ESI) m/z: 536.4 [M+H]⁺

¹H NMR (400 MHz, DMSO-d₆) δ 8.00 (d, J=8.4 Hz, 1H), 7.79 (d, J=11.2 Hz, 1H), 7.31 (s, 1H), 6.52 (s, 1H), 5.59-5.54 (m, 1H), 5.42 (s, 2H), 5.18 (q, J=19.2 Hz, 2H), 4.87 (t, J=5.2 Hz, 1H), 3.45 (dd, J=10.2, 4.8 Hz, 1H), 3.41-3.28 (m, 1H), 3.15 (t, J=5.6 Hz, 2H), 2.40 (s, 3H), 2.24-2.07 (m, 2H), 1.92-1.80 (m, 2H), 1.11 (d, J=7.6 Hz, 6H), 0.87 (t, J=7.2 Hz, 3H).

### Synthetic Procedure for P4

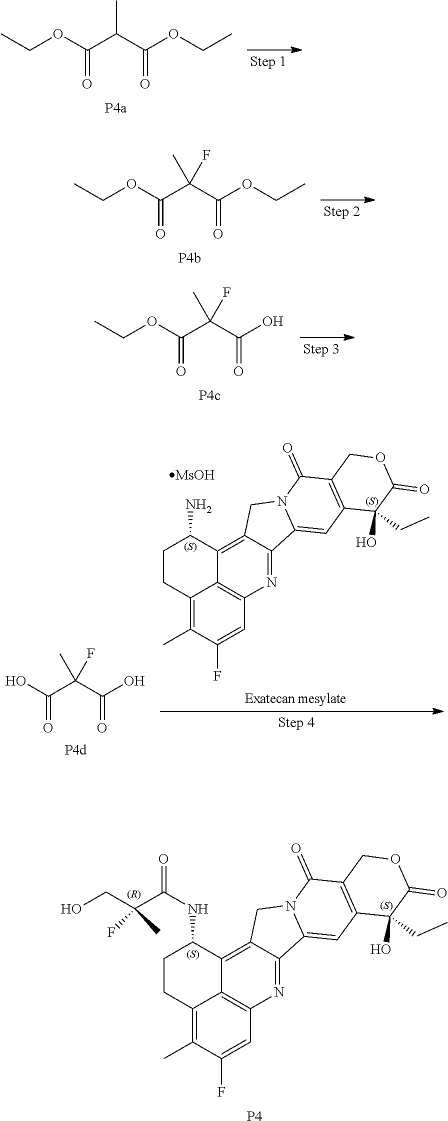

### Step 1

### Diethyl 2-fluoro-2-methylmalonate (P4b)

A solution of compound P4a (10.00 g, 57.40 mmol) in THF (200 mL) was cooled to 0° C. 60% of NaH in oil (3.21 g, 80.37 mmol) was added to the mixture portion wise, stirred at 0° C. for 30 min. Then N-fluoro-N-(phenylsulfonyl)benzenesulfonamide (NSFI, 19.91 g, 63.20 mmol) was added to the mixture portion wise at 0° C., then warmed to r.t. and stirred for 16 h. After the reaction completed, the suspension was filtered and the filtrate was concentrated. PE (100 mL) was added to the residue, the precipitate was filtered, and the filtrate was concentrated to give compound P4b (12.50 g, crude) as a light-yellow oil.

¹H NMR (400 MHz, CDCl₃) δ 4.30 (q, J=7.2 Hz, 4H), 1.79 (d, J=22.0 Hz, 3H), 1.31 (t, J=7.2 Hz, 6H).

¹⁹F NMR (376 MHz, CDCl₃) δ −157.50.

### Step 2

### 3-ethoxy-2-fluoro-2-methyl-3-oxopropanoic Acid (P4c)

To a solution of compound P4b (1.00 g, 5.20 mmol) in EtOH (5 mL) was added KOH solution (321 mg) in H₂O (50 μL) and EtOH (2 mL) dropwise at 0° C. The mixture was stirred at r.t. for 2 h. The mixture was diluted with water (20 mL), and washed with DCM (20 mL*3). The aqueous solution was adjusted to pH=3 by 1 N HCl, then extracted by EtOAc (50 mL*3). The organic layer was dried, combined, and dried over anhydrous Na₂SO₄, filtered, and concentrated to give compound P4c (470 mg, 55.0% yield) as a colorless oil.

¹H NMR (400 MHz, CDCl₃) δ 8.31 (br s, 1H), 4.32 (q, J=7.2 Hz, 2H), 1.83 (d, J=22.0 Hz, 3H), 1.33 (t, J=7.2 Hz, 3H).

¹⁹F NMR (376 MHz, CDCl₃) δ −157.59.

### Step 3

### 2-fluoro-3-hydroxy-2-methylpropanoic Acid (P4d)

To a solution of compound P4c (200 mg, 1.22 mmol) in isopropanol (4 mL) was added 2 M LiBH₄ (1.22 mL, 2.44 mmol) at 0° C. The mixture was stirred at r.t. for 2 h. The mixture was quenched by 2 N HCl (1.22 mL) dropwise at 0° C., then diluted with H₂O (10 mL), and extracted with EtOAc (50 mL*3). The organic layer was combined and dried over anhydrous Na₂SO₄, filtered, and concentrated to give compound P4d (92 mg, 61.7% yield) as a colorless oil.

¹H NMR (400 MHz, CDCl₃) δ 4.01-3.81 (m, 2H), 1.58 (d, J=21.2 Hz, 3H).

¹⁹F NMR (376 MHz, CDCl₃) δ −163.98.

### Step 4

### N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-fluoro-3-hydroxy-2-methylpropanamide (P4)

To a solution of compound P4d (23 mg, 0.19 mmol) in DMF (2 mL) were added exatecan mesylate (50 mg, 0.094 mmol), HATU (54 mg, 141 mmol), and DIEA (36 mg, 0.28 mmol). The mixture was stirred at r.t. for 1 h. The mixture was purified by prep-HPLC (FA) (Method: column: XBridge Prep C18 OBD 5 um 19*150 mm; Mobile phase: A-water (0.1% formic acid): B-acetonitrile; Flow rate: 20 mL/min). The fraction was lyophilized to give the following two isomers:

Isomer 1: P4: white solid, (11 mg, 21.9% yield). UPLC-MS, RT=3.52 min.

¹H NMR (400 MHz, DMSO-d₆) δ 9.06 (dd, J=9.0, 2.8 Hz, 1H), 8.00 (d, J=10.9 Hz, 1H), 7.54 (s, 1H), 6.75 (s, 1H), 5.82 (d, J=8.0 Hz, 1H), 5.65 (s, 2H), 5.43 (dt, J=77.8, 12.4 Hz, 3H), 4.17-3.91 (m, 1H), 3.83 (ddd, J=18.0, 12.4, 5.6 Hz, 1H), 3.40-3.27 (m, 1H), 2.62 (s, 3H), 2.50-2.34 (m, 2H), 2.22-1.98 (m, 2H), 1.81 (d, J=21.4 Hz, 3H), 1.11 (t, J=7.2 Hz, 3H); MS (ESI) m/z: 540.3 [M+H]⁺.

Isomer 2: P4-1: white solid, (8.4 mg, 16.6% yield). UPLC-MS, RT=3.86 min.

¹H NMR (400 MHz, DMSO-d₆) δ 8.72 (dd, J=8.4, 2.4 Hz, 1H), 7.78 (d, J=11.2 Hz, 1H), 7.31 (s, 1H), 6.52 (s, 1H), 5.58 (d, J=8.0 Hz, 1H), 5.42 (s, 2H), 5.32-5.05 (m, 3H), 3.83 (dd, J=26.8, 12.0 Hz, 1H), 3.61 (dd, J=21.6, 12.0 Hz, 1H), 3.22-3.07 (m, 2H), 2.46-2.30 (m, 3H), 2.28-2.05 (m, 2H), 2.02-1.74 (m, 2H), 1.45 (d, J=21.4 Hz, 3H), 0.87 (t, J=7.2 Hz, 3H); MS (ESI) m/z: 540.3 [M+H]⁺.

### Example 20: Synthesis of Examples LD2-3˜LD2-8

Provided herein are the synthetic procedures to prepare LD2-3 through LD2-8, which are conjugator-linker-payloads shown in Table 6, the components of which, and the full structures of which, are useful in the immunoconjugates of the present disclosure.

### Example LD2-3

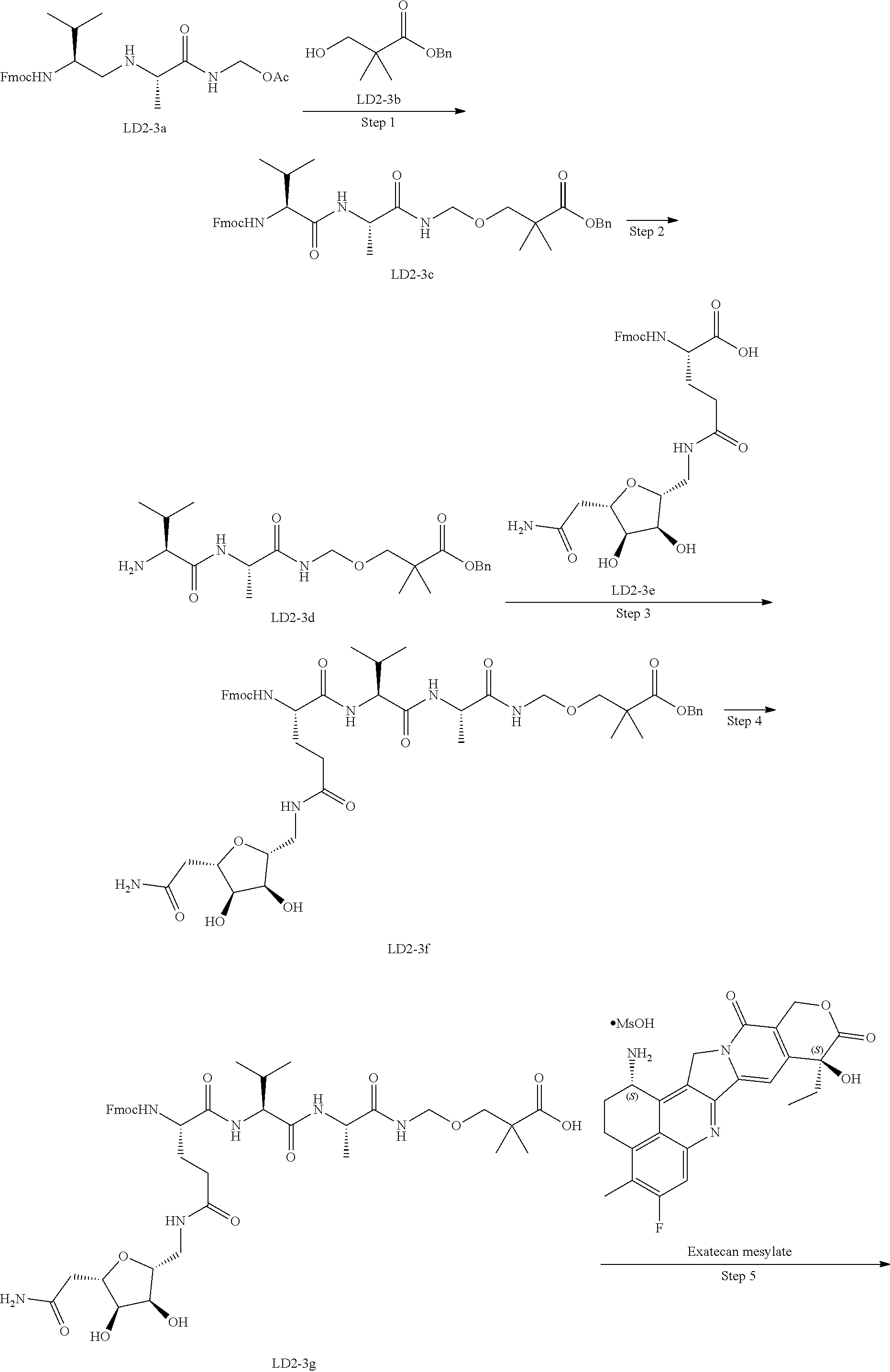

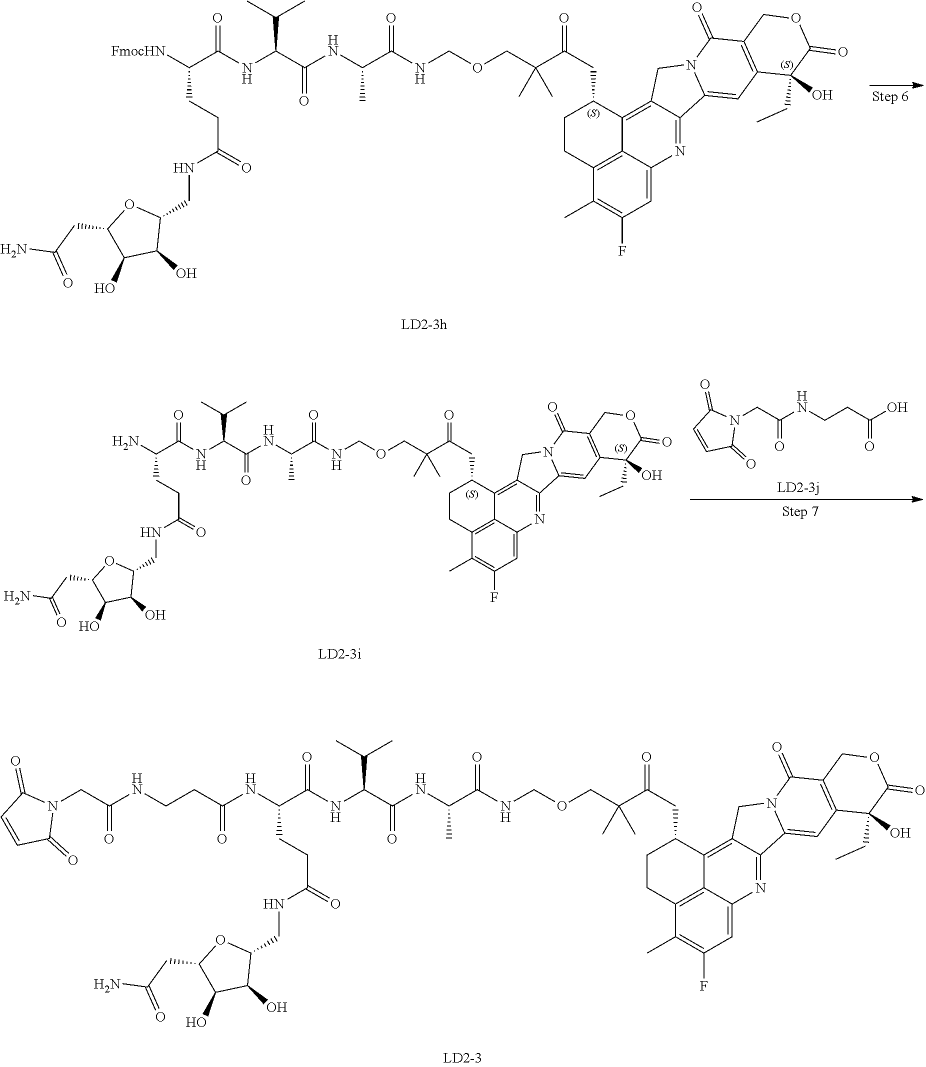

### Step 1

### benzyl (5S,8S)-1-(9H-fluoren-9-yl)-5-isopropyl-8,14,14-trimethyl-3,6,9-trioxo-2,12-dioxa-4,7,10-triazapentadecan-15-oate (LD2-3c)

A white suspension mixture of LD2-3a (300 mg, 0.62 mmol, synthesized according to reported procedures: ACS Med. Chem. Lett. 2019, 10, 1386-1392 and U.S. Pat. No. 9,808,537B2), LD2-3b (260 mg, 1.25 mmol) and 4 Å molecular sieve in anhydrous THF (10 mL) was stirred at r.t. for 10 min. Sc(OTf)₃ (368 mg, 0.75 mmol) was added and the resulting yellow suspension was stirred at r.t. for 4 hr. The yellow suspension mixture was filtered through a pad of celite and washed with EtOAc (30 mL). Combined organic layers were washed with sat. NaHCO₃ (30 mL) and brine (30 mL), dried over Na₂SO₄, filtered, and the filtrate was concentrated under vacuum to give a residue. It was purified by silica gel column (MeOH/DCM=0%˜5%), and the fraction was concentrated under vacuum to give LD2-3c (274 mg, 69.8% yield) as a white solid.

MS (ESI) m/z: 652.6 [M+Na]⁺

### Step 2

### benzyl 3-(((S)-2-((S)-2-amino-3-methylbutanamido)propanamido)methoxy)-2,2-dimethylpropanoate (LD2-3d)

To a solution of LD2-3c (274 mg, 0.44 mmol) in DMF (5 mL) was added Et₂NH (477 mg, 5.53 mmol). The mixture was stirred at r.t. for 20 min. The reaction mixture was concentrated under vacuum and co-evaporated with Tol twice to give LD2-3d (275 mg, crude) as a brown oil.

MS (ESI) m/z: 430.4 [M+Na]⁺.

### Step 3

### benzyl (5S,8S,11S)-5-(3-((((2R,3S,4R,5S)-5-(2-amino-2-oxoethyl)-3,4-dihydroxytetrahydrofuran-2-yl)methyl)amino)-3-oxopropyl)-1-(9H-fluoren-9-yl)-8-isopropyl-11,17,17-trimethyl-3,6,9,12-tetraoxo-2,15-dioxa-4,7,10,13-tetraazaoctadecan-18-oate (LD2-3f)

To a solution of LD2-3d (275 mg, crude) and LD2-3e (282 mg, 0.52 mmol, purchased from WuXi AppTec) in DMF (5 mL) were added HATU (198 mg, 0.52 mmol) and DIPEA (168 mg, 1.30 mmol). The mixture was stirred at r.t. for 10 min. The mixture was purified by reverse phase (C18, 60 g, 30%˜70%), and the fraction was freeze-dried to give LD2-3f (370 mg, 91.5% yield) as a brown solid.

MS (ESI) m/z: 953.8 [M+Na]⁺

### Step 4

### (5S,8S,11S,17R)-5-(3-((((2R,3S,4R,5S)-5-(2-amino-2-oxoethyl)-3,4-dihydroxytetrahydrofuran-2-yl)methyl)amino)-3-oxopropyl)-1-(9H-fluoren-9-yl)-17-fluoro-8-isopropyl-11,17-dimethyl-3,6,9,12-tetraoxo-2,15-dioxa-4,7,10,13-tetraazaoctadecan-18-oic Acid (LD2-3g)

To a solution of compound LD2-3f (3.01 g, 3.21 mmol) in co-solvent DMF-MeOH (40 mL, 1:1, v:v) was added Pd/C (10%, 600 mg). The mixture was stirred in H₂ atmosphere (15 psi) for 7 h. The mixture was filtered through a pad of celite, and concentrated to give compound LD2-3g (2.50 g, crude) as a white solid.

MS (ESI) m/z: 863.7 [M+Na]⁺

### Step 5

### (9H-fluoren-9-yl)methyl ((6S,9S,12S)-1-((2R,3S,4R,5S)-5-(2-amino-2-oxoethyl)-3,4-dihydroxytetrahydrofuran-2-yl)-19-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-9-isopropyl-12,18,18-trimethyl-3,7,10,13,19-pentaoxo-16-oxa-2,8,11,14-tetraazanonadecan-6-yl)carbamate (LD2-3h)

To a solution of compound exatecan mesylate (1000 mg, 1.18 mmol, purchased from MedChemExpress CO. LTD) in DMF (20 mL) were added compound LD2-3g (692 mg, 1.30 mmol), HATU (675 mg, 1.78 mmol), and DIEA (459 mg, 3.55 mmol). The mixture was stirred at r.t. for 30 min. The mixture was concentrated and purified by a silica gel column chromatography (eluent: DCM/MeOH=0% to 20%) to give the title compound LD2-3h (1.32 g, 88.6% yield) as an off-white solid.

MS (ESI) m/z: 1282.1 [M+Na]⁺

### Step 6

### (S)-2-amino-N5-(((2R,3S,4R,5S)-5-(2-amino-2-oxoethyl)-3,4-dihydroxytetrahydrofuran-2-yl)methyl)-N1-((S)-1-(((S)-1-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-1-oxopropan-2-yl)amino)-3-methyl-1-oxobutan-2-yl)pentanediamide (LD2-3i)

To a solution of compound LD2-3h (1000 mg, 0.79 mmol) in DMF (20 mL) was added Et₂NH (580 mg, 7.93 mmol). The mixture was stirred at r.t. for 30 min. The mixture was concentrated under high vacuum to give compound LD2-3i (825 mg, crude) as an off-white solid, which was used directly without further purification.

MS (ESI) m/z: 1036.9 [M+H]⁺

### Step 7

### (S)—N5-(((2R,3S,4R,5S)-5-(2-amino-2-oxoethyl)-3,4-dihydroxytetrahydrofuran-2-yl)methyl)-2-(3-(2-(2,5-dioxo-2,5-dihydro-1H-pyrrol-1-yl)acetamido)propanamido)-N1-((S)-1-(((S)-1-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-1-oxopropan-2-yl)amino)-3-methyl-1-oxobutan-2-yl)pentanediamide (LD2-3)

To the solution of LD2-3j (15 mg, 0.067 mmol) in dry DMF (1.0 mL) were added HATU (26 mg, 0.067 mmol) and DIEA (0.017 mL, 0.097 mmol), stirred at r.t. for 15 min. Then to the above mixture was added LD2-3i (50 mg, 0.048 mmol), stirred at r.t. for 10 min. The resulting solution was purified by prep-HPLC (Method: column XBridge Prep C18 OBD 5 um 19*150 mm; Mobile phase: A-water (0.1% TFA): B-acetonitrile; Flow rate: 20 mL/min). The fraction was lyophilized to give LD2-3 (37 mg, 50.9% yield) as a yellow solid.

MS (ESI) m/z: 1266.7 [M+Na]⁺

### Example LD2-4

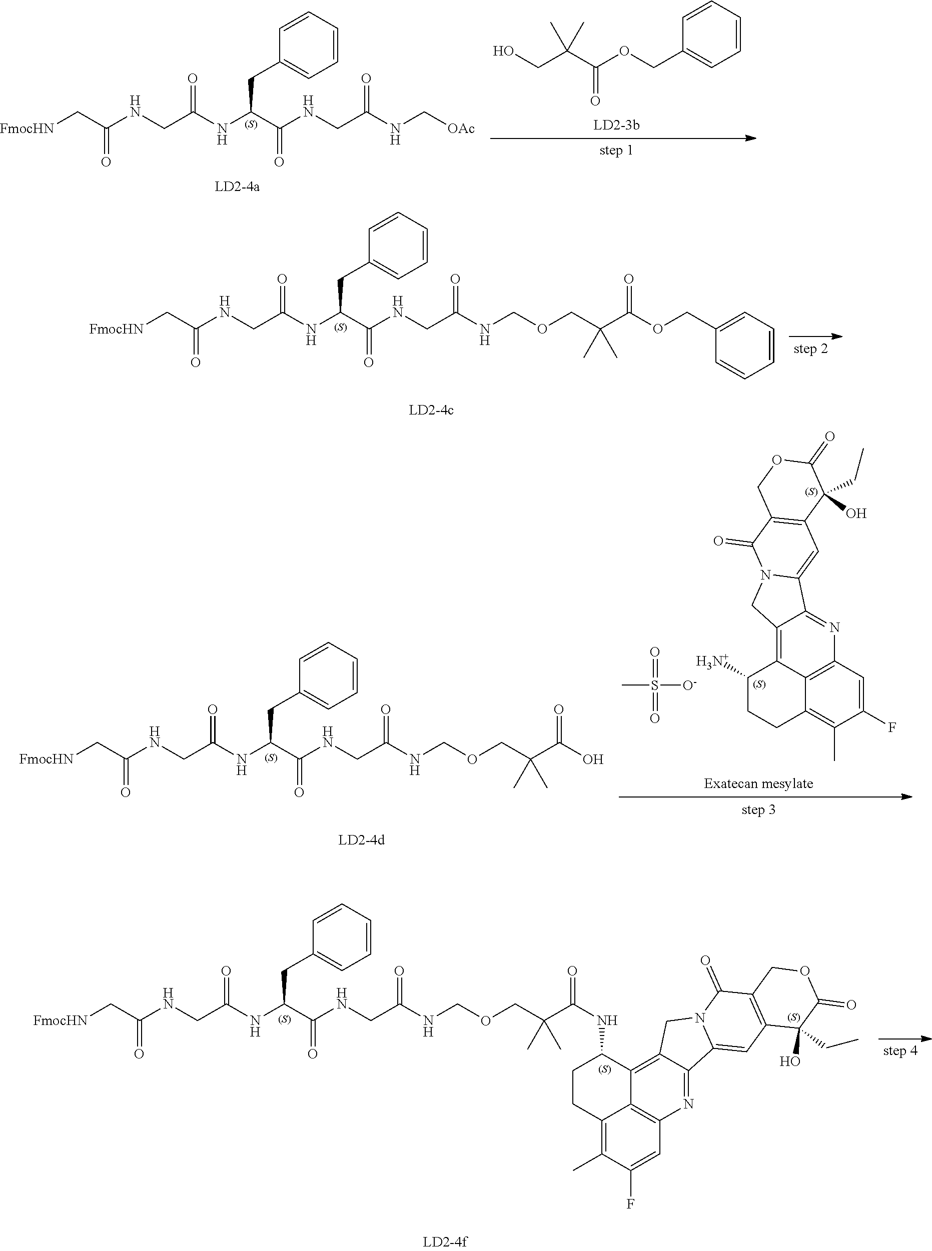

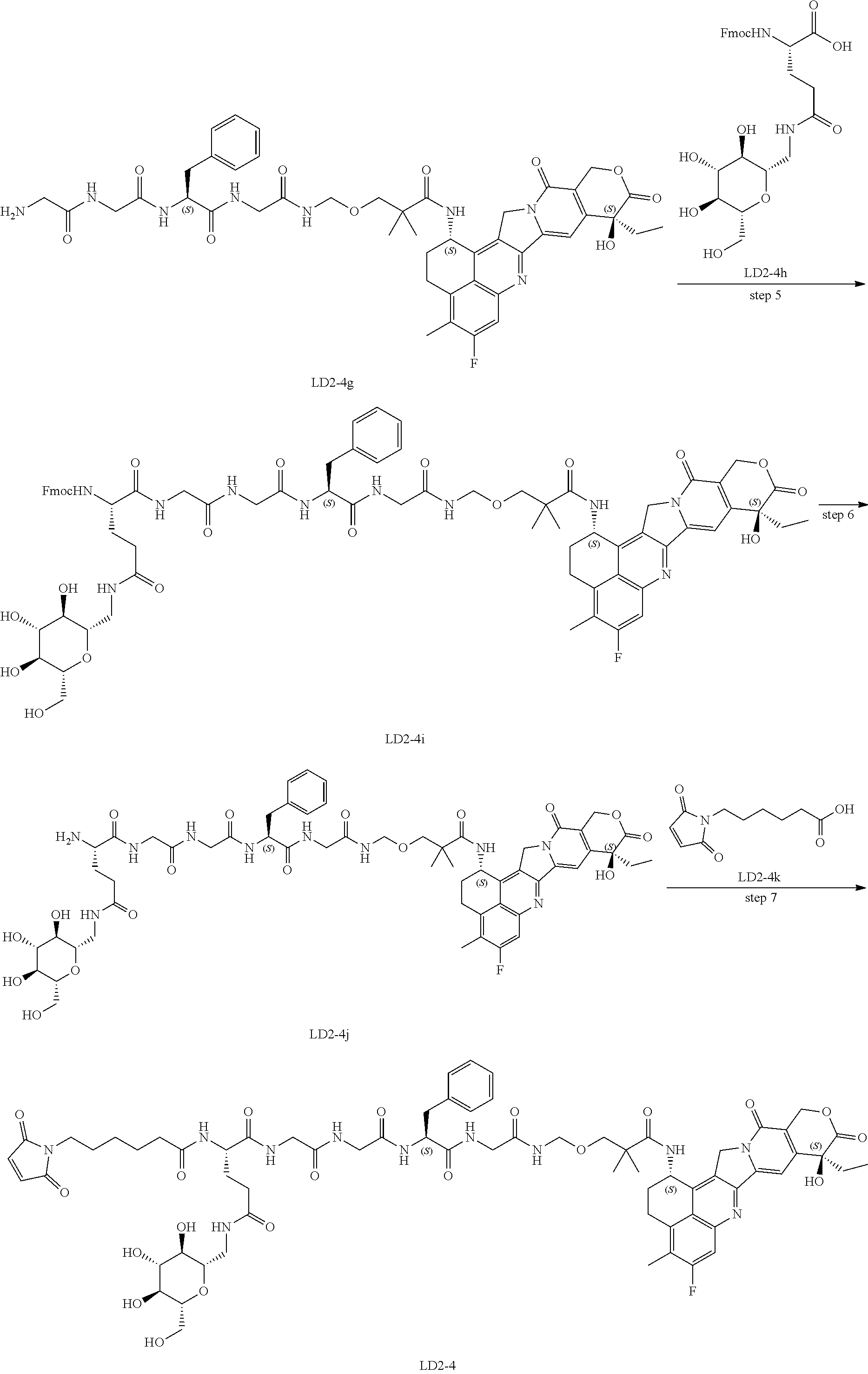

### Step 1

### Benzyl (S)-11-benzyl-1-(9H-fluoren-9-yl)-20,20-dimethyl-3,6,9,12,15-pentaoxo-2,18-dioxa-4,7,10,13,16-pentaazahenicosan-21-oate (LD2-4c)

To the solution of LD2-4a (250 mg, 0.40 mmol) and LD2-3b (83 mg, 0.40 mmol) in THF (5 mL) was added 4 Å molecular sieve. The mixture was stirred at r.t. for 10 min then Sc(OTf)₃ (195 mg, 0.40 mmol) was added and further reacted at r.t. for another 16 h. The suspension mixture was filtered through a pad of celite, and the cake was washed with THF (10 mL), then the filtrate was quenched by addition of sat. NaHCO₃ (10 mL), extracted with EtOAc (30 mL*2). After separation, the combined organic layers were washed with brine (50 mL), dried over Na₂SO₄, filtered, and the filtrate was concentrated under vacuum to give a residue which was further purified by silica gel column chromatography (A-DCM; B-MeOH, MeOH/DCM=0%˜5%) to provide LD2-4c (90 mg, 29.2% yield) as a white solid.

MS (ESI) m/z: 800.5 [M+Na]⁺

### Step 2

### (S)-11-benzyl-1-(9H-fluoren-9-yl)-20,20-dimethyl-3,6,9,12,15-pentaoxo-2,18-dioxa-4,7,10,13,16-pentaazahenicosan-21-oic Acid (LD2-4d)

To a solution of LD2-4c (80 mg, 0.10 mmol) in MeOH (3 mL) was added wet Pd/C (20 mg). The black suspension was purged with H₂ balloon three times then reacted at r.t. for 2 h under H₂ balloon. After the reaction was completed, the black suspension was filtered off through a pad of celite, the cake washed with MeOH, and the combined organic layers were concentrated under vacuum to provide LD2-4d (61 mg, 84.8% yield).

MS (ESI) m/z: 710.4 [M+Na]⁺

### Step 3

### (9H-fluoren-9-yl)methyl ((S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-16,16-dimethyl-2,5,8,11,17-pentaoxo-14-oxa-3,6,9,12-tetraazaheptadecyl)carbamate (LD2-4f)

To a mixture of LD2-4d (60 mg, 0.087 mmol) and HATU (33 mg, 0.087 mmol) in DMF (2 mL) was added DIEA (43 μL, 34 mg, 0.26 mmol). The mixture was reacted at r.t. for 10 min. Exatecan mesylate (46 mg, 0.087 mmol) was added and reacted at the same temperature for another 1 hr. After the reaction was completed, the mixture was filtered and the filtrate was purified using prep-HPLC (Method; column: XBridge Prep C18 OBD 5 um 19*150 mm; Mobile phase: A-water (0.1% formic acid): B-acetonitrile; Flow rate: 20 mL/min) to provide LD2-4f (85 mg, 88.2% yield).

MS (ESI) m/z: 1105.5 [M+H]⁺

### Step 4

### 3-(((S)-13-amino-7-benzyl-3,6,9,12-tetraoxo-2,5,8,11-tetraazatridecyl)oxy)-N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2,2-dimethylpropanamide (LD2-4g)

To a solution of LD2-4f (85 mg, 0.062 mmol) in DMF (2 mL) was added Et₂NH (64 μL, 46 mg, 0.62 mmol). The mixture was stirred at r.t. for 0.5 h. On completion of the reaction, the mixture was concentrated under vacuum to give LD2-4g (86 mg, crude) as a yellow solid.

MS (ESI) m/z: 883.5 [M+H]⁺

### Step 5

### (9H-fluoren-9-yl)methyl ((6S,15S)-15-benzyl-25-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-24,24-dimethyl-3,7,10,13,16,19,25-heptaoxo-1-((2S,3R,4R,5S,6R)-3,4,5-trihydroxy-6-(hydroxymethyl)tetrahydro-2H-pyran-2-yl)-22-oxa-2,8,11,14,17,20-hexaazapentacosan-6-yl)carbamate (LD2-4i)

To a solution of LD2-4g (86 mg, crude) and LD2-4h (43 mg, 0.079 mmol, purchased from WuXi AppTec) in DMF (1.5 mL) was added DIEA (26 μL, 21 mg, 0.16 mmol). The mixture was stirred at r.t. for 1.5 h. On completion of the reaction, the mixture was purified by prep-HPLC (FA) (Method: column: XBridge Prep C18 OBD 5 um 19*150 mm; Mobile phase: A-water (0.1% formic acid): B-acetonitrile; Flow rate: 20 mL/min). The fraction was lyophilized to give LD2-4i (70 mg, 62.6% yield) as a white powder.

MS (ESI) m/z: 1410.7 [M+H]⁺

### Step 6

### (S)-2-amino-N1-((S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-16,16-dimethyl-2,5,8,11,17-pentaoxo-14-oxa-3,6,9,12-tetraazaheptadecyl)-N5-(((2S,3R,4R,5S,6R)-3,4,5-trihydroxy-6-(hydroxymethyl)tetrahydro-2H-pyran-2-yl)methyl)pentanediamide (LD2-4j)

To a solution of LD2-4i (70 mg, 0.050 mmol) in DMF (1 mL) was added Et₂NH (51 μL, 36 mg, 0.50 mmol). The mixture was stirred at r.t. for 0.5 h. On completion of the reaction, the mixture was concentrated under vacuum to give LD2-4j (71 mg, crude) as a yellow solid.

MS (ESI) m/z: 1188.2 [M+H]⁺

### Step 7

### (S)—N¹—((S)-7-benzyl-17-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-16,16-dimethyl-2,5,8,11,17-pentaoxo-14-oxa-3,6,9,12-tetraazaheptadecyl)-2-(6-(2,5-dioxo-2,5-dihydro-1H-pyrrol-1-yl)hexanamido)-N-(((2S,3R,4R,5S,6R)-3,4,5-trihydroxy-6-(hydroxymethyl)tetrahydro-2H-pyran-2-yl)methyl)pentanediamide (LD2-4)

To a solution of LD2-4k (19 mg) in DMF (3 mL) were added HATU (34 mg, 0.088 mmol) and DIEA (10 μL, 7.6 mg, 0.059 mmol). The resulting yellow solution was stirred at r.t. for 5 min then LD2-4j (71 mg, crude) was added. The mixture was stirred at r.t. for 60 min. On completion of the reaction, the mixture was purified by prep-HPLC (FA) (Method: column: XBridge Prep C18 OBD 5 um 19*150 mm; Mobile phase: A-water (0.1% formic acid): B-acetonitrile; Flow rate: 20 mL/min). The fraction was lyophilized to give LD2-4 (32 mg, 26.3% yield) as a white powder.

MS (ESI) m/z: 1381.1 [M+H]⁺

### Example LD2-5

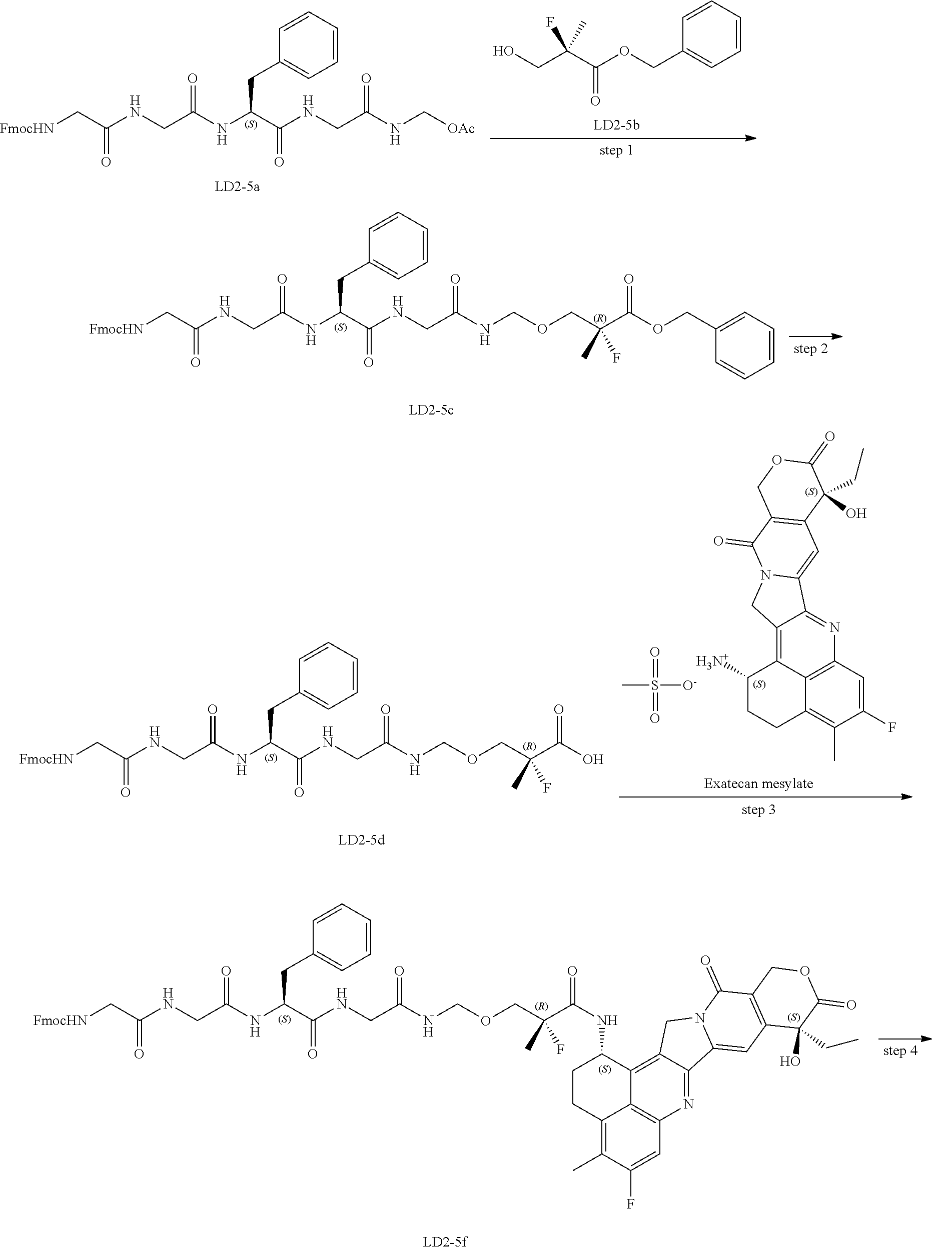

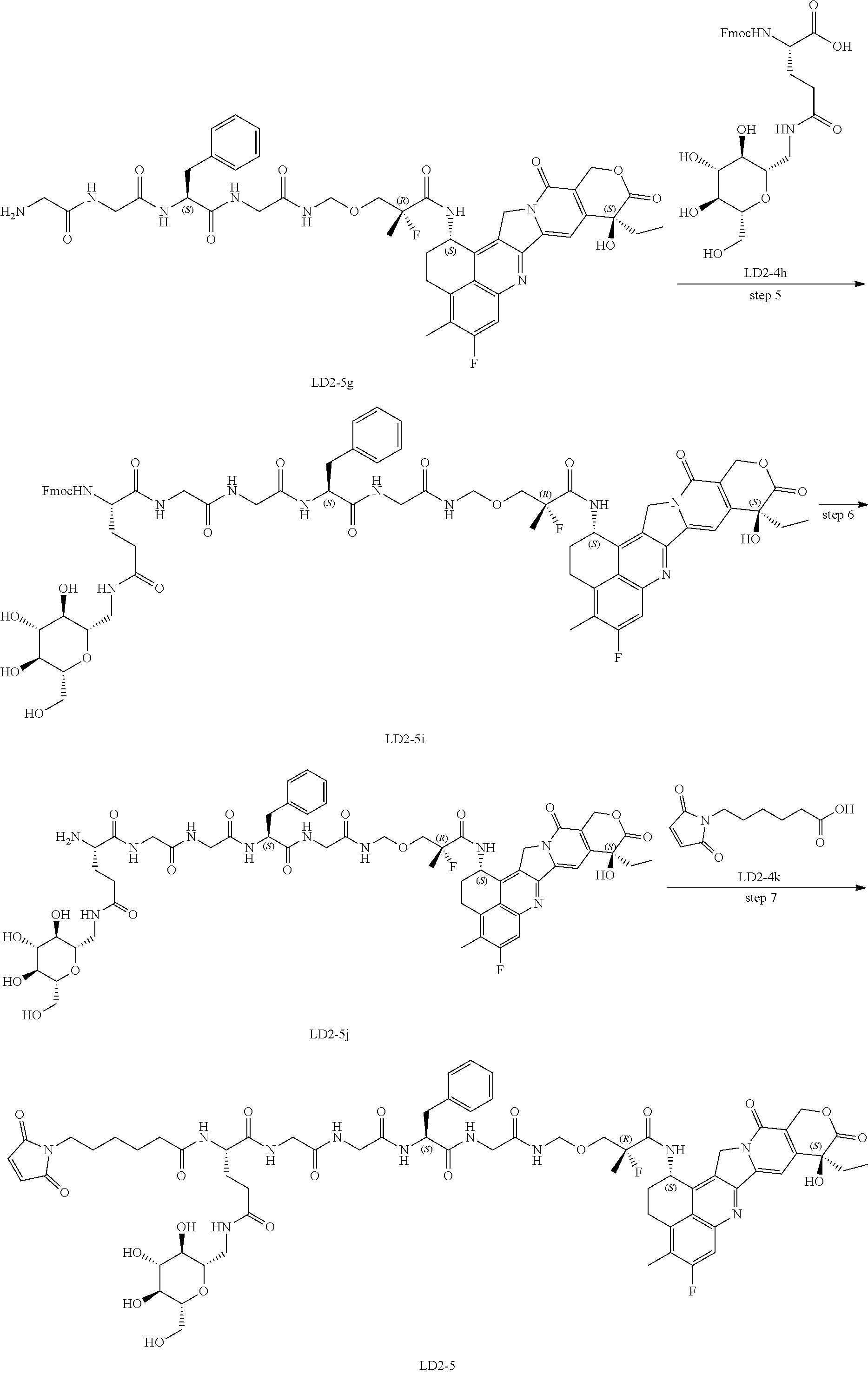

LD2-5 (30 mg, 50.7% yield) was synthesized according to the synthetic procedures of LD2-4.

MS (ESI) m/z: 1408.1 [M+Na]⁺

### Example LD2-6

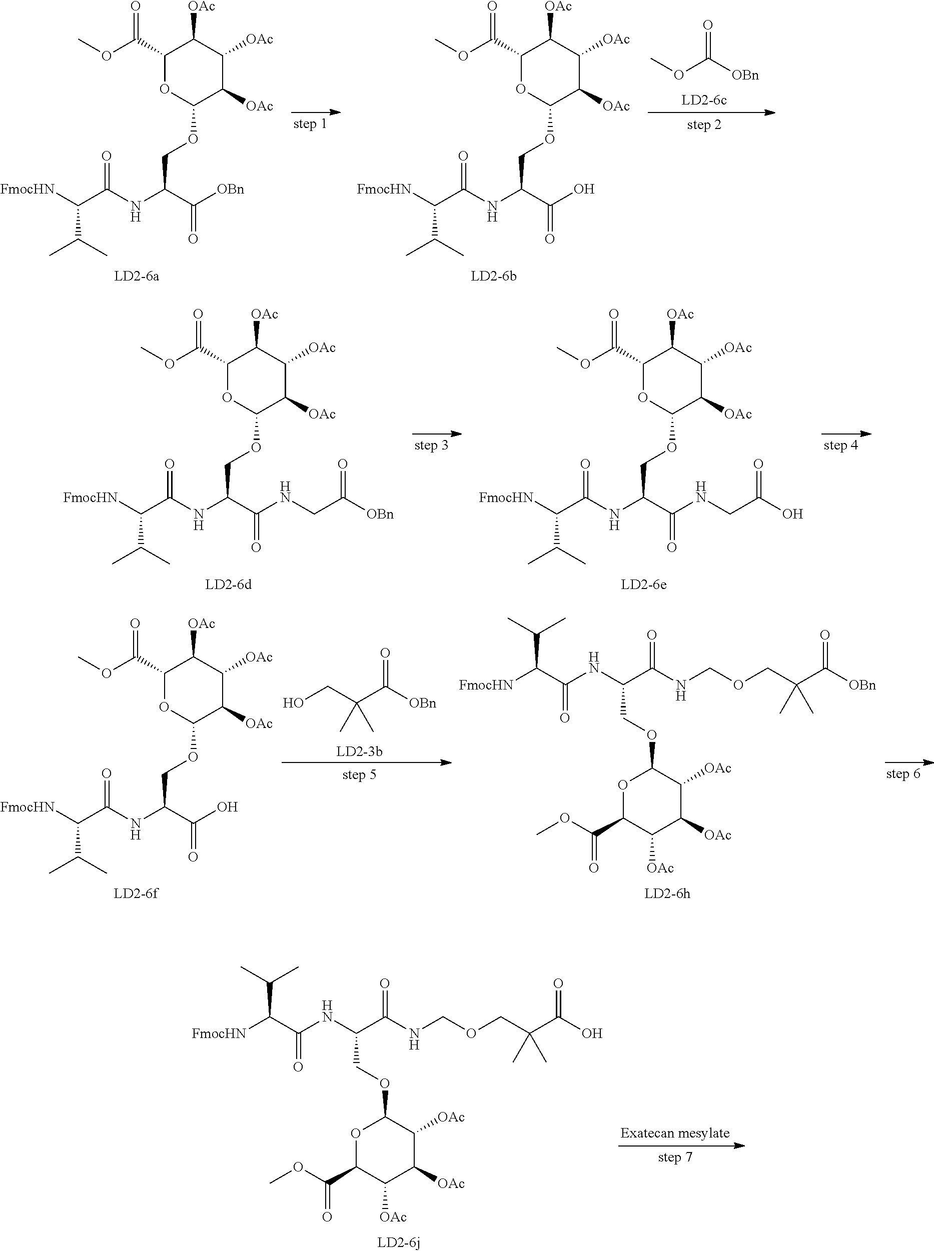

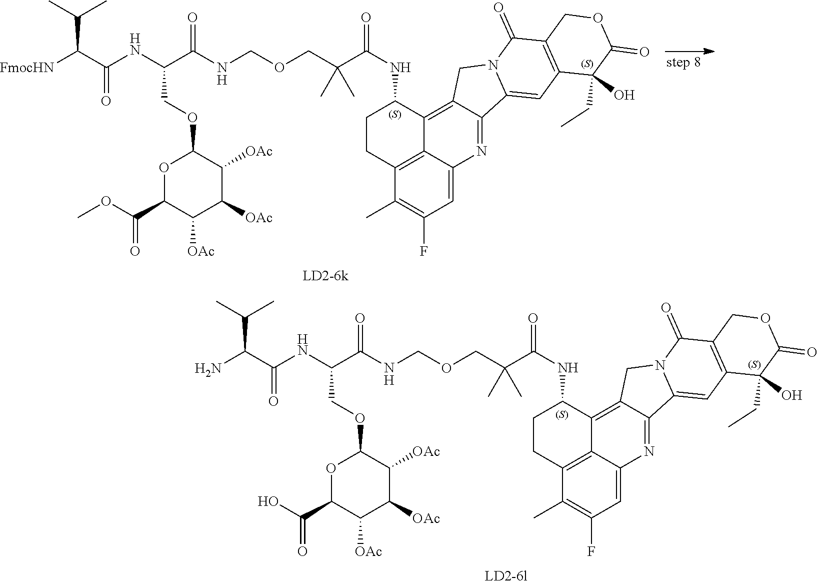

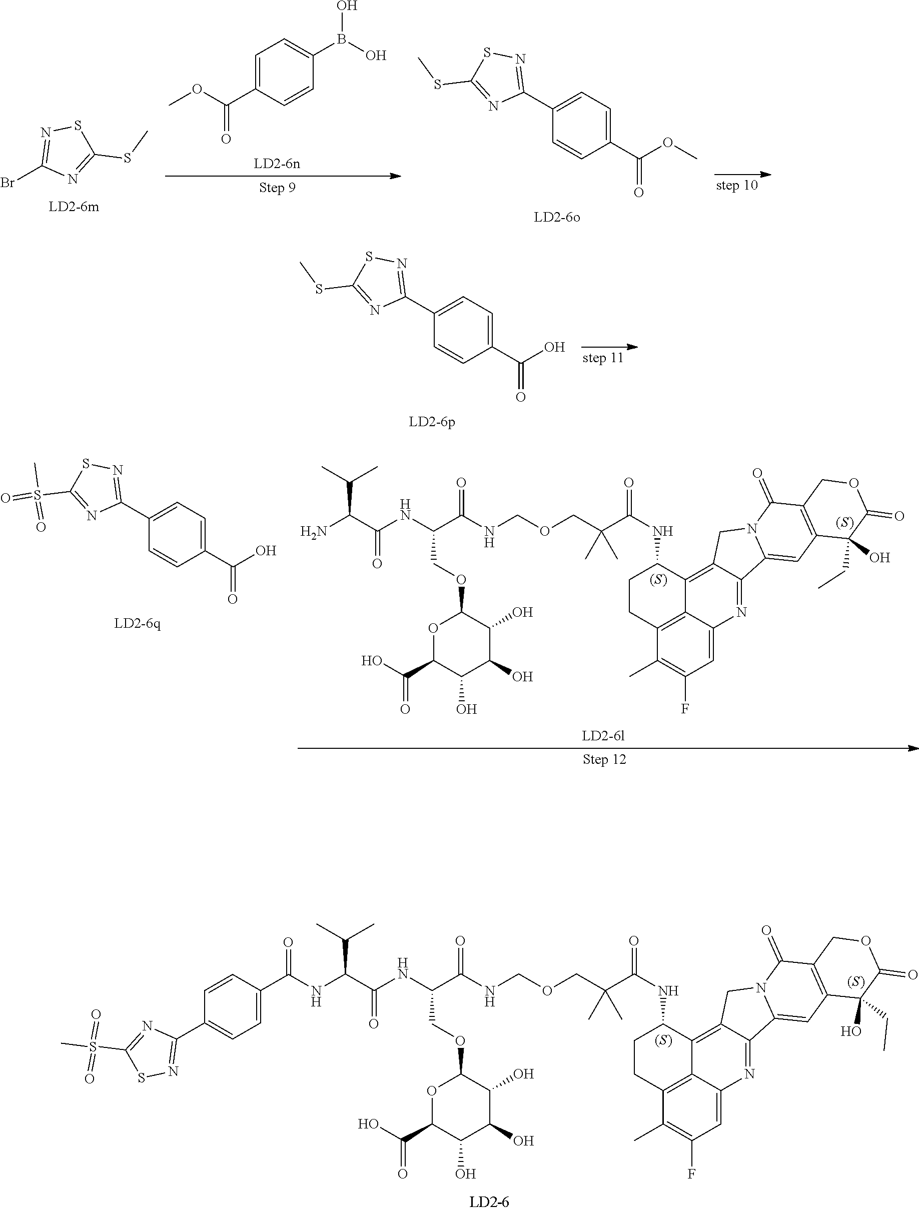

### Step 1

### N-((((9H-fluoren-9-yl)methoxy)carbonyl)-L-valyl)-O-((2R,3R,4S,5S,6S)-3,4,5-triacetoxy-6-(methoxycarbonyl)tetrahydro-2H-pyran-2-yl)-L-serine (LD2-6b)

To a mixture of LD2-6a (4.10 g, 4.92 mmol, purchase from MedChemExpress CO. LTD) in MeOH (50 mL), THF (100 mL) and DCM (20 mL) was added wet Pd/C (400 mg, 10% purity). The black suspension was purged with H₂ balloon three times and then stirred at r.t. for 1 hr. The black suspension was filtered through a pad of celite, washed with MeOH (200 mL). Combined organic layers were concentrated under vacuum to give LD2-6b (3.65 g, 99.8% yield) as an off-white solid.

MS (ESI) m/z: 743.6 [M+H]⁺

### Step 2

### (2R,3R,4S,5S,6S)-2-((S)-2-((S)-2-((((9H-fluoren-9-yl)methoxy)carbonyl)amino)-3-methylbutanamido)-3-((2-(benzyloxy)-2-oxoethyl)amino)-3-oxopropoxy)-6-(methoxycarbonyl)tetrahydro-2H-pyran-3,4,5-triyl Triacetate (LD2-6d)

To a solution of LD2-6b (3.65 g, 4.92 mmol) and LD2-6c (1.66 g, 4.92 mmol) in DMF (50 mL) were added HATU (1.87 g, 4.92 mmol) and DIEA (1.59 g, 12.29 mmol). The mixture was stirred at r.t. for 30 min. The mixture was purified by FCC (MeOH/DCM=0%˜10%), the fraction was concentrated under vacuum to give LD2-6d (3.80 g, 86.9% yield) as an off-white foamed solid.

MS (ESI) m/z: 890.7 [M+H]⁺

### Step 3

### N-((((9H-fluoren-9-yl)methoxy)carbonyl)-L-valyl)-O-((2R,3R,4S,5S,6S)-3,4,5-triacetoxy-6-(methoxycarbonyl)tetrahydro-2H-pyran-2-yl)-L-serylglycine (LD2-6e)

To a mixture of LD2-6d (3.80 g, 4.27 mmol) in MeOH (150 mL) and DCM (50 mL) was added wet Pd/C (400 mg, 10% purity). The black suspension was purged with H₂ balloon three times and then stirred at r.t. for 40 min. The black suspension was filtered through a pad of celite, washed with MeOH (150 mL). Combined organic layers were concentrated under vacuum to give LD2-6e (3.30 g, 96.6% yield) as an off-white solid.

MS (ESI) m/z: 800.7 [M+H]⁺

### Step 4

### (2R,3R,4S,5S,6S)-2-((S)-2-((S)-2-((((9H-fluoren-9-yl)methoxy)carbonyl)amino)-3-methylbutanamido)-3-((acetoxymethyl)amino)-3-oxopropoxy)-6-(methoxycarbonyl)tetrahydro-2H-pyran-3,4,5-triyl Triacetate (LD2-6f)

To a solution of LD2-6e (3.30 g, 4.13 mmol) in DMF (30 mL) were added Pb(OAc)₄ (2.74 g, 6.19 mmol), Cu(OAc)₂ (74.9 mg, 0.41 mmol), and HOAc (247.8 mg, 4.13 mmol). The resulting dark-color mixture was purged with N₂ balloon three times and then stirred at 65° C. for 40 min and the mixture turned deep blue. The mixture was diluted with EtOAc (300 mL), washed with brine (100 mL*3), dried over Na₂SO₄, filtered, and concentrated under vacuum to give a residue. It was purified by FCC (MeOH/DCM=0˜10%), and the fraction was concentrated under vacuum to give LD2-6f (2.81 g, 83.4% yield) as a pale-yellow solid.

MS (ESI) m/z: 836.6 [M+Na]⁺

### Step 5

### (2R,3R,4S,5S,6S)-2-((S)-2-((S)-2-((((9H-fluoren-9-yl)methoxy)carbonyl)amino)-3-methylbutanamido)-3-(((3-(benzyloxy)-2,2-dimethyl-3-oxopropoxy)methyl)amino)-3-oxopropoxy)-6-(methoxycarbonyl)tetrahydro-2H-pyran-3,4,5-triyl Triacetate (LD2-6h)

A white suspension mixture of LD2-6f (300 mg, 0.37 mmol), LD2-3b (154 mg, 0.74 mmol) and 4 Å molecular sieve (200 mg) in anhydrous THF (10 mL) was stirred at r.t. for 10 min. Sc(OTf)₃ (218 mg, 0.44 mmol) was added and the resulting yellow suspension was stirred at r.t. for 4 hr. The yellow suspension mixture was filtered through a pad of celite and washed with EtOAc. Combined organic layers were washed with sat. NaHCO₃ (30 mL) and brine (30 mL), dried over Na₂SO₄, filtered, and the filtrate was concentrated under vacuum to give a residue. It was purified by silica gel column (MeOH/DCM=0%˜5%), and the fraction was concentrated under vacuum to give LD2-6h (275 mg, 77.5% yield) as a white foam solid.

MS (ESI) m/z: 984.8 [M+Na]⁺

### Step 6

### (5S,8S)-1-(9H-fluoren-9-yl)-5-isopropyl-14,14-dimethyl-3,6,9-trioxo-8-((((2R,3R,4S,5S,6S)-3,4,5-triacetoxy-6-(methoxycarbonyl)tetrahydro-2H-pyran-2-yl)oxy)methyl)-2,12-dioxa-4,7,10-triazapentadecan-15-oic Acid (LD2-6j)

To a solution of LD2-6h (275 mg, 0.29 mmol) in MeOH (10 mL) was added wet Pd/C (55 mg, 10% purity). The black suspension was purged with H₂ balloon three times then stirred at r.t. for 2 hr. The mixture was filtered through a syringe head, washed with MeOH (15 mL), and concentrated under vacuum to give LD2-6j (230 mg, crude) as a white foam solid.

MS (ESI) m/z: 894.6 [M+Na]⁺

### Step 7

### (2R,3R,4S,5S,6S)-2-((S)-2-((S)-2-((((9H-fluoren-9-yl)methoxy)carbonyl)amino)-3-methylbutanamido)-3-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-3-oxopropoxy)-6-(methoxycarbonyl)tetrahydro-2H-pyran-3,4,5-triyl Triacetate (LD2-6k)

To a mixture of LD2-6j (230 mg, crude), exatecan mesylate (140 mg, 0.26 mmol) and HATU (100 mg, 0.26 mmol) in DMF (5 mL) was added DIEA (102 mg, 0.79 mmol). The resulting brown mixture was stirred at r.t. for 1 hr. The mixture was diluted with EtOAc (20 mL), washed with brine (20 mL*3), dried over Na₂SO₄, filtered, and concentrated under vacuum to give a residue. It was purified by FCC (MeOH/DCM=0%˜3%), and concentrated under vacuum to give LD2-6k (325 mg, 95.6% yield) as an off-white foam solid.

MS (ESI) m/z: 1289.9 [M+H]⁺

### Step 8

### (2S,3S,4S,5R,6R)-6-((S)-2-((S)-2-amino-3-methylbutanamido)-3-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-3-oxopropoxy)-3,4,5-trihydroxytetrahydro-2H-pyran-2-carboxylic Acid (LD2-6l)

To a solution of LD2-6k (325 mg, 0.25 mmol) in DMF (5 mL) was added Et₂NH (523 mg, 5.06 mmol). The mixture was stirred at r.t. for 20 min. LCMS showed reaction was completed, then the reaction product was concentrated under vacuum to give a crude product. The crude product was dissolved in MeOH (6 mL) to which K₂CO₃ (174.7 mg, 1.26 mmol) was added and stirred at r.t. for 10 min. Then H₂O (2 mL) was added to the mixture and stirred at r.t. for 30 min. The mixture was acidified with sat. KHSO₄ at 0° C. to pH=3, filtered, and purified by prep-HPLC (0.1% FA). The fraction was lyophilized to give LD2-6l (140 mg, 59.7% yield) as a pale yellow solid.

MS (ESI) m/z: 927.4 [M+H]⁺

¹H NMR (400 MHz, d₆-DMSO) δ 9.56 (s, 1H), 8.39 (s, 1H), 8.07 (d, J=8.4 Hz, 1H), 7.77 (d, J=11.2 Hz, 1H), 7.31 (s, 1H), 6.52 (s, 1H), 5.54 (dd, J=13.2, 7.2 Hz, 1H), 5.43 (s, 2H), 5.18 (dd, J=41.6, 18.8 Hz, 2H), 5.09-5.02 (m, 1H), 4.96 (s, 1H), 4.62 (dd, J=10.0, 6.8 Hz, 1H), 4.56-4.44 (m, 2H), 4.19 (d, J=7.6 Hz, 1H), 3.82 (dd, J=10.8, 6.8 Hz, 1H), 3.61 (dd, J=11.6, 6.4 Hz, 2H), 3.17-3.05 (m, 4H), 2.94 (t, J=8.0 Hz, 1H), 2.39 (s, 3H), 2.11 (dt, J=21.3, 7.6 Hz, 2H), 2.03-1.93 (m, 2H), 1.92-1.78 (m, 3H), 1.12 (d, J=8.0 Hz, 6H), 0.87 (dd, J=13.0, 6.6 Hz, 9H).

### Step 9

### methyl 4-(5-(methylthio)-1,2,4-thiadiazol-3-yl)benzoate (LD2-6o)

To a solution of compound LD2-6m (100 mg, 0.47 mmol) in toluene (4 mL) and H₂O (1 mL) were added compound LD2-6n (110 mg, 0.57 mmol), K₂CO₃ (168 mg, 0.95 mmol) and Pd(dppf)Cl₂·DCM (35 mg, 0.047 mmol). The mixture was stirred at 110° C. for 3 h under N₂ atmosphere. The mixture was filtered through a pad of celite, diluted with EtOAc (100 mL), and washed by brine (50 mL*4). The organic layer was dried over anhydrous Na₂SO₄, filtered, and concentrated. The crude product was purified by flash column chromatography (eluted with PE/EtOAc=0%˜40%). Compound LD2-6o (56 mg, 44.4% yield) was obtained as an off-white solid.

MS (ESI) m/z: 267.1 [M+H]⁺

### Step 10

### 4-(5-(methylthio)-1,2,4-thiadiazol-3-yl)benzoic Acid (LD2-6p)

To a solution of compound LD2-6o (54 mg, 0.20 mmol) in MeOH (3 mL) and H₂O (1 mL) was added LiOH (17 mg, 0.41 mmol). The mixture was stirred at r.t. for 2 h. The mixture was adjusted to pH 7 and purified by prep-HPLC (FA condition) to give compound LD2-6p (36 mg, 70.3% yield) as a white solid.

MS (ESI) m/z: 253.1 [M+H]⁺

### Step 11

### 4-(5-(methylsulfonyl)-1,2,4-thiadiazol-3-yl)benzoic Acid (LD2-6q)

To a solution of compound LD2-6p (35 mg, 0.14 mmol) in DCM (3 mL) and THF (3 mL) was added m-CPBA (96 mg, 0.55 mmol). The mixture was stirred at room temperature for 16 h. The mixture was concentrated and purified by prep-HPLC (Method: column: XBridge Prep C18 OBD 5 um 19*150 mm; Mobile phase: A-water (0.1% TFA): B-acetonitrile; Flow rate: 20 mL/min). Compound LD2-6q (12 mg, 99% purity) was obtained as a white solid.

MS (ESI) m/z: 284.8 [M+H]⁺

### Step 12

### (2S,3S,4S,5R,6R)-6-((S)-3-(((3-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2,2-dimethyl-3-oxopropoxy)methyl)amino)-2-((S)-3-methyl-2-(4-(5-(methylsulfonyl)-1,2,4-thiadiazol-3-yl)benzamido)butanamido)-3-oxopropoxy)-3,4,5-trihydroxytetrahydro-2H-pyran-2-carboxylic Acid (LD2-6)

To a solution of compound LD2-6q (7.4 mg, 0.026 mmol) in DMF (2 mL) were added HATU (9. mg, 0.024 mmol) and DIEA (5.6 mg, 0.043 mmol). The mixture was stirred at r.t. for 30 min. Compound LD2-6l (20 mg, 0.022 mmol) was added to the mixture and stirred at r.t. for 15 min. The reaction was purified by prep-HPLC (Method: column: XBridge Prep C18 OBD 5 um 19*150 mm; Mobile phase: A-water (0.1% TFA): B-acetonitrile; Flow rate: 20 mL/min) to give compound LD2-6 (7.6 mg, 29.5% yield) as a white solid.

MS (ESI) m/z: 1193.5 [M+H]⁺

### Example LD2-7

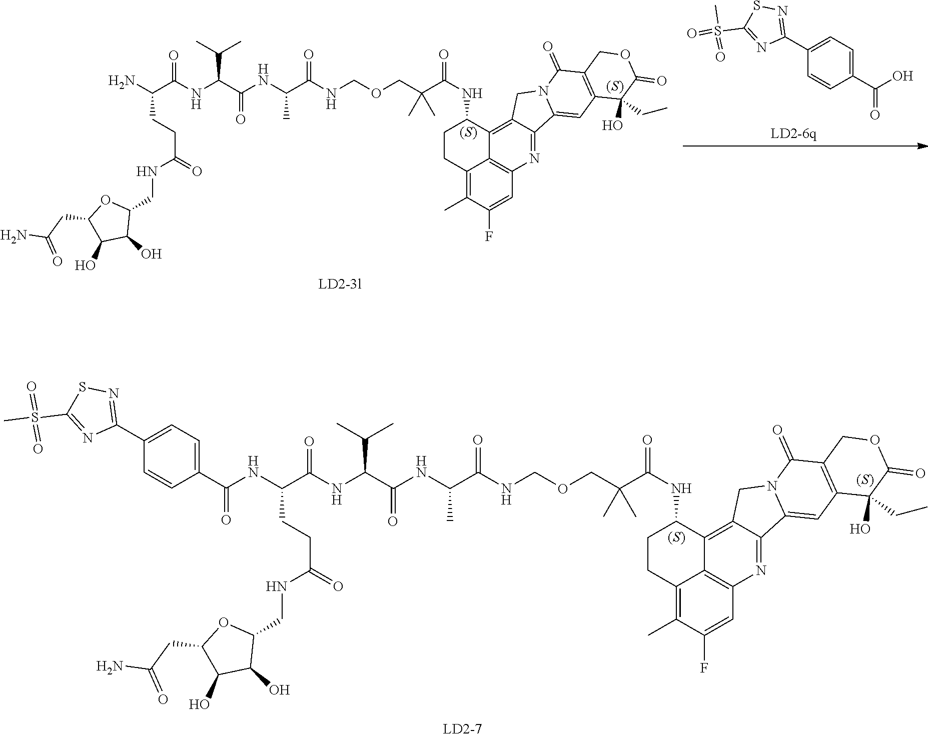

LD2-7 (32 mg, 50.9% yield) was synthesized according to the procedure of Step 7 of LD2-3.

MS (ESI) m/z: 1303.0 [M+H]⁺

### Example LD2-8

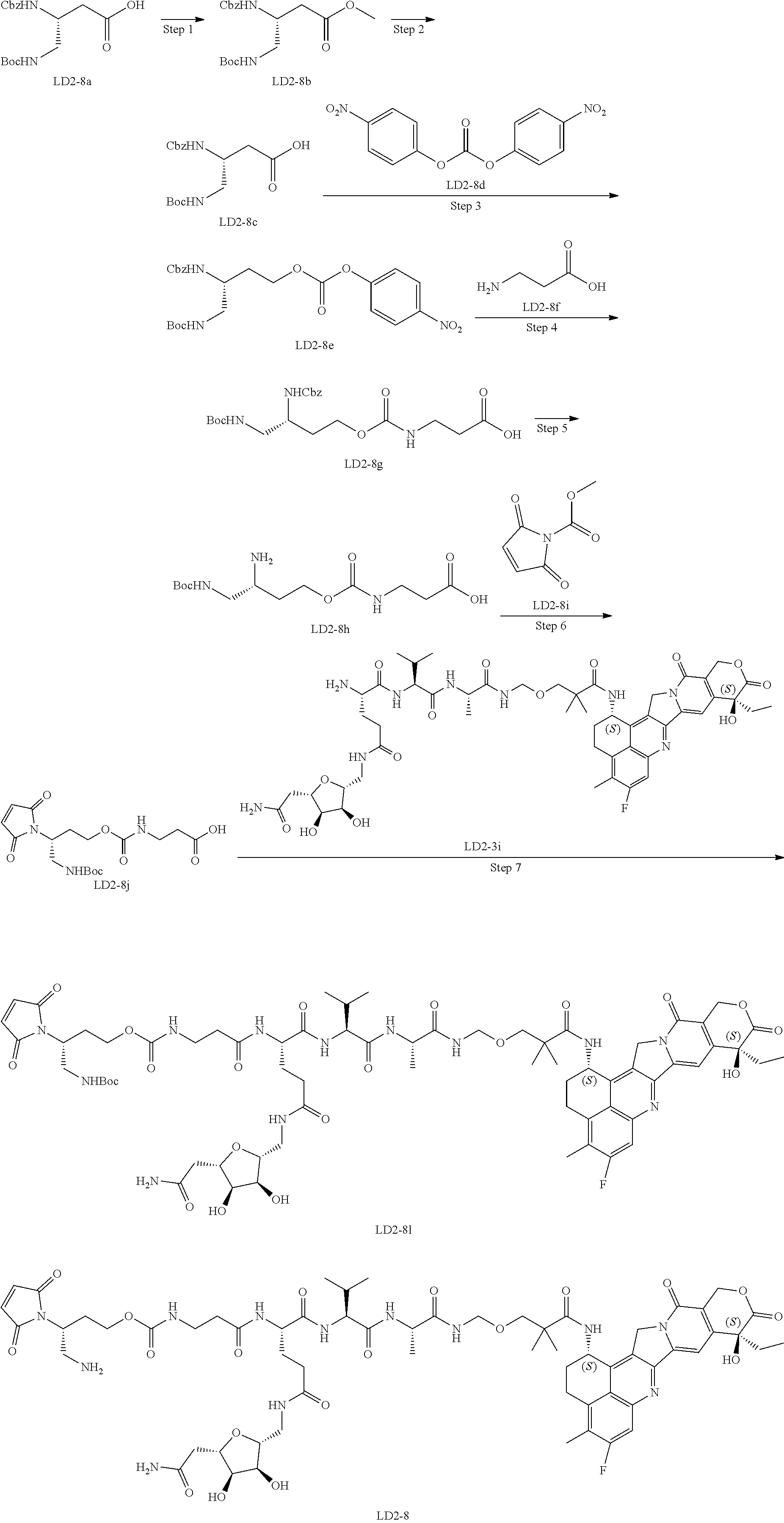

### Step 1

### Methyl (R)-3-(((benzyloxy)carbonyl)amino)-4-((tert-butoxycarbonyl)amino)butanoate (LD2-8b)

LD2-8a (2.00 g, 5.68 mmol) and K₂CO₃ (863 mg, 6.24 mmol) were added to DMF (10 mL) followed by the dropwise addition of CH₃I (1.61 g, 11.35 mmol) at 0° C. The resulting mixture was stirred at 0° C. for 20 min and allowed to warm to 25° C. and further stirred at 25° C. for 60 min. The reaction process was monitored by TLC (PE/EA) and LCMS. After complete reaction, the reaction mixture was diluted with EA (80 mL) and washed with brine (30 mL*3) and H₂O (30 mL*2). The organic layer was dried over anhydrous Na₂SO₄, filtered, and concentrated under reduced pressure to afford the methyl ester of LD2-8b (2.08 g, quant.) as a light-yellow solid.

MS (ESI) m/z: 267.2 [M-Boc+H]⁺

### Step 2

### Benzyl tert-butyl (4-hydroxybutane-1,2-diyl)(R)-dicarbamate (LD2-8c)

LD2-8b (1.00 g, 2.73 mmol) was dissolved in MeOH (15 mL) followed by the addition of LiBH₄ (2 M stock solution in THF, 6.80 mL) at 0° C. The resulting mixture was stirred at 25° C. for 2 h. Reaction process was monitored by LCMS and TLC. After complete reaction, saturated aqueous NH₄Cl (10 mL) was added to quench the reaction. The reaction mixture was diluted with H₂O (80 mL) and extracted with EA (50 mL*3). The combined organic layers were washed with brine (40 mL*2) and water (40 mL*2), dried over anhydrous Na₂SO₄, filtered, concentrated under reduced pressure, and further purified by flash column chromatography (PE/EA) to afford LD2-8c (760 mg, 82.3% yield) as a white solid.

MS (ESI) m/z: 239.2 [M-Boc+H]⁺

### Step 3

### Benzyl tert-butyl (4-(((4-nitrophenoxy)carbonyl)oxy)butane-1,2-diyl)(R)-dicarbamate (LD2-8e)

LD2-8c (300 mg, 0.89 mmol) and LD2-8d (405 mg, 1.33 mmol) were dissolved in DMF (5 mL) followed by the addition of DIEA (229 mg, 1.77 mmol). The resulting mixture was stirred at 25° C. for 1.5 h. After complete reaction, the reaction mixture was diluted with EA (100 mL) and washed with brine (35 mL*2) and water (35 mL*2). The organic layer was dried over anhydrous Na₂SO₄, filtered, and concentrated under reduced pressure to afford LD2-8e as a white solid (371 mg, 83.1% yield).

MS (ESI) m/z: 404.4 [M-Boc+H]⁺

### Step 4

### (9H-fluoren-9-yl)methyl tert-butyl (4-(((3-(dimethylamino)-3-oxopropyl)carbamoyl)oxy)butane-1,3-diyl)(S)-dicarbamate (LD2-8g)

LD2-8e (420 mg, 0.83 mmol) and LD2-8f (149 mg, 1.67 mmol) were dissolved in DMF (5 mL) followed by the addition of aqueous NaHCO₃ (1M, 5 mL). The resulting mixture was stirred at 25° C. for 2.5 h. After complete reaction, the reaction mixture was concentrated and purified by flash column chromatography (DCM/MeOH) to afford LD2-8g as a pale-yellow solid (365 mg, 96.5% yield).

MS (ESI) m/z: 354.4 [M-Boc+H]⁺

### Step 5

### (R)-7-(2,5-dioxo-2,5-dihydro-1H-pyrrol-1-yl)-2,2-dimethyl-4,11-dioxo-3,10-dioxa-5,12-diazapentadecan-15-oic Acid (LD2-8h)

LD2-8g (360 mg, 0.79 mmol) was dissolved in MeOH (18 mL) followed by the addition of Pd/C (wet base, 108 mg). The resulting mixture was stirred at r.t. under H₂ (15 psi) for 2 h. After complete reaction, the reaction mixture was filtered and concentrated under reduced pressure to afford LD2-8h as a clear syrup (252 mg, 99.4% yield). The crude product was used directly in the next step without purification.

MS (ESI) m/z: 320.3 [M+H]⁺

### Step 6

### (R)-7-(2,5-dioxo-2,5-dihydro-1H-pyrrol-1-yl)-2,2-dimethyl-4,11-dioxo-3,10-dioxa-5,12-diazapentadecan-15-oic Acid (LD2-8j)

LD2-8h (250 mg, 0.78 mmol) and LD2-8i (243 mg, 1.57 mmol) were dissolved in a mixed solvent of ACN (8 mL) and aqueous NaHCO₃ (1 M, 16 mL). The resulting mixture was stirred at 0° C. for 1 h and further stirred at 25° C. until the reaction was complete. Then the reaction mixture was acidified with aq. KHSO₄ (20 mL) and extracted with EA (35 mL*3). The combined organic layers were dried over anhydrous Na₂SO₄, filtered, and concentrated under reduced pressure to afford a yellow oil as the crude product, which was purified by flash column chromatography to afford LD2-8j (280 mg, 89.6% yield) as a white solid.

MS (ESI) m/z: 422.3 [M+Na]⁺

¹H NMR (400 MHz, d₆-DMSO) δ 12.48 (s, 1H), 7.03-7.01 (m, 2H), 6.99 (s, 2H), 4.08-4.03 (m, 3H), 3.86-3.83 (m, 2H), 3.14-3.11 (m, 2H), 2.35 (t, J=7.2 Hz, 2H), 2.16-2.09 (m, 1H), 1.9LD2-8.84 (m, 1H), 1.32 (s, 9H).

### Step 7

### (R)-4-((tert-butoxycarbonyl)amino)-3-(2,5-dioxo-2,5-dihydro-1H-pyrrol-1-yl)butyl ((8S,11S,14S)-14-(3-((((2R,3S,4R,5S)-5-(2-amino-2-oxoethyl)-3,4-dihydroxytetrahydrofuran-2-yl)methyl)amino)-3-oxopropyl)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-11-isopropyl-2,2,8-trimethyl-1,7,10,13,16-pentaoxo-4-oxa-6,9,12,15-tetraazaoctadecan-18-yl)carbamate (LD2-8l)

LD2-8l (32 mg, 50.9% yield) was synthesized according to the procedure of Step 7 of LD2-3.

MS (ESI) m/z: 1418.1 [M+H]⁺

### Step 8

### (R)-4-amino-3-(2,5-dioxo-2,5-dihydro-1H-pyrrol-1-yl)butyl ((8S,11S,14S)-14-(3-((((2R,3S,4R,5S)-5-(2-amino-2-oxoethyl)-3,4-dihydroxytetrahydrofuran-2-yl)methyl)amino)-3-oxopropyl)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-11-isopropyl-2,2,8-trimethyl-1,7,10,13,16-pentaoxo-4-oxa-6,9,12,15-tetraazaoctadecan-18-yl)carbamate (LD2-8)

LD2-8l (22 mg, 0.016 mmol) was dissolved in a mixed solvent of DCM (2 mL) followed by the addition of ZnBr2 (151 mg, 0.67 mmol). The resulting suspension was stirred at 40° C. for 12 h. After complete reaction, the reaction mixture was filtered and concentrated. The residue was diluted with a mixed solvent of CH3CN/aqueous 0.1% FA and purified by prep-HPLC (Method: column: XBridge Prep C18 OBD 5 um 19*150 mm; Mobile phase: A-water (0.1% TFA): B-acetonitrile; Flow rate: 20 mL/min) to give compound LD2-8 (13 mg, 61.7% yield) as a white solid.

MS (ESI) m/z: 1318.1 [M+H]⁺

### Example 21. Immunoconjugate Preparation

An antibody in a conjugation buffer (with concentration 0.5-25 mg/mL, PBS buffer pH 6.0-8.5) was incubated under reduction temperature (0-40° C.) for 10 min. 8-15 eq. TECP solution (5 mM stock in PBS buffer) was added into the reaction mixture and the reduction reaction was left for 1-8 hours at reduction temperature. Organic solvent (e.g., DMSO, DMF, DMA, PG, acetonitrile, 0-25% v/v) and linker-toxin stock (LD2-3, see Table 6) (2-25 eq, 10 mM stock in organic solvent) were added stepwise in reaction buffer (PBS buffer pH 6.0-9.0) with anti-FGFR2B antibody (BGAh9179 for BGA3457; BGAh9239 for BGA9823) (1-20 mg/mL) under 0-37° C. for 0.5-48 h. The solution was submitted to buffer exchange (spin desalting column, ultrafiltration, and dialysis) into storage buffer (for example: pH 5.5-6.5 histidine acetate buffer, with optional additive such as sucrose, trehalose, tween 20, 60, 80).

See also Example 23 for immunoconjugate preparation.

### Example 22. BGA3457 DAR (Drug-To-Antibody-Ratio) Determination

Drug to antibody ratios (DAR) of the ADCs were determined by LCMS method or HIC method.

LCMS method: LC-MS analysis was carried out under the following measurement conditions:
LC-MS system: Vanquish Flex UHPLC and Orbitrap Exploris 240 Mass Spectrometer
Column: MAbPac™ RP, 2.1*50 mm, 4 μm, 1,500 Å, Thermo Scientific™
Column temperature: 80° C.
Mobile phase A: 0.1% formic acid (FA) aqueous solution
Mobile phase B: Acetonitrile solution containing 0.1% formic acid (FA)
Gradient program: 25% B-25% B (0 min-2 min), 25% B-50% B (2 min-18 min), 50% B-90% B (18 min-18.1 min), 90% B-90% B (18.1 min-20 min), 90% B-25% B (20 min-20.1 min), 25% B-25% B (20.1 min-25 min)
Injected sample amount: 1 or 2 μg
MS parameters: Intact and denaturing MS data were acquired in HMR mode at setting of R=15 k and deconvolved using the ReSpect™ algorithm and Sliding Window integration in Thermo Scientific™ BioPharma Finder™ 4.0 software.

HIC method: HPLC analysis was carried out under the following measurement conditions:
HPLC system: Waters ACQUITY ARC HPLC System
Detector: measurement wavelength: 280 nm
Column: Tosoh Bioscience 4.6 μm ID×3.5 cm, 2.5 μm butyl-nonporous resin column
Column temperature: 25° C.
Mobile phase A: 1.5 M ammonium sulfate, 50 mM phosphate buffer, pH 7.0
Mobile phase B: 50 mM phosphate buffer, 25% (v/v) isopropanol, pH 7.0
Gradient program: 0% B-0% B (0 min-2 min), 0% B-100% B (2 min-15 min), 100% B-100% B (15 min-16 min), 100% B-0% B (16 min-17 min), 0% B-0% B (17 min-20 min)
Injected sample amount: 20 μg

### Example 23. DAR8 Immunoconjugate Preparation

To prepare an immunoconjugate having 8 units of drug (toxin) per antibody (“DAR8”), antibody in conjugation buffer (with concentration 0.5-25 mg/mL, PBS buffer pH 6.0-8.5) was incubated under reduction temperature (0-40° C.) for 10 min and 8-15 eq. TECP solution (5 mM stock in PBS buffer) was added into the reaction mixture and left the reduction reaction for 1-8 hours at reduction temperature. Organic solvent (e.g.: DMSO, DMF, DMA, PG, acetonitrile, 0-25% v/v) and linker-payload stock (10-25 eq, 10 mM stock in organic solvent) were added stepwise after reduction mixture was cooled down to 0-25° C. Conjugation solution was left for 1-3 h at 0-25° C. and the reaction was quenched with N-acetyl cysteine (1 mM stock). The solution was submitted to buffer exchange (spin desalting column, ultrafiltration, and dialysis) into storage buffer (for example: pH 5.5-6.5 Histidine acetate buffer, with optional additive such as sucrose, trehalose, tween 20, 60, 80).

Maleimide Hydrolysis after Conjugation

After the conjugation step, the immunoconjugate underwent buffer exchange into ring opening buffer (pH 7.0˜9.0, PBS, borate or tris buffer) and the solution was left at 22 or 37° C. for 5˜48 h. The maleimide ring opening process was monitored via reduced LCMS. Once the conjugated maleimide hydrolysis was completed, the resulting immunoconjugates were submitted to buffer exchange into basic tris pH 8.0-8.5 buffer or acidic histidine-acetate pH 5.0-6.5 buffer via dialysis.

### Immunoconjugate Characterization

Immunoconjugates were prepared by following the above procedures with a DAR8 profile. All immunoconjugates were characterized via the following analytical methods. DAR of the immunoconjugates were determined according to the methods described in Example 22.

### SEC Method to Determine Immunoconjugate Purity

HPLC analysis was carried out under the following measurement conditions:
HPLC system: Waters H-Class UPLC System
Detector: measurement wavelength: 280 nm
Column: ACQUITY UPLC BEH200 SEC 1.7 um 4.6×150 mm, Waters
Column temperature: room temperature
Mobile phase A: 200 mM phosphate buffer, 250 mM potassium chloride, 15% isopropyl alcohol, pH 7.0
Gradient program: under 10 min isocratic elutions with a flow rate of 0.3 mL/min
Injected sample amount: 20 μg

The SEC purity of constructed immunoconjugates was >95%.

### Example 23. Immunoconjugate Hydrophobicity Evaluation

Immunoconjugates with greater hydrophobic properties appear with later retention times from HIC (hydrophobicity interaction column) chromatography. The DAR8 (antibody with 8 drug units loaded onto it) was the peak of the example immunoconjugates for this comparison.

HIC Method 1: HPLC analysis was carried out under the following measurement conditions:
HPLC system: Waters ACQUITY ARC HPLC System
Detector: measurement wavelength: 280 nm
Column: Tosoh Bioscience 4.6 μm ID×3.5 cm, 2.5 μm butyl-nonporous resin column
Column temperature: 25° C.
Mobile phase A: 1.5 M ammonium sulfate, 50 mM phosphate buffer, pH 7.0
Mobile phase B: 50 mM phosphate buffer, 25% (v/v) isopropanol, pH 7.0
Gradient program: 0% B-0% B (0 min-2 min), 0% B-100% B (2 min-15 min), 100% B-100% B (15 min-16 min), 100% B-0% B (16 min-17 min), 0% B-0% B (17 min-20 min)
Injected sample amount: 20 μg

HIC Method 2: HPLC analysis was carried out under the following measurement conditions:
HPLC system: Waters ACQUITY ARC HPLC System
Detector: measurement wavelength: 280 nm
Column: MABPac HIC-10, 5 μm, 4.6×10 mm (Thermo)
Column temperature: 25° C.
Mobile phase A: 1.5 M ammonium sulfate, 50 mM sodium phosphate, pH 7.0
Mobile phase B: 50 mM sodium phosphate, pH 7.0
Gradient program: 20% B-20% B (0 min-1 min), 0% B-0% B (1 min-35 min), 20% B-20% B (35 min-40 min)
Flow rate. 0.5 mL/min
Sample preparation: The sample was diluted from the initial mobile phase to 0.5 mg/mL

TABLE 16
ADCs

| | | | HIC |
|---|---|---|---|
| | | | DAR8 |
| ADC | Antibody | | RT (min) |
| Ref. No. | Ref. No. | ADC structure | Method 1 |
| BGA 9823 | BGA h9239 | 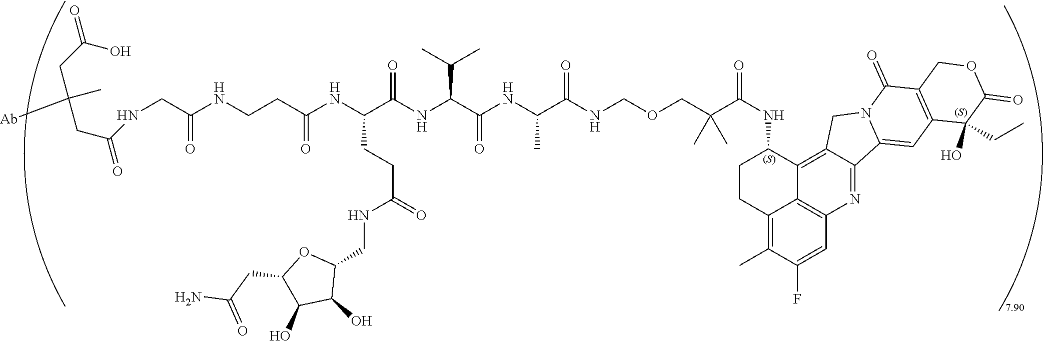 | 9.052 |
| BGA 3457 | BGA h9179 | 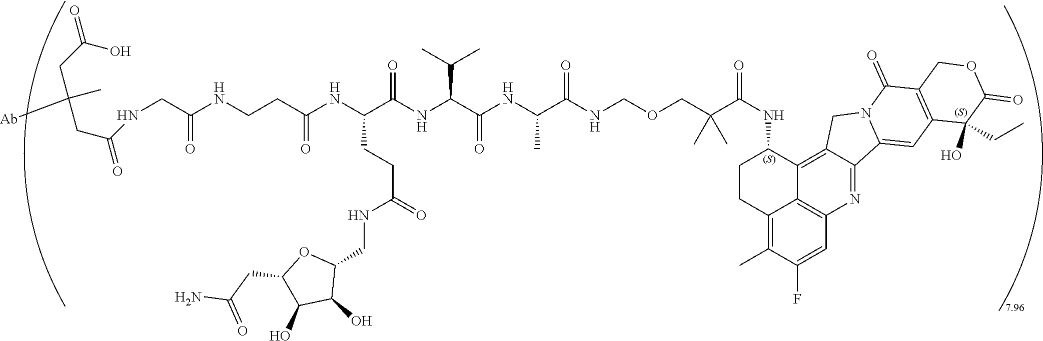 | 9.215 |

Both ADCs shown in Table 16 have >95% SEC purities.

### Example 25. Immunoconjugate Plasma Stability Evaluation and PK Profile Analysis

Plasma immunoconjugate and total therapeutic antibody (Ab) concentrations were determined under the following measurement conditions:
Assay: Ligand binding assay (ELISA)
Capture reagent: FGFR2b ECD
Detection reagent: Anti-toxin antibody for the immunoconjugate and anti-human IgG polyclonal Ab for Total Ab.

Blood samples were collected at 0, 2, 4, 8, 24, 72, and 168 h after one dose of intravenously administered immunoconjugate to FGFR2b expressing tumor bearing mice (i.e., SNU-16 human gastric cancer xenograft model in BALB/c nude mice) or non-tumor bearing mice, followed by centrifugation (4° C., 3000×g, 7 min) to separate plasma. The concentrations of immunoconjugates and total therapeutic antibodies were measured by in-house developed Meso Scale Discovery (MSD) ligand binding methods. Briefly, the ECD of FGFR2b was used as a capture reagent, and biotin-labelled anti-toxin antibody, or goat anti-human kappa antibody, were used as the detection reagents for immunoconjugates and total antibody measurement, respectively.

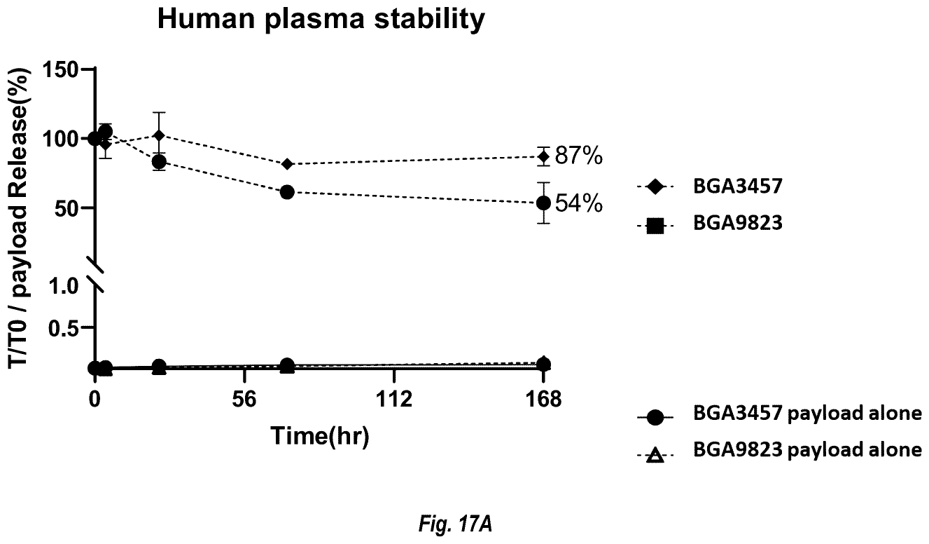

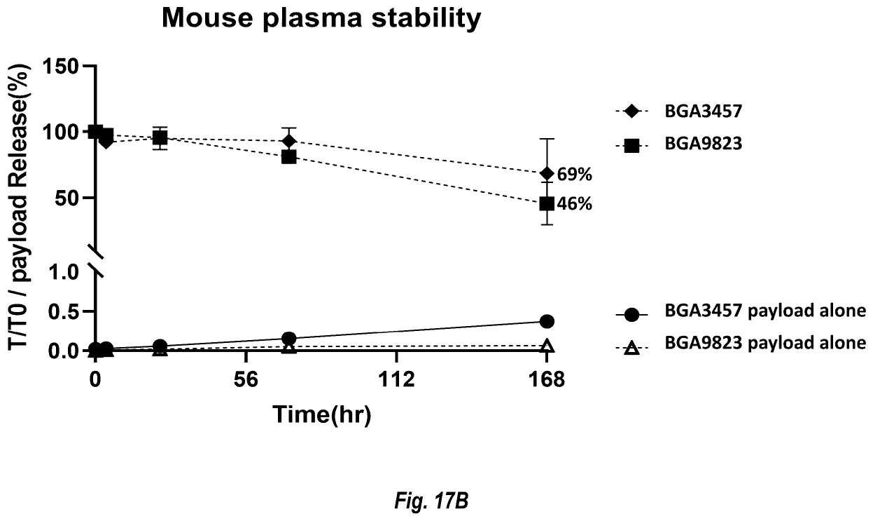

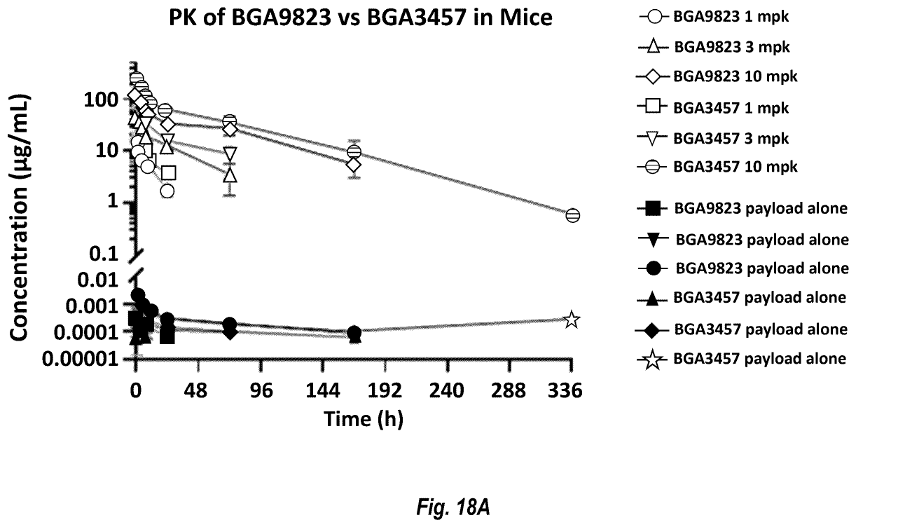

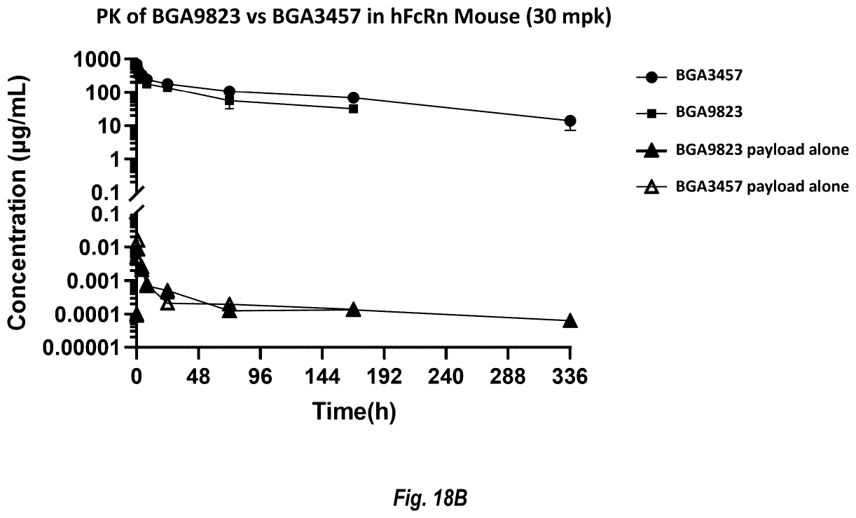

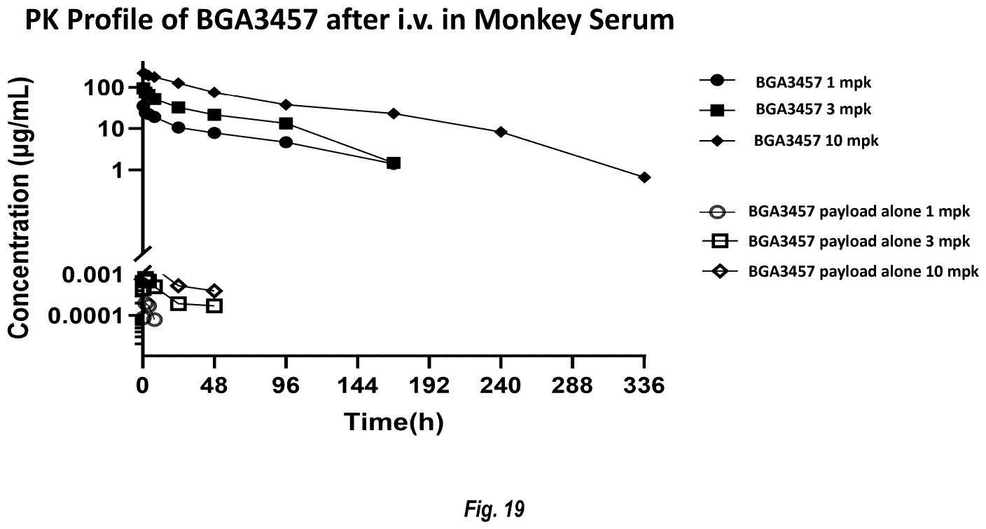

The PK profiles of BGA3457 and BGA9823 were evaluated in tumor-bearing and hFcRn mice (FIGS. 17A-17B). BGA3457 and BGA9823 showed good plasma stability in human (FIG. 17A) and mouse (FIG. 17B) serum samples. FIGS. 17A-17B show low free payload release (<1%) and a stable DAR value of 8 across all time points. BGA3457 showed nonlinear PK from 1-10 mg/kg in mice, suggesting target-mediated clearance (TMDD) (FIGS. 18A-18B). Comparator BGA8723 demonstrated nonlinear PK from 1-10 mg/kg in mice while linear PK from 1 mg/kg to 15 mg/kg in patients, suggesting TMDD is less severe in humans (data not shown). As shown in FIG. 19, BGA3457 demonstrated good PK profiles in monkey serum at each of 1, 3, and 10 mpk.

Although the invention has been described with reference to presently preferred embodiments, it should be understood that various modifications can be made without departing from the scope of the disclosure. Unless otherwise apparent from the context, any step, element, embodiment, feature, or aspect of the disclosure can be used with any other. All publications, patents, patent applications, accession numbers, and the like cited are herein incorporated by reference in their entireties for all purposes to the same extent as if each individual publication, patent, patent application, or accession number was specifically and individually indicated to be incorporated by reference in its entirety for all purposes.

1. An antibody or antigen-binding fragment thereof that specifically binds to human FGFR2b and comprises heavy chain complementarity determining region (HCDR) 1, HCDR2, and HCDR3, and light chain complementarity determining region (LCDR) 1, LCDR2, and LCDR3, wherein the HCDRs and the LCDRs comprise the following sequences (I) or (II):
(I) HCDR1 comprises the sequence IYWX₁N (SEQ ID NO: 77),
HCDR2 comprises the sequence IYPENX₂DTNYX₃GKFKG (SEQ ID NO: 78),
HCDR3 comprises the sequence GGFDY (SEQ ID NO: 5),
LCDR1 comprises the sequence RASENIYSNLA (SEQ ID NO: 6),
LCDR2 comprises the sequence TATNLAX₄ (SEQ ID NO: 79), and
LCDR3 comprises the sequence QHFYGILYT (SEQ ID NO: 8);
wherein X₁ is M or L; X₂ is G or A; X₃ is S or N; and X₄ is D or E; or
(II) HCDR1 comprises the sequence DTYIH (SEQ ID NO: 15),
HCDR2 comprises the sequence RIDPAX₁GNTMFASEFQG (SEQ ID NO: 80),
HCDR3 comprises the sequence SKIHYDYDEGFAY (SEQ ID NO: 17),
LCDR1 comprises the sequence RASESVDDYGYSFLH (SEQ ID NO: 18),
LCDR2 comprises the sequence RASNLES (SEQ ID NO: 19), and
LCDR3 comprises the sequence QQSNQNPRT (SEQ ID NO: 20);
wherein X₁ is E or N.

2.-10. (canceled)

11. A pharmaceutical composition comprising the antibody or antigen-binding fragment thereof of claim 1 and a pharmaceutically acceptable carrier.

12. An isolated nucleic acid that encodes the antibody or antigen-binding fragment of claim 1.

13. A vector comprising the nucleic acid of claim 12.

14. A host cell comprising the nucleic acid of claim 12.

15. A process for producing an antibody or antigen-binding fragment thereof comprising cultivating the host cell of claim 14 in culture media and recovering the antibody or antigen-binding fragment thereof from the culture media.

16. (canceled)

17. An immunoconjugate, or a pharmaceutically acceptable salt, solvate, or hydrate thereof, comprising the antibody or antigen-binding fragment thereof of claim 1, and a cytotoxic agent.

18.-26. (canceled)

27. An immunoconjugate represented by one of the following formulas: 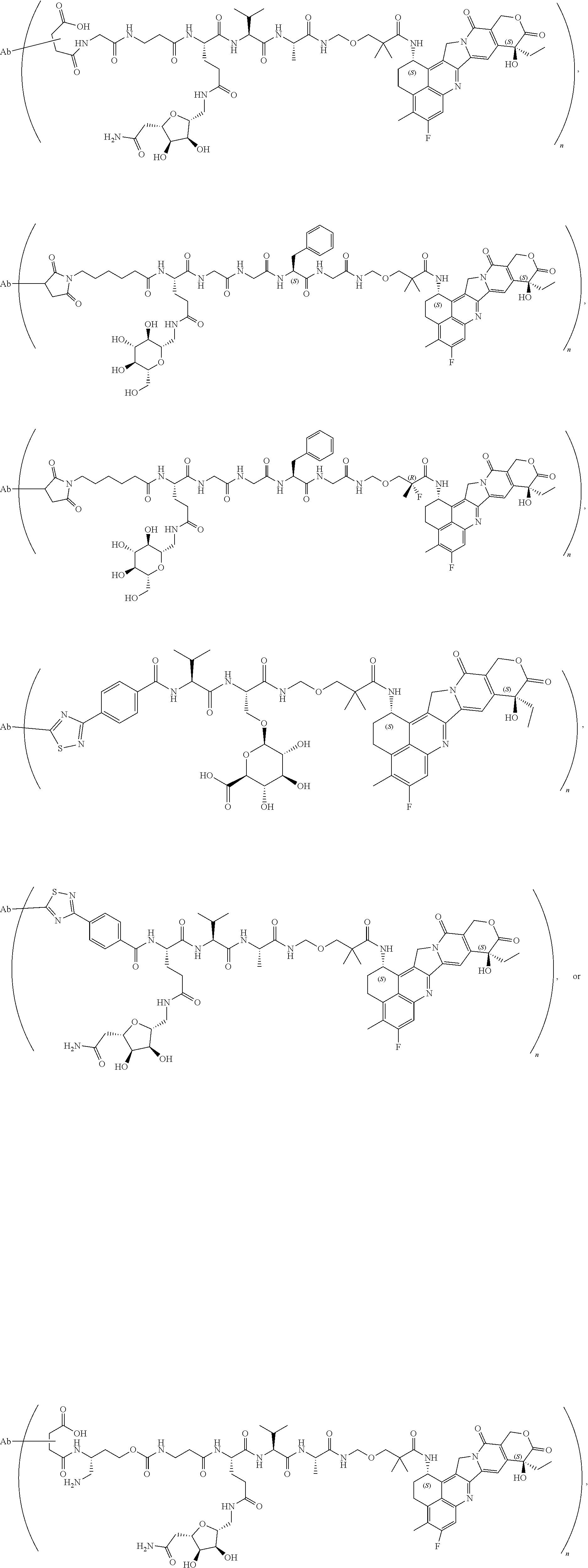
or a pharmaceutically acceptable salt, solvate, and/or stereoisomer thereof, wherein
Ab is an antibody or antigen-binding fragment thereof that specifically binds to human FGFR2b and comprises:
(i) a heavy chain variable region that comprises:
(a) a heavy chain complementarity determining region (HCDR) 1 having the sequence IYWLN (SEQ ID NO: 3),
(b) a HCDR2 having the sequence QIYPENADTNYSGKFKG (SEQ ID NO: 4), and
(c) a HCDR3 having the sequence GGFDY (SEQ ID NO: 5); and
(ii) a light chain variable region that comprises:
(d) a light chain complementarity determining region (LCDR) 1 having the sequence RASENIYSNLA (SEQ ID NO: 6),
(e) a LCDR2 having the sequence TATNLAE (SEQ ID NO: 7), and
(f) a LCDR3 having the sequence QHFYGILYT (SEQ ID NO: 8);
or
(i) a heavy chain variable region that comprises:
(a) a HCDR1 having the sequence DTYIH (SEQ ID NO: 15),
(b) a HCDR2 having the sequence RIDPAEGNTMFASEFQG (SEQ ID NO: 16), and
(c) a HCDR3 having the sequence SKIHYDYDEGFAY (SEQ ID NO: 17), and
(ii) a light chain variable region that comprises:
(d) a LCDR1 having the sequence RASESVDDYGYSFLH (SEQ ID NO: 18),
(e) a LCDR2 having the sequence RASNLES (SEQ ID NO: 19), and
(f) a LCDR3 having the sequence QQSNQNPRT (SEQ ID NO: 20); and
n is from 1 to 10.

28. The immunoconjugate of claim 27, wherein n is 3, 4, 5, 6, 7, 8, 9, or 10.

29. The immunoconjugate of claim 27, wherein n is about 8.

30. The immunoconjugate of claim 27, wherein the immunoconjugate is represented by one of the following formulas: 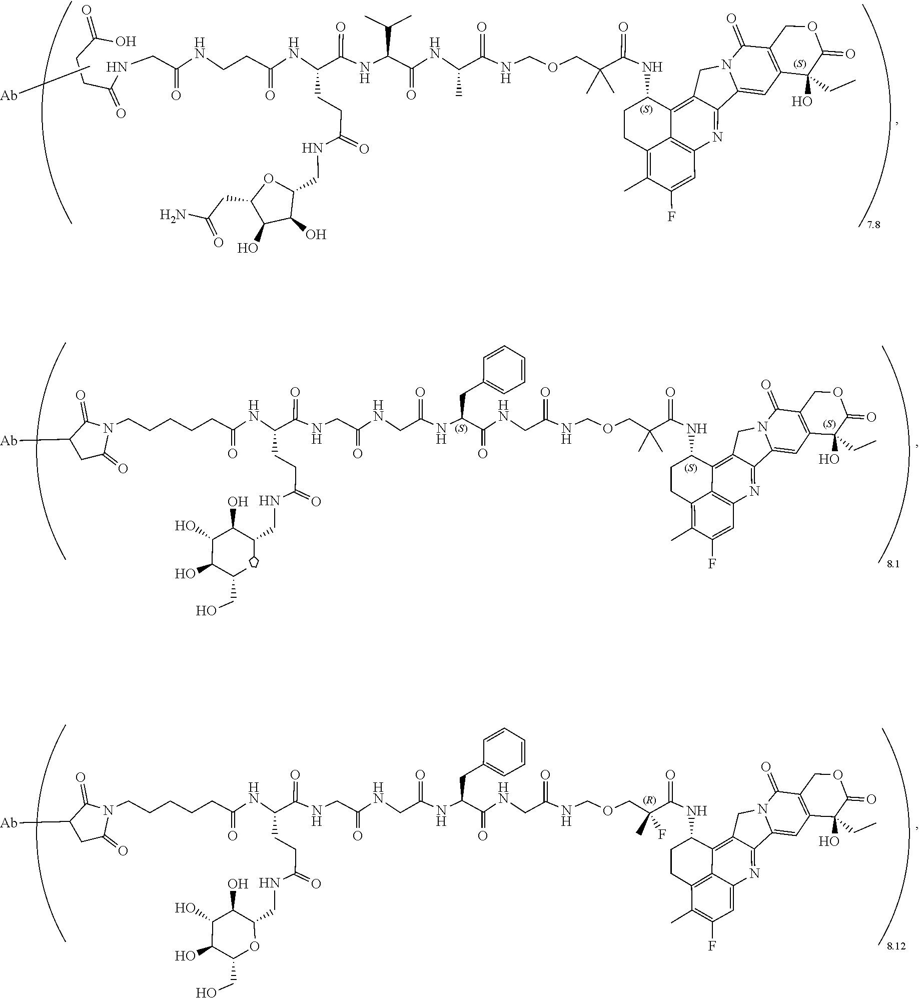 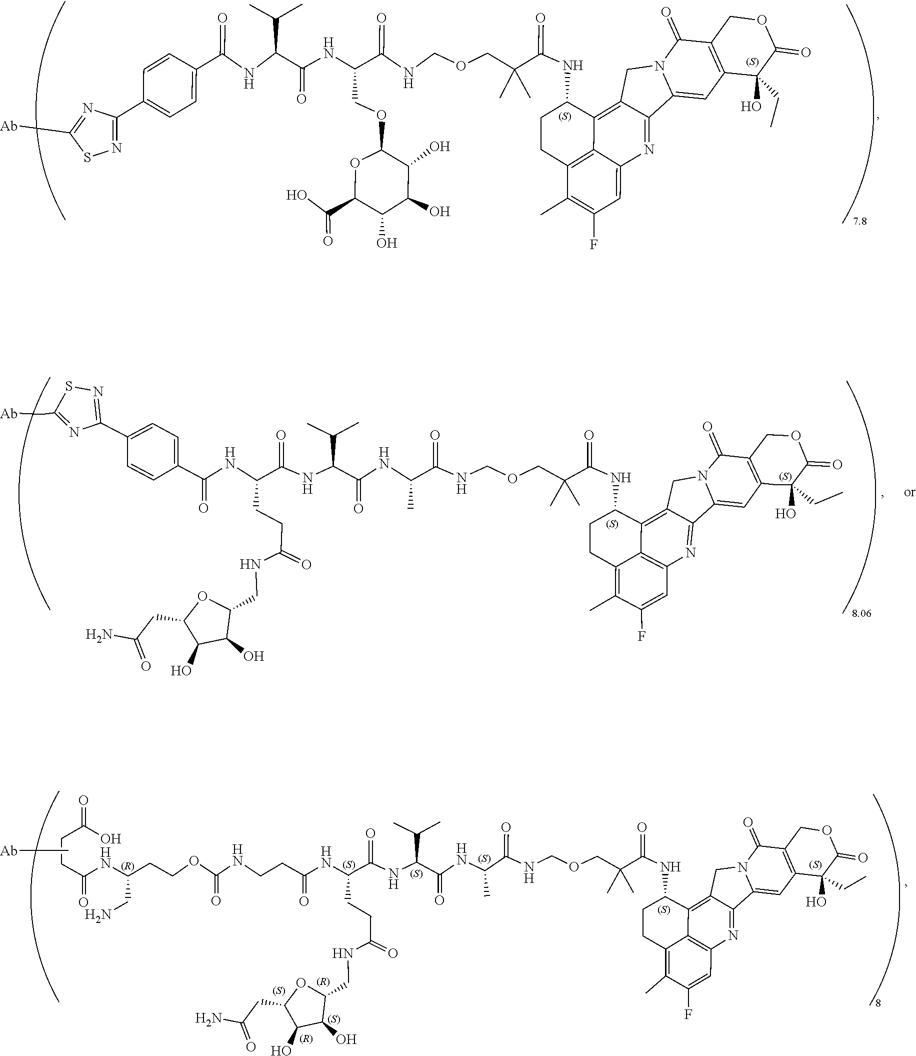
or a pharmaceutically acceptable salt, solvate, and/or stereoisomer thereof.

31. The immunoconjugate of claim 27, wherein the immunoconjugate is represented by the following formula: 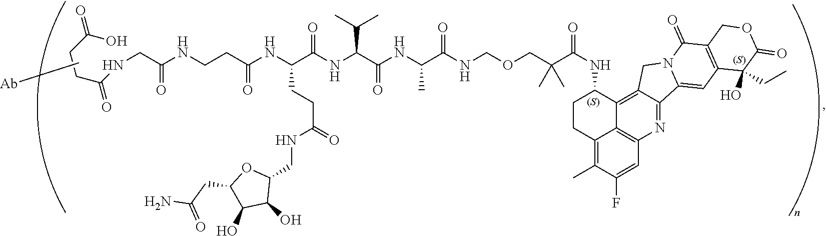
or a pharmaceutically acceptable salt, solvate, and/or stereoisomer thereof, wherein n is about 8 and Ab comprises:
a heavy chain variable region (VH) comprising the amino acid of SEQ ID NO: 9, and a light chain variable region (VL) comprising the amino acid sequence of SEQ ID NO: 10.

32. The immunoconjugate of claim 27, wherein the immunoconjugate is represented by the following formula: 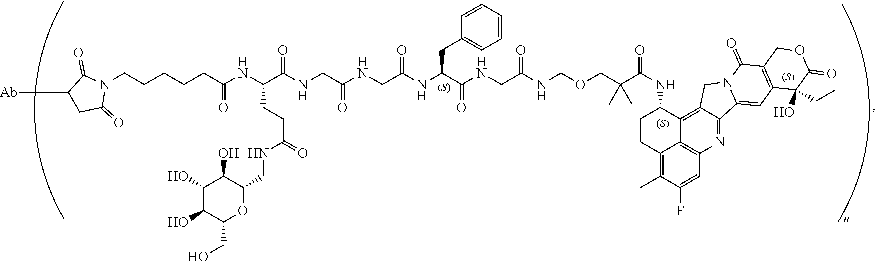
or a pharmaceutically acceptable salt, solvate, and/or stereoisomer thereof, wherein n is about 8 and Ab comprises:
a heavy chain variable region (VH) comprising the amino acid of SEQ ID NO: 9, and a light chain variable region (VL) comprising the amino acid sequence of SEQ ID NO: 10.

33. The immunoconjugate of claim 27, wherein the immunoconjugate is represented by the following formula: 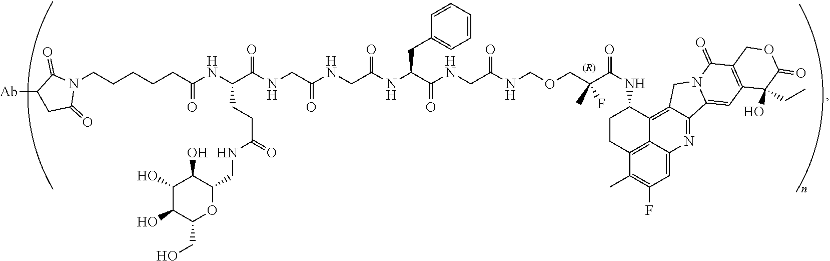
or a pharmaceutically acceptable salt, solvate, and/or stereoisomer thereof, wherein n is about 8 and Ab comprises:
a heavy chain variable region (VH) comprising the amino acid of SEQ ID NO: 9, and a light chain variable region (VL) comprising the amino acid sequence of SEQ ID NO: 10.

34. The immunoconjugate of claim 27, wherein the immunoconjugate is represented by the following formula: 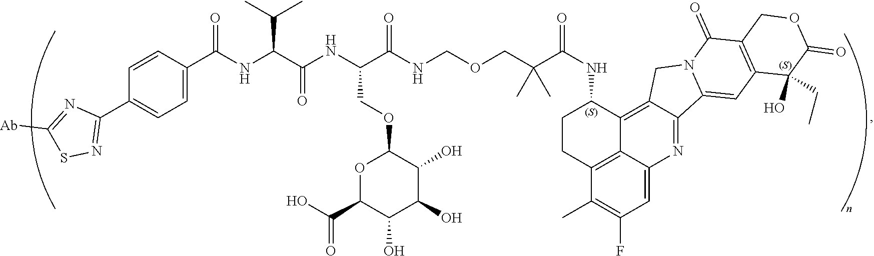
or a pharmaceutically acceptable salt, solvate, and/or stereoisomer thereof, wherein n is about 8 and Ab comprises:
a heavy chain variable region (VH) comprising the amino acid of SEQ ID NO: 9, and a light chain variable region (VL) comprising the amino acid sequence of SEQ ID NO: 10.

35. The immunoconjugate of claim 27, wherein the immunoconjugate is represented by the following formula: 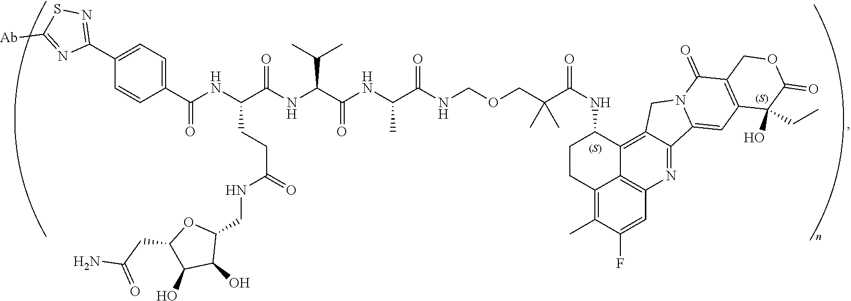
or a pharmaceutically acceptable salt, solvate, and/or stereoisomer thereof, wherein n is about 8 and Ab comprises:
a heavy chain variable region (VH) comprising the amino acid of SEQ ID NO: 9, and a light chain variable region (VL) comprising the amino acid sequence of SEQ ID NO: 10.

36. The immunoconjugate of claim 27, wherein the immunoconjugate is represented by the following formula: 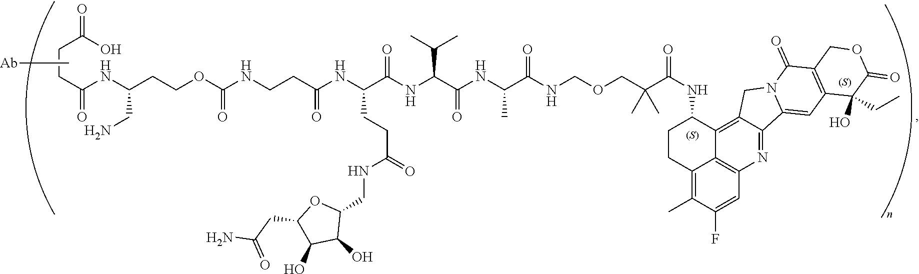
or a pharmaceutically acceptable salt, solvate, and/or stereoisomer thereof, wherein n is about 8 and Ab comprises:
a heavy chain variable region (VH) comprising the amino acid of SEQ ID NO: 9, and a light chain variable region (VL) comprising the amino acid sequence of SEQ ID NO: 10.

37. A pharmaceutical composition comprising the immunoconjugate of claim 27 and a pharmaceutically acceptable carrier.

38. (canceled)

39. (canceled)

40. A method of treating small cell lung cancer or gastric cancer comprising administering to a patient in need thereof an effective amount of the immunoconjugate of claim 27.

41. A method of treating a tubulin inhibitor-resistant tumor comprising administering an effective amount of the immunoconjugate of claim 27 to a subject having an accumulation or overexpression of FGFR2b in a biological sample.

42.-48. (canceled)

49. A method of producing the immunoconjugate of claim 27 comprising:
(i) culturing a host cell which has been transformed by an isolated nucleic acid comprising a sequence encoding an anti-FGFR2b antibody or antigen-binding fragment thereof, wherein the antibody comprises a heavy chain comprising an amino acid sequence of SEQ ID NO: 9 and a light chain comprising an amino acid of SEQ ID NO: 10, or a heavy chain comprising an amino acid sequence of SEQ ID NO: 21 and a light chain comprising an amino acid of SEQ ID NO: 22;
(ii) expressing said antibody or antigen-binding fragment thereof;
(iii) recovering the expressed antibody or antigen-binding fragment thereof; and
(iv) conjugating at least one cytotoxic agent to the antibody or antigen-binding fragment thereof such that an immunoconjugate is formed.

50. A method of treating small cell lung cancer or gastric cancer comprising administering to a patient in need thereof an effective amount of a pharmaceutical composition comprising the antibody or antigen-binding fragment thereof of claim 1.

51. A method of treating a tubulin inhibitor-resistant tumor comprising administering an effective amount of a pharmaceutical composition comprising the antibody or antigen-binding fragment thereof of claim 1 to a subject having an accumulation or overexpression of FGFR2b in a biological sample.

52. The immunoconjugate of claim 27, wherein the immunoconjugate is represented by the following formula: 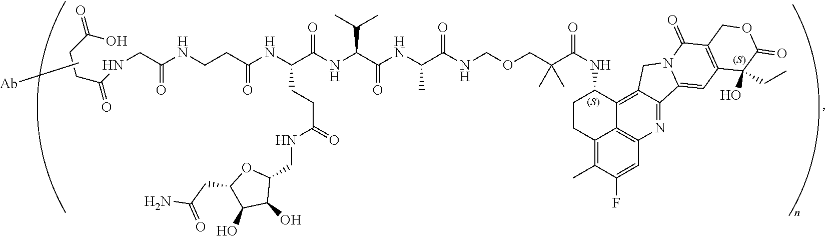
or a pharmaceutically acceptable salt, solvate, and/or stereoisomer thereof, wherein n is about 8 and Ab comprises: a heavy chain variable region (VH) comprising the amino acid sequence of SEQ ID NO: 21, and a light chain variable region (VL) comprising the amino acid sequence of SEQ ID NO: 22.

53. The immunoconjugate of claim 27, wherein the immunoconjugate is represented by the following formula: 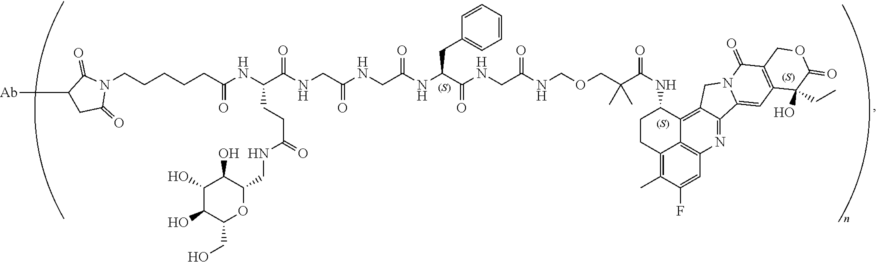
or a pharmaceutically acceptable salt, solvate, and/or stereoisomer thereof, wherein n is about 8 and Ab comprises: a heavy chain variable region (VH) comprising the amino acid sequence of SEQ ID NO: 21, and a light chain variable region (VL) comprising the amino acid sequence of SEQ ID NO: 22.

54. The immunoconjugate of claim 27, wherein the immunoconjugate is represented by the following formula: 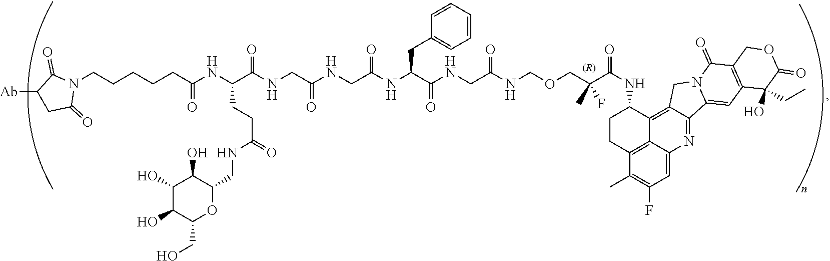
or a pharmaceutically acceptable salt, solvate, and/or stereoisomer thereof, wherein n is about 8 and Ab comprises: a heavy chain variable region (VH) comprising the amino acid sequence of SEQ ID NO: 21, and a light chain variable region (VL) comprising the amino acid sequence of SEQ ID NO: 22.

55. The immunoconjugate of claim 27, wherein the immunoconjugate is represented by the following formula: 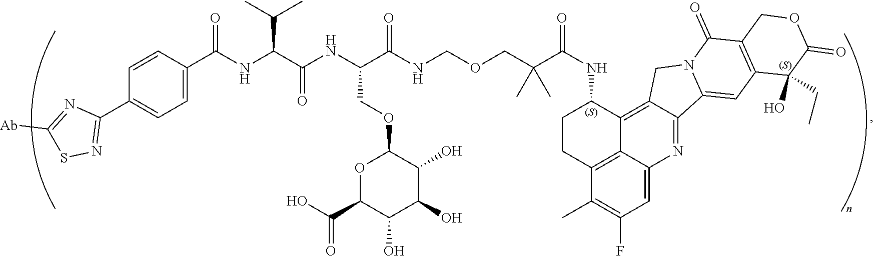
or a pharmaceutically acceptable salt, solvate, and/or stereoisomer thereof, wherein n is about 8 and Ab comprises: a heavy chain variable region (VH) comprising the amino acid sequence of SEQ ID NO: 21, and a light chain variable region (VL) comprising the amino acid sequence of SEQ ID NO: 22.

56. The immunoconjugate of claim 27, wherein the immunoconjugate is represented by the following formula: 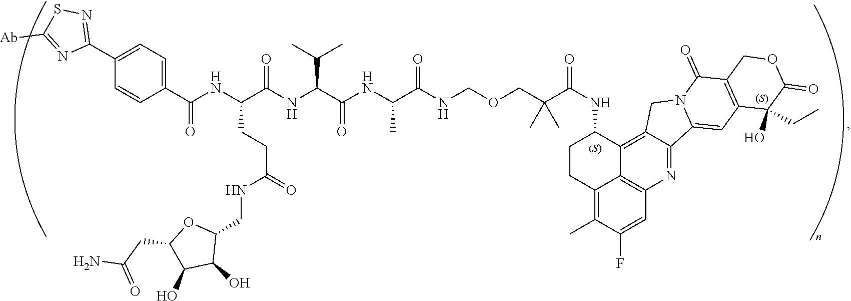
or a pharmaceutically acceptable salt, solvate, and/or stereoisomer thereof, wherein n is about 8 and Ab comprises: a heavy chain variable region (VH) comprising the amino acid sequence of SEQ ID NO: 21, and a light chain variable region (VL) comprising the amino acid sequence of SEQ ID NO: 22.

57. The immunoconjugate of claim 27, wherein the immunoconjugate is represented by the following formula: 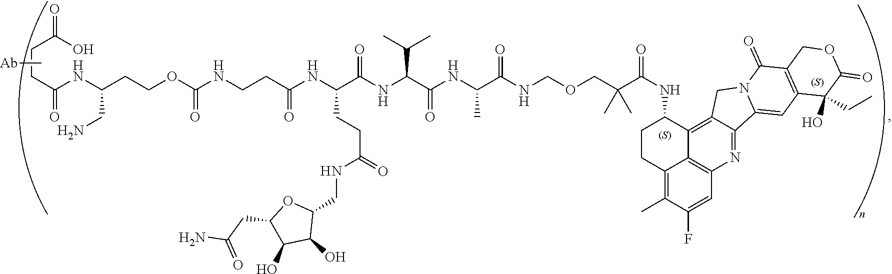
or a pharmaceutically acceptable salt, solvate, and/or stereoisomer thereof, wherein n is about 8 and Ab comprises: a heavy chain variable region (VH) comprising the amino acid sequence of SEQ ID NO: 21, and a light chain variable region (VL) comprising the amino acid sequence of SEQ ID NO: 22.


SEQUENCE LISTING (80 sequences)

SEQ ID NO: 1 - protein, 821 aa, Homo sapiens
  REGION 1..821: note = misc_feature - amino acid sequence of human FGFR2b
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 2 - protein, 194 aa, Homo sapiens
  REGION 1..194: note = misc_feature - ligand for FGFR2b is known as keratinocyte growth factor (KGF)
MHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 3 - protein, 5 aa, synthetic construct
  REGION 1..5: note = Synthetic: HCDR1 (Kabat)
IYWLN

SEQ ID NO: 4 - protein, 17 aa, synthetic construct
  REGION 1..17: note = Synthetic: HCDR2 (Kabat)
QIYPENADTNYSGKFKG

SEQ ID NO: 5 - protein, 5 aa, synthetic construct
  REGION 1..5: note = Synthetic: HCDR3 (Kabat)
GGFDY

SEQ ID NO: 6 - protein, 11 aa, synthetic construct
  REGION 1..11: note = Synthetic: LCDR1 (Kabat)
RASENIYSNLA

SEQ ID NO: 7 - protein, 7 aa, synthetic construct
  REGION 1..7: note = Synthetic: LCDR2 (Kabat)
TATNLAE

SEQ ID NO: 8 - protein, 9 aa, synthetic construct
  REGION 1..9: note = Synthetic: LCDR3 (Kabat)
QHFYGILYT

SEQ ID NO: 9 - protein, 114 aa, synthetic construct
  REGION 1..114: note = Synthetic: VH AA
QVQLVQSGAEVKKPGSSVKVSCKASGYTFSIYWLNWVRQAPGQGLEWMGQIYPENADTNYSGKFKGRVTITADKSTSTAYMELSSLRSEDTAVYFCARGGFDYWGQGTLVTVSS

SEQ ID NO: 10 - protein, 107 aa, synthetic construct
  REGION 1..107: note = Synthetic: VL AA
DIQMTQSPSSLSASVGDRVTITCRASENIYSNLAWYQQKPGKAPKLLIYTATNLAEGVPSRFSGSGSGTDFTLTISSLQPEDFATYYCQHFYGILYTFGQGTRLEIK

SEQ ID NO: 11 - protein, 444 aa, synthetic construct
  REGION 1..444: note = Synthetic: HC AA
QVQLVQSGAEVKKPGSSVKVSCKASGYTFSIYWLNWVRQAPGQGLEWMGQIYPENADTNYSGKFKGRVTITADKSTSTAYMELSSLRSEDTAVYFCARGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 214 aa, synthetic construct
  REGION 1..214: note = Synthetic: LC AA
DIQMTQSPSSLSASVGDRVTITCRASENIYSNLAWYQQKPGKAPKLLIYTATNLAEGVPSRFSGSGSGTDFTLTISSLQPEDFATYYCQHFYGILYTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1332 bp, synthetic construct
  misc_feature 1..1332: note = Synthetic: HC DNA
  source 1..1332: mol_type = other DNA
caagtgcagctggtgcagagcggcgccgaggtgaagaagcccggcagcagcgtgaaggtgagctgcaaggctagcggctacaccttcagcatctactggctgaactgggtgagacaagcccccggccaaggcctggagtggatggggcagatctaccccgagaacgccgacaccaactacagcggcaagttcaagggcagagtgaccatcaccgccgacaagagcacaagcaccgcctacatggagctgagcagcctgagaagcgaggacaccgccgtgtacttctgcgctagaggcggcttcgactactggggccaaggcaccctggtgaccgtgagcagcgcttcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 14 - DNA, 642 bp, synthetic construct
  misc_feature 1..642: note = Synthetic: LC DNA
  source 1..642: mol_type = other DNA
gacattcagatgacacagagccctagcagcctgtccgctagcgtgggcgacagagtgaccatcacctgcagagctagcgagaacatctacagcaacctggcctggtatcagcagaagcccggcaaggcccccaagctgctgatctacaccgccaccaacctggccgagggcgtgcctagcagattcagcggcagcggcagcggcaccgacttcaccctgaccatcagcagcctgcagcccgaggacttcgccacctactactgtcagcacttctacggcatcctgtacaccttcggccaaggcacaagactggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 15 - protein, 5 aa, synthetic construct
  REGION 1..5: note = Synthetic: HCDR1 (Kabat)
DTYIH

SEQ ID NO: 16 - protein, 17 aa, synthetic construct
  REGION 1..17: note = Synthetic: HCDR2 (Kabat)
RIDPAEGNTMFASEFQG

SEQ ID NO: 17 - protein, 13 aa, synthetic construct
  REGION 1..13: note = Synthetic: HCDR3 (Kabat)
SKIHYDYDEGFAY

SEQ ID NO: 18 - protein, 15 aa, synthetic construct
  REGION 1..15: note = Synthetic: LCDR1 (Kabat)
RASESVDDYGYSFLH

SEQ ID NO: 19 - protein, 7 aa, synthetic construct
  REGION 1..7: note = Synthetic: LCDR2 (Kabat)
RASNLES

SEQ ID NO: 20 - protein, 9 aa, synthetic construct
  REGION 1..9: note = Synthetic: LCDR3 (Kabat)
QQSNQNPRT

SEQ ID NO: 21 - protein, 122 aa, synthetic construct
  REGION 1..122: note = Synthetic: VH AA
QVQLVQSGAEVKKPGASVKVSCKASGYTITDTYIHWVRQAPGQGLEWMGRIDPAEGNTMFASEFQGRVTMTADTSTSTVYMELSSLRSEDTAVYYCARSKIHYDYDEGFAYWGQGTLVTVSS

SEQ ID NO: 22 - protein, 111 aa, synthetic construct
  REGION 1..111: note = Synthetic: VL AA
DIVLTQSPDSLAVSLGERATINCRASESVDDYGYSFLHWYQQKPGQPPKLLIYRASNLESGVPDRFSGSGSGTDFTLTISSLQAEDVAVYYCQQSNQNPRTFGGGTKVEIK

SEQ ID NO: 23 - protein, 452 aa, synthetic construct
  REGION 1..452: note = Synthetic: HC AA
QVQLVQSGAEVKKPGASVKVSCKASGYTITDTYIHWVRQAPGQGLEWMGRIDPAEGNTMFASEFQGRVTMTADTSTSTVYMELSSLRSEDTAVYYCARSKIHYDYDEGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 218 aa, synthetic construct
  REGION 1..218: note = Synthetic: LC AA
DIVLTQSPDSLAVSLGERATINCRASESVDDYGYSFLHWYQQKPGQPPKLLIYRASNLESGVPDRFSGSGSGTDFTLTISSLQAEDVAVYYCQQSNQNPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - DNA, 1356 bp, synthetic construct
  misc_feature 1..1356: note = Synthetic: HC DNA
  source 1..1356: mol_type = other DNA
caagtgcagctggtgcagagcggcgccgaggtgaagaagcccggcgctagcgtgaaggtgagctgcaaggctagcggctacaccatcaccgacacctacatccactgggtgagacaagcccccggccaaggcctggagtggatgggcagaatcgaccccgccgagggcaacaccatgttcgctagcgagttccaaggcagagtgaccatgaccgccgacacaagcacaagcaccgtgtacatggagctgagcagcctgagaagcgaggacaccgccgtgtactactgcgctagaagcaagatccactacgactacgacgagggcttcgcctactggggccaaggcaccctggtgaccgtgagcagcgcttcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 26 - DNA, 654 bp, synthetic construct
  misc_feature 1..654: note = Synthetic: LC DNA
  source 1..654: mol_type = other DNA
gacatcgtgctgacacagagccccgacagcctggccgtgagcctgggcgagagagccaccatcaactgcagagctagcgagagcgtggacgactacggctacagcttcctgcactggtatcagcagaagcccgggcagccccccaagctgctgatctacagagctagcaacctggagagcggcgtgcccgacagattcagcggcagcggcagcggcaccgacttcaccctgaccatcagcagcctgcaagccgaggacgtggccgtgtactactgtcagcagagcaatcagaaccctagaaccttcggcgggggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 27 - DNA, 342 bp, synthetic construct
  misc_feature 1..342: note = Synthetic: VH DNA
  source 1..342: mol_type = other DNA
gaggtccagctgcagcagtctggggctgagctggtgaagcctggggcctcagtgaagatttcctgcaaagcttctggctacgcattcagtatctactggatgaactgggtgaagcagaggcctggaaagggtcttgaatggattggacagatttatcctgaaaatggtgatactaactacaacggaaagttcaagggcaaggccacactgactgcagacaaatcctccagcacagcctacatgcacctcagcagcctgacctctgaggactctgcggtctatttctgtgcaagaggggggtttgactactggggccaaggcaccactctcaccgtctcgagc

SEQ ID NO: 28 - protein, 114 aa, synthetic construct
  REGION 1..114: note = Synthetic: VH AA
EVQLQQSGAELVKPGASVKISCKASGYAFSIYWMNWVKQRPGKGLEWIGQIYPENGDTNYNGKFKGKATLTADKSSSTAYMHLSSLTSEDSAVYFCARGGFDYWGQGTTLTVSS

SEQ ID NO: 29 - DNA, 321 bp, synthetic construct
  misc_feature 1..321: note = Synthetic: VL DNA
  source 1..321: mol_type = other DNA
aatattgtgatgacccagtctccagcctccctatctgcatctgtgggagaaactgtcaccatcacatgtcgagcaagtgagaatatttacagtaatttagcatggtatcagcagaaacagggaaaatctcctcagctcctggtctatactgcaacaaatttagcagatggtgtgccatcaaggttcagtggcagtggatcaggcacacagttttctctgaagatcaacagcctgcagcctgaagattttgggagttattactgtcaacatttttatggtattttgtatacgttcggatcggggaccagactggaaataaaa

SEQ ID NO: 30 - protein, 107 aa, synthetic construct
  REGION 1..107: note = Synthetic: VL AA
NIVMTQSPASLSASVGETVTITCRASENIYSNLAWYQQKQGKSPQLLVYTATNLADGVPSRFSGSGSGTQFSLKINSLQPEDFGSYYCQHFYGILYTFGSGTRLEIK

SEQ ID NO: 31 - DNA, 366 bp, synthetic construct
  misc_feature 1..366: note = Synthetic: VH DNA
  source 1..366: mol_type = other DNA
caggtgcaactgaagcagtctggggcagagcttgtgaagccaggggcctcagtcaagttgtcctgcacagcttctggcttcaacattaaagacacctatatacactgggtgaagcagaggcctgaacagggcctggagtggattggaaggattgatcctgcgaatggtaatactatgtttgcctcagagttccagggcaaggccactataacagcatacacatcatccaacacagcctacatgcagctcagcagcctgacatctggggacactgccgtctattattgtgctagatccaagatccactatgattacgacgaggggtttgcttactggggccaagggactctggtcaccgtctcgagc

SEQ ID NO: 32 - protein, 122 aa, synthetic construct
  REGION 1..122: note = Synthetic: VH AA
QVQLKQSGAELVKPGASVKLSCTASGFNIKDTYIHWVKQRPEQGLEWIGRIDPANGNTMFASEFQGKATITAYTSSNTAYMQLSSLTSGDTAVYYCARSKIHYDYDEGFAYWGQGTLVTVSS

SEQ ID NO: 33 - DNA, 333 bp, synthetic construct
  misc_feature 1..333: note = Synthetic: VL DNA
  source 1..333: mol_type = other DNA
aacattgtgctgacccaatctccaccttctttggctgtgtctctagggcagagggccaccgtatcctgcagagccagtgaaagtgttgatgattatggctatagttttttgcactggtaccagcagaaaccaggacagccacccaaactcctcatctatcgtgcatccaacctagaatctgggatccctgccaggttcagtggcagtggctctaggacagacttcaccctcaccattaatcctgtggagactgatgacgttgcaacctactactgtcagcaaagtaatcaaaatcctcggacgttcggtggaggcacaaagttggaaatcaaa

SEQ ID NO: 34 - protein, 111 aa, synthetic construct
  REGION 1..111: note = Synthetic: VL AA
NIVLTQSPPSLAVSLGQRATVSCRASESVDDYGYSFLHWYQQKPGQPPKLLIYRASNLESGIPARFSGSGSRTDFTLTINPVETDDVATYYCQQSNQNPRTFGGGTKLEIK

SEQ ID NO: 35 - protein, 444 aa, synthetic construct
  REGION 1..444: note = Synthetic: HC AA
QVQLVQSGAEVKKPGSSVKVSCKASGYIFTTYNVHWVRQAPGQGLEWIGSIYPDNGDTSYNQNFKGRATITADKSTSTAYMELSSLRSEDTAVYYCARGDFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 214 aa, synthetic construct
  REGION 1..214: note = Synthetic: LC AA
DIQMTQSPSSLSASVGDRVTITCKASQGVSNDVAWYQQKPGKAPKLLIYSASYRYTGVPSRFSGSGSGTDFTFTISSLQPEDIATYYCQQHSTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 448 aa, synthetic construct
  REGION 1..448: note = Synthetic: HC AA
EVQLLESGGGLVQPGGSLRLSCAASGFTFRTSGMHWVRQAPGKGLEWVAVISYDGSNKYYADSVKGRFTISRDNSKNTLYLQMNSLRAEDTAVYYCARESPGYDFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 216 aa, synthetic construct
  REGION 1..216: note = Synthetic: LC AA
QSVLTQPPSASGTPGQRVTISCSGSSSNIGSNTVNWYQQLPGTAPKLLIYGNSNRPSGVPDRFSGSKSGTSASLAISGLRSEDEADYYCAAWDDSLNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 39 - protein, 452 aa, synthetic construct
  REGION 1..452: note = Synthetic: HC AA
EVQLLESGGGLVQPGGSLRLSCAASGFTFSSYAMSWVRQAPGKGLEWVSAISGSGTSTYYADSVKGRFTISRDNSKNTLYLQMNSLRAEDTAVYYCARVRYNWNHGDWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 216 aa, synthetic construct
  REGION 1..216: note = Synthetic: LC AA
QSVLTQPPSASGTPGQRVTISCSGSSSNIGNNYVSWYQQLPGTAPKLLIYENYNRPAGVPDRFSGSKSGTSASLAISGLRSEDEADYYCSSWDDSLNYWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 41 - protein, 115 aa, synthetic construct
  REGION 1..115: note = Synthetic: VH Consensus
EVQLXQSGXELVKPGASVKXSCKASGYXFTXYXXHWVKQXPGKGLEWIGXIXPDNGDTXYNXXFKGKATITXDXSSSTAYMQLSSLTSEDTAVYYCARGXXFDYWGQGTTVTVSS

SEQ ID NO: 42 - protein, 114 aa, synthetic construct [sequence omitted]

SEQ ID NO: 43 - protein, 114 aa, synthetic construct
  REGION 1..114: note = Synthetic: VH 083BH148-1
QVQMKQSGPELVKPGASVKMSCKASGYTFTNYNMHWVKQSHGKSIEWIGYIYPDNGDTNYNQKFRGKATLTLDKSSSTAYMELRSLTSDDSAVYYCTKGAMDYWGQGTSVTVSS

SEQ ID NO: 44 - protein, 122 aa, synthetic construct; identical to SEQ ID NO: 32
  REGION 1..122: note = Synthetic: VH 083BH137

SEQ ID NO: 45 - protein, 114 aa, synthetic construct
  REGION 1..114: note = Synthetic: VH 083BH155-1
EVQLQQSGPELVKPGASVKMSCKTSGYTFTNYYMHWVKQSHGKSIEWIGYIYPDNGDTTYNQKFKGKATLTVDKSSSTAYMELRSLTSEDSAVYYCTRGGFDYWGQGTTLTVSS

SEQ ID NO: 46 - protein, 119 aa, synthetic construct
  REGION 1..119: note = Synthetic: VH 081BH018
EVMLVESGPGLVKPSQSLSLTCSVTGYSITSDYAWNWIRQFPGNKLEWMGYISYSGSTSYNPSLKSRISITRDTSKNQFFLQLNSVTTEDTATYYCARSGDYYGSLDYWGQGTTLTVSS

SEQ ID NO: 47 - protein, 120 aa, synthetic construct
  REGION 1..120: note = Synthetic: VH 081BH053
EVQLVESGPGLVKPSQSLSLTCTVTGYSITSDYAWNWIRQFPGNKLEWMGYINYSGYTSYSPSLKSRISITRDTSKNRFFLQLNSVTTEDTATYFCARGWIRASYNFDFWGQGTTLTVSS

SEQ ID NO: 48 - protein, 113 aa, synthetic construct
  REGION 1..113: note = Synthetic: VH 083BH035
EVQLQQSGPELVKPGASVKMSCKASGYTFTDYYIHWVKQSHGKSIEWIGNIYPDNGDTTYNQKFKGKTTLTVDKSSTTAYMELRSLTSDDSAVYYCERGGDYWGQGTSVTVSS

SEQ ID NO: 49 - protein, 119 aa, synthetic construct
  REGION 1..119: note = Synthetic: VH 081BH005-2
EVQMKESGPGLVAPSQSLSITCTVSGFSLTVYGINWVRQPPGKGLEWLGMIWGDGTTDYNSALKSRLSISKDNSKSQVFLKMNSLQTDDTARYYCAGWLLPDYFAMDYWGQGTSVTVSS

SEQ ID NO: 50 - protein, 122 aa, synthetic construct
  REGION 1..122: note = Synthetic: VH 082BH007
EVQLLESEGGLVQPGTSMKLSCTASGFTFSDYYMAWVRQVPEKGLEWVANINYDGSSTYYLDSLKSRFIISRDNAKNILYLQMSSLRSEDTATYYCARPSTMVSTDYAMDYWGQGTSVTVSS

SEQ ID NO: 51 - protein, 114 aa, synthetic construct
  REGION 1..114: note = Synthetic: VH 082BH035
QVQLQQSGAELVKPGASVKISCKASGYAFSNYWMYWVKQRPGKGLEWIGQNYPENGDTDYNGKFRGKATLTADKSSSTAYMQLSSLTSEDSEVYFCTRGGFDYWGQGTTLTVSS

SEQ ID NO: 52 - protein, 114 aa, synthetic construct; identical to SEQ ID NO: 28
  REGION 1..114: note = Synthetic: VH 082BH018

SEQ ID NO: 53 - protein, 114 aa, synthetic construct
  REGION 1..114: note = Synthetic: VH 081BH055
EVQLQQPGAELVKPGASVRMSCKTSGYTFNTYNIHWIKQTPGQGLEWIGAIYPDNGDIFYNQKFKGKATLTADKSSSTADMQLSSLTSEDSAVYYCARGAIDYWGQGTTLTVSS

SEQ ID NO: 54 - protein, 121 aa, synthetic construct
  REGION 1..121: note = Synthetic: VH 081BH048
EVMLVESGPGILQPSQTLSLTCSFSGFSLITSGMGVSWIRQPSGKGLEWLAHIFWDDDKRYNPSLKSRLTISKDTSSNQVFLKITSVDTSDTATYYCTRSGGITAGVLDFWGQGTSVTVSS

SEQ ID NO: 55 - protein, 118 aa, synthetic construct
  REGION 1..118: note = Synthetic: VH M047
EVQLLESGGGLVQPGGSLRLSCAASGFTFRTSGMHWVRQAPGKGLEWVAVISYDGSNKYYADSVKGRFTISRDNSKNTLYLQMNSLRAEDTAVYYCARESPGYDFEYWGQGTLVTVSS

SEQ ID NO: 56 - protein, 113 aa, synthetic construct
  REGION 1..113: note = Synthetic: VH 21C-mFc
EVQLHQSGTVLARPGASVKMSCKTSGYTFTNYWMHWIKQRPGQGLEWIGAIYPENGDINYNQKFKGKAKLTAVTSASTAYMDLSSLTNEDSAVYYCTSRGDYWGQGTTLTVSS

SEQ ID NO: 57 - protein, 120 aa, synthetic construct
  REGION 1..120: note = Synthetic: VH 26c
DVHLQESGPGLVKPSQSLSLTCSVTGYSITSGYYWNWIRQFPGNKLEWMGYITYDGSNNYNPSLKNRLSITRDTSKNQFFLQLSSLTTEDTATYFCARVYYYGSGNFDVWGTGTTVTVSS

SEQ ID NO: 58 - protein, 114 aa, synthetic construct
  REGION 1..114: note = Synthetic: VH 36c
QPYLQQSGAELVRPGASVKMSCKASGYTFTSYNMHWVKQTPRQGLEWIGAIFPENGDTSYNQKFKGKATLTVDKSSSTAYMQLSSLTSEDSAVYFCARGGFDYWGQGTTLTVSS

SEQ ID NO: 59 - protein, 107 aa, synthetic construct
  REGION 1..107: note = Synthetic: VK Consensus
DIVMTQSPXXLSXSXGXRVTITCXASXXXXXXXAWYQQKPGXSPKLLIYXASXLXXGVPXRFSGSGSGTDFSLTISSXQXEDXAXYYCQQXXXXPXTFGXGTKLEIK

SEQ ID NO: 60 - protein, 107 aa, synthetic construct [sequence omitted]

SEQ ID NO: 61 - protein, 107 aa, synthetic construct
  REGION 1..107: note = Synthetic: VK 083BK148
DIVMTQSHKFMSTSVGDRVNITCKASQDVNTDVAWYQQKPGQSPKLLIYSASYRYTGVPDRFTGSGSGTDFTFTISSVQAEDLAVYYCQQHYSTPYTFGGGTRLEIK

SEQ ID NO: 62 - protein, 111 aa, synthetic construct; identical to SEQ ID NO: 34
  REGION 1..111: note = Synthetic: VK 083BK137-2

SEQ ID NO: 63 - protein, 107 aa, synthetic construct
  REGION 1..107: note = Synthetic: VK 083BK155-2
DILLTQSPTSLSASVGETVTITCGASENIYGALHWYQRKQGKSPQLLIFGATNLADGMSSRFSGSGSGRQYSLKISSLHPDDVATYYCQNVLSTPFTFGSGTKLEIK

SEQ ID NO: 64 - protein, 107 aa, synthetic construct
  REGION 1..107: note = Synthetic: VK 081BK018
NIVMTQTPKFLLVSAGDRVTITCKASQSVSNDVAWYQQKPGQSPKLLIYYASDRYTGVPDRFTGSGYGTDFTFTISTVQAEDLAVYFCQQDYSSPWTFGGGTKLEIK

SEQ ID NO: 65 - protein, 107 aa, synthetic construct
  REGION 1..107: note = Synthetic: VK 081BK053
DVLVTQSPAILSVSPGERVSFSCRASQSIGTSLHWYQQRRNGSPRLLIKYASESISGIPSRFSGSGSGTDFTLSINSVESEDIADYYCQQSNSWPTSFGSGTKLEMK

SEQ ID NO: 66 - protein, 107 aa, synthetic construct
  REGION 1..107: note = Synthetic: VK 083BK035
DIVMTQSPASLSTSVGETVTITCRASENIYSYLAWYQQKQGKSPQLLVYNAKTLAEGVPSRFSGSGSGTQFSLKINSLQPEDFGNYYCQHHYGTPYTLGGGTKLEIK

SEQ ID NO: 67 - protein, 107 aa, synthetic construct
  REGION 1..107: note = Synthetic: VK 081BK005
DVQMTQTTSSLSVSLGDRVTISCRASHDITNYLTWYQQKPDGTIKLLISYTSKLHSGVPSRFSGSGSGTDYSLTISNLEQEDIATYFCQQGKTLPFTFGGGTRLEIK

SEQ ID NO: 68 - protein, 108 aa, synthetic construct
  REGION 1..108: note = Synthetic: VK 082BK007
QIVLTQSPAIMSASPGEKVTMTCSASSGVNSGFLHWYQLKSGASPKLWIYGTSNLASGVPVRFSGSGSGTSYSLTISSVEAEDAATYYCQQYQSDPLTFGAGTKLEIK

SEQ ID NO: 69 - protein, 107 aa, synthetic construct
  REGION 1..107: note = Synthetic: VK 082BK035
DILLTQSPASLSASVGESVTITCRASENINSNFAWYQQKQGKSPQLLVYLATNLADGVPSRFSGSGSGTQFSLRINSLQREDFGIYYCQHFYDIPYTFGSGTKLEIK

SEQ ID NO: 70 - protein, 107 aa, synthetic construct; identical to SEQ ID NO: 30
  REGION 1..107: note = Synthetic: VK 082BK018

SEQ ID NO: 71 - protein, 107 aa, synthetic construct
  REGION 1..107: note = Synthetic: VK 081BK055
DIVMTQSQRFMSKSAGDRVSVTCKASQNVGNNVAWYQQRPGQSPKALIYSASYRYSGVPDRFTGSGSGTDFTLTISNVQSEDLAEYFCQQYYSYPLTFGTGTKLELK

SEQ ID NO: 72 - protein, 107 aa, synthetic construct
  REGION 1..107: note = Synthetic: VK 081BK048
DIQMTQSPASLSASVGETVTITCRANENIYSYLAWYQQRQGKSPQFLVYDAKTLAEGVPSRFSGSGSGTQFSLKIKSLQPEDFGSYYCQHHYGTPYTFGGGTRLEIK

SEQ ID NO: 73 - protein, 98 aa, synthetic construct
  REGION 1..98: note = Synthetic: VK M047
QSVLTQPPSASGTPGQRVTISCSGSSSNIGSNTVNWYQQLPGTAPKLLIYGNSNRPSGVPDRFSGSKSGTSASLAISGLRSEDEADYYCAAWDDSLNG

SEQ ID NO: 74 - protein, 106 aa, synthetic construct
  REGION 1..106: note = Synthetic: VK 21C-mFc
QIVLTQSPAIMSASPGEKVTMTCSASSGLSYMSWYQQKPGSSPRLLIYDTSNLASGVPVRFSGSGSGTSYSLTISRMEAEDAATYYCQQWSSYPYTFGGGTKLEIK

SEQ ID NO: 75 - protein, 107 aa, synthetic construct
  REGION 1..107: note = Synthetic: VK 26c
SIVMTQTPKILLVSAGDRVTITCKASQSVSNDVAWYQQKPGQSPKLLIYYASNRYTGVPDRFTGSGYGTDFTFTISTVQAEDLAVYFCHQDHTSPFTFGSGTKLEIK

SEQ ID NO: 76 - protein, 107 aa, synthetic construct
  REGION 1..107: note = Synthetic: VK 36c
DIVMTQSHKFMSTSVGDRVSITCKASQDVSTNVAWYQQKPGQSPKLLIFSASYRYTGVPDRFAGSGSGTDFTFTISSVQAEDLAVYYCQQHYSSPYTFGGGTKLDIK

SEQ ID NO: 77 - protein, 5 aa, synthetic construct
  REGION 1..5: note = Synthetic: HCDR1
  VARIANT 4: note = X is M or L
IYWXN

SEQ ID NO: 78 - protein, 16 aa, synthetic construct
  REGION 1..16: note = Synthetic: HCDR2
  VARIANT 6: note = X is G or A
  VARIANT 11: note = X is S or N
IYPENXDTNYXGKFKG

SEQ ID NO: 79 - protein, 7 aa, synthetic construct
  REGION 1..7: note = Synthetic: LCDR2
  VARIANT 7: note = X is D or E
TATNLAX

SEQ ID NO: 80 - protein, 17 aa, synthetic construct
  REGION 1..17: note = Synthetic: HCDR2
  VARIANT 6: note = X is E or N
RIDPAXGNTMFASEFQG